(19) United States
(12) Patent Application Publication
(10) Pub. No.: US 2025/0101008 A1
(43) Pub. Date: Mar. 27, 2025

(54) HETEROBIFUNCTIONAL MOLECULES FOR BINDING CCR2 AND METHODS OF TREATING MEDICAL CONDITIONS USING SAME

(71) Applicant: Solu Therapeutics, Inc., Medford, MA (US)

(72) Inventor: Brandon James Turunen, Boston, MA (US)

(21) Appl. No.: 18/813,364

(22) Filed: Aug. 23, 2024

Related U.S. Application Data
(60) Provisional application No. 63/578,530, filed on Aug. 24, 2023.

(51) Int. Cl. C07D 405/14; A61K 31/444; A61K 31/496; A61K 31/506; A61K 39/395; C07D 471/04; C07D 487/08

(52) U.S. Cl. CPC C07D 405/14; A61K 31/444; A61K 31/496; A61K 31/506; A61K 39/3955; C07D 471/04; C07D 487/08

(57) ABSTRACT

The invention provides heterobifunctional cotinine-containing compounds, pharmaceutical compositions, and methods of using same to treat medical conditions, such as cancer.

### CROSS-REFERENCE TO RELATED APPLICATIONS

This application claims the benefit of and priority to U.S. Provisional Patent Application Ser. No. 63/578,530, filed Aug. 24, 2023, the contents of which are hereby incorporated by reference in their entirety.

### REFERENCE TO AN ELECTRONIC SEQUENCE LISTING

This application contains a Sequence Listing which has been submitted electronically via Patent Center in XML format and is hereby incorporated by reference in its entirety. Said XML copy, created on Aug. 22, 2024, is named 211954_seglist.xml and is 15,199 bytes in size.

### FIELD OF THE INVENTION

The invention provides heterobifunctional cotinine-containing compounds, pharmaceutical compositions, and methods of using same to treat medical conditions, such as cancer.

### BACKGROUND

Cancer continues to be a significant health problem despite the substantial research efforts and scientific advances reported in the literature for treating this disease. Solid tumors, such as prostate cancer, colon, rectum, skin cancer, breast cancer, and lung cancer remain highly prevalent among the world population. Existing therapies for treating cancer include localized therapies, such as surgery, radiation therapy, cryotherapy, and systemic therapies (e.g., chemotherapy, hormonal therapy, immune therapy, and targeted therapy) used alone or in combination. Support therapies are also used in some contexts, where supportive therapies are additional treatments that do not directly treat cancer but are used to reduce side effects and address patient quality of life. However, current treatment options for cancer are not effective for all patients and/or can have substantial adverse side effects. New therapies are needed to address this unmet need in cancer therapy.

The use of cytotoxicity targeting chimeras (CyTaCs) that are able to simultaneously bind a target cell-surface protein as well as an exogenous antibody protein are described in, for example, international patent application publication nos. WO 2018/134731, WO 2023/017484, and WO 2023/017483. CyTaCs are used with an anti-cotinine antibody to treat a variety of medical diseases and conditions, such as cancer. Additional heterobifunctional cotinine-containing compounds that bind C—C motif chemokine receptor 2 (CCR2) and can function as a CyTaC are needed and would provide benefits to patients suffering from diseases and conditions, such as cancer.

The present invention addresses the foregoing needs and provides other related advantages.

### SUMMARY

The invention provides heterobifunctional cotinine-containing compounds, pharmaceutical compositions, and methods of using same to treat medical conditions, such as cancer. In particular, one aspect of the invention provides a collection of heterobifunctional cotinine-containing compounds represented by Formula I.

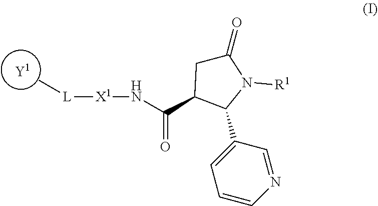

or a pharmaceutically acceptable salt thereof, where the variables are as defined in the detailed description. Further description of additional collections of heterobifunctional cotinine-containing compounds are described in the detailed description. The compounds may be part of a pharmaceutical composition comprising a pharmaceutically acceptable carrier.

The heterobifunctional compounds bind to C—C motif chemokine receptor 2 (CCR2). The heterobifunctional compounds also bind to an anti-cotinine antibody or fragment thereof that binds cotinine. In this way, the heterobifunctional compounds may be characterized as an antibody recruiting molecule (ARM).

Another aspect of the invention provides a method of treating or preventing a disease or disorder in a patient in need thereof, wherein the method comprises administering to the patient a therapeutically effective amount of a heterobifunctional compound described herein (such as a compound of Formula I) and an anti-cotinine antibody, or antigen-binding fragment thereof, wherein the disease or disorder is selected from a cancer, an inflammatory disease, an autoimmune disease, a viral infection, or a bacterial infection.

Another aspect of the invention provides a method of increasing antibody-dependent cell cytotoxicity (ADCC) of C—C motif chemokine receptor 2 (CCR2)-expressing cells, wherein the method comprises contacting the cells with an effective amount of a heterobifunctional compound described herein (such as a compound of Formula I) and an anti-cotinine antibody, or antigen-binding fragment thereof, wherein the CCR2-binding moiety of the compound binds the CCR2 expressed on the cells.

Another aspect of the invention provides a method of depleting C—C motif chemokine receptor 2 (CCR2)-expressing cells, wherein the method comprises contacting the cells with an effective amount of a heterobifunctional compound described herein (such as a compound of Formula I) and an anti-cotinine antibody, or antigen-binding fragment thereof, wherein the CCR2-binding moiety of the compound binds the CCR2 expressed on the cells.

### DETAILED DESCRIPTION

The invention provides heterobifunctional cotinine-containing compounds, pharmaceutical compositions, and methods of using same to treat medical conditions, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

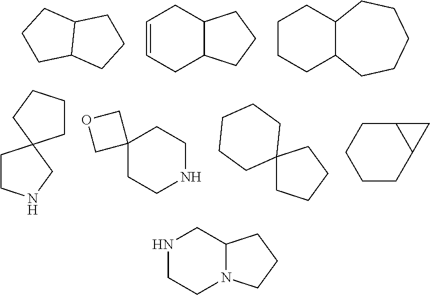

Exemplary bridged bicyclics include:

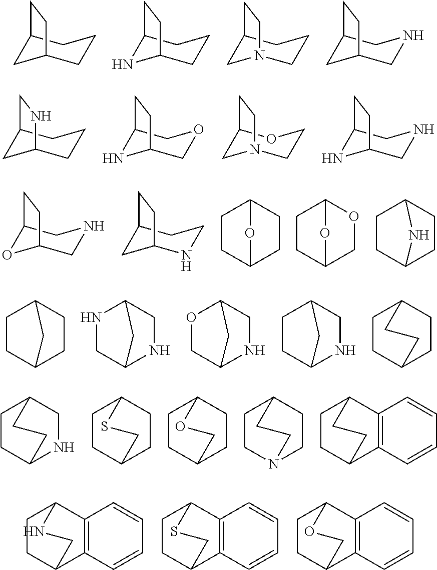

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ⁿ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

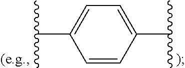 [SMILES: *c1ccc(*)cc1]

“phenylene” is a trivalent phenyl group when it has three groups attached to it

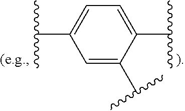 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by one or more oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it. The term “oxo-heterocyclylene” refers to a multivalent oxo-heterocyclyl group having the appropriate number of open valences to account for groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N=C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂PH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “=O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterobifunctional Cotinine-Containing Compounds

One aspect of the invention provides heterobifunctional cotinine-containing compounds. The compounds may be used in the pharmaceutical compositions and therapeutic methods described herein. Exemplary compounds are described in the following sections, along with exemplary procedures for making the compounds.

One aspect of the invention provides a compound represented by Formula I:

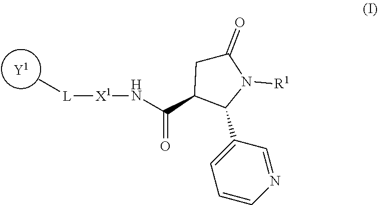

or a pharmaceutically acceptable salt thereof, wherein:
X¹ is C₁₋₅ alkylene or a covalent bond;
Y¹ is one of the following:

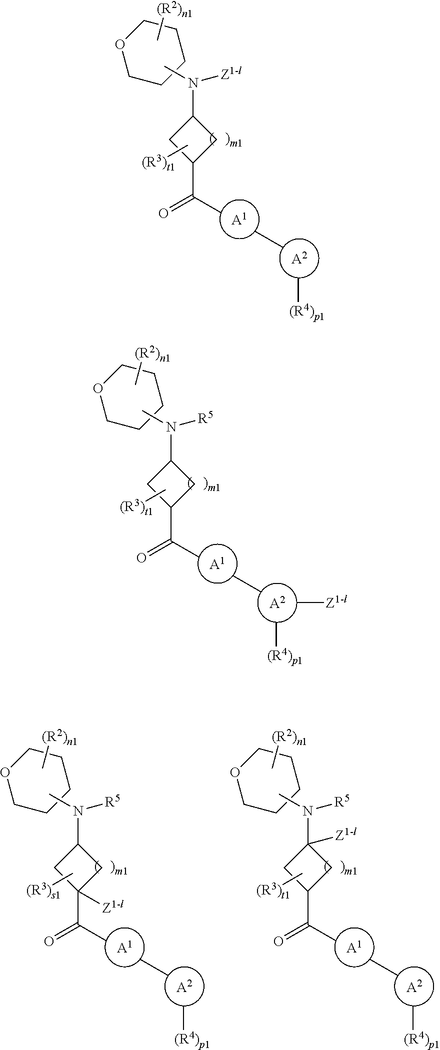

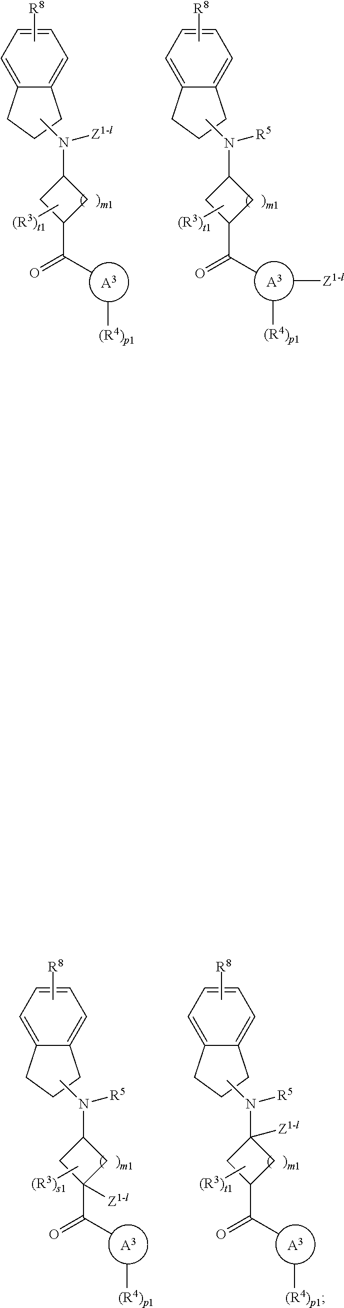

Z^{1-I} is —(C₀₋₆ alkylene)-N(R⁶)—R⁷, —(C₁₋₆ alkylene)-(4-6 membered saturated heterocyclylene containing 1 or 2 heteroatoms selected from nitrogen)-R⁷, or —(C₀₋₆ alkylene)-O—R⁷;
R¹ is C₁₋₄ alkyl or C₃₋₆ cycloalkyl;
R² represents independently for each occurrence C₁₋₄ alkoxyl or C₁₋₄ alkyl;
R³ and R⁹ each represent independently for each occurrence C₁₋₄ alkyl;
R⁵ and R⁶ are independently hydrogen or C₁₋₄ alkyl;
R⁴ and R⁸ each represent independently for each occurrence C₁₋₄ haloalkyl, halo, or C₁₋₄ alkyl;
R⁷ is a bond to L;
A¹ is (i) a 5-7 membered saturated heterocyclylene containing 1 or 2 heteroatoms independently selected from nitrogen and oxygen or (ii) a 6-8 membered bridged bicyclic saturated heterocyclylene containing 1 or 2 heteroatoms independently selected from nitrogen and oxygen; wherein the heterocyclylene is substituted with w1 occurrences of R⁹;
A² is a 5-6 membered heteroarylene containing 1 or 2 heteroatoms independently selected from nitrogen, oxygen, or sulfur;
A³ is an 8-10 membered bicyclic heteroarylene containing 1, 2, or 3 heteroatoms selected from nitrogen;
n1, p1, and t1 are independently 1, 2, or 3;
m1 is 1 or 2;
w1 and s1 are independently 0, 1, or 2; and
L is a divalent linker selected from:
(i) a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, optionally substituted 3-10 membered heterocyclyl containing 1, 2, 3, or 4 heteroatoms independently selected from nitrogen, oxygen, and sulfur, or optionally substituted 5-6 membered heteroaryl containing 1, 2, or 3 heteroatoms independently selected from nitrogen, oxygen, and sulfur;
(ii)

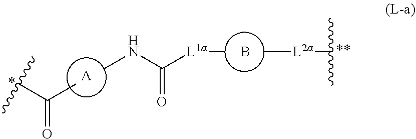

wherein Ring A and Ring B are each independently C₄₋₆ cycloalkylene; L¹a is C₃₋₅ linear alkylene, wherein 1 or 2 methylene units are replaced with —O— or —NR^{a}—; each R^{a} is independently hydrogen or C₁₋₃ alkyl; and L^{2a} is —O—, —NHC(O)—, or —CH₂—O—;
(iii)

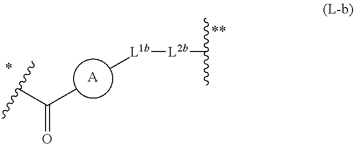

wherein Ring A is C₄₋₆ cycloalkylene or C₇₋₉ bridged bicyclic cycloalkylene; L^{1b} is —CH₂—NH—C(O)—, —NHC(O)—, or —C(O)NH—; L^{2b} is C₆-12 linear alkylene, wherein 1, 2, 3, or 4 methylene units are replaced with —O—, —NR^{1b}—, —C(O)NR^{1b}—, or —NR^{1b}C(O)—; or L^{2b} is

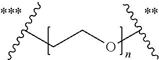

wherein n is 1, 2, 3, or 4, and

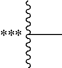

represents a covalent bond to L^{1b}; and each R^{1b} is independently hydrogen or C₁₋₃ alkyl;
(iv)

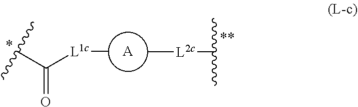

wherein L^{1c} is C₂₋₁₀ linear alkylene, wherein 1, 2, or 3 methylene units are replaced with —O—, —NH—, —NHC(O)—, or —C(O)NH—; Ring A is C₄₋₆ cycloalkylene or C₇₋₉ bridged bicyclic cycloalkylene; and L^{2c} is —O— or a saturated C₂₋₁₀ linear alkylene, wherein 1, 2, or 3 methylene units are replaced with —O—, —NH—, —NHC(O)—, or —C(O)NH—;
(v)

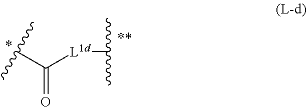

wherein L^{1d} is C₁₂₋₂₂ linear alkylene, wherein 1, 2, 3, 4, or 5 methylene units are replaced with —NH—, —O—, —C(O)NH—, —NHC(O)—, or —NHC(O)—NH—;
(vi)

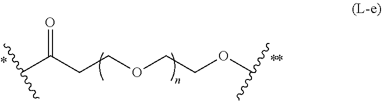

wherein n is an integer of 3 to 50;
(vii)

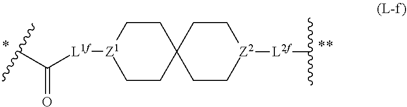

wherein L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—; L^{2f} is a bond, —NHC(O)—, —C(O)NH—, or a C₁₋₆ linear alkylene, wherein 0, 1, or 2 methylene units are replaced with —O—; and each of Z¹ and Z² is independently N or CH;
(viii)

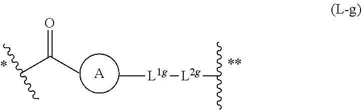

wherein Ring A is a 5 to 6 membered heteroarylene having 1 or 2 nitrogen ring atoms; L^{1g} is a bond, —CH₂—, —NH—, or —O—; and L^{2g} is

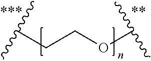

wherein n is 1, 2, 3, 4, or 5, and

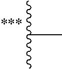

represents a covalent bond to L^{1g};
(ix)

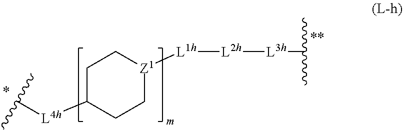

wherein each Z¹ is independently N or CH; L^{1h} is a bond, —C(O)—, —C(O)—NH—, or —NHC(O)—; L^{2h} is C₂₋₁₀ linear alkylene or

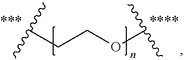

wherein n is 1, 2, 3, or 4, and

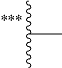

represents a covalent bond to L^{1h} and

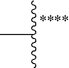

represents a covalent bond to L^{3h}; L^{3h} is a bond, —C(O)CH₂—, —O—(C₃₋₆ cycloalkylene)-O—, or —C(O)NH(CH₂)₃OCH₂—; L^{4h} is a bond, —C(O)—, —CH₂C(O)—, or —C(O)CH₂—; and m is 1, 2, or 3;
(x)

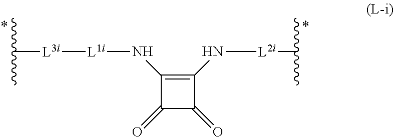

wherein L¹ⁱ is a bond, C₁₋₁₂ linear alkylene, or

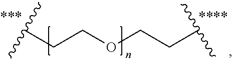

wherein n is 1, 2, 3, 4, or 5, and

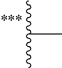

represents a covalent bond to L³ⁱ and

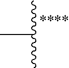

represents a covalent bond to NH; L²ⁱ is a bond, C₁₋₁₂ linear alkylene, or

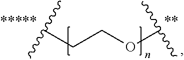

wherein n is 1, 2, 3, 4, or 5, and

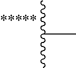

represents a covalent bond to HN; and L³ⁱ is a bond or —C(O)—;
(xi)

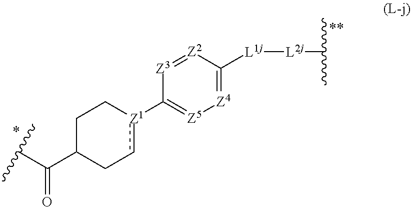

wherein Z¹ is C, CH, or N; each of Z², Z³, Z⁴ and Z⁵ is independently CH or N, provided that no more than two of Z², Z³, Z⁴ and Z⁵ are N; L^{1j} is —NH—, —C(O)NH—, —NHC(O)—, or —O—; L^{2j} is C₁₋₆ linear alkylene or

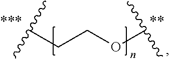

wherein n is 1 or 2, and

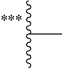

represents a covalent bond to L^{1j}; and [symbol] represents a single bond or a double bond;
(xii)

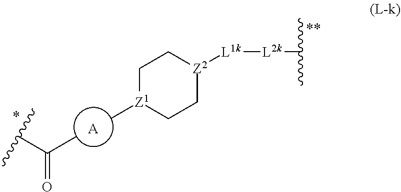

wherein Ring A is phenyl or a 5 or 6 membered heteroarylene having 1 or 2 nitrogen ring atoms; each of Z¹ and Z² is independently CH or N; L^{1k} is a bond, —C(O)—, —C(O)NH— or —NHC(O)—; and L^{2k} is a C₃₋₈ straight chain alkylene or

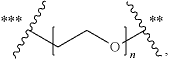

wherein n is 1, 2, or 3, and

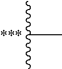

represents a covalent bond to L^{1k};
(xiii)

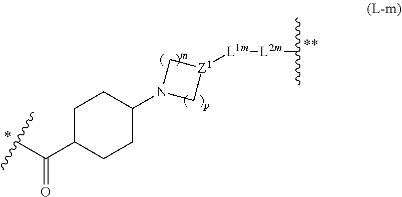

wherein Z¹ is CH or N; m is 1 or 2; p is 1 or 2; 0, 1, or 2 hydrogen atoms of

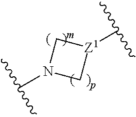

are replaced with F; L^{1m} is a bond, —C(O)—, —C(O)NH—, —NHC(O)—, —S(O)₂NH—, or —NHS(O)₂—; and L^{2m} is C₃₋₆ linear alkylene, C₃₋₆ cycloalkylene, or

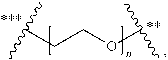

wherein n is 1 or 2, and

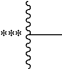

represents a covalent bond to L^{1m};
(xiv) one of

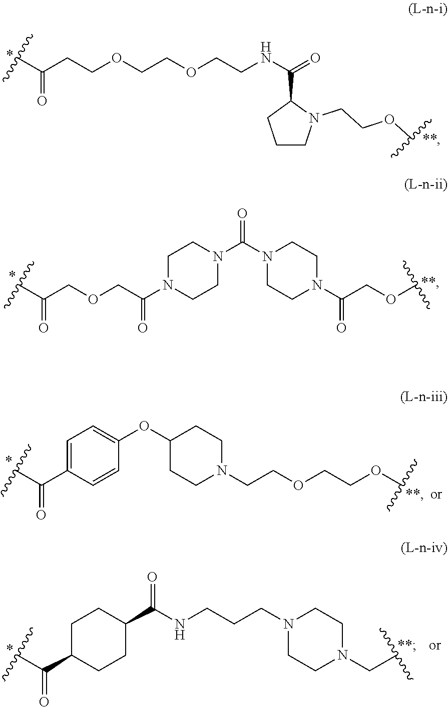

(xv)

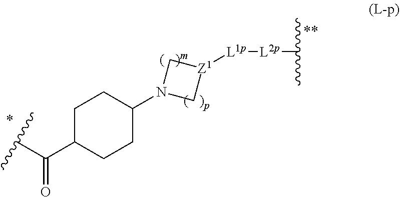

wherein Z¹ is CH or N; m is 1 or 2; p is 1 or 2; 0, 1, or 2 hydrogen atoms of

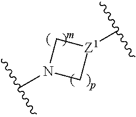

are replaced with F; L^{1p} is a bond, —C(O)—, —C(O)NH—, —NHC(O)—, —S(O)₂—, —S(O)₂NH—, or —NHS(O)₂—; and L^{2p} is -(4-6 membered saturated heterocyclylene containing 1 or 2 heteroatoms independently selected from nitrogen, oxygen, and sulfur)-C(O))—;
wherein each

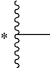

represents a covalent bond to Y¹, and each

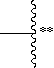

represents a covalent bond to X¹.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

In certain embodiments, X¹ is C₁₋₅ alkylene. In certain embodiments, X¹ is C₂₋₄ alkylene. In certain embodiments, X¹ is C₂₋₃ alkylene. In certain embodiments, X¹ is —CH₂CH₂—. In certain embodiments, X¹ is —C(H)(CH₃)—. In certain embodiments, X¹ is a covalent bond. In certain embodiments, X¹ is selected from the groups depicted in the compounds in Table 1, below. In certain embodiments, X¹ is selected from the groups depicted in the compounds in Table 2, below.

In certain embodiments, Y¹ is

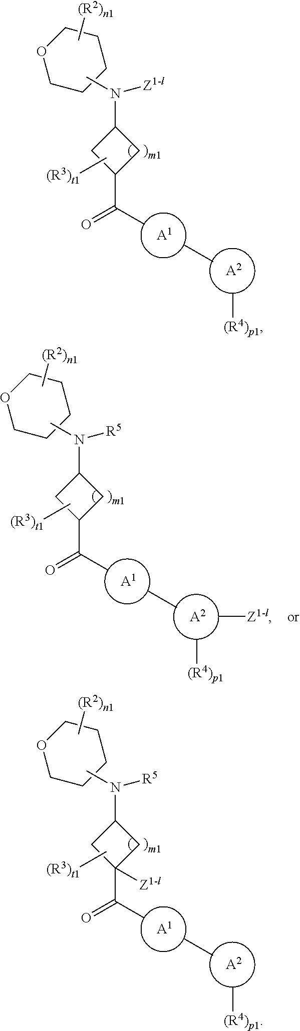

In certain embodiments, Y¹ is

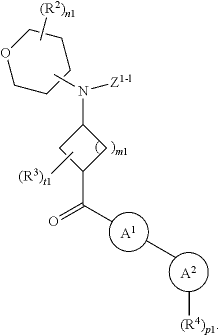

In certain embodiments Y¹ is

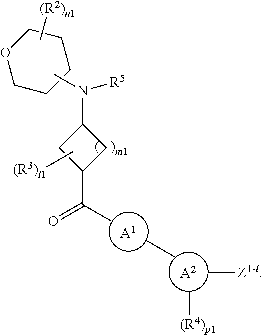

In certain embodiments, Y¹ is

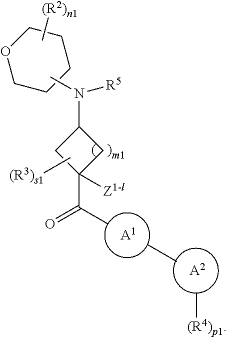

In certain embodiments Y¹ is

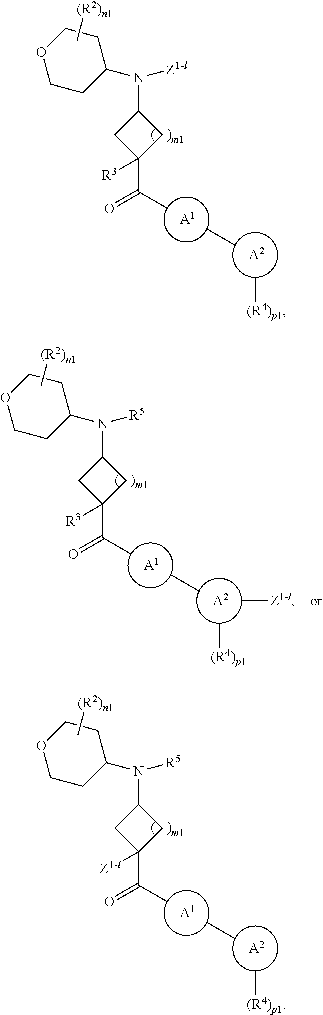

In certain embodiments, Y¹ is represented by

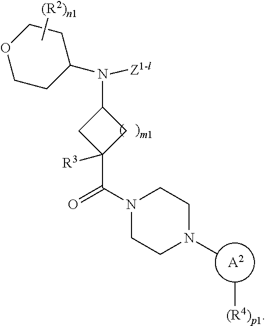

In certain embodiments, Y¹ is represented by

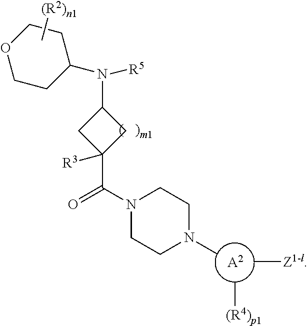

In certain embodiments, Y¹ is represented by

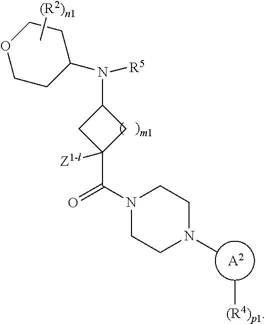

In certain embodiments, Y¹ is

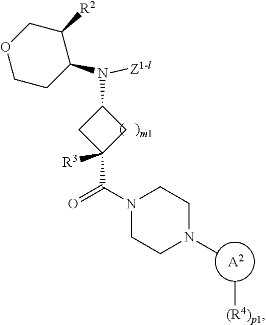

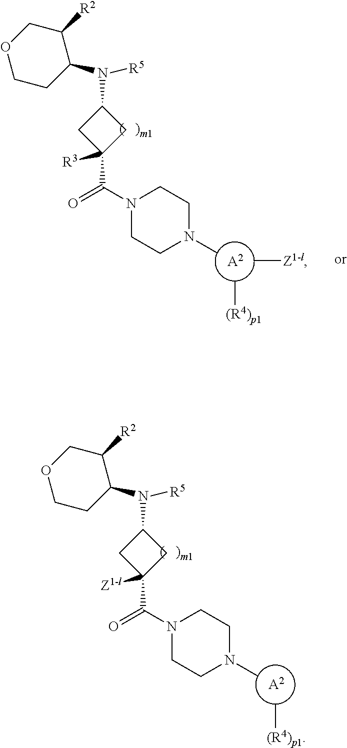

In certain embodiments, Y¹ is represented by

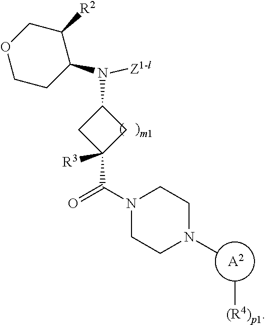

In certain embodiments, Y¹ is represented by

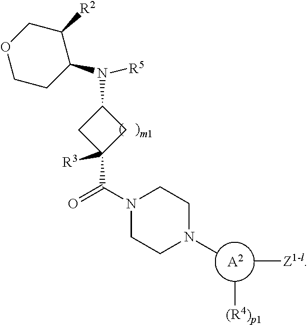

In certain embodiments, Y¹ is represented by

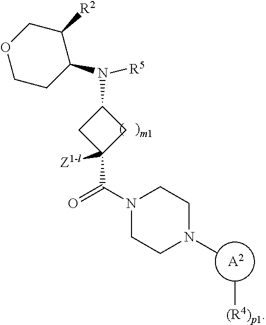

In certain embodiments, Y¹ is

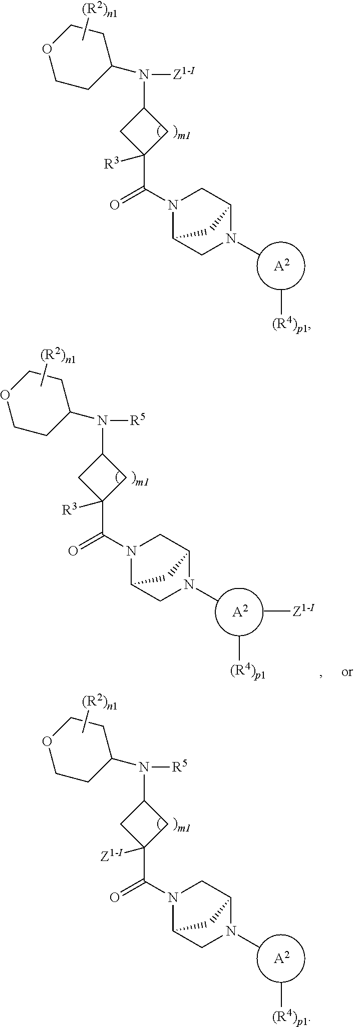

In certain embodiments, Y¹ is represented by

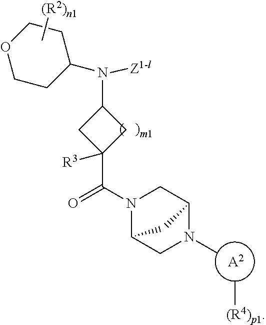

In certain embodiments, Y¹ is represented by

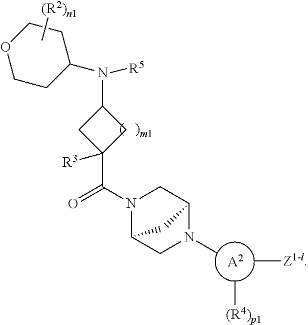

In certain embodiments, Y¹ is represented by

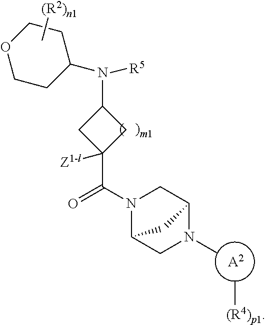

In certain embodiments, Y¹ is

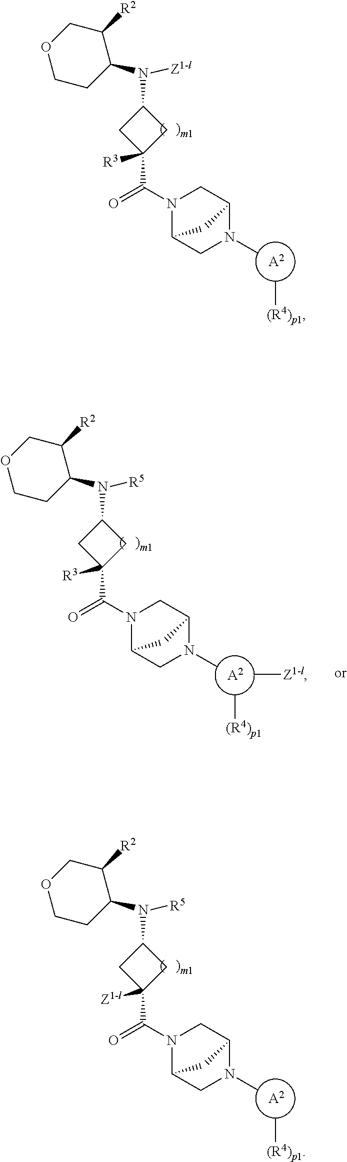

In certain embodiments, Y¹ is represented by

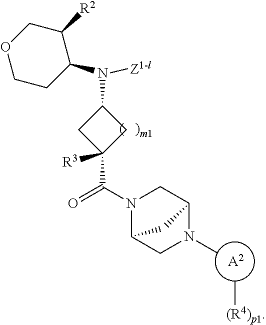

In certain embodiments, Y¹ is represented by

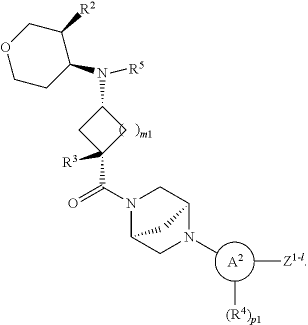

In certain embodiments, Y¹ is represented by

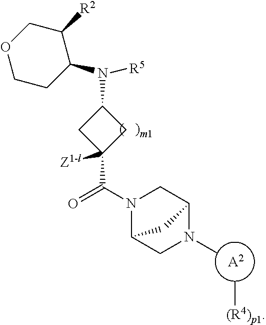

In certain embodiments, Y¹ is one of the following:

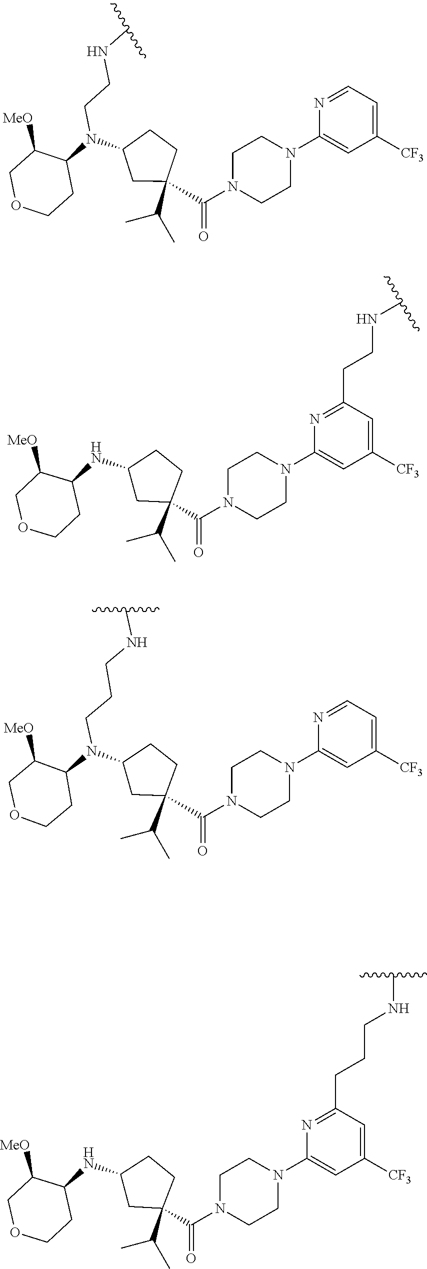 [SMILES: *NCCN([C@@H]1CC[C@@](C(=O)N2CCN(c3cc(C(F)(F)F)ccn3)CC2)(C(C)C)C1)[C@H]1CCOC[C@H]1OC ; *NCCc1cc(C(F)(F)F)cc(N2CCN(C(=O)[C@@]3(C(C)C)CC[C@@H](N[C@H]4CCOC[C@H]4OC)C3)CC2)n1 ; *NCCCN([C@@H]1CC[C@@](C(=O)N2CCN(c3cc(C(F)(F)F)ccn3)CC2)(C(C)C)C1)[C@H]1CCOC[C@H]1OC ; *NCCCc1cc(C(F)(F)F)cc(N2CCN(C(=O)[C@@]3(C(C)C)CC[C@@H](N[C@H]4CCOC[C@H]4OC)C3)CC2)n1]

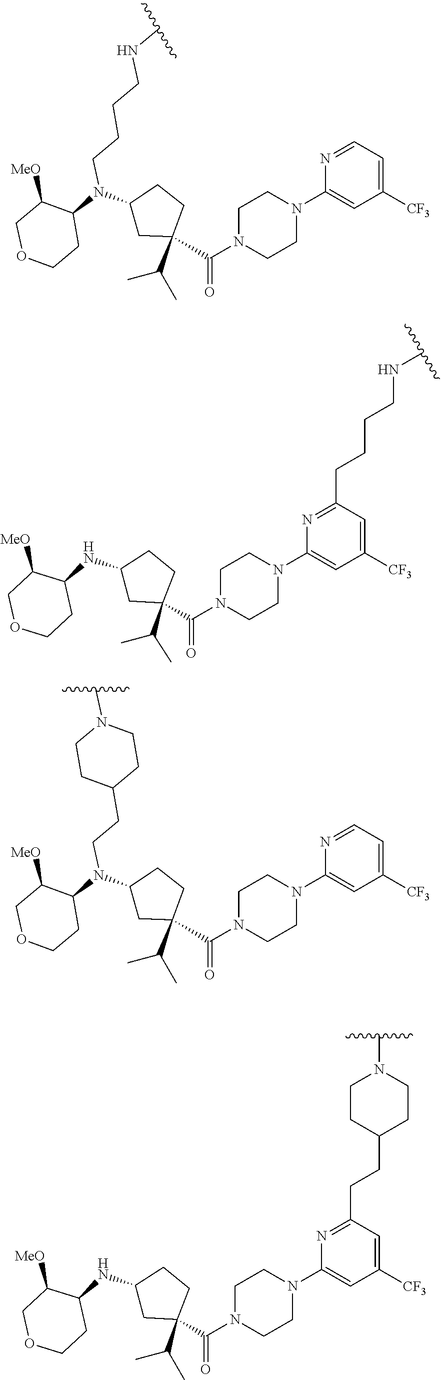 [SMILES: *NCCCCN([C@@H]1CC[C@@](C(=O)N2CCN(c3cc(C(F)(F)F)ccn3)CC2)(C(C)C)C1)[C@H]1CCOC[C@H]1OC ; *NCCCCc1cc(C(F)(F)F)cc(N2CCN(C(=O)[C@@]3(C(C)C)CC[C@@H](N[C@H]4CCOC[C@H]4OC)C3)CC2)n1 ; *N1CCC(CCN([C@@H]2CC[C@@](C(=O)N3CCN(c4cc(C(F)(F)F)ccn4)CC3)(C(C)C)C2)[C@H]2CCOC[C@H]2OC)CC1 ; *N1CCC(CCc2cc(C(F)(F)F)cc(N3CCN(C(=O)[C@@]4(C(C)C)CC[C@@H](N[C@H]5CCOC[C@H]5OC)C4)CC3)n2)CC1]

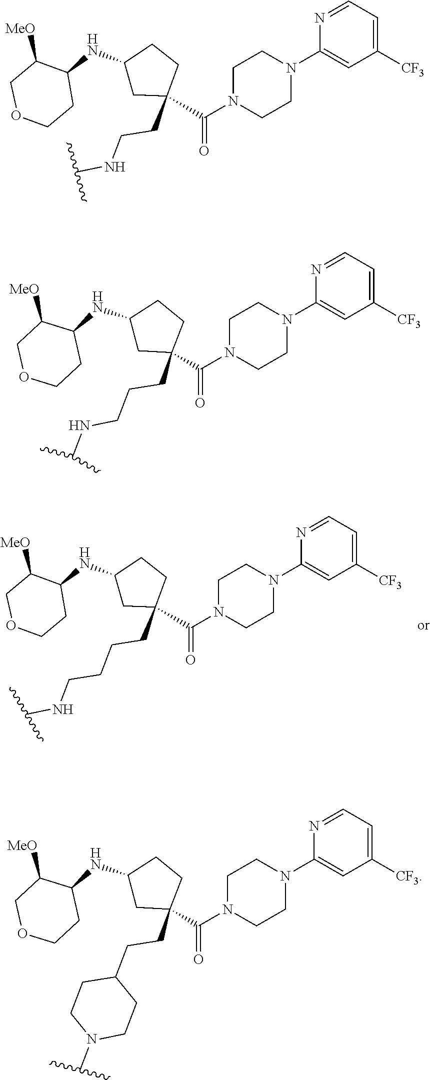 [SMILES: *NCC[C@]1(C(=O)N2CCN(c3cc(C(F)(F)F)ccn3)CC2)CC[C@@H](N[C@H]2CCOC[C@H]2OC)C1 ; *NCCC[C@]1(C(=O)N2CCN(c3cc(C(F)(F)F)ccn3)CC2)CC[C@@H](N[C@H]2CCOC[C@H]2OC)C1 ; *NCCCC[C@]1(C(=O)N2CCN(c3cc(C(F)(F)F)ccn3)CC2)CC[C@@H](N[C@H]2CCOC[C@H]2OC)C1 ; *N1CCC(CC[C@]2(C(=O)N3CCN(c4cc(C(F)(F)F)ccn4)CC3)CC[C@@H](N[C@H]3CCOC[C@H]3OC)C2)CC1]

In certain embodiments, Y¹ is one of the following:

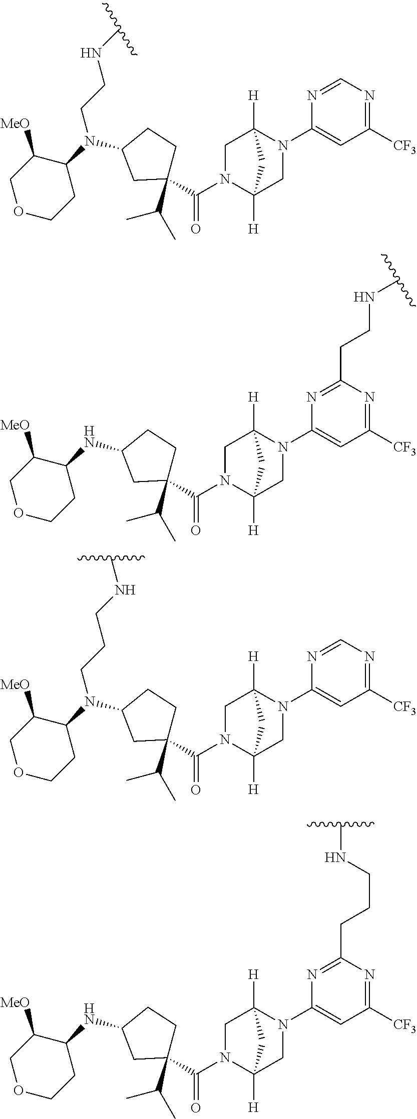

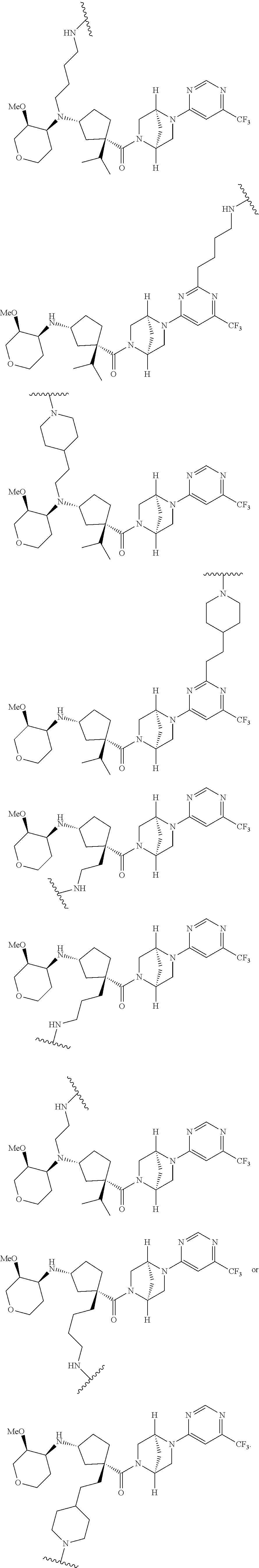

In certain embodiments, Y¹ is selected from the groups depicted in the compounds in Table 1, below.

In certain embodiments, Y¹ is

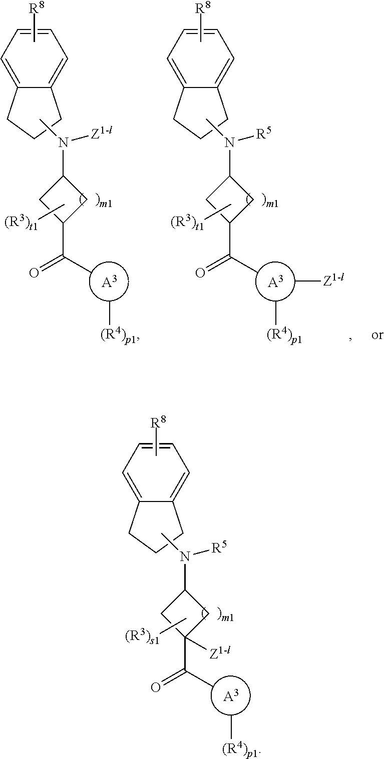

In certain embodiments, Y¹ is

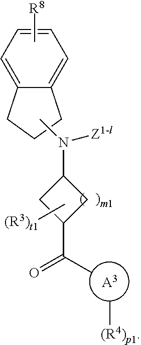

In certain embodiments, Y¹ is

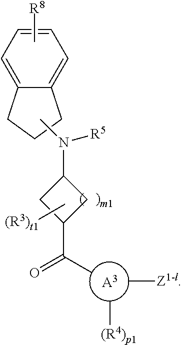

In certain embodiments, Y¹ is

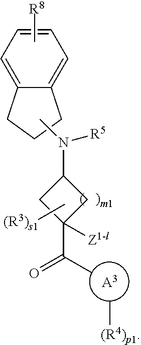

In certain embodiments, Y¹ is

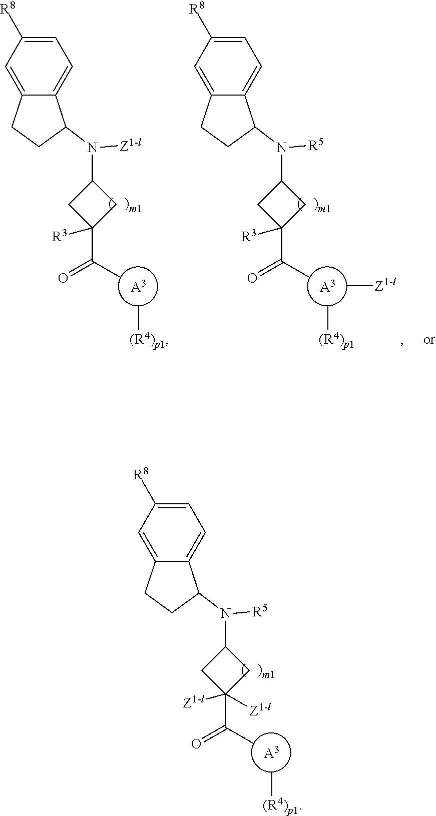

In certain embodiments, Y¹ is represented by

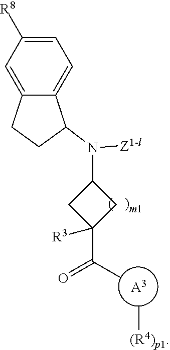

In certain embodiments, Y¹ is represented by

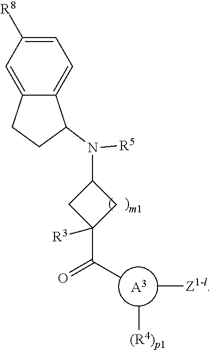

In certain embodiments, Y¹ is represented by

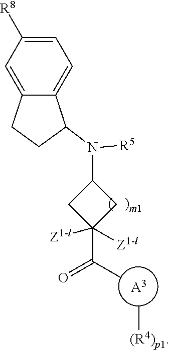

In certain embodiments, Y¹ is

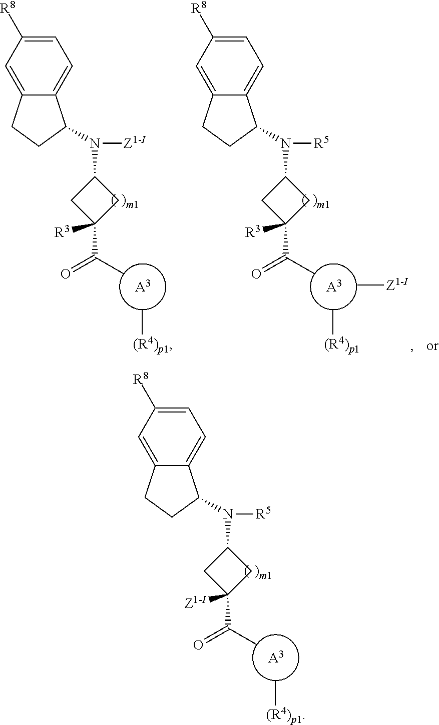

In certain embodiments, Y¹ is represented by

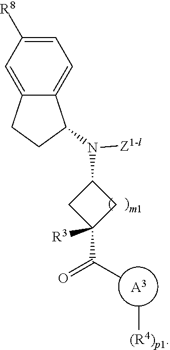

In certain embodiments, Y¹ is represented by

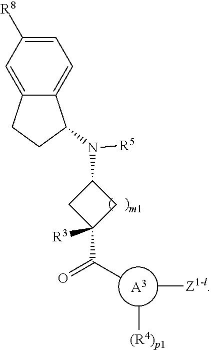

In certain embodiments, Y¹ is represented by

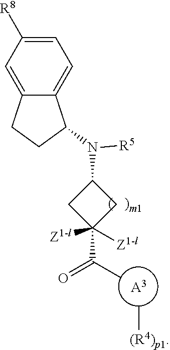

In certain embodiments, Y¹ is one of the following:

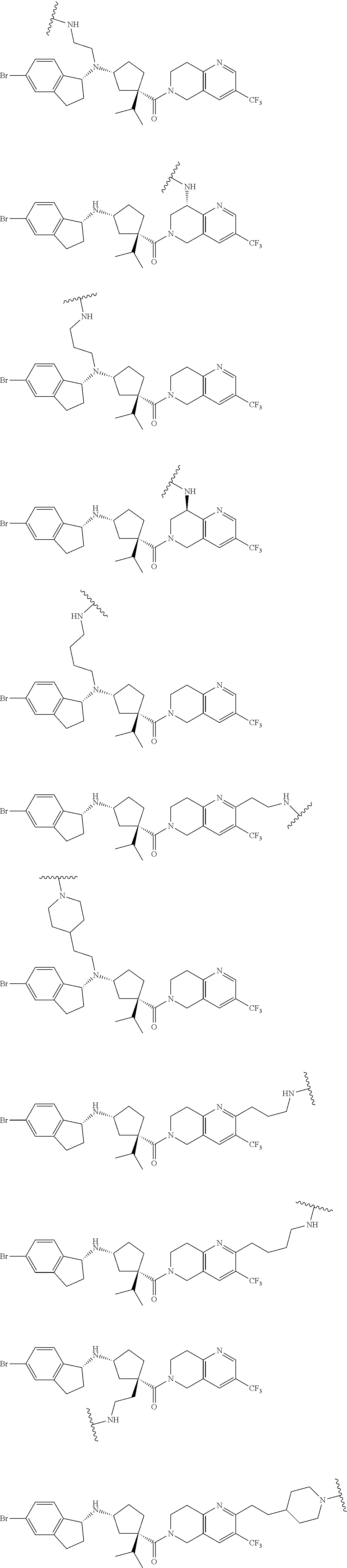

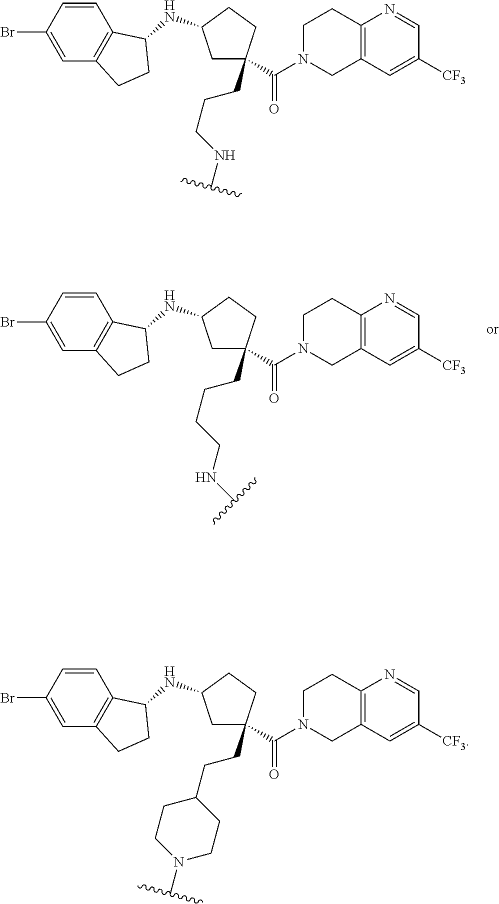 [SMILES: *NCCC[C@]1(C(=O)N2CCc3ncc(C(F)(F)F)cc3C2)CC[C@@H](N[C@@H]2CCc3cc(Br)ccc32)C1 ; *NCCCC[C@]1(C(=O)N2CCc3ncc(C(F)(F)F)cc3C2)CC[C@@H](N[C@@H]2CCc3cc(Br)ccc32)C1 ; *N1CCC(CC[C@]2(C(=O)N3CCc4ncc(C(F)(F)F)cc4C3)CC[C@@H](N[C@@H]3CCc4cc(Br)ccc43)C2)CC1]

In certain embodiments, Y¹ is selected from the groups depicted in the compounds in Table 2, below.

In certain embodiments, Z^{1-I} is —(C₀₋₆ alkylene)-N(R⁶)—R⁷ or —(C₁₋₆ alkylene)-(4-6 membered saturated heterocyclylene containing 1 or 2 heteroatoms selected from nitrogen)-R⁷. In certain embodiments, Z^{1-I} is —(C₁₋₆ alkylene)-N(R⁶)—R⁷ or —(C₁₋₆ alkylene)-(4-6 membered saturated heterocyclylene containing 1 or 2 heteroatoms selected from nitrogen)-R⁷.

In certain embodiments, Z^{1-I} is —(C₀₋₆ alkylene)-N(R⁶)—R⁷. In certain embodiments, Z^{1-I} is —(C₁₋₆ alkylene)-N(R⁶)—R⁷. In certain embodiments, Z^{1-I} is —(C₁₋₃ alkylene)-N(R⁶)—R⁷. In certain embodiments, Z^{1-I} is —CH₂—N(R⁶)—R⁷. In certain embodiments, Z^{1-I} is —CH₂CH₂—N(R⁶)—R⁷. In certain embodiments, Z^{1-I} is —CH₂CH₂CH₂—N(R⁶)—R⁷. In certain embodiments, Z^{1-I} is —N(R⁶)—R⁷.

In certain embodiments, Z^{1-I} is —(C₁₋₆ alkylene)-(4-6 membered saturated heterocyclylene containing 1 or 2 heteroatoms selected from nitrogen)-R⁷. In certain embodiments, Z^{1-I} is —(C₁₋₆ alkylene)-(piperidinylene)-R⁷. In certain embodiments, Z^{1-I} is —(C₂₋₃ alkylene)-(piperidinylene)-R⁷. In certain embodiments, Z^{1-I} is -(ethylene)-(piperidinylene)-R⁷. In certain embodiments, Z^{1-I} is

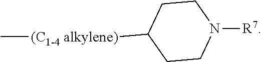

In certain embodiments, Z^{1-I} is

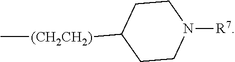

In certain embodiments, Z^{1-I} is —(C₀₋₆ alkylene)-O—R⁷. In certain embodiments, Z^{1-I} is —(C₁₋₆ alkylene)-O—R⁷. In certain embodiments, Z^{1-I} is —O—R⁷. In certain embodiments, Z^{1-I} is selected from the groups depicted in the compounds in Table 1, below. In certain embodiments, Z^{1-I} is selected from the groups depicted in the compounds in Table 2, below.

In certain embodiments, R¹ is C₁₋₄ alkyl. In certain embodiments, R¹ is C₃₋₆ cycloalkyl. In certain embodiments, R¹ is —CH₃. In certain embodiments, R¹ is methyl, ethyl, 1-propyl, 2-propyl, 1-butyl, 2-butyl, or t-butyl. In certain embodiments, R¹ is cyclopropyl, cyclobutyl, cyclopentyl, or cyclohexyl. In certain embodiments, R¹ is selected from the groups depicted in the compounds in Table 1, below. In certain embodiments, R¹ is selected from the groups depicted in the compounds in Table 2, below.

In certain embodiments, R² represents independently for each occurrence C₁₋₄ alkoxyl. In certain embodiments, R² is C₁₋₄ alkoxyl. In certain embodiments, R² is methoxy. In certain embodiments, R² represents independently for each occurrence C₁₋₄ alkyl. In certain embodiments, R² is methyl. In certain embodiments, R² represents independently for each occurrence methoxy or methyl. In certain embodiments, R² is selected from the groups depicted in the compounds in Table 1, below.

In certain embodiments, R³ is C₂₋₃ alkyl. In certain embodiments, R³ is isopropyl. In certain embodiments, R³ is propyl. In certain embodiments, R³ is ethyl. In certain embodiments, R³ is butyl. In certain embodiments, R³ is selected from the groups depicted in the compounds in Table 1, below. In certain embodiments, R³ is selected from the groups depicted in the compounds in Table 2, below.

In certain embodiments, R⁴ represents independently for each occurrence C₁₋₄ haloalkyl. In certain embodiments, R⁴ is C₁₋₄ haloalkyl. In certain embodiments, R⁴ is C₁₋₂ haloalkyl. In certain embodiments, R⁴ is C₁₋₃ fluoroalkyl. In certain embodiments, R⁴ is trifluoromethyl. In certain embodiments, R⁴ represents independently for each occurrence halo. In certain embodiments, R⁴ represents independently for each occurrence C₁₋₄ alkyl. In certain embodiments, R⁴ is methyl. In certain embodiments, R⁴ is selected from the groups depicted in the compounds in Table 1, below. In certain embodiments, R⁴ is selected from the groups depicted in the compounds in Table 2, below.

In certain embodiments, R⁵, if present, is hydrogen. In certain embodiments, R⁵ is hydrogen. In certain embodiments, R⁵ is C₁₋₄ alkyl. In certain embodiments, R⁵ is methyl. In certain embodiments, R⁵ is selected from the groups depicted in the compounds in Table 1, below. In certain embodiments, R⁵ is selected from the groups depicted in the compounds in Table 2, below.

In certain embodiments, R⁶ is hydrogen. In certain embodiments, R⁶ is C₁₋₄ alkyl. In certain embodiments, R⁶ is methyl. In certain embodiments, R⁶ is selected from the groups depicted in the compounds in Table 1, below. In certain embodiments, R⁶ is selected from the groups depicted in the compounds in Table 2, below.

In certain embodiments, R⁸ is C₁₋₄ haloalkyl. In certain embodiments, R⁸ is C₁₋₂ haloalkyl. In certain embodiments, R⁸ is C₁₋₃ fluoroalkyl. In certain embodiments, R⁸ is trifluoromethyl. In certain embodiments, R⁸ is halo. In certain embodiments, R⁸ is bromo. In certain embodiments, R⁸ is C₁₋₄ alkyl. In certain embodiments, R⁸ is methyl. In certain embodiments, R⁸ is selected from the groups depicted in the compounds in Table 2, below.

In certain embodiments, R⁹ represents independently for each occurrence C₂₋₃ alkyl. In certain embodiments, R⁹ is propyl. In certain embodiments, R⁹ is methyl. In certain embodiments, R⁹ is selected from the groups depicted in the compounds in Table 1, below.

In certain embodiments, A¹ is a 5-7 membered saturated heterocyclylene containing 1 or 2 heteroatoms independently selected from nitrogen and oxygen, wherein the heterocyclylene is substituted with w1 occurrences of R⁹.

In certain embodiments, A¹ is a 6-membered saturated heterocyclylene containing 1 or 2 heteroatoms that are nitrogen, wherein the heterocyclylene is substituted with w1 occurrences of R⁹. In certain embodiments, A¹ is a 6-membered saturated heterocyclylene containing 1 or 2 heteroatoms that are nitrogen. In certain embodiments, A¹ is piperazinylene. In certain embodiments, A¹ is

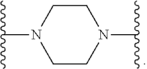 [SMILES: *N1CCN(*)CC1]

In certain embodiments, A¹ is a 6-8 membered bridged bicyclic saturated heterocyclylene containing 1 or 2 heteroatoms independently selected from nitrogen and oxygen, wherein the heterocyclylene is substituted with w1 occurrences of R⁹.

In certain embodiments, A¹ is a 7-membered bridged bicyclic saturated heterocyclylene containing 1 or 2 heteroatoms that are nitrogen, wherein the heterocyclylene is substituted with w1 occurrences of R⁹. In certain embodiments, A¹ is a 7-membered bridged bicyclic saturated heterocyclylene containing 1 or 2 heteroatoms that are nitrogen. In certain embodiments, A¹ is

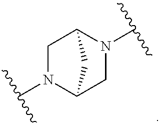 [SMILES: *N1C[C@@H]2C[C@H]1CN2*]

In certain embodiments, A¹ is selected from the groups depicted in the compounds in Table 1, below.

In certain embodiments, A² is a 6-membered heteroarylene containing 1 or 2 heteroatoms independently selected from nitrogen, oxygen, or sulfur. In certain embodiments, A² is pyridiylene. In certain embodiments, A² is pyrimidinylene. In certain embodiments, A² is selected from the groups depicted in the compounds in Table 1, below.

In certain embodiments, A³ is an 8-10 membered bicyclic heteroarylene containing 2 heteroatoms that are nitrogen. In certain embodiments, A³ is a 10-membered bicyclic heteroarylene containing 2 heteroatoms that are nitrogen. In certain embodiments, A³ is a 5,6,7,8-tetrahydro-1,6-naphthyridinylene. In certain embodiments, A³ is

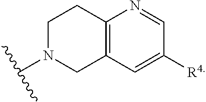

In certain embodiments, A³ is

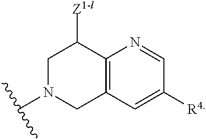

In certain embodiments, A³ is selected from the groups depicted in the compounds in Table 2, below.

In certain embodiments, n1 is 1. In certain embodiments, n1 is 2. In certain embodiments, n1 is 3. In certain embodiments, n1 is selected from the values represented in the compounds in Table 1, below.

In certain embodiments, p1 is 1. In certain embodiments, p1 is 2. In certain embodiments, p1 is 3. In certain embodiments, p1 is selected from the values represented in the compounds in Table 1, below. In certain embodiments, p1 is selected from the values represented in the compounds in Table 2, below.

In certain embodiments, t1 is 1. In certain embodiments, t1 is 2. In certain embodiments, t1 is 3. In certain embodiments, t1 is selected from the values represented in the compounds in Table 1, below. In certain embodiments, t1 is selected from the values represented in the compounds in Table 2, below.

In certain embodiments, m1 is 2. In certain embodiments, m1 is 1. In certain embodiments, m1 is selected from the values represented in the compounds in Table 1, below. In certain embodiments, m1 is selected from the values represented in the compounds in Table 2, below.

In certain embodiments, w1 is 0. In certain embodiments, w1 is 1. In certain embodiments, w1 is 2. In certain embodiments, w1 is selected from the values represented in the compounds in Table 1, below.

In certain embodiments, s1 is 0. In certain embodiments, s1 is 1. In certain embodiments, s1 is 2. In certain embodiments, s1 is selected from the values represented in the compounds in Table 1, below. In certain embodiments, s1 is selected from the values represented in the compounds in Table 2, below.

The description above describes multiple embodiments relating to compounds of Formula I. The patent application specifically contemplates all combinations of the embodiments.

Another aspect of the invention provides a compound represented by Formula I-1:

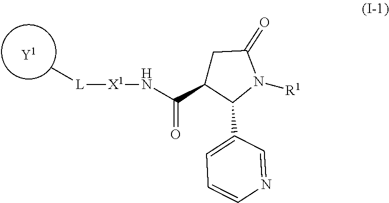

or a pharmaceutically acceptable salt thereof, wherein:
X¹ is C₁₋₅ alkylene or a covalent bond;
Y¹ is one of the following:

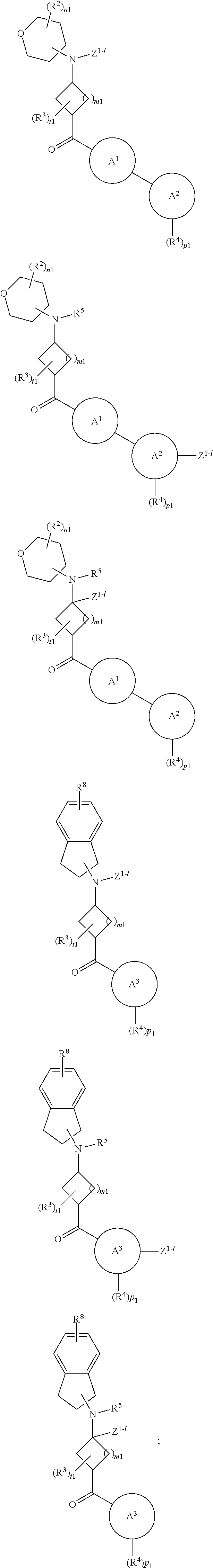

Z^{1-I} is C₁₋₆ alkylene-N(R⁶)—R⁷ or —(C₁₋₆ alkylene)-(4-6 membered saturated heterocyclylene containing 1 or 2 heteroatoms selected from nitrogen)-R⁷;
R¹ is C₁₋₄ alkyl or C₃₋₆ cycloalkyl;
R² represents independently for each occurrence C₁₋₄ alkoxyl or C₁₋₄ alkyl;
R³ and R⁹ each represent independently for each occurrence C₁₋₄ alkyl;
R⁵ and R⁶ are independently hydrogen or C₁₋₄ alkyl;
R⁴ and R⁸ each represent independently for each occurrence C₁₋₄ haloalkyl, halo, or C₁₋₄ alkyl;
R⁷ is a bond to L;
A¹ is (i) a 5-7 membered saturated heterocyclylene containing 1 or 2 heteroatoms independently selected from nitrogen and oxygen or (ii) a 6-8 membered bridged bicyclic saturated heterocyclylene containing 1 or 2 heteroatoms independently selected from nitrogen and oxygen; wherein the heterocyclylene is substituted with w1 occurrences of R⁹;
A² is a 5-6 membered heteroarylene containing 1 or 2 heteroatoms independently selected from nitrogen, oxygen, or sulfur;
A³ is an 8-10 membered bicyclic partially unsaturated ring containing 1, 2, or 3 heteroatoms selected from nitrogen;
n1, p1, and t1 are independently 1, 2, or 3;
m1 is 1 or 2;
w1 is 0, 1, or 2; and
L is a divalent linker selected from:
(i) a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, optionally substituted 3-10 membered heterocyclyl containing 1, 2, 3, or 4 heteroatoms independently selected from nitrogen, oxygen, and sulfur, or optionally substituted 5-6 membered heteroaryl containing 1, 2, or 3 heteroatoms independently selected from nitrogen, oxygen, and sulfur;
(ii)

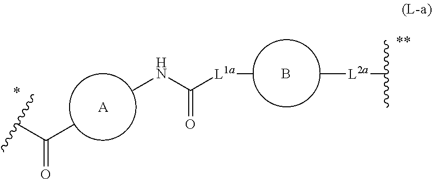

wherein Ring A and Ring B are each independently C₄₋₆ cycloalkylene; L^{1a} is C₃₋₅ linear alkylene, wherein 1 or 2 methylene units are replaced with —O— or —NR^{a}—; each R^{a} is independently hydrogen or C₁₋₃ alkyl; and L^{2a} is —O—, —NHC(O)—, or —CH₂—O—;
(iii)

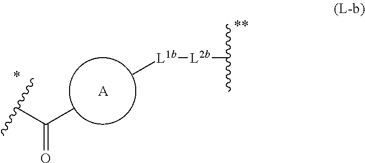

wherein Ring A is C₄₋₆ cycloalkylene or C₇₋₉ bridged bicyclic cycloalkylene; L^{1b} is —CH₂—NH—C(O)—, —NHC(O)—, or —C(O)NH—; L^{2b} is C₆-12 linear alkylene, wherein 1, 2, 3, or 4 methylene units are replaced with —O—, —NR^{1b}—, —C(O)NR^{b}—, or —NR^{1b}C(O)—; or L^{2b} is

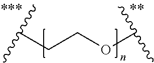

wherein n is 1, 2, 3, or 4, and

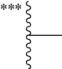

represents a covalent bond to L^{1b}; and each R^{1b} is independently hydrogen or C₁₋₃ alkyl;
(iv)

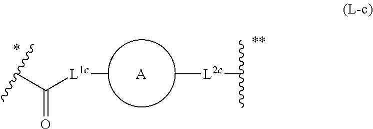

wherein L^{1d} is C₂₋₁₀ linear alkylene, wherein 1, 2, or 3 methylene units are replaced with —O—, —NH—, —NHC(O)—, or —C(O)NH—; Ring A is C₄₋₆ cycloalkylene or C₇₋₉ bridged bicyclic cycloalkylene; and L^{2c} is —O— or a saturated C₂₋₁₀ linear alkylene, wherein 1, 2, or 3 methylene units are replaced with —O—, —NH—, —NHC(O)—, or —C(O)NH—;
(v)

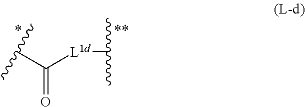

wherein L^{1d} is C₁₂₋₂₂ linear alkylene, wherein 1, 2, 3, 4, or 5 methylene units are replaced with —NH—, —O—, —C(O)NH—, —NHC(O)—, or —NHC(O)—NH—;
(vi)

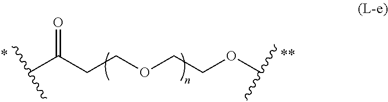

wherein n is an integer of 3 to 50;
(vii)

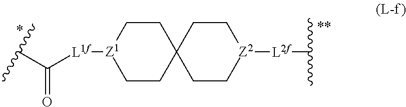

wherein L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—; L^{2f} is a bond, —NHC(O)—, —C(O)NH—, or a C₁₋₆ linear alkylene, wherein 0, 1, or 2 methylene units are replaced with —O—; and each of Z¹ and Z² is independently N or CH;
(viii)

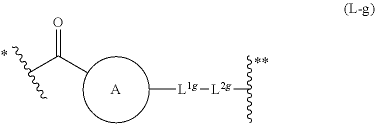

wherein Ring A is a 5 to 6 membered heteroarylene having 1 or 2 nitrogen ring atoms; L^{1g} is a bond, —CH₂—, —NH—, or —O—; and
L^{2g} is

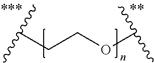

wherein n is 1, 2, 3, 4, or 5, and

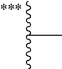

represents a covalent bond to L^{1g};
(ix)

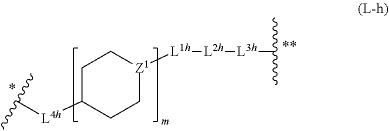

wherein each Z¹ is independently N or CH; L^{1h} is a bond, —C(O)—, —C(O)—NH—, or —NHC(O)—; L^{2h} is C₂₋₁₀ linear alkylene or

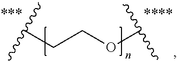

wherein n is 1, 2, 3, or 4, and

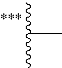

represents a covalent bond to L^{1h} and

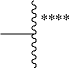

represents a covalent bond to L^{3h}; L^{3h} is a bond, —C(O)CH₂—, —O—(C₃₋₆ cycloalkylene)-O—, or —C(O)NH(CH₂)₃OCH₂—; L^{4h} is a bond, —C(O)—, —CH₂C(O)—, or —C(O)CH₂—; and m is 1, 2, or 3;
(x)

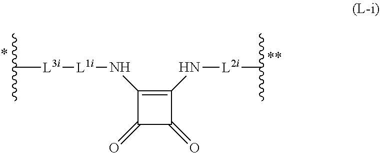

wherein L¹ⁱ is a bond, C₁₋₁₂ linear alkylene, or

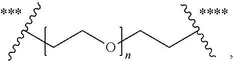

wherein n is 1, 2, 3, 4, or 5, and

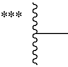

represents a covalent bond to L³ⁱ and

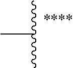

represents a covalent bond to NH; L²ⁱ is a bond, C₁₋₁₂ linear alkylene, or

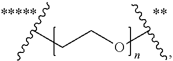

wherein n is 1, 2, 3, 4, or 5, and

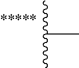

represents a covalent bond to HN; and L³ⁱ is a bond or —C(O)—;
(xi)

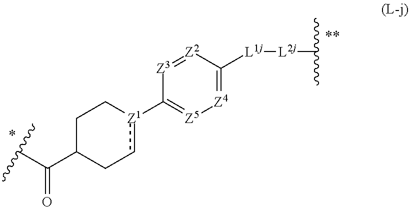

wherein Z¹ is C, CH, or N; each of Z², Z³, Z⁴ and Z⁵ is independently CH or N, provided that no more than two of Z², Z³, Z⁴ and Z⁵ are N; L^{1j} is —NH—, —C(O)NH—, —NHC(O)—, or —O—; L^{2j} is C₁₋₆ linear alkylene or

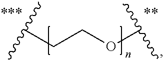

wherein n is 1 or 2, and

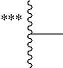

represents a covalent bond to L^{1j}; and [symbol] represents a single bond or a double bond;
(xii)

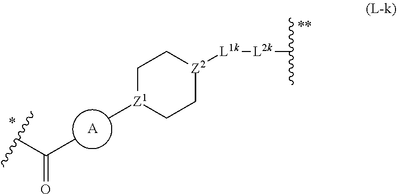

wherein Ring A is phenyl or a 5 or 6 membered heteroarylene having 1 or 2 nitrogen ring atoms; each of Z¹ and Z² is independently CH or N; L^{1k} is a bond, —C(O)—, —C(O)NH— or —NHC(O)—; and L^{2k} is a C₃₋₈ straight chain alkylene or

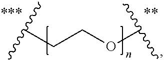

wherein n is 1, 2, or 3, and

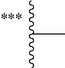

represents
(xiii)

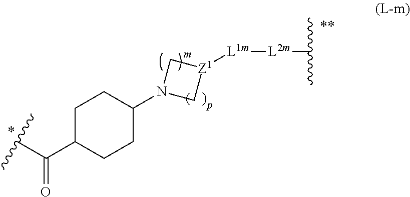

wherein Z¹ is CH or N; m is 1 or 2; p is 1 or 2; 0, 1, or 2 hydrogen atoms of

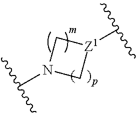

are replaced with F; L^{1m} is a bond, —C(O)—, —C(O)NH—, —NHC(O)—, —S(O)₂NH— or —NHS(O)₂—; and L^{2m} is C₃₋₆ linear alkylene, C₃₋₆ cycloalkylene, or

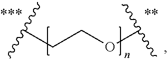

wherein n is 1 or 2, and

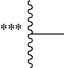

represents a covalent bond to L^{1m};
(xiv) one of

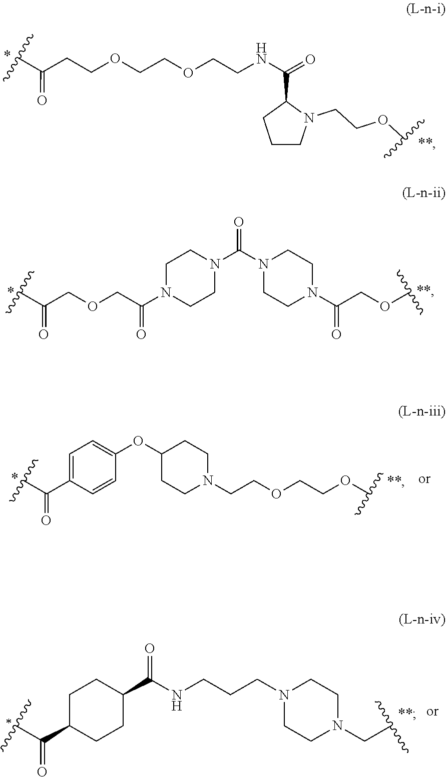

(xv)

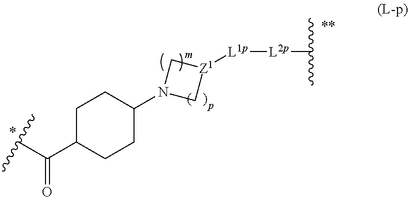

wherein Z¹ is CH or N; m is 1 or 2; p is 1 or 2; 0, 1, or 2 hydrogen atoms of

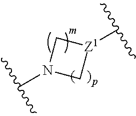

are replaced with F; L^{1p} is a bond, —C(O)—, —C(O)NH—, —NHC(O)—, —S(O)₂NH— or —NHS(O)₂—; and L^{2p} is -(4-6 membered saturated heterocyclylene containing 1 or 2 heteroatoms independently selected from nitrogen oxygen, and sulfur)-C(O))—;
wherein each

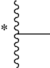

represents a covalent bond to Y¹, and each

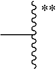

represents a covalent bond to X¹.

The definitions of variables in Formula I-1 above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-1.

In certain embodiments, R¹ is C₁₋₄ alkyl. In certain embodiments, R¹ is C₃₋₆ cycloalkyl. In certain embodiments, R¹ is —CH₃. In certain embodiments, R¹ is methyl, ethyl, 1-propyl, 2-propyl, 1-butyl, 2-butyl, or t-butyl. In certain embodiments, R¹ is methyl. In certain embodiments, R¹ is cyclopropyl, cyclobutyl, cyclopentyl, or cyclohexyl. In certain embodiments, R¹ is selected from the groups depicted in the compounds in Table 1, below.

In certain embodiments, X¹ is C₁₋₅ alkylene. In certain embodiments, X¹ is C₂₋₄ alkylene. In certain embodiments, X¹ is C₂₋₃ alkylene. In certain embodiments, X¹ is —CH₂CH₂—. In certain embodiments, X¹ is —C(H)(CH₃)—. In certain embodiments, X¹ is a covalent bond. In certain embodiments, X¹ is selected from the groups depicted in the compounds in Table 1, below. In certain embodiments, X¹ is selected from the groups depicted in the compounds in Table 2, below.

In certain embodiments, Y¹ is

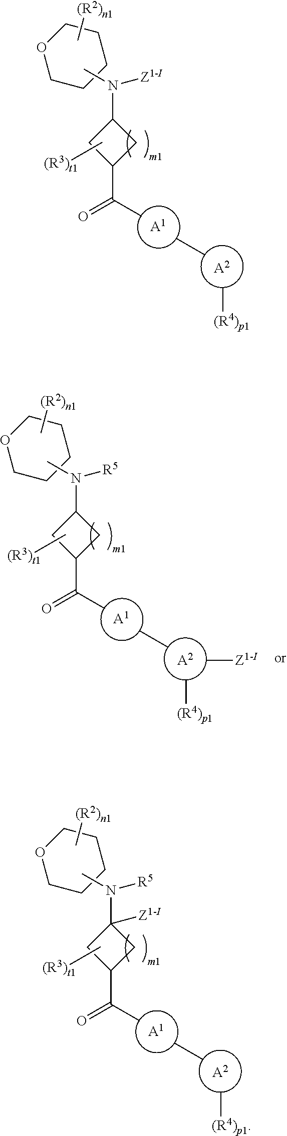

In certain embodiments, Y¹ is

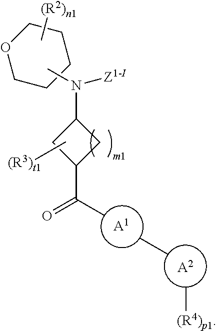

In certain embodiments, Y¹ is

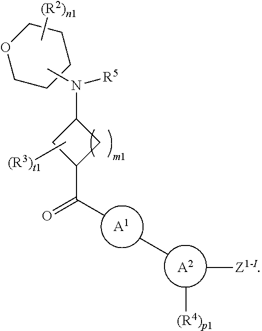

In certain embodiments, Y¹ is

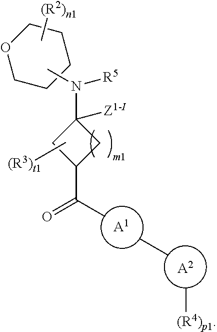

In certain embodiments, Y¹ is represented by

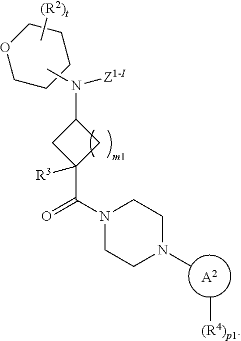

In certain embodiments, Y¹ is represented by

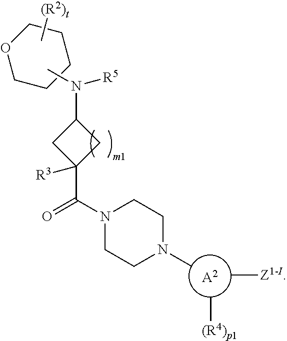

In certain embodiments, Y¹ is represented by

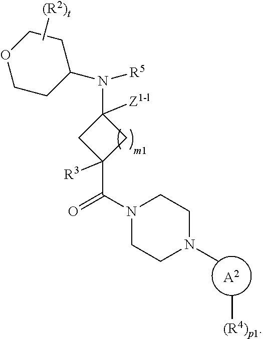

In certain embodiments, Y¹ is represented by

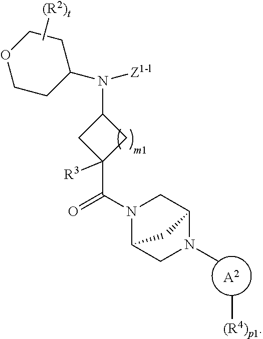

In certain embodiments, Y¹ is represented by

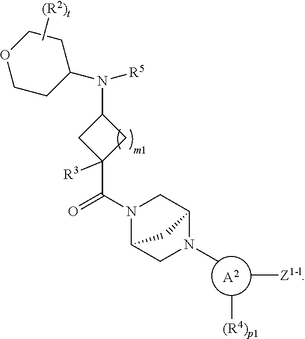

In certain embodiments, Y¹ is represented by

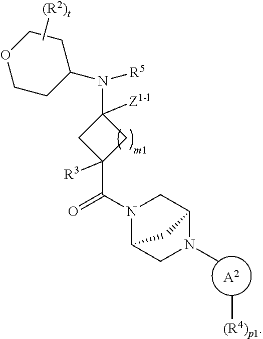

In certain embodiments, Y¹ is represented by

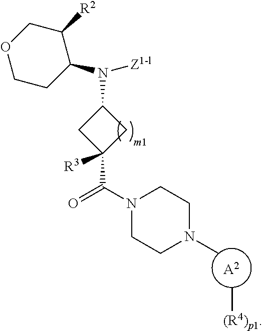

In certain embodiments, Y¹ is represented by

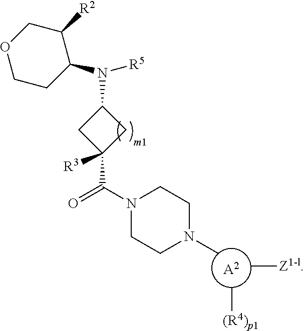

In certain embodiments, Y¹ is represented by

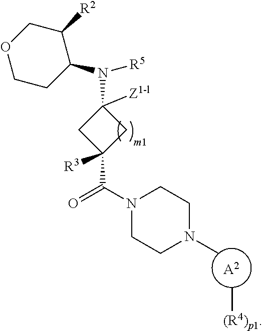

In certain embodiments, Y¹ is represented by

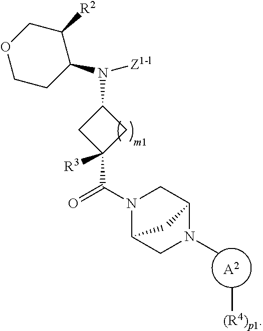

In certain embodiments, Y¹ is represented by

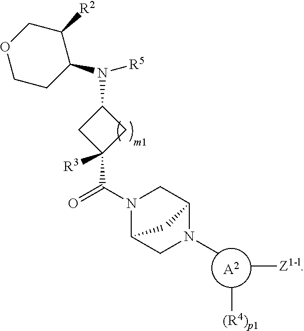

In certain embodiments, Y¹ is represented by

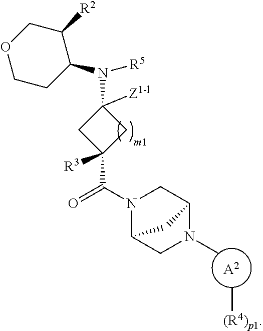

In certain embodiments, Y¹ is selected from the groups depicted in the compounds in Table 1, below

In certain embodiments, R² is C₁₋₄ alkoxyl. In certain embodiments, R² is methoxy. In certain embodiments, R² is C₁₋₄ alkyl. In certain embodiments, R² is methyl. In certain embodiments, R² is selected from the groups depicted in the compounds in Table 1, below.

In certain embodiments, A¹ is (i) a 5-7 membered saturated heterocyclylene containing 1 or 2 heteroatoms independently selected from nitrogen and oxygen or (ii) a 6-8 membered bridged bicyclic saturated heterocyclylene containing 1 or 2 heteroatoms independently selected from nitrogen and oxygen; wherein the heterocyclylene is substituted with w1 occurrences of R⁹.

In certain embodiments, A¹ is (i) a 6 membered saturated heterocyclylene containing 1 or 2 heteroatoms independently selected from nitrogen, wherein the heterocyclylene is substituted with w1 occurrences of R⁹. In certain embodiments, A¹ is (i) a 6 membered saturated heterocyclylene containing 1 or 2 heteroatoms independently selected from nitrogen. In certain embodiments, A¹ is piperazinylene. In certain embodiments, A¹ is

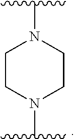 [SMILES: *N1CCN(*)CC1]

In certain embodiments, A¹ is a 7-membered bridged bicyclic saturated heterocyclylene containing 1 or 2 heteroatoms independently selected from nitrogen, wherein the heterocyclylene is substituted with w1 occurrences of R⁹. In certain embodiments, A¹ is a 7-membered bridged bicyclic saturated heterocyclylene containing 1 or 2 heteroatoms independently selected from nitrogen. In certain embodiments, A¹ is

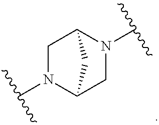 [SMILES: *N1C[C@@H]2C[C@H]1CN2*]

In certain embodiments, A¹ is selected from the groups depicted in the compounds in Table 1, below.

In certain embodiments, A² is a 6 membered heteroarylene containing 1 or 2 heteroatoms independently selected from nitrogen, oxygen, or sulfur. In certain embodiments, A² is pyridiylene. In certain embodiments, A² is pyrimidinylene. In certain embodiments, A² is selected from the groups depicted in the compounds in Table 1, below.

In certain embodiments, R³ is C₁₋₄ alkyl. In certain embodiments, R³ is C₂₋₃ alkyl. In certain embodiments, R³ is isopropyl. In certain embodiments, R³ is propyl. In certain embodiments, R³ is ethyl. In certain embodiments, R³ is butyl. In certain embodiments, R³ is selected from the groups depicted in the compounds in Table 1, below. In certain embodiments, R³ is selected from the groups depicted in the compounds in Table 2, below.

In certain embodiments, R⁴ is C₁₋₄ haloalkyl. In certain embodiments, R⁴ is C₁₋₃ haloalkyl. In certain embodiments, R⁴ is C₁₋₂ haloalkyl. In certain embodiments, R⁴ is C₁₋₃ fluoroalkyl. In certain embodiments, R⁴ is C₁₋₂ fluoroalkyl. In certain embodiments, R⁴ is trifluoromethyl. In certain embodiments, R⁴ is halo. In certain embodiments, R⁴ is C₁₋₄ alkyl. In certain embodiments, R⁴ is methyl. In certain embodiments, R⁴ is selected from the groups depicted in the compounds in Table 1, below. In certain embodiments, R⁴ is selected from the groups depicted in the compounds in Table 2, below.

In certain embodiments, R⁵ is hydrogen or C₁₋₄ alkyl. In certain embodiments, R⁵ is hydrogen. In certain embodiments, R⁵ is C₁₋₄ alkyl. In certain embodiments, R⁵ is methyl. In certain embodiments, R⁵ is selected from the groups depicted in the compounds in Table 1, below. In certain embodiments, R⁵ is selected from the groups depicted in the compounds in Table 2, below.

In certain embodiments, R⁶ is hydrogen or C₁₋₄ alkyl. In certain embodiments, R⁶ is hydrogen. In certain embodiments, R⁶ is C₁₋₄ alkyl. In certain embodiments, R⁶ is methyl. In certain embodiments, R⁶ is selected from the groups depicted in the compounds in Table 1, below. In certain embodiments, R⁶ is selected from the groups depicted in the compounds in Table 2, below.

In certain embodiments, R⁸ is C₁₋₄ haloalkyl. In certain embodiments, R⁸ is C₁₋₃ haloalkyl. In certain embodiments, R⁸ is C₁₋₂ haloalkyl. In certain embodiments, R⁸ is C₁₋₃ fluoroalkyl. In certain embodiments, R⁸ is C₁₋₂ fluoroalkyl. In certain embodiments, R⁸ is trifluoromethyl. In certain embodiments, R⁸ is halo. In certain embodiments, R⁸ is C₁₋₄ alkyl. In certain embodiments, R⁸ is methyl. In certain embodiments, R⁸ is selected from the groups depicted in the compounds in Table 2, below.

In certain embodiments, R⁹ is C₁₋₄ alkyl. In certain embodiments, R⁹ is C₂₋₃ alkyl. In certain embodiments, R⁹ is isopropyl. In certain embodiments, R⁹ is propyl. In certain embodiments, R⁹ is ethyl. In certain embodiments, R⁹ is butyl. In certain embodiments, R⁹ is selected from the groups depicted in the compounds in Table 1, below.

In certain embodiments, Y¹ is one of the following:

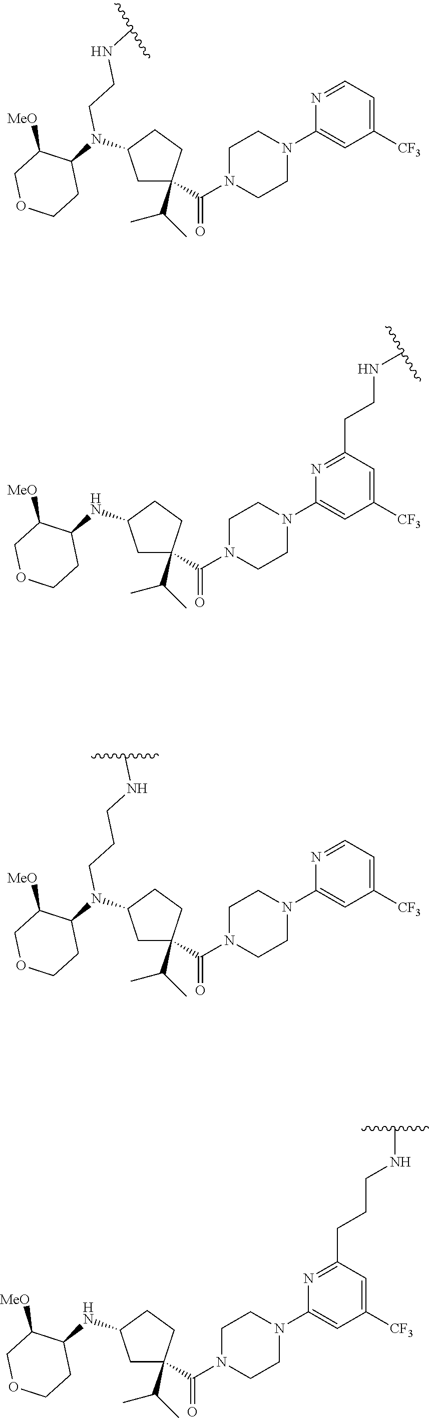 [SMILES: *NCCN([C@@H]1CC[C@@](C(=O)N2CCN(c3cc(C(F)(F)F)ccn3)CC2)(C(C)C)C1)[C@H]1CCOC[C@H]1OC ; *NCCc1cc(C(F)(F)F)cc(N2CCN(C(=O)[C@@]3(C(C)C)CC[C@@H](N[C@H]4CCOC[C@H]4OC)C3)CC2)n1 ; *NCCCN([C@@H]1CC[C@@](C(=O)N2CCN(c3cc(C(F)(F)F)ccn3)CC2)(C(C)C)C1)[C@H]1CCOC[C@H]1OC ; *NCCCc1cc(C(F)(F)F)cc(N2CCN(C(=O)[C@@]3(C(C)C)CC[C@@H](N[C@H]4CCOC[C@H]4OC)C3)CC2)n1]

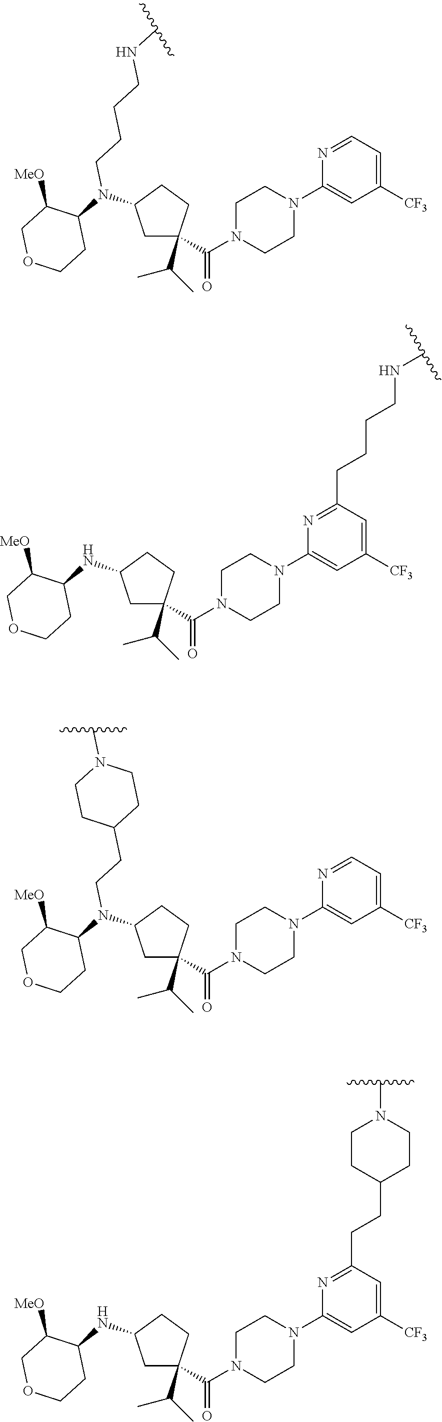 [SMILES: *NCCCCN([C@@H]1CC[C@@](C(=O)N2CCN(c3cc(C(F)(F)F)ccn3)CC2)(C(C)C)C1)[C@H]1CCOC[C@H]1OC ; *NCCCCc1cc(C(F)(F)F)cc(N2CCN(C(=O)[C@@]3(C(C)C)CC[C@@H](N[C@H]4CCOC[C@H]4OC)C3)CC2)n1 ; *N1CCC(CCN([C@@H]2CC[C@@](C(=O)N3CCN(c4cc(C(F)(F)F)ccn4)CC3)(C(C)C)C2)[C@H]2CCOC[C@H]2OC)CC1 ; *N1CCC(CCc2cc(C(F)(F)F)cc(N3CCN(C(=O)[C@@]4(C(C)C)CC[C@@H](N[C@H]5CCOC[C@H]5OC)C4)CC3)n2)CC1]

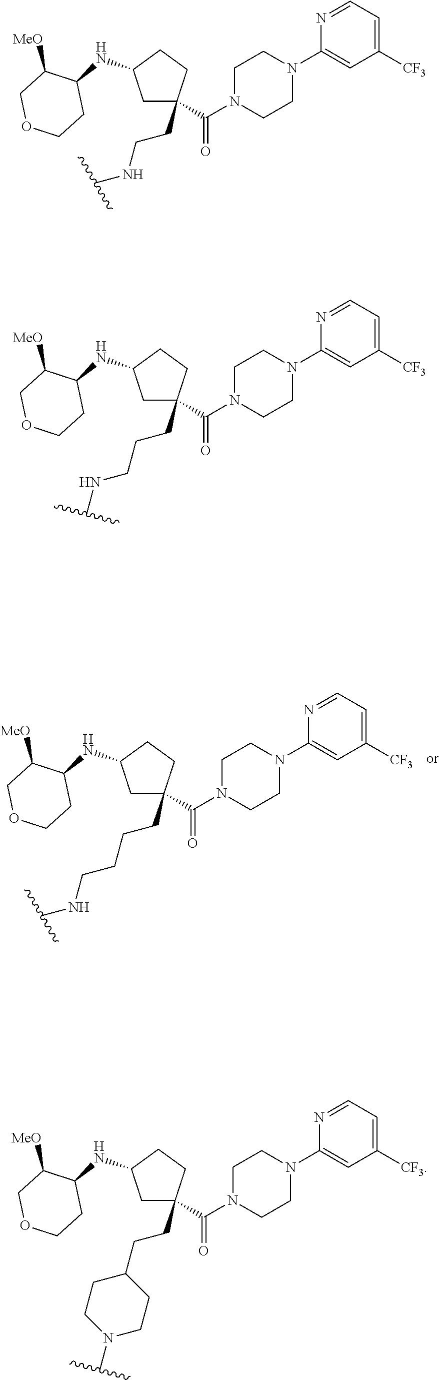 [SMILES: *NCC[C@]1(C(=O)N2CCN(c3cc(C(F)(F)F)ccn3)CC2)CC[C@@H](N[C@H]2CCOC[C@H]2OC)C1 ; *NCCC[C@]1(C(=O)N2CCN(c3cc(C(F)(F)F)ccn3)CC2)CC[C@@H](N[C@H]2CCOC[C@H]2OC)C1 ; *NCCCC[C@]1(C(=O)N2CCN(c3cc(C(F)(F)F)ccn3)CC2)CC[C@@H](N[C@H]2CCOC[C@H]2OC)C1 ; "or" ; *N1CCC(CC[C@]2(C(=O)N3CCN(c4cc(C(F)(F)F)ccn4)CC3)CC[C@@H](N[C@H]3CCOC[C@H]3OC)C2)CC1]

In certain embodiments, Y¹ is one of the following:

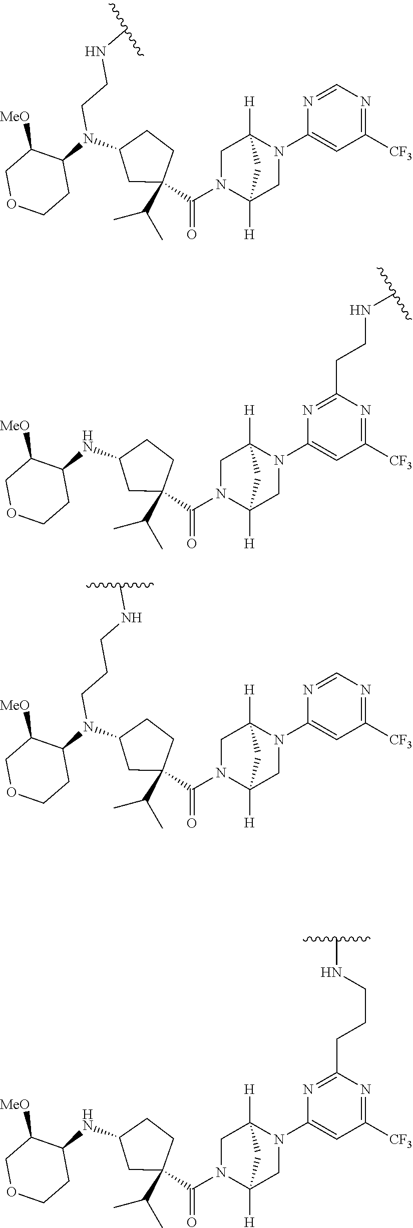 [SMILES: *NCCN([C@@H]1CC[C@@](C(=O)N2C[C@@H]3C[C@H]2CN3c2cc(C(F)(F)F)ncn2)(C(C)C)C1)[C@H]1CCOC[C@H]1OC ; *NCCc1nc(N2C[C@@H]3C[C@H]2CN3C(=O)[C@@]2(C(C)C)CC[C@@H](N[C@H]3CCOC[C@H]3OC)C2)cc(C(F)(F)F)n1 ; *NCCCN([C@@H]1CC[C@@](C(=O)N2C[C@@H]3C[C@H]2CN3c2cc(C(F)(F)F)ncn2)(C(C)C)C1)[C@H]1CCOC[C@H]1OC ; *NCCCc1nc(N2C[C@@H]3C[C@H]2CN3C(=O)[C@@]2(C(C)C)CC[C@@H](N[C@H]3CCOC[C@H]3OC)C2)cc(C(F)(F)F)n1]

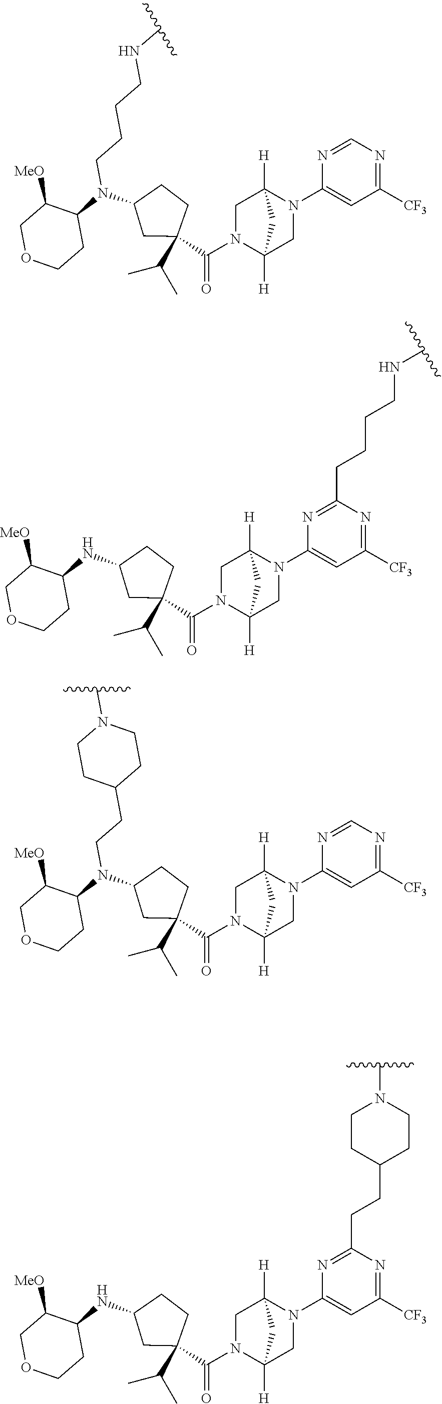 [SMILES: *NCCCCN([C@@H]1CC[C@@](C(=O)N2C[C@@H]3C[C@H]2CN3c2cc(C(F)(F)F)ncn2)(C(C)C)C1)[C@H]1CCOC[C@H]1OC ; *NCCCCc1nc(N2C[C@@H]3C[C@H]2CN3C(=O)[C@@]2(C(C)C)CC[C@@H](N[C@H]3CCOC[C@H]3OC)C2)cc(C(F)(F)F)n1 ; *N1CCC(CCN([C@@H]2CC[C@@](C(=O)N3C[C@@H]4C[C@H]3CN4c3cc(C(F)(F)F)ncn3)(C(C)C)C2)[C@H]2CCOC[C@H]2OC)CC1 ; *N1CCC(CCc2nc(N3C[C@@H]4C[C@H]3CN4C(=O)[C@@]3(C(C)C)CC[C@@H](N[C@H]4CCOC[C@H]4OC)C3)cc(C(F)(F)F)n2)CC1]

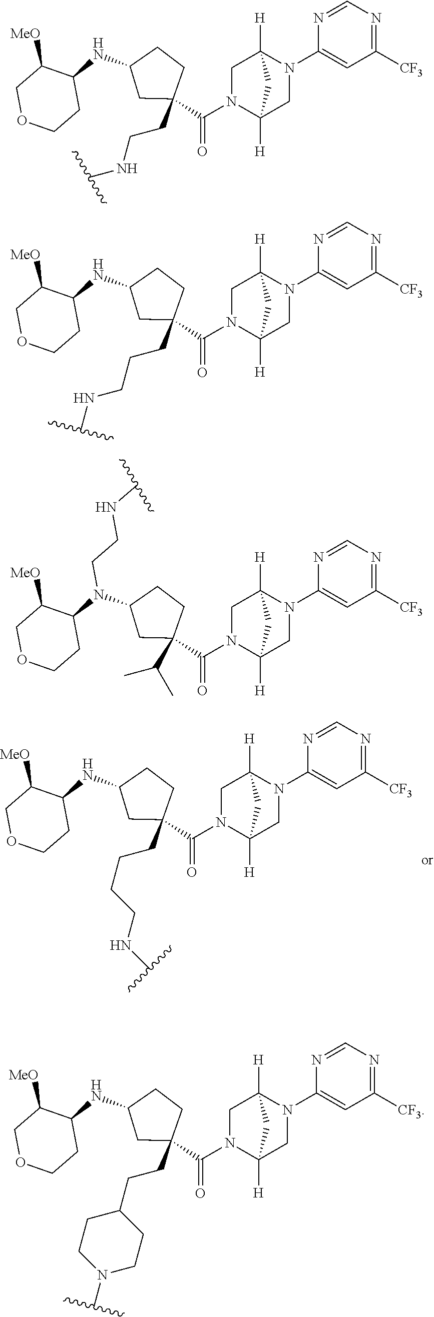 [SMILES: *NCC[C@]1(C(=O)N2C[C@@H]3C[C@H]2CN3c2cc(C(F)(F)F)ncn2)CC[C@@H](N[C@H]2CCOC[C@H]2OC)C1 ; *NCCC[C@]1(C(=O)N2C[C@@H]3C[C@H]2CN3c2cc(C(F)(F)F)ncn2)CC[C@@H](N[C@H]2CCOC[C@H]2OC)C1 ; *NCCN([C@@H]1CC[C@@](C(=O)N2C[C@@H]3C[C@H]2CN3c2cc(C(F)(F)F)ncn2)(C(C)C)C1)[C@H]1CCOC[C@H]1OC ; *NCCCC[C@]1(C(=O)N2C[C@@H]3C[C@H]2CN3c2cc(C(F)(F)F)ncn2)CC[C@@H](N[C@H]2CCOC[C@H]2OC)C1 ; *N1CCC(CC[C@]2(C(=O)N3C[C@@H]4C[C@H]3CN4c3cc(C(F)(F)F)ncn3)CC[C@@H](N[C@H]3CCOC[C@H]3OC)C2)CC1]

In certain embodiments, Y¹ is

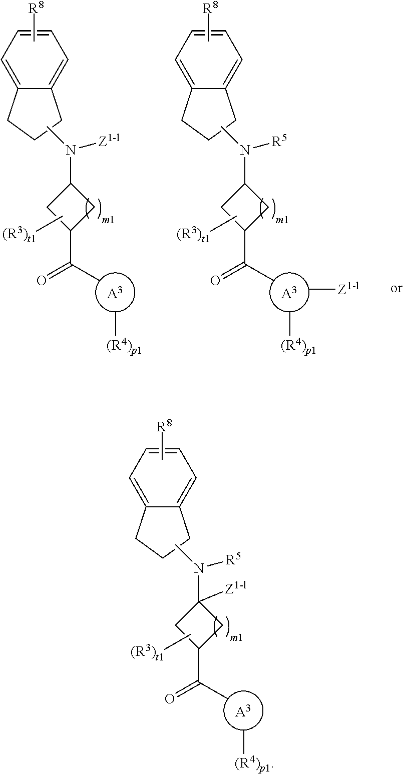

In certain embodiments, Y¹ is represented by

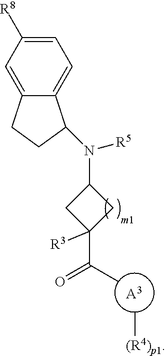

In certain embodiments, Y¹ is represented by

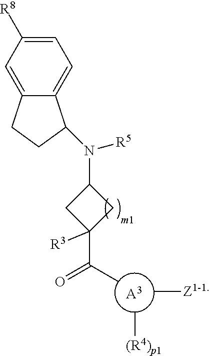

In certain embodiments, Y¹ is represented by

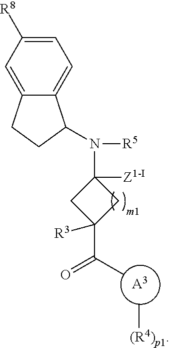

In certain embodiments, Y¹ is represented by

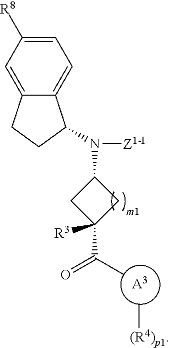

In certain embodiments, Y¹ is represented by

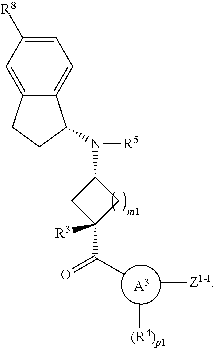

In certain embodiments, Y¹ is represented by

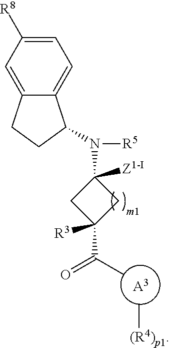

In certain embodiments, Y¹ is selected from the groups depicted in the compounds in Table 2, below.

In certain embodiments, R⁸ is halo. In certain embodiments, R⁸ is bromo. In certain embodiments, R⁸ is selected from the groups depicted in the compounds in Table 2, below

In certain embodiments, A³ is an 8-10 membered bicyclic partially unsaturated ring containing 2 heteroatoms selected from nitrogen. In certain embodiments, A³ is an 10-membered bicyclic partially unsaturated ring containing 2 heteroatoms selected from nitrogen. In certain embodiments, A³ is a 5,6,7,8-tetrahydro-1,6-naphthyridinylene. In certain embodiments, A³ is

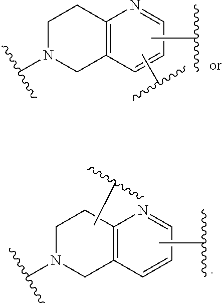

In certain embodiments, A³ is selected from the groups depicted in the compounds in Table 2, below.

In certain embodiments, n1 is 1. In certain embodiments, n1 is 2. In certain embodiments, n1 is 3. In certain embodiments, p1 is 1. In certain embodiments, p1 is 2. In certain embodiments, p1 is 3. In certain embodiments, t1 is 1. In certain embodiments, t1 is 2. In certain embodiments, t1 is 3. In certain embodiments, n1 is a value corresponding to that depicted by compounds in Table 1, below. In certain embodiments, p1 is a value corresponding to that depicted by compounds in Table 1 or 2, below. In certain embodiments, t1 is a value corresponding to that depicted by compounds in Table 1 or 2, below

In certain embodiments, m1 is 2. In certain embodiments, m1 is 1. In certain embodiments, m1 is a value corresponding to that depicted by compounds in Table 1 or 2, below

In certain embodiments, w1 is 0. In certain embodiments, w1 is 1. In certain embodiments, w1 is 2. In certain embodiments, w1 is a value corresponding to that depicted by compounds in Table 1, below

In certain embodiments, R⁵, if present, is hydrogen. In certain embodiments, R⁵, if present, is C₁₋₄ alkyl. In certain embodiments, R⁵, if present, is methyl. In certain embodiments, R⁵, if present, is selected from the groups depicted in the compounds in Table 1, below. In certain embodiments, R⁵, if present, is selected from the groups depicted in the compounds in Table 2, below.

In certain embodiments, Z^{1-I} is C₁₋₆ alkylene-N(R⁶)—R⁷. In certain embodiments, Z^{1-I} is C₁₋₃ alkylene-N(R⁶)—R⁷. In certain embodiments, Z^{1-I} is —CH₂—N(R⁶)—R⁷. In certain embodiments, Z¹—I is —CH₂CH₂—N(R⁶)—R⁷. In certain embodiments, Z^{1-I} is —CH₂CH₂CH₂—N(R⁶)—R⁷. In certain embodiments, Z^{1-I} is a bond to L.

In certain embodiments, Z^{1-I} is —(C₁₋₆ alkylene)-(4-6 membered saturated heterocyclylene containing 1 or 2 heteroatoms selected from nitrogen)-R⁷. In certain embodiments, Z^{1-I} is —(C₁₋₆ alkylene)-(6-membered saturated heterocyclylene containing 1 heteroatom selected from nitrogen)-R⁷. In certain embodiments, Z^{1-I} is —(C₁₋₆ alkylene)-(piperidinylene)-R⁷. In certain embodiments, Z^{1-I} is —(C₂₋₃ alkylene)-(piperidinylene)-R⁷. In certain embodiments, Z^{1-I} is -(ethylene)-(piperidinylene)-R⁷. In certain embodiments, Z^{1-I} is

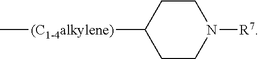

In certain embodiments, Z^{1-I} is

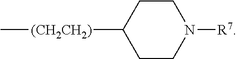

In certain embodiments, Z^{1-I} is selected from the groups depicted in the compounds in Table 1. In certain embodiments, Z^{1-I} is selected from the groups depicted in the compounds in Table 2, below.

The description above describes multiple embodiments relating to compounds of Formula I-1. The patent application specifically contemplates all combinations of the embodiments.

### Linker

As generally defined above for Formula I, L is a divalent linker selected from:
(i) a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, optionally substituted 3-10 membered heterocyclyl containing 1, 2, 3, or 4 heteroatoms independently selected from nitrogen, oxygen, and sulfur, or optionally substituted 5-6 membered heteroaryl containing 1, 2, or 3 heteroatoms independently selected from nitrogen, oxygen, and sulfur;
(ii)

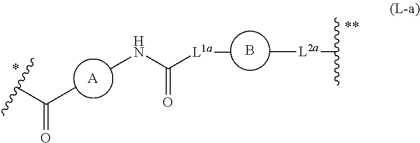

wherein Ring A and Ring B are each independently C₄₋₆ cycloalkylene; L^{1a} is C₃₋₅ linear alkylene, wherein 1 or 2 methylene units are replaced with —O— or —NR^{a}—; each R^{a} is independently hydrogen or C₁₋₃ alkyl; and L^{2a} is —O—, —NHC(O)—, or —CH₂—O—;
(iii)

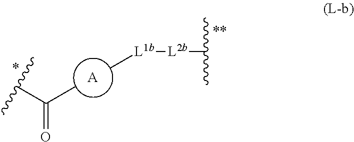

wherein Ring A is C₄₋₆ cycloalkylene or C₇₋₉ bridged bicyclic cycloalkylene; L^{1b} is —CH₂—NH—C(O)—, —NHC(O)—, or —C(O)NH—; L^{2b} is C₆₋₁₂ linear alkylene, wherein 1, 2, 3, or 4 methylene units are replaced with —O—, —NR^{1b}—, —C(O)NR^{1b}, or —NR^{1b}C(O)—; or L^{2b} is

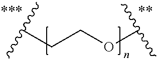

wherein n is 1, 2, 3, or 4, and

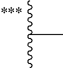

represents a covalent bond to L^{1b}; and each R^{1b} is independently hydrogen or C₁₋₃ alkyl;
(iv)

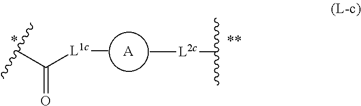

wherein L^{1c} is C₂₋₁₀ linear alkylene, wherein 1, 2, or 3 methylene units are replaced with —O—, —NH—, —NHC(O)—, or —C(O)NH—; Ring A is C₄₋₆ cycloalkylene or C₇₋₉ bridged bicyclic cycloalkylene; and L^{2c} is —O— or a saturated C₂₋₁₀ linear alkylene, wherein 1, 2, or 3 methylene units are replaced with —O—, —NH—, —NHC(O)—, or —C(O)NH—;
(v)

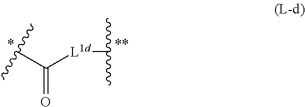

wherein L^{1d} is C₁₂₋₂₂ linear alkylene, wherein 1, 2, 3, 4, or 5 methylene units are replaced with —NH—, —O—, —C(O)NH—, —NHC(O)—, or —NHC(O)—NH—;
(vi)

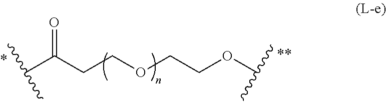

wherein n is an integer of 3 to 50;
(vii)

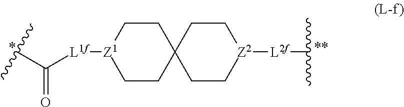

wherein L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—; L^{2f} is a bond, —NHC(O)—, —C(O)NH—, or a C₁₋₆ linear alkylene, wherein 0, 1, or 2 methylene units are replaced with —O—; and each of Z¹ and Z² is independently N or CH;
(viii)

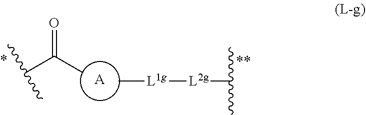

wherein Ring A is a 5 to 6 membered heteroarylene having 1 or 2 nitrogen ring atoms; L^{1g} is a bond, —CH₂—, —NH—, or —O—; and L^{2g} is

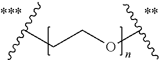

wherein n is 1, 2, 3, 4, or 5, and

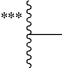

represents a covalent bond to L^{1g};
(ix)

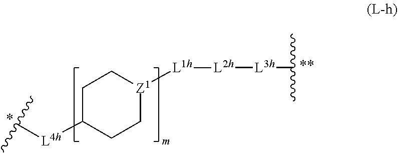

wherein each Z¹ is independently N or CH; L^{1h} is a bond, —C(O)—, —C(O)—NH—, or —NHC(O)—; L^{2h} is C₂₋₁₀ linear alkylene or

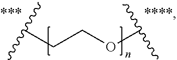

wherein n is 1, 2, 3, or 4, and

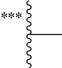

represents a covalent bond to L^{1h} and

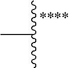

represents a covalent bond to L^{3h}; L^{3h} is a bond, —C(O)CH₂—, —O—(C₃₋₆ cycloalkylene)-O—, or —C(O)NH(CH₂)₃OCH₂—; L^{4h} is a bond, —C(O)—, —CH₂C(O)—, or —C(O)CH₂—; and m is 1, 2, or 3;
(x)

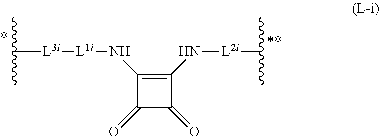

wherein L¹ⁱ is a bond, C₁₋₁₂ linear alkylene, or

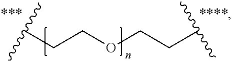

wherein n is 1, 2, 3, 4, or 5, and

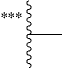

represents a covalent bond to L³ⁱ and

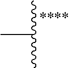

represents a covalent bond to NH; L²ⁱ is a bond, C₁₋₁₂ linear alkylene, or

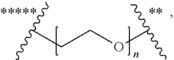

wherein n is 1, 2, 3, 4, or 5, and

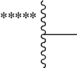

represents a covalent bond to HN; and L³ⁱ is a bond or —C(O)—;
(xi)

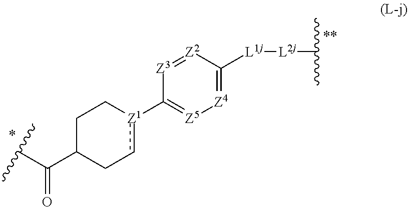

wherein Z¹ is C, CH, or N; each of Z², Z³, Z⁴ and Z⁵ is independently CH or N, provided that no more than two of Z², Z³, Z⁴ and Z⁵ are N; L^{1j} is —NH—, —C(O)NH—, —NHC(O)—, or —O—; L^{2j} is C₁₋₆ linear alkylene or

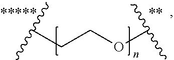

wherein n is 1 or 2, and

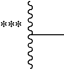

represents a covalent bond to L^{1j}; and [symbol] represents a single bond or a double bond;
(xii)

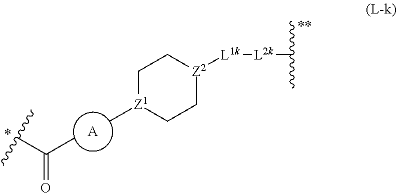

wherein Ring A is phenyl or a 5 or 6 membered heteroarylene having 1 or 2 nitrogen ring atoms; each of Z¹ and Z² is independently CH or N; L^{1k} is a bond, —C(O)—, —C(O)NH— or —NHC(O)—; and L^{2k} is a C₃₋₈ straight chain alkylene or

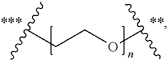

wherein n is 1, 2, or 3, and

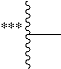

represents a covalent bond to L^{1k};
(xiii)

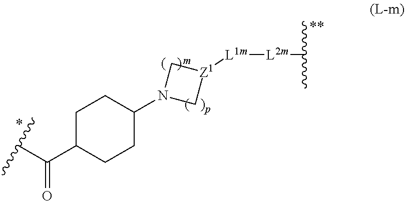

wherein Z¹ is CH or N; m is 1 or 2; p is 1 or 2; 0, 1, or 2 hydrogen atoms of

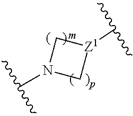

are replaced with F; L^{1m} is a bond, —C(O)—, —C(O)NH—, —NHC(O)—, —S(O)₂NH— or —NHS(O)₂—; and L^{2m} is C₃₋₆ linear alkylene, C₃₋₆ cycloalkylene, or

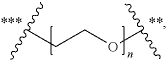

wherein n is 1 or 2, and

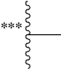

represents a covalent bond to L^{1m};
(xiv) one of

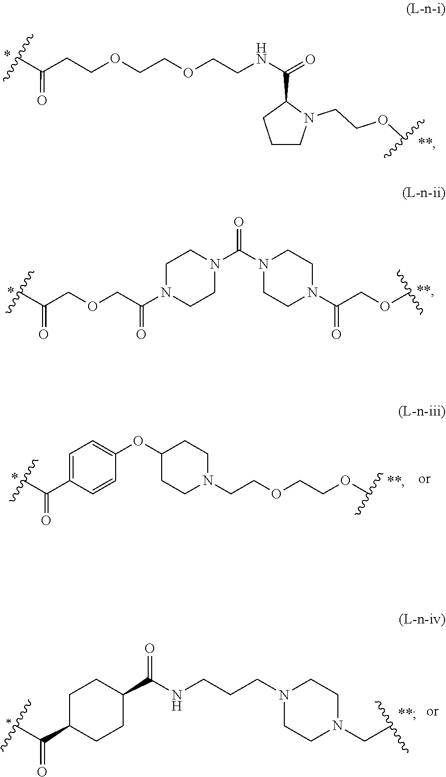

(xv)

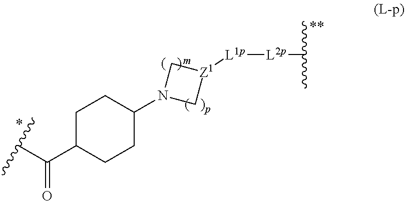

wherein Z¹ is CH or N; m is 1 or 2; p is 1 or 2; 0, 1, or 2 hydrogen atoms of

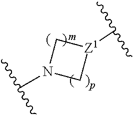

are replaced with F; L^{1p} is a bond, —C(O)—, —C(O)NH—, —NHC(O)—, —S(O)₂—, —S(O)₂NH—, or —NHS(O)₂—; and L^{2p} is -(4-6 membered saturated heterocyclylene containing 1 or 2 heteroatoms independently selected from nitrogen oxygen, and sulfur)-C(O))—;
wherein each

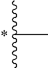

represents a covalent bond to Y¹, and each

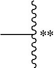

represents a covalent bond to X¹.

As generally defined above for Formula I-1, L is a divalent linker selected from: (i) a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, optionally substituted 3-10 membered heterocyclyl containing 1, 2, 3, or 4 heteroatoms independently selected from nitrogen, oxygen, and sulfur, or optionally substituted 5-6 membered heteroaryl containing 1, 2, or 3 heteroatoms independently selected from nitrogen, oxygen, and sulfur;
(ii)

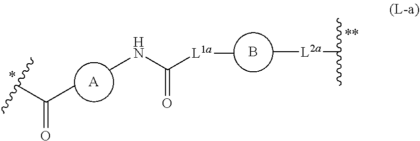

wherein Ring A and Ring B are each independently C₄₋₆ cycloalkylene; L^{1a} is C₃₋₅ linear alkylene, wherein 1 or 2 methylene units are replaced with —O— or —NR^{a}—; each R^{a} is independently hydrogen or C₁₋₃ alkyl; and L^{2a} is —O—, —NHC(O)—, or —CH₂—O—;
(iii)

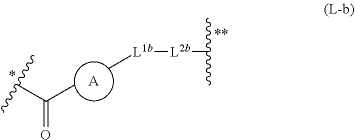

wherein Ring A is C₄₋₆ cycloalkylene or C₇₋₉ bridged bicyclic cycloalkylene; L^{1b} is —CH₂—NH—C(O)—, —NHC(O)—, or —C(O)NH—; L^{2b} is C₆₋₁₂ linear alkylene, wherein 1, 2, 3, or 4 methylene units are replaced with —O—, —NR^{1b}—, —C(O)NR^{1b}, or —NR^{1b}C(O)—; or L^{2b} is

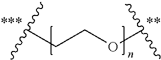

wherein n is 1, 2, 3, or 4, and

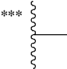

represents a covalent bond to L^{1b}; and each R^{1b} is independently hydrogen or C₁₋₃ alkyl;
(iv)

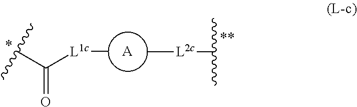

wherein L^{1c} is C₂₋₁₀ linear alkylene, wherein 1, 2, or 3 methylene units are replaced with —O—, —NH—, —NHC(O)—, or —C(O)NH—; Ring A is C₄₋₆ cycloalkylene or C₇₋₉ bridged bicyclic cycloalkylene; and L^{2c} is —O— or a saturated C₂₋₁₀ linear alkylene, wherein 1, 2, or 3 methylene units are replaced with —O—, —NH—, —NHC(O)—, or —C(O)NH—;
(v)

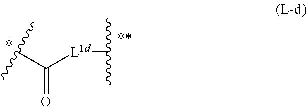

wherein L^{1d} is C₁₂₋₂₂ linear alkylene, wherein 1, 2, 3, 4, or 5 methylene units are replaced with —NH—, —O—, —C(O)NH—, —NHC(O)—, or —NHC(O)—NH—;
(vi)

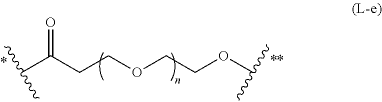

wherein n is an integer of 3 to 50;
(vii)

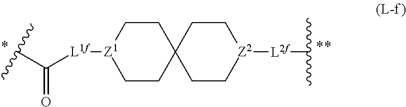

wherein L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—; L^{2f} is a bond, —NHC(O)—, —C(O)NH—, or a C₁₋₆ linear alkylene, wherein 0, 1, or 2 methylene units are replaced with —O—; and each of Z¹ and Z² is independently N or CH;
(viii)

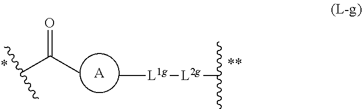

wherein Ring A is a 5 to 6 membered heteroarylene having 1 or 2 nitrogen ring atoms; L^{1g} is a bond, —CH₂—, —NH—, or —O—; and L²-g is

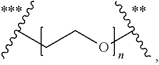

wherein n is 1, 2, 3, 4, or 5, and

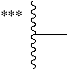

represents a covalent bond to L^{1g};
(ix)

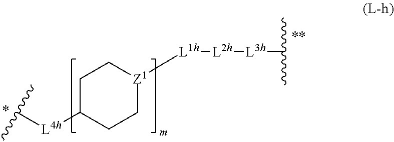

wherein each Z¹ is independently N or CH; L^{1h} is a bond, —C(O)—, —C(O)—NH—, or —NHC(O)—; L^{2h} is C₂-10 linear alkylene or

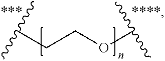

wherein n is 1, 2, 3, or 4, and

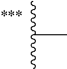

represents a covalent bond to L^{1h} and

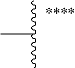

represents a covalent bond to L^{3h}; L^{3h} is a bond, —C(O)CH₂—, —O—(C₃₋₆ cycloalkylene)-O—, or —C(O)NH(CH₂)₃OCH₂—; L^{4h} is a bond, —C(O)—, —CH₂C(O)—, or —C(O)CH₂—; and m is 1, 2, or 3;
(x)

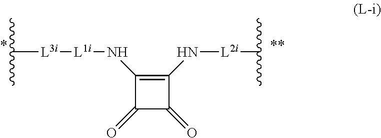

wherein L¹ⁱ is a bond, C₁₋₁₂ linear alkylene, or

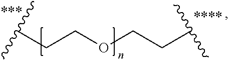

wherein n is 1, 2, 3, 4, or 5, and

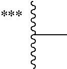

represents a covalent bond to L³ⁱ and

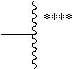

represents a covalent bond to NH; L²ⁱ is a bond, C₁₋₁₂ linear alkylene, or

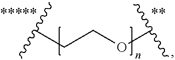

wherein n is 1, 2, 3, 4, or 5, and

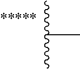

represents a covalent bond to HN; and L³ⁱ is a bond or —C(O)—;
(xi)

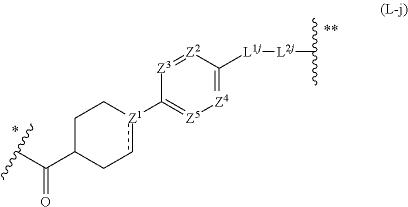

wherein Z¹ is C, CH, or N; each of Z², Z³, Z⁴ and Z⁵ is independently CH or N, provided that no more than two of Z², Z³, Z⁴ and Z⁵ are N; L^{1j} is —NH—, —C(O)NH—, —NHC(O)—, or —O—; L^{2j} is C₁₋₆ linear alkylene or

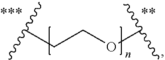

wherein n is 1 or 2, and

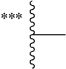

represents a covalent bond to L¹ⁱ; and [symbol] represents a single bond or a double bond;
(xii)

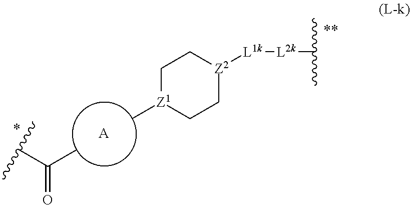

wherein Ring A is phenyl or a 5 or 6 membered heteroarylene having 1 or 2 nitrogen ring atoms; each of Z¹ and Z² is independently CH or N; L^{1k} is a bond, —C(O)—, —C(O)NH— or —NHC(O)—; and L^{2k} is a C₃₋₈ straight chain alkylene or

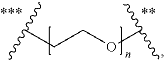

wherein n is 1, 2, or 3, and

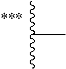

represents a covalent bond to L^{1k};
(xiii)

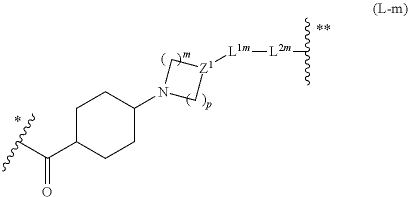

wherein Z¹ is CH or N; m is 1 or 2; p is 1 or 2; 0, 1, or 2 hydrogen atoms of

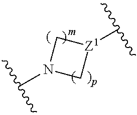

are replaced with F; L^{1m} is a bond, —C(O)—, —C(O)NH—, —NHC(O)—, —S(O)₂NH— or —NHS(O)₂—; and L^{2m} is C₃₋₆ linear alkylene, C₃₋₆ cycloalkylene, or

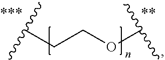

wherein n is 1 or 2, and

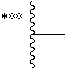

represents a covalent bond to L^{1m};
(xiv) one of

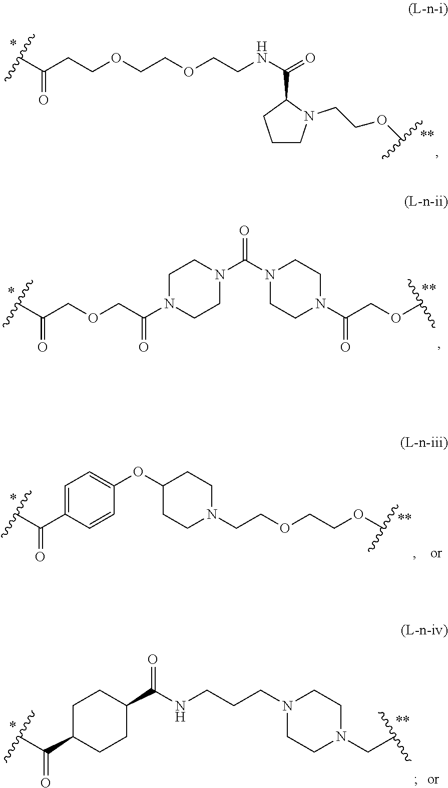

(xv)

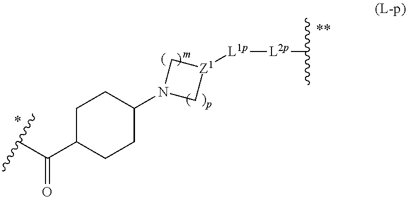

wherein Z¹ is CH or N; m is 1 or 2; p is 1 or 2; 0, 1, or 2 hydrogen atoms of

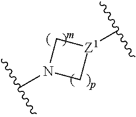

are replaced with F; L^{1p} is a bond, —C(O)—, —C(O)NH—, —NHC(O)—, —S(O)₂NH— or —NHS(O)₂—; and L^{2p} is -(4-6 membered saturated heterocyclylene containing 1 or 2 heteroatoms independently selected from nitrogen, oxygen, and sulfur)-C(O))—; wherein each

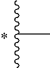

represents a covalent bond to Y¹, and each

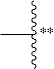

represents a covalent bond to X¹.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, optionally substituted 3-10 membered heterocyclyl containing 1, 2, 3, or 4 heteroatoms independently selected from nitrogen, oxygen, and sulfur, or optionally substituted 5-6 membered heteroaryl containing 1, 2, or 3 heteroatoms independently selected from nitrogen, oxygen, and sulfur.

In certain embodiments, L is a divalent linker of Formula (L-a), (L-b), (L-c), (L-d), (L-e), (L-f), (L-g), (L-h), (L-i), (L-j), (L-k), (L-m), (L-n-i), (L-n-ii), (L-n-iii), or (L-n-iv).

In certain embodiments, L is a divalent linker of Formula (L-a):

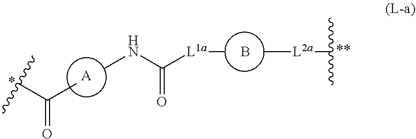

wherein:
Ring A and Ring B are each independently C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

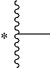

represents a covalent bond to Y¹, and

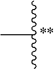

represents a covalent bond to X¹.

In certain embodiments, Ring A and Ring B of Formula (L-a) are each independently

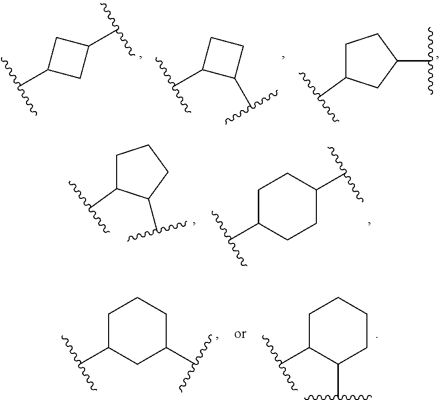

In certain embodiments, L is a divalent linker of Formula (L-a-i):

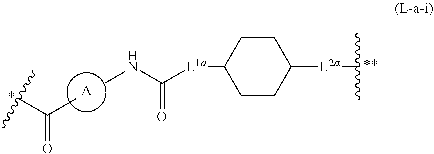

wherein:
Ring A is C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

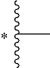

represents a covalent bond to Y¹, and

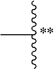

represents a covalent bond to X¹.

In certain embodiments, Ring A of Formula (L-a-i) is

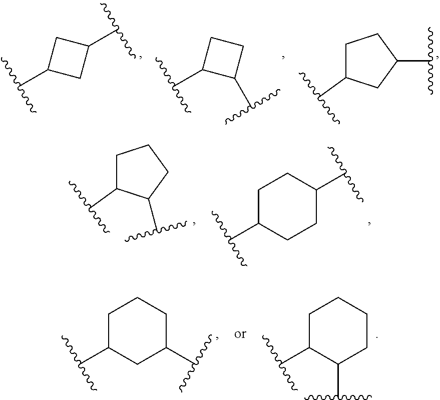

In certain embodiments, L is a divalent linker of Formula (L-a-ii):

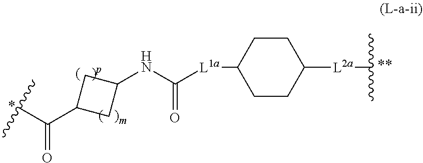

wherein:
L^{1a} is C₃₋₅ linear alkylene, wherein 1 or 2 methylene units are replaced with —O— or —NR^{a}—;
each R^{a} is independently hydrogen or C₁₋₃ alkyl;
L^{2a} is —O—, —NHC(O)—, or —CH₂—O—;
p is 1 or 2; and
m is 1 or 2;
wherein

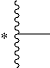

represents a covalent bond to Y¹, and

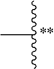

represents a covalent bond to X¹.

In certain embodiments, L^{1a} of Formula (L-a), (L-a-i), or (L-a-ii) is selected from

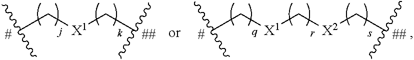

wherein:
j is 1, 2, 3, or 4;
k is 0, 1, 2, or 3;
the sum of j and k is 2, 3, or 4;
q is 1 or 2;
r is 1 or 2;
s is 0 or 1;
the sum of q, r, and s is 2 or 3;
X¹ and X² are independently —O— or NR^{a}; and
each R^{a} is independently hydrogen or C₁₋₃ alkyl;
wherein

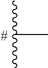

represents a covalent bond to the C(O) group of Formula (L-a), (L-a-i), or (L-a-ii), and

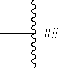

represents a covalent bond to Ring B of Formula (L-a) or to the cyclohexylene group of Formula (L-a-i) or (L-a-ii).
In certain embodiments, L^{1a} of Formula (L-a), (L-a-i), or (L-a-ii) is selected from —(CH₂)₂O—, —(CH₂)₃O—, —(CH₂)₄O—, —(CH₂)₂OCH₂—, —(CH₂)₃OCH₂—, —(CH₂)₂O(CH₂)₂—, —CH₂OCH₂—, —CH₂O(CH₂)₂—, —CH₂O(CH₂)₃—, —CH₂OCH₂O—, or —CH₂OCH₂OCH₂—. In certain embodiments, L^{1a} of Formula (L-a), (L-a-i), or (L-a-ii) is selected from —(CH₂)₂O—, —(CH₂)₃O—, —(CH₂)₂OCH₂—, or —(CH₂)₃OCH₂—. In certain embodiments, L^{1a} of Formula (L-a), (L-a-i), or (L-a-ii) is selected from —(CH₂)₂NR^{a}—, —(CH₂)₃NR^{a}—, —(CH₂)₄NR^{a}—, —(CH₂)₂NR^{a}CH₂—, —(CH₂)₃NR^{a}CH₂—, —(CH₂)₂NR^{a}(CH₂)₂—, —CH₂NR^{a}CH₂—, —CH₂NR^{a}(CH₂)₂—, —CH₂NR^{a}(CH₂)₃—, —CH₂NR^{a}CH₂NR^{a}—, or —CH₂NR^{a}CH₂NR^{a}CH₂—, wherein each R^{a} is independently hydrogen or C₁₋₃ alkyl. In certain embodiments, L^{1a} of Formula (L-a), (L-a-i), or (L-a-ii) is selected from —(CH₂)₂NR^{a}_, —(CH₂)₃NR^{a}—, —(CH₂)₂NR^{a}CH₂—, or —(CH₂)₃NR^{a}CH₂—, wherein R^{a} is hydrogen or C₁₋₃ alkyl. In certain embodiments, L^{1a} of Formula (L-a), (L-a-i), or (L-a-ii) is selected from —(CH₂)₂NH—, —(CH₂)₃NH—, —(CH₂)₄NH—, —(CH₂)₂NHCH₂—, —(CH₂)₃NHCH₂—, —(CH₂)₂NH(CH₂)₂—, —CH₂NHCH₂—, —CH₂NH(CH₂)₂—, —CH₂NH(CH₂)₃—, —CH₂NHCH₂NH—, or —CH₂NHCH₂NHCH₂—. In certain embodiments, L^{1a} of Formula (L-a), (L-a-i), or (L-a-ii) is selected from —(CH₂)₂NH—, —(CH₂)₃NH—, —(CH₂)₂NHCH₂—, or —(CH₂)₃NHCH₂—. In certain embodiments, L^{1a} of Formula (L-a), (L-a-i), or (L-a-ii) is selected from —CH₂OCH₂NR^{a}—, —CH₂NR^{a}CH₂O—, —CH₂OCH₂NR^{a}CH₂—, —CH₂NR^{a}CH₂OCH₂—, wherein R^{a} is independently hydrogen or C₁₋₃ alkyl. In certain embodiments, L^{1a} of Formula (L-a), (L-a-i), or (L-a-ii) is selected from —CH₂OCH₂NH—, —CH₂NHCH₂O—, —CH₂OCH₂NHCH₂—, —CH₂NHCH₂OCH₂—.

In certain embodiments, L is a divalent linker of Formula (L-a-iii):

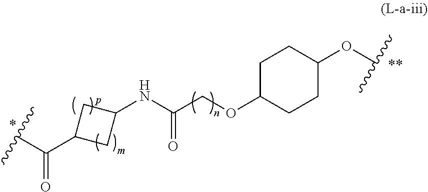

wherein:
p is 1 or 2;
m is 1 or 2; and
n is 1, 2, or 3;
wherein

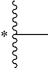

represents a covalent bond to Y¹, and

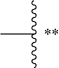

represents a covalent bond to X¹.

In certain embodiments, L is a divalent linker of Formula (L-a) selected from the group consisting of:

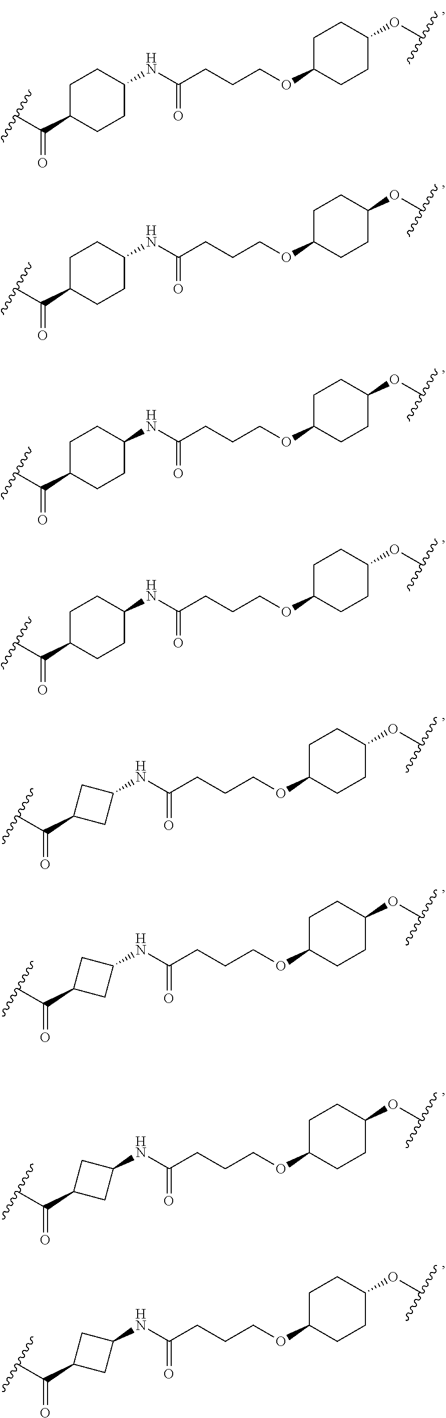

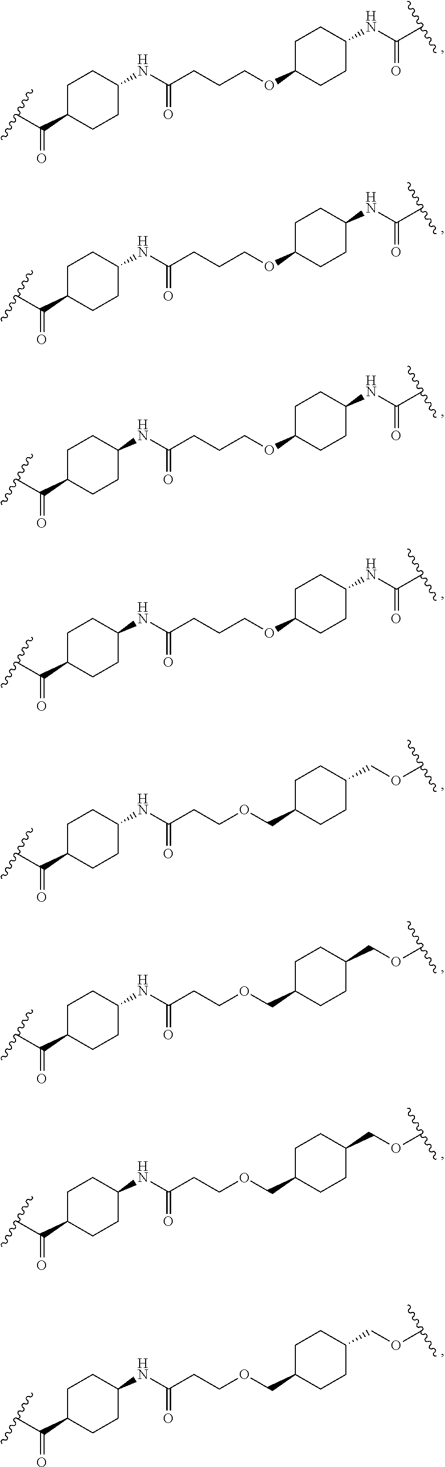

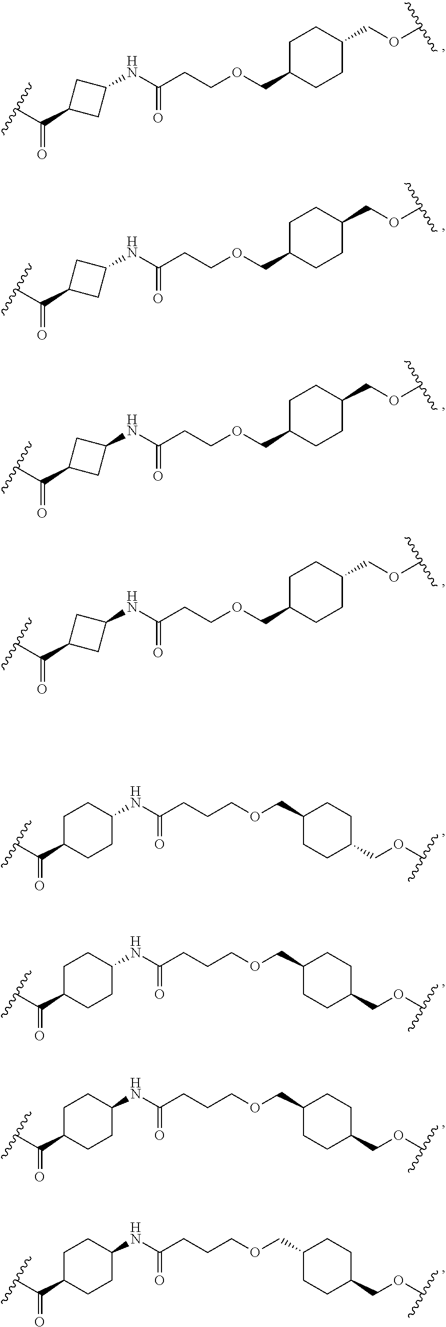

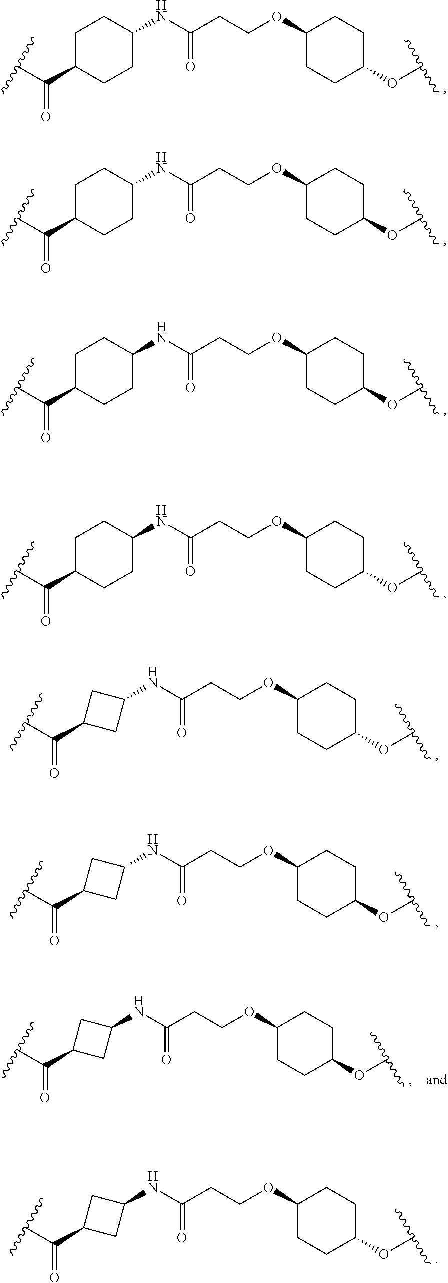

In the preceding embodiment, it is understood that for linkers of Formula (L-a), the point of attachment indicated on the cycloalkyl-bound carbonyl group is the attachment point to Y¹.

In certain embodiments, L is selected from the group consisting of:

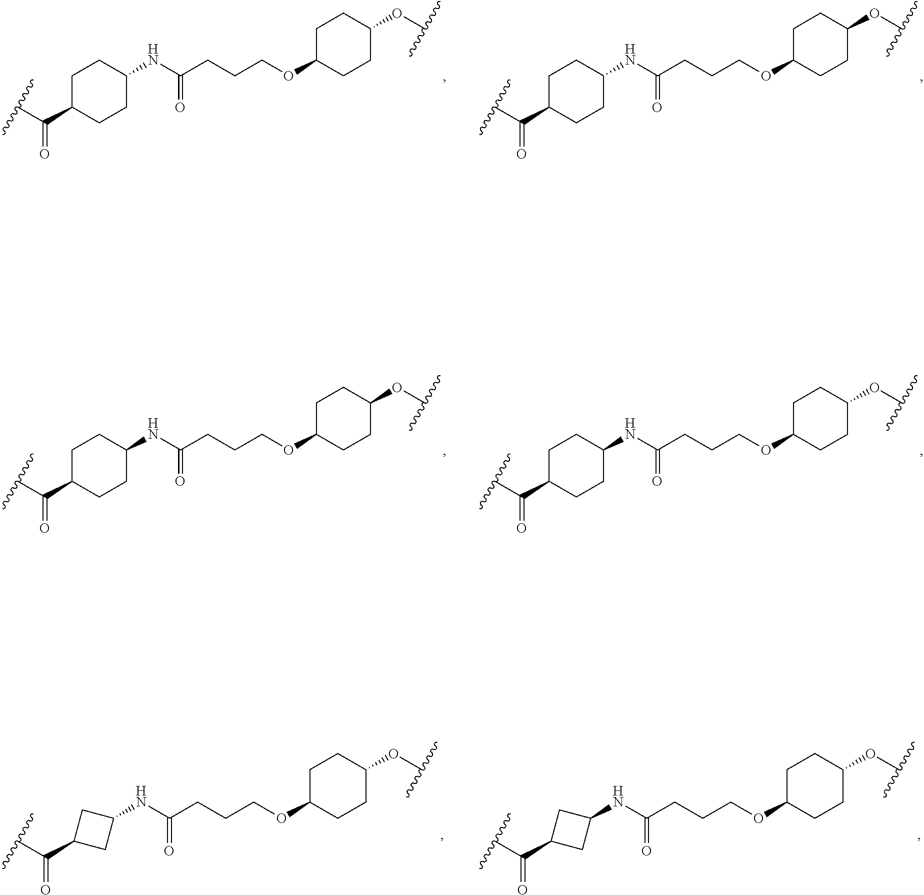 [SMILES: *O[C@H]1CC[C@H](OCCCC(=O)N[C@H]2CC[C@H](C(*)=O)CC2)CC1 ; *O[C@H]1CC[C@@H](OCCCC(=O)N[C@H]2CC[C@H](C(*)=O)CC2)CC1 ; *O[C@H]1CC[C@@H](OCCCC(=O)N[C@H]2CC[C@@H](C(*)=O)CC2)CC1 ; *O[C@H]1CC[C@H](OCCCC(=O)N[C@H]2CC[C@@H](C(*)=O)CC2)CC1 ; *O[C@H]1CC[C@H](OCCCC(=O)N[C@H]2C[C@H](C(*)=O)C2)CC1 ; *O[C@H]1CC[C@H](OCCCC(=O)N[C@H]2C[C@@H](C(*)=O)C2)CC1]

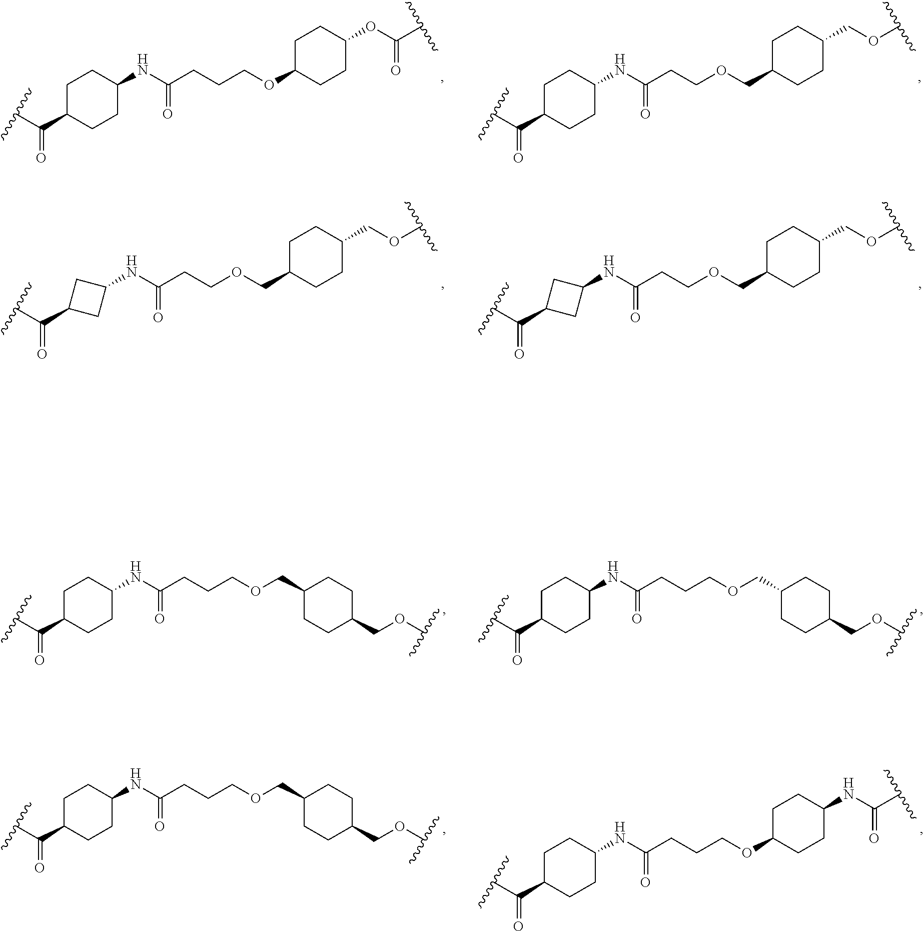

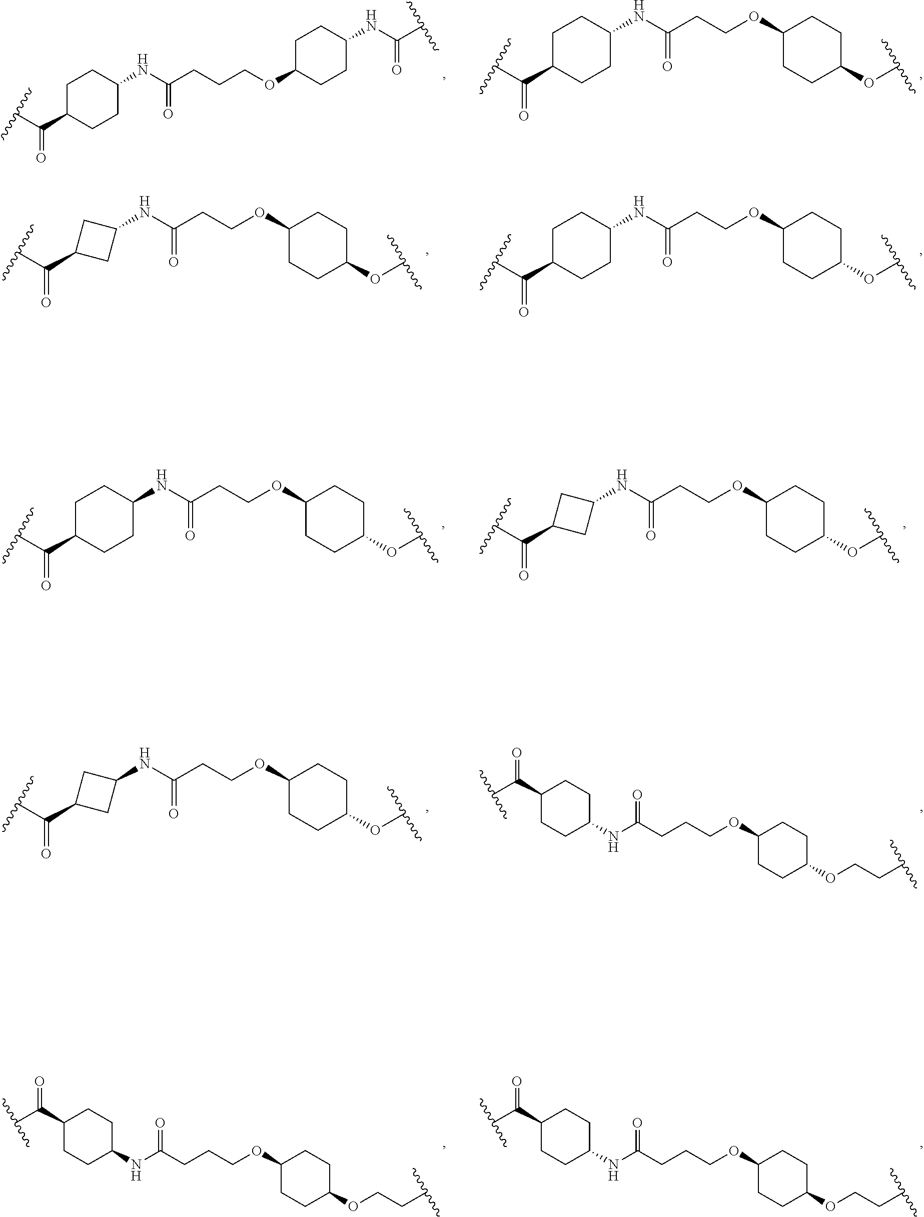

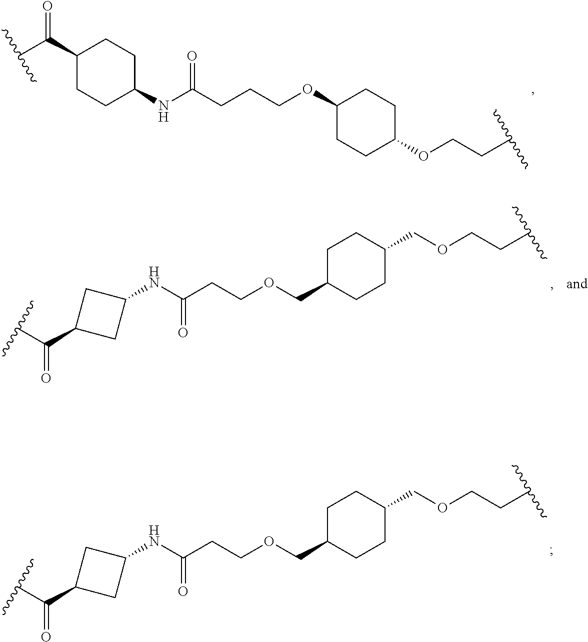 [SMILES: *CCO[C@H]1CC[C@H](OCCCC(=O)N[C@H]2CC[C@@H](C(*)=O)CC2)CC1 ; *CCOC[C@H]1CC[C@H](COCCC(=O)N[C@H]2C[C@H](C(*)=O)C2)CC1 ; *CCOC[C@H]1CC[C@H](COCCC(=O)N[C@H]2C[C@H](C(*)=O)C2)CC1]

wherein the point of attachment indicated on the cycloalkyl-bound carbonyl group is the attachment point to Y¹.

In certain embodiments, L is

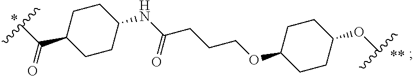 [SMILES: *O[C@H]1CC[C@H](OCCCC(=O)N[C@H]2CC[C@H](C(*)=O)CC2)CC1]

wherein

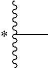

a covalent bond to Y¹, and

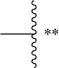

represents a covalent bond to X¹.

In certain embodiments, L is a divalent linker of Formula (L-b):

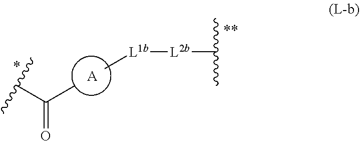

wherein:
Ring A is C₄₋₆ cycloalkylene or C₇₋₉ bridged bicyclic cycloalkylene;
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}, —C(O)NR^{1b}—, or —N^{1b}C(O)—; or
L^{2b} is

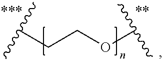

wherein n is 1, 2, 3, or 4, and

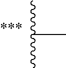

represents a covalent bond to L^{1b}; and
each R^{1b} is independently hydrogen or C₁₋₃ alkyl;
wherein

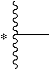

represents a covalent bond to Y¹, and

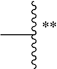

represents a covalent bond to X¹.

In certain embodiments, Ring A of Formula (L-b) is

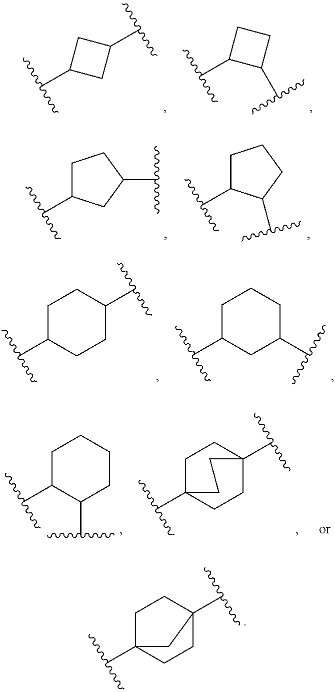

In certain embodiments, L is a divalent linker of Formula (L-b-i):

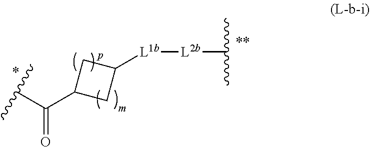

wherein:
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}, —C(O)NR^{1b}—, or —NR C(O)—; or
L^{2b} is

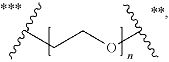

wherein n is 1, 2, 3, or 4, and

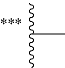

represents a covalent bond to L^{1b};
each R^{1b} is independently hydrogen or C₁₋₃ alkyl;
p is 1 or 2; and
m is 1 or 2;
wherein

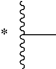

represents a covalent bond to Y¹, and

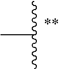

represents a covalent bond to X¹.

In certain embodiments, L^{2b} of Formula (L-b) or (L-b-i) is selected from

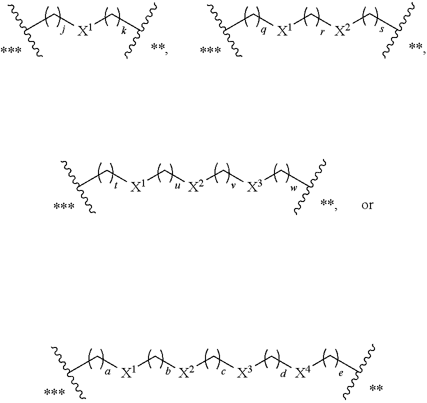

wherein:
j is 1, 2, 3, 4, 5, 6, 7, 8, 9, or 10;
k is 1, 2, 3, 4, 5, 6, 7, 8, 9, or 10;
the sum of j and k is 5, 6, 7, 8, 9, 10, or 11;
q is 1, 2, 3, 4, 5, 6, 7, 8, or 9;
r is 1, 2, 3, 4, 5, 6, 7, 8, or 9;
s is 0, 1, 2, 3, 4, 5, 6, 7, or 8;
the sum of q, r, and s is 4, 5, 6, 7, 8, 9, or 10;
tis 1, 2, 3, 4, 5, 6, or 7;
u is 1, 2, 3, 4, 5, 6, or 7;
v is 1, 2, 3, 4, 5, 6, or 7;
w is 0, 1, 2, 3, 4, 5, or 6;
the sum of t, u, v, and w is 3, 4, 5, 6, 7, 8, or 9;
a is 1, 2, 3, 4, or 5;
b is 1, 2, 3, 4, or 5;
c is 1, 2, 3, 4, or 5;
d is 1, 2, 3, 4, or 5;
e is 0, 1, 2, 3, or 4;
the sum of a, b, c, d, and e is 4, 5, 6, 7, or 8;
X¹, X², X³, and X⁴ are independently —O—, —NR^{1b}—, —C(O)NR^{1b}—, or —NR^{1b}C(O)—; and
each R^{1b} is independently hydrogen or C₁₋₃ alkyl;
wherein

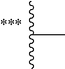

represents a covalent bond to L^{1b} of Formula (L-b) or (L-b-i), and

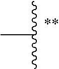

represents a covalent bond to X¹.

In certain embodiments, L is a divalent linker of Formula (L-b) selected from the group consisting of:

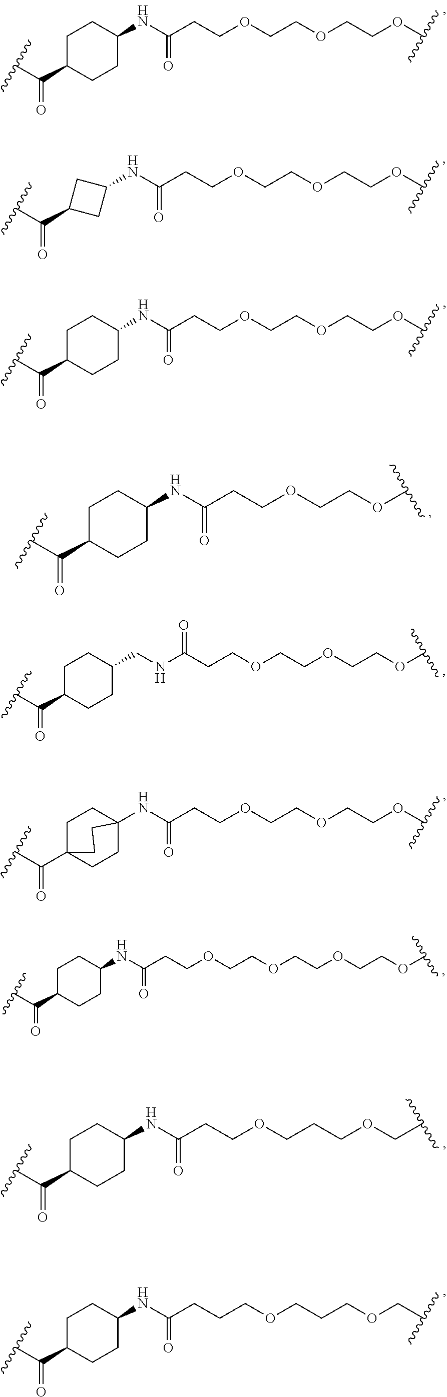

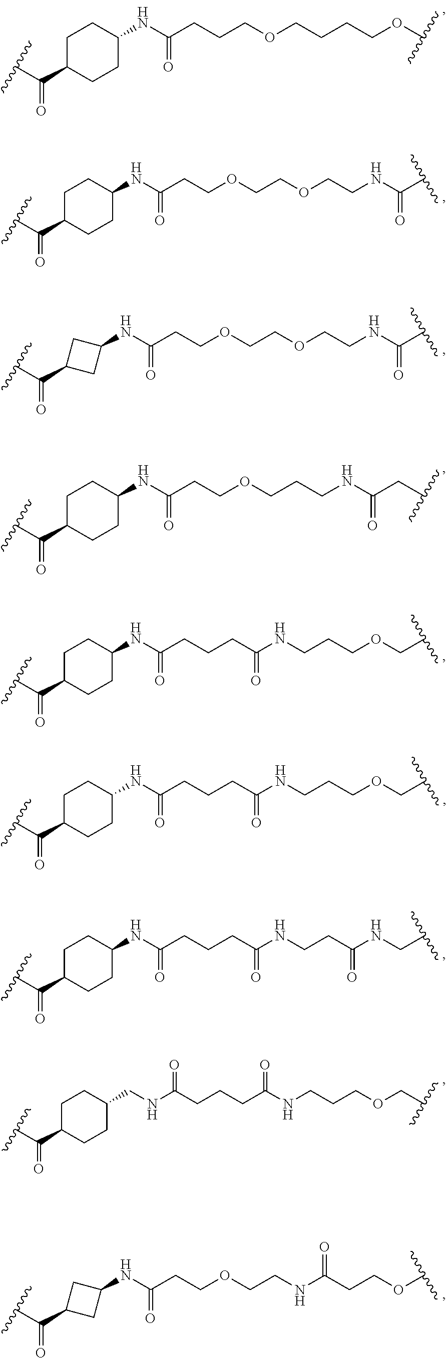

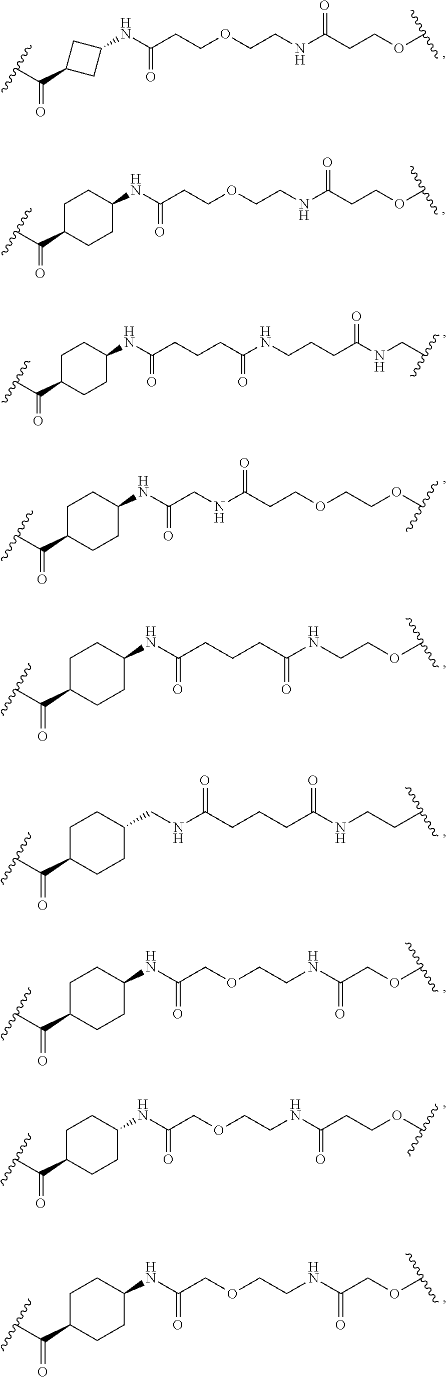

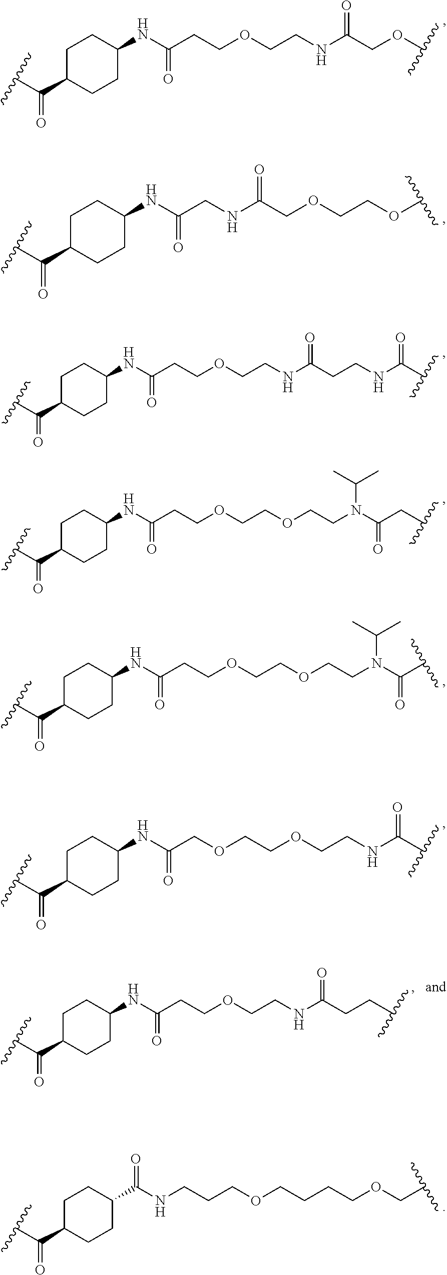

In the preceding embodiment, it is understood that for linkers of Formula (L-b), the point of attachment indicated on the cycloalkyl-bound carbonyl group is the attachment point to Y¹.

In certain embodiments, L is selected from the group consisting of:

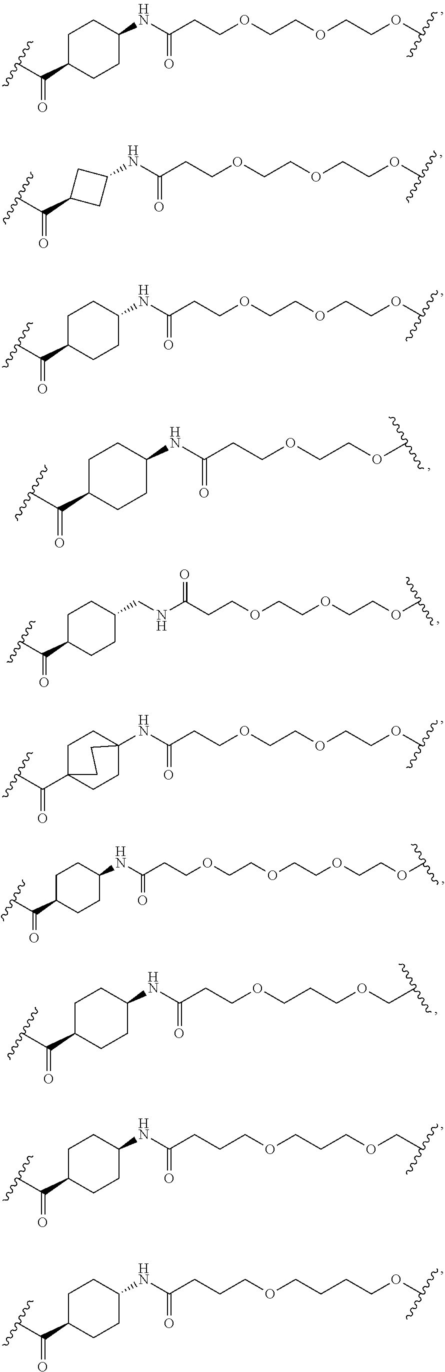

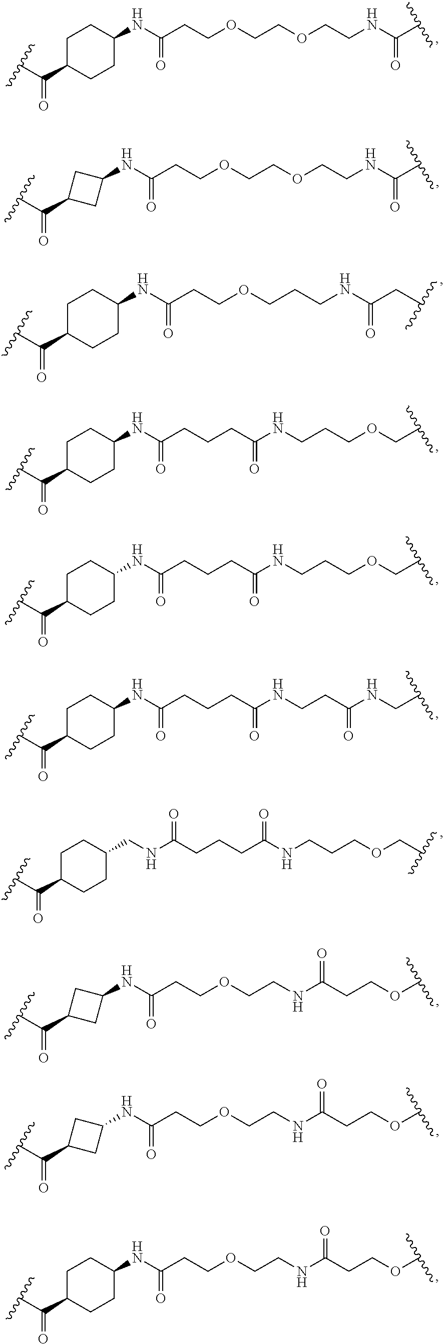

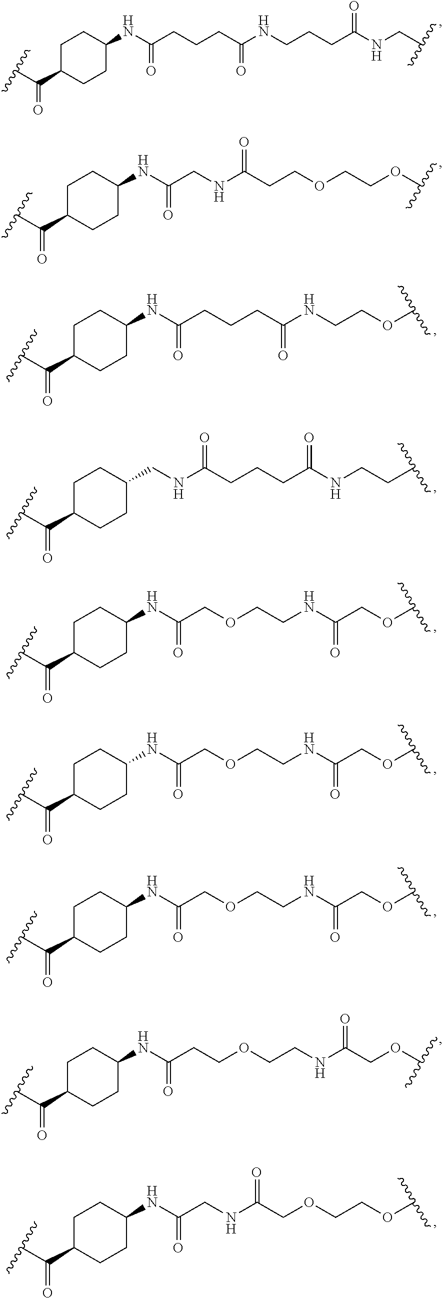

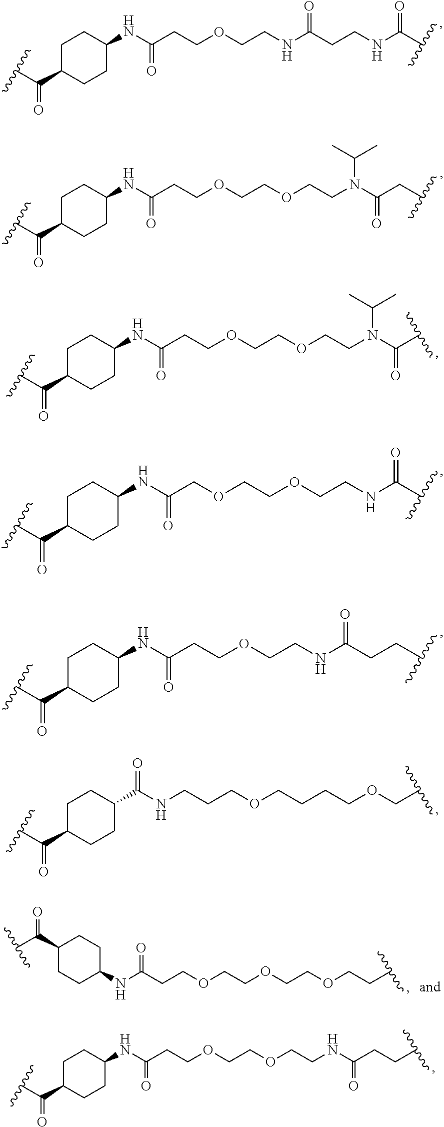

wherein the point of attachment indicated on the cycloalkyl-bound carbonyl group is the attachment point to Y¹.

In certain embodiments, L is a divalent linker of Formula (L-c):

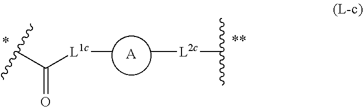

wherein:
L^{1c} is C₂₋₁₀ linear alkylene, wherein 1, 2, or 3 methylene units are replaced with —O—, —NH—, —NHC(O)—, or —C(O)NH—;
Ring A is C₄₋₆ cycloalkylene or C₇₋₉ bridged bicyclic cycloalkylene; and
L^{2c} is —O— or a saturated C₂₋₁₀ linear alkylene, wherein 1, 2, or 3 methylene units are replaced with —O—, —NH—, —NHC(O)—, or —C(O)NH—;
wherein

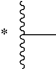

represents a covalent bond to Y¹, and

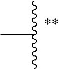

represents a covalent bond to X¹.

In certain embodiments, Ring A of Formula (L-c) is

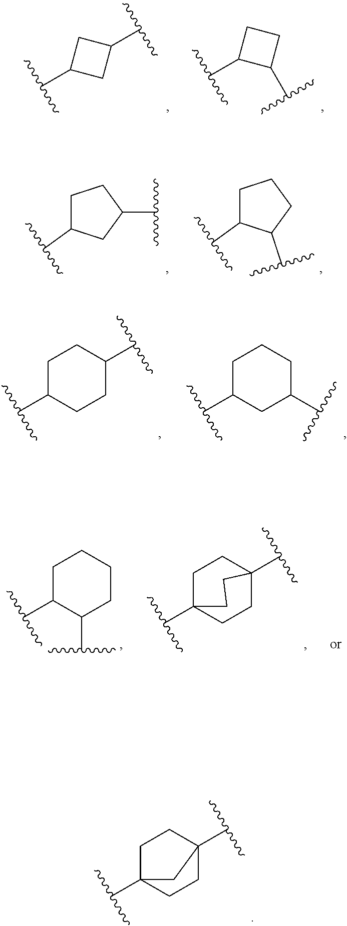

In certain embodiments, L is a divalent linker of Formula (L-c-i):

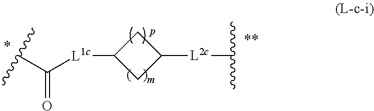

wherein:
L^{1c} is C₂₋₁₀ linear alkylene, wherein 1, 2, or 3 methylene units are replaced with —O—, —NH—, —NHC(O)—, or —C(O)NH—;
L^{2c} is —O— or a saturated C₂₋₁₀ linear alkylene, wherein 1, 2, or 3 methylene units are replaced with —O—, —NH—, —NHC(O)—, or —C(O)NH—;
p is 1 or 2; and
m is 1 or 2;
wherein

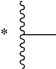

represents a covalent bond to Y¹, and

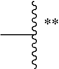

represents a covalent bond to X¹.

In certain embodiments, L^{1c} of Formula (L-c) or (L-c-i) is selected from

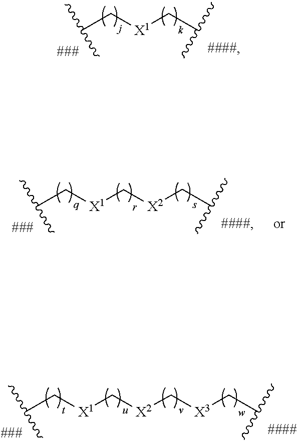

wherein:
j is 1, 2, 3,4,5,6,7,8, or 9;
k is 0, 1, 2, 3, 4, 5, 6, 7, or 8;
the sum of j and k is 1, 2, 3, 4, 5, 6, 7, 8, or 9;
q is 1, 2, 3, 4, 5, 6, or 7;
r is 1, 2, 3, 4, 5, 6, or 7;
s is 0, 1, 2, 3, 4, 5, or 6;
the sum of q, r, and s is 2, 3, 4, 5, 6, 7, or 8;
t is 1, 2, 3, 4, or 5;
u is 1, 2, 3, 4, or 5;
v is 1, 2, 3, 4, or 5;
w is 0, 1, 2, 3, or 4;
the sum of t, u, v, and w is 3, 4, 5, 6, or 7; and
X¹, X² and X³ are independently —O—, —NH—, —NHC(O)—, or —C(O)NH—;
wherein

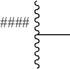

represents a covalent bond to the C(O) group of Formula (L-c) or (L-c-i), and

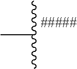

represents a covalent bond to the ring of Formula (L-c) or (L-c-i).

In certain embodiments, L^{2c} of Formula (L-c) or (L-c-i) is selected from

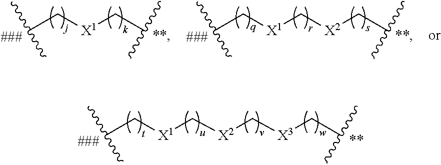

wherein:
j is 0, 1, 2, 3,4,5,6,7,8, or 9;
k is 0, 1, 2, 3,4,5,6,7,8, or 9;
the sum of j and k is 1, 2, 3, 4, 5, 6, 7, 8, or 9;
q is 0, 2, 3, 4, 5, 6, or 7;
r is 1, 2, 3, 4, 5, 6, 7, or 8;
s is 0, 1, 2, 3, 4, 5, 6, or 7;
the sum of q, r, and s is 1, 2, 3, 4, 5, 6, 7, or 8;
tis 0, 1, 2, 3, 4, or 5;
u is 1, 2, 3, 4, 5, or 6;
v is 1, 2, 3, 4, 5, or 6;
w is 0, 1, 2, 3, 4, or 5;
the sum of t, u, v, and w is 2, 3, 4, 5, 6, or 7; and
X¹, X² and X³ are independently —O—, —NH—, —NHC(O)—, or —C(O)NH—;
wherein

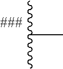

represents a covalent bond to the ring of Formula (L-c) or (L-c-i), and

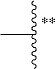

represents a covalent bond to X¹.

In certain embodiments, L is a divalent linker of Formula (L-c) selected from the group consisting of:

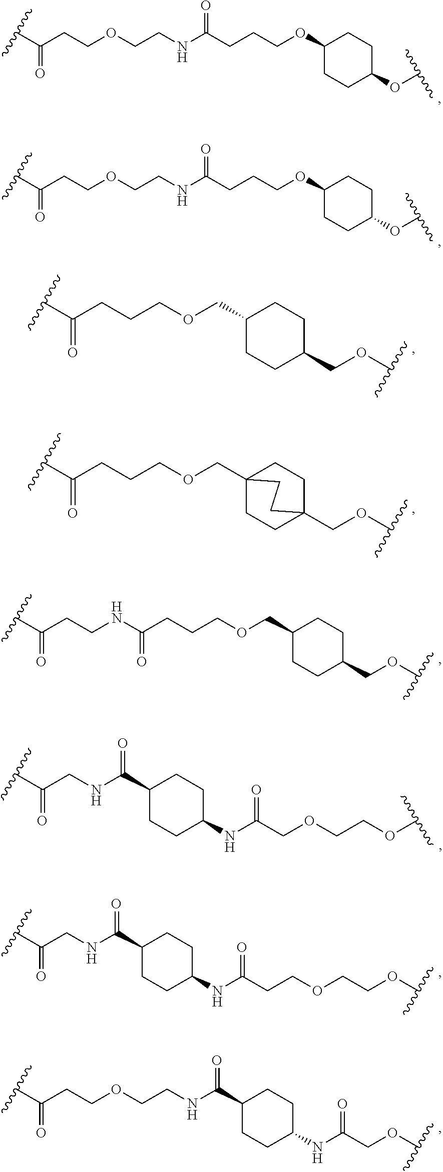

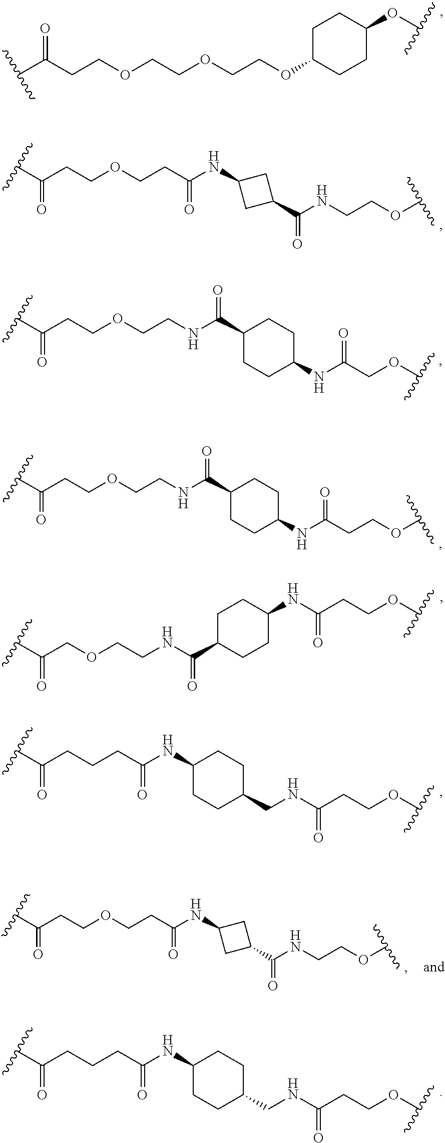

In the preceding embodiment, it is understood that for the depicted linkers of Formula (L-c), the point of attachment indicated on the carbonyl group is the attachment point to Y¹.

In certain embodiments, L is selected from the group consisting of

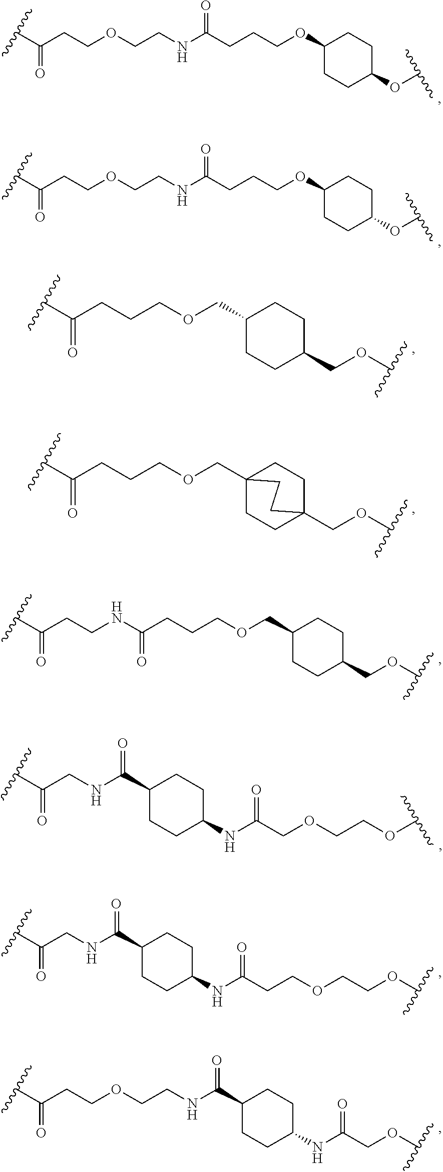

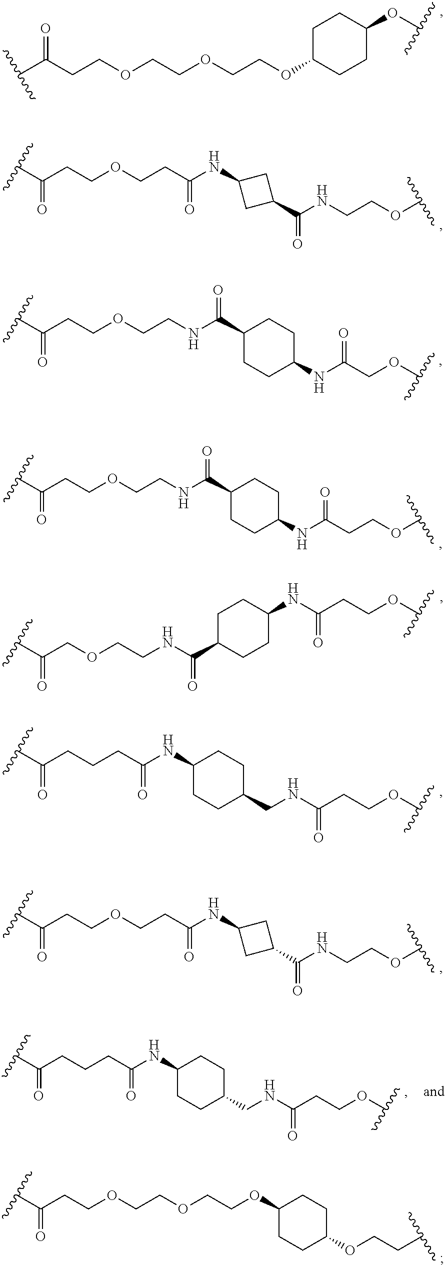

wherein the point of attachment indicated on the carbonyl group is the attachment point to Y¹.

In certain embodiments, L is a divalent linker of Formula (L-d):

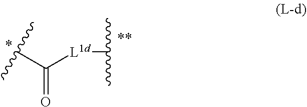

wherein:
L^{1d} is C₁₂₋₂₂ linear alkylene, wherein 1, 2, 3, 4, or 5 methylene units are replaced with —NH—, —O—, —C(O)NH—, —NHC(O)—, or —NHC(O)—NH—;
wherein

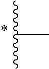

represents a covalent bond to Y¹, and

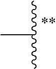

represents a covalent bond to X¹.

In certain embodiments, L^{1d} of Formula (L-d) is selected from

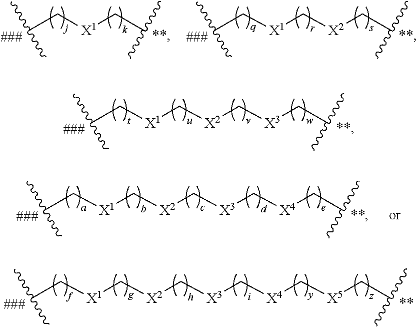

wherein:
j is 1, 2, 3, 4, 5, 6, 7, 8, 9, 10, 11, 12, 13, 14, 15, 16, 17, 18, 19, or 20;
k is 1, 2, 3, 4, 5, 6, 7, 8, 9, 10, 11, 12, 13, 14, 15, 16, 17, 18, 19, or 20;
the sum of j and k is 11, 12, 13, 14, 15, 16, 17, 18, 19, 20, or 21;
q is 1, 2, 3, 4, 5, 6, 7, 8, 9, 10, 11, 12, 13, 14, 15, 16, 17, 18, or 19;
r is 1, 2, 3, 4, 5, 6, 7, 8, 9, 10, 11, 12, 13, 14, 15, 16, 17, 18, or 19;
s is 0, 1, 2, 3, 4, 5, 6, 7, 8, 9, 10, 11, 12, 13, 14, 15, 16, 17, or 18;
the sum of q, r, and s is 10, 11, 12, 13, 14, 15, 16, 17, 18, 19, or 20;
tis 1, 2, 3, 4, 5, 6, 7, 8, 9, 10, 11, 12, 13, 14, 15, 16, or 17;
u is 1, 2, 3, 4, 5, 6, 7, 8, 9, 10, 11, 12, 13, 14, 15, 16, or 17;
v is 1, 2, 3, 4, 5, 6, 7, 8, 9, 10, 11, 12, 13, 14, 15, 16, or 17;
w is 0, 1, 2, 3, 4, 5, 6, 7, 8, 9, 10, 11, 12, 13, 14, 15, or 16;
the sum of t, u, v, and w is 9, 10, 11, 12, 13, 14, 15, 16, 17, 18, or 19;
a is 1, 2, 3, 4, 5, 6, 7, 8, 9, 10, 11, 12, 13, 14, or 15;
b is 1, 2, 3, 4, 5, 6, 7, 8, 9, 10, 11, 12, 13, 14, or 15;
c is 1, 2, 3, 4, 5, 6, 7, 8, 9, 10, 11, 12, 13, 14, or 15;
d is 1, 2, 3, 4, 5, 6, 7, 8, 9, 10, 11, 12, 13, 14, or 15;
e is 0, 1, 2, 3, 4, 5, 6, 7, 8, 9, 10, 11, 12, 13, or 14;
the sum of a, b, c, d, and e is 8, 9, 10, 11, 12, 13, 14, 15, 16, 17, or 18;
f is 1, 2, 3, 4, 5, 6, 7, 8, 9, 10, 11, 12, or 13;
g is 1, 2, 3, 4, 5, 6, 7, 8, 9, 10, 11, 12, or 13;
his 1, 2, 3, 4, 5, 6, 7, 8, 9, 10, 11, 12, or 13;
i is 1, 2, 3, 4, 5, 6, 7, 8, 9, 10, 11, 12, or 13;
y is 1, 2, 3, 4, 5, 6, 7, 8, 9, 10, 11, 12, or 13;
z is 0, 1, 2, 3, 4, 5, 6, 7, 8, 9, 10, 11, or 12;
the sum of f, g, h, i, y, and z is 7, 8, 9, 10, 11, 12, 13, 14, 15, 16, or 17; and
X¹, X², X³, X⁴, and X⁵ are independently —NH—, —O—, —C(O)NH—, —NHC(O)—, or —NHC(O)—NH—;
wherein

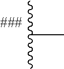

represents a covalent bond to the C(O) group of Formula (L-d), and

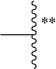

represents a covalent bond to X¹.

In certain embodiments, L is a divalent linker of Formula (L-d) selected from the group consisting of:

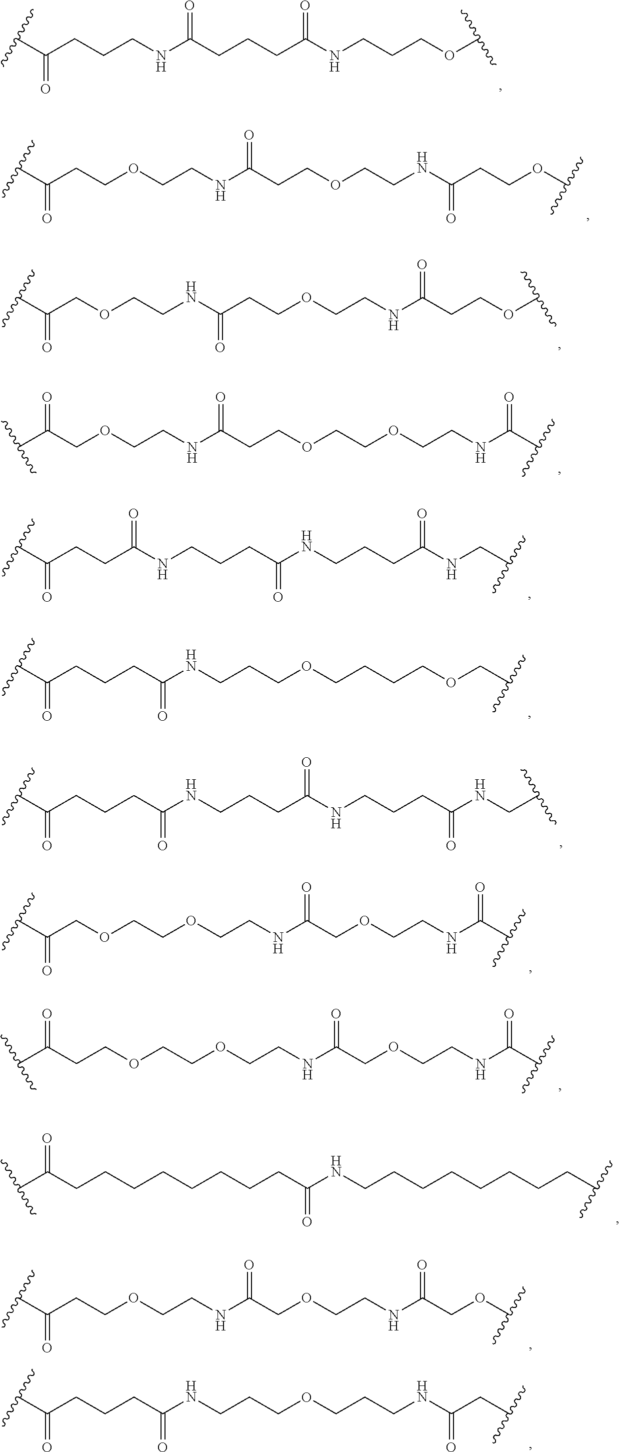

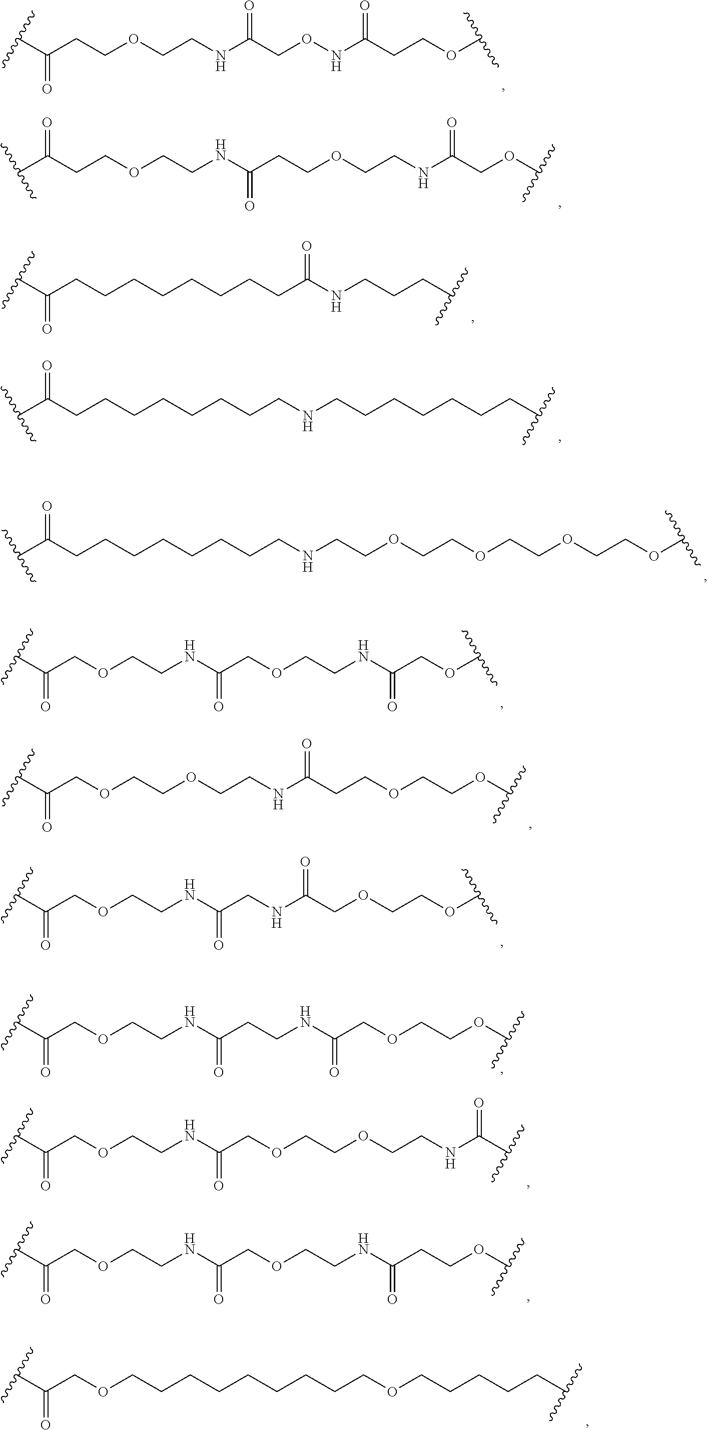

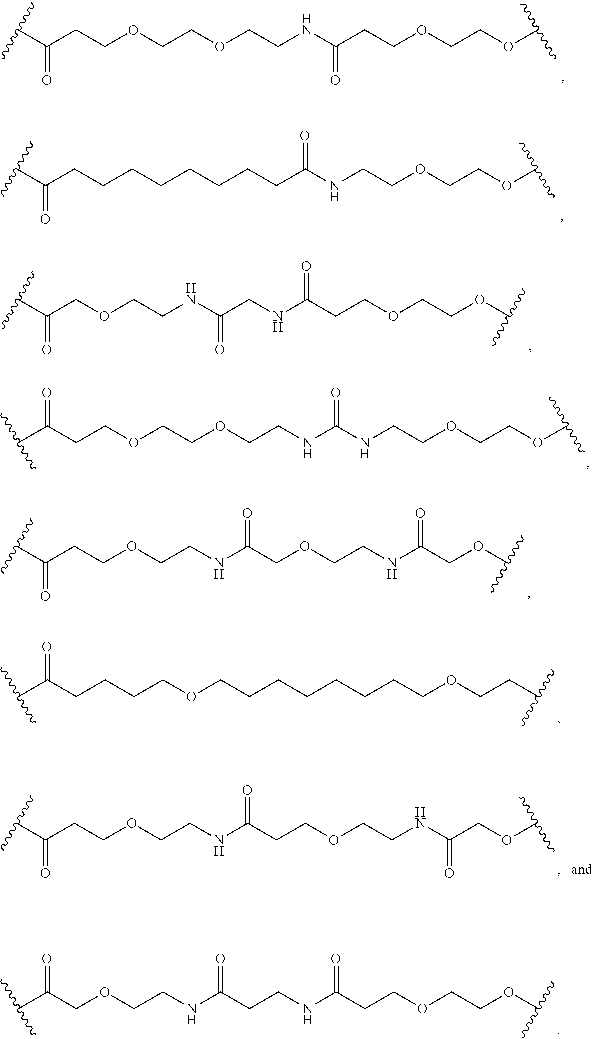

In the preceding embodiment, it is understood that for the depicted linkers of Formula (L-d), the point of attachment indicated on the carbon-bound carbonyl group is the attachment point to Y¹.

In certain embodiments, L is a divalent linker of Formula (L-e):

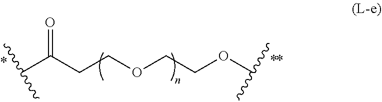

wherein:
n is an integer of 3 to 50;
wherein

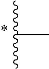

represents a covalent bond to Y¹, and

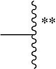

represents a covalent bond to X¹.

In certain embodiments, n of Formula (L-e) is 3 to 25, 3 to 10, 3 to 8, 3 to 7, 3 to 5, or 3 to 4. In certain embodiments, n of Formula (L-e) is 3, 4, 5, 7, 8, 22, or 50.

In certain embodiments, L is a divalent linker of Formula (L-f):

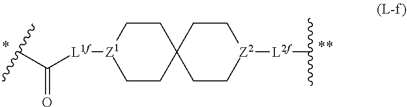

wherein:
L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—;
L^{2f} is a bond, —NHC(O)—, —C(O)NH—, or a C₁₋₆ linear alkylene, wherein 0, 1, or 2 methylene units are replaced with —O—; and
each of Z¹ and Z² is independently N or CH;
wherein

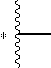

represents a covalent bond to Y¹, and

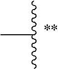

represents a covalent bond to X¹.

In certain embodiments, L^{1f} of Formula (L-f) is selected from

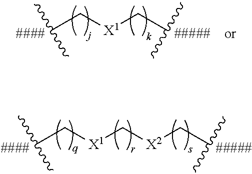

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2;
the sum of q, r, and s is 2, 3, or 4; and
X¹ and X² are independently —O—, —NH—, or —C(O)—; or —(C₃₋₆ cycloalkylene)-NHC(O)—;
wherein

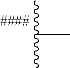

represents a covalent bond to the C(O) group of Formula (L-f), and

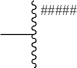

represents a covalent bond to the ring of Formula (L-f).

In certain embodiments, L^{2r} of Formula (L-f) is selected from

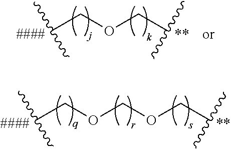

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2; and
the sum of q, r, and s is 2, 3, or 4;
wherein

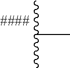

represents a covalent bond to the ring of Formula (L-f), and

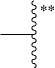

represents a covalent bond to X¹.

In certain embodiments, L is a divalent linker of Formula (L-f) selected from the group consisting of:

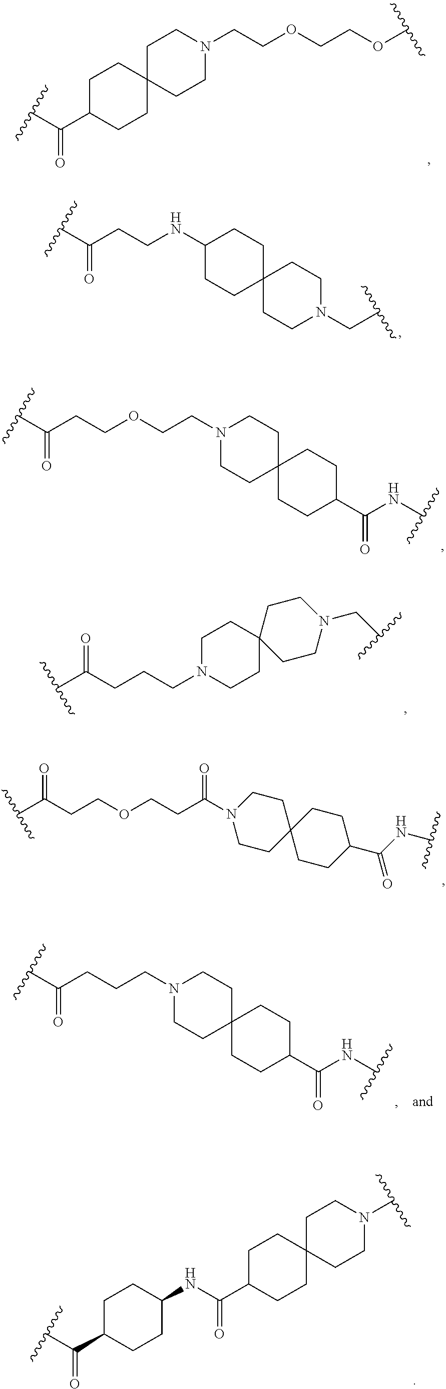

In the preceding embodiment, it is understood that for the depicted linkers of Formula (L-f), the point of attachment indicated on the carbonyl group is the attachment point to Y¹.

In certain embodiments, L is selected from the group consisting of:

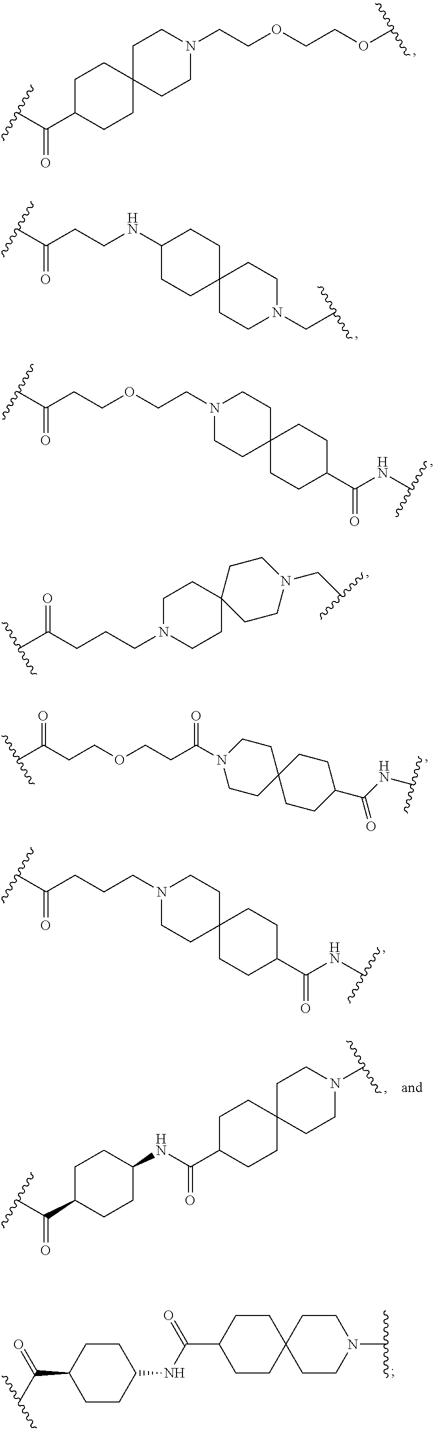

wherein the point of attachment indicated on the carbonyl group is the attachment point to Y¹.

In certain embodiments, L is a divalent linker of Formula (L-g):

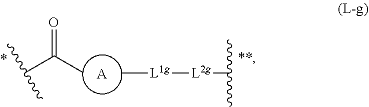

wherein:
Ring A is a 5 to 6 membered heteroarylene having 1 or 2 nitrogen ring atoms;
L^{1g} is a bond, —CH₂—, —NH—, or —O—; and

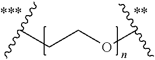

L^{2g} is n wherein n is 1, 2, 3, 4, or 5, and

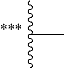

represents a covalent bond to L^{1g};
wherein

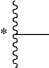

represents a covalent bond to Y¹, and

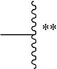

represents a covalent bond to X¹.

In certain embodiments, L is a divalent linker of Formula (L-g-i):

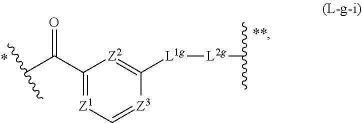

wherein:
L^{1g} is a bond, —CH₂—, —NH—, or —O—;
L^{2g} is

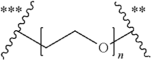

wherein n is 1, 2, 3, 4, or 5, and

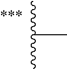

represents a covalent bond to L^{1g};
Z¹, Z², and Z³ are each independently selected from N or CH, provided that one or two of Z¹, Z², and Z³ is N;
wherein

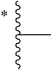

represents a covalent bond to Y¹, and

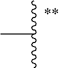

represents a covalent bond to X¹.

In certain embodiments, L is a divalent linker of Formula (L-g) selected from the group consisting of:

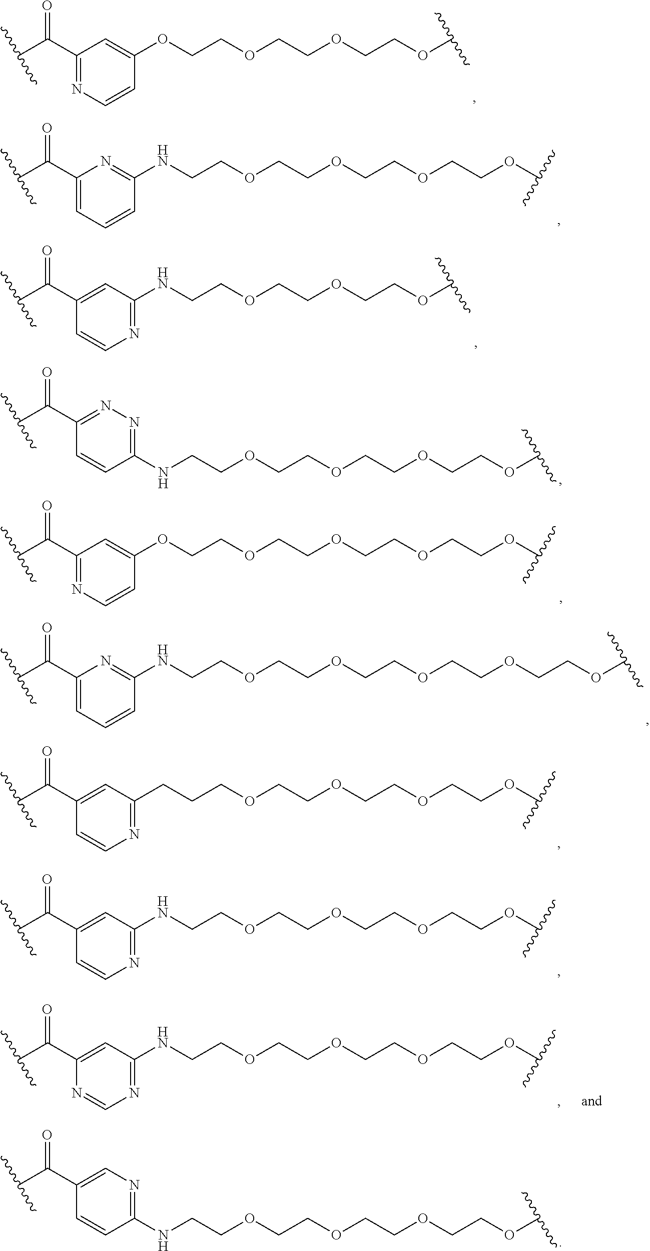

In the preceding embodiment, it is understood that for linkers of Formula (L-g), the point of attachment indicated on the carbonyl group is the attachment point to Y¹.

In certain embodiments, L is a divalent linker of Formula (L-h):

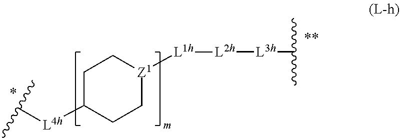

wherein:
each Z¹ is independently N or CH;
L^{1h} is a bond, —C(O)—, —C(O)—NH—, or —NHC(O)—;
L^{2h} is C₂₋₁₀ linear alkylene or

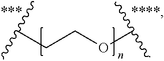

wherein n is 1, 2, 3, or 4, and

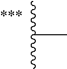

represents a covalent bond to L^{1h} and

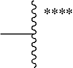

represents a covalent bond to L^{3h};
L^{3h} is a bond, —C(O)CH₂—, —O—(C₃₋₆ cycloalkylene)-O—, or —C(O)NH(CH₂)₃OCH₂—;
L^{4h} is a bond, —C(O)—, —CH₂C(O)—, or —C(O)CH₂—; and
m is 1, 2, or 3;
wherein

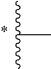

represents a covalent bond to Y¹, and

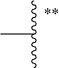

represents a covalent bond to X¹.

In certain embodiments, L is a divalent linker of Formula (L-h) selected from the group consisting of:

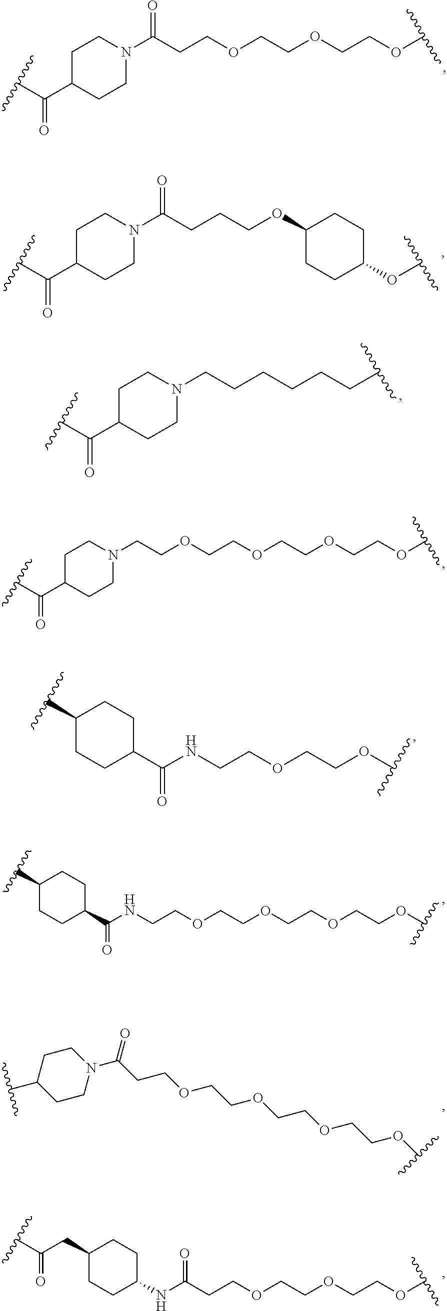

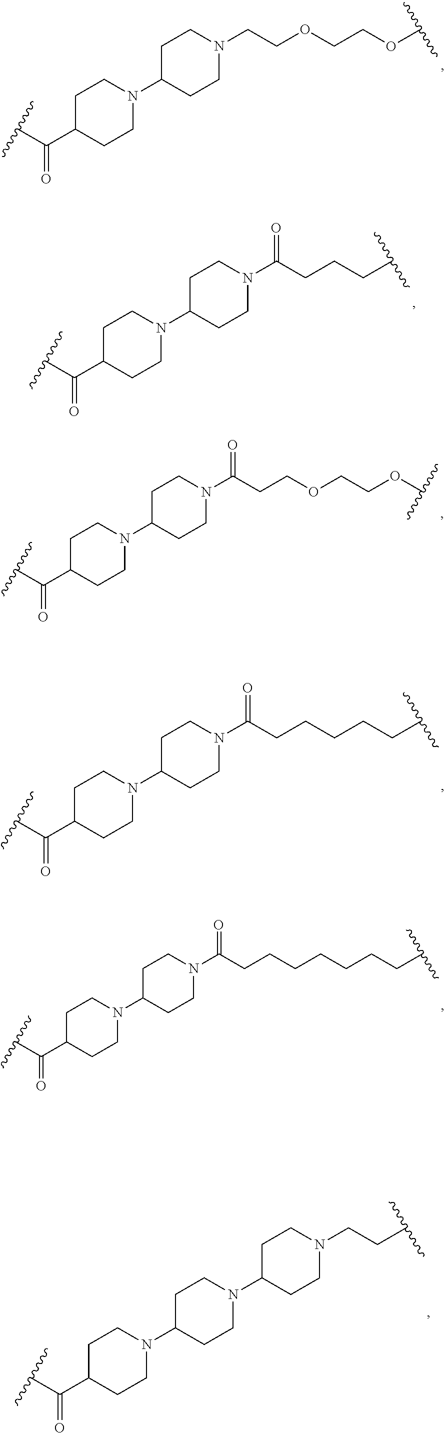 [SMILES: *OCCOCCN1CCC(N2CCC(C(*)=O)CC2)CC1 ; *CCCC(=O)N1CCC(N2CCC(C(*)=O)CC2)CC1 ; *OCCOCCC(=O)N1CCC(N2CCC(C(*)=O)CC2)CC1 ; *CCCCCC(=O)N1CCC(N2CCC(C(*)=O)CC2)CC1 ; *CCCCCCCC(=O)N1CCC(N2CCC(C(*)=O)CC2)CC1 ; *CCN1CCC(N2CCC(N3CCC(C(*)=O)CC3)CC2)CC1]

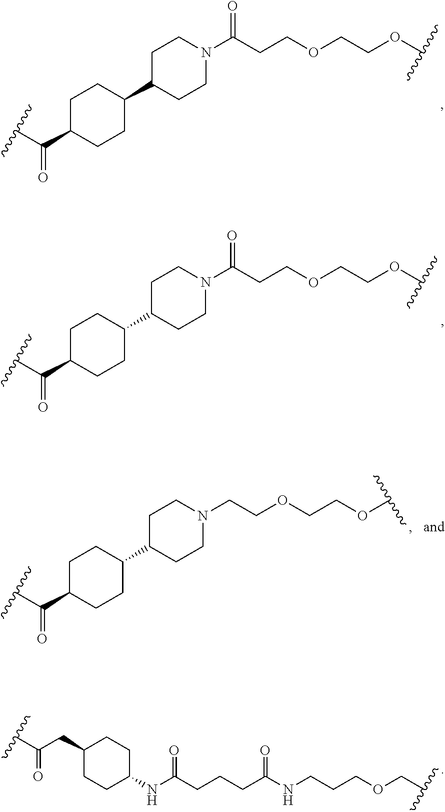 [SMILES: *OCCOCCC(=O)N1CCC([C@H]2CC[C@@H](C(*)=O)CC2)CC1 ; *OCCOCCC(=O)N1CCC([C@H]2CC[C@H](C(*)=O)CC2)CC1 ; *OCCOCCN1CCC([C@H]2CC[C@H](C(*)=O)CC2)CC1 ; *COCCCNC(=O)CCCC(=O)N[C@H]1CC[C@H](CC(*)=O)CC1]

In the preceding embodiment, it is understood that for the depicted linkers of Formula (L-h), the point of attachment indicated on the carbonyl group or cyclic group is the attachment point to Y¹.

In certain embodiments, L is a divalent linker of Formula (L-i):

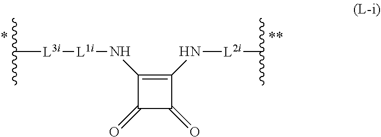

wherein:
L¹ⁱ is a bond, C₁₋₁₂ linear alkylene, or

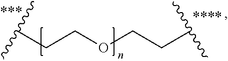

wherein n is 1, 2, 3, 4, or 5, and

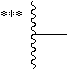

represents a covalent bond to L³ⁱ and

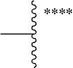

represents a covalent bond to NH;
L²ⁱ is a bond, C₁₋₁₂ linear alkylene, or

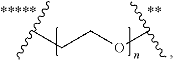

wherein n is 1, 2, 3, 4, or 5, and

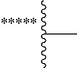

represents a covalent bond to HN; and
L³ⁱ is a bond or —C(O)—;
wherein

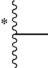

represents a covalent bond to Y¹, and

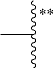

represents a covalent bond to X¹.

In certain embodiments, L is a divalent linker of Formula (L-i) selected from the group consisting of:

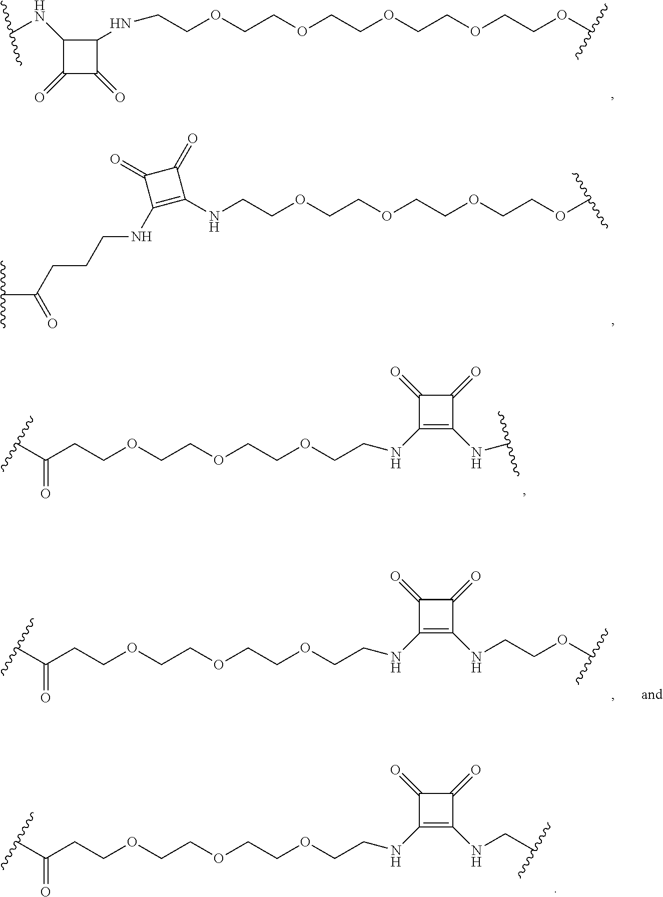 [SMILES: *NC1C(=O)C(=O)C1NCCOCCOCCOCCOCCO* ; *OCCOCCOCCOCCNc1c(NCCCC(*)=O)c(=O)c1=O ; *Nc1c(NCCOCCOCCOCCC(*)=O)c(=O)c1=O ; *OCCNc1c(NCCOCCOCCOCCC(*)=O)c(=O)c1=O ; *CNc1c(NCCOCCOCCOCCC(*)=O)c(=O)c1=O]

In certain embodiments, L is a divalent linker of Formula (L-j):

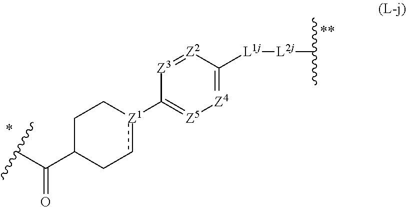

wherein:
Z is C, CH, or N;
each of Z², Z³, Z⁴ and Z⁵ is independently CH or N, provided that no more than two of Z², Z³, Z⁴ and Z⁵ are N;
L^{1j} is —NH—, —C(O)NH—, —NHC(O)—, or —O—;
L^{2j} is C₁₋₆ linear alkylene or

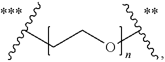

wherein n is 1 or 2, and

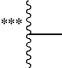

represents a covalent bond to L^{1j}; and
[symbol] represents a single bond or a double bond;
wherein

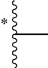

represents a covalent bond to Y¹, and

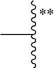

represents a covalent bond to X¹.

In certain embodiments, L is a divalent linker of Formula (L-j) selected from the group consisting of:

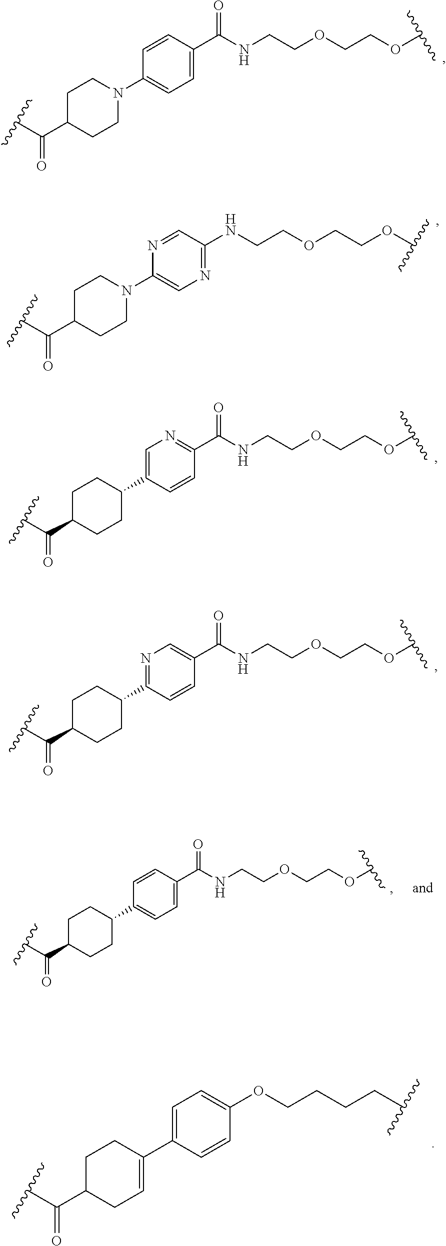 [SMILES: *OCCOCCNC(=O)c1ccc(N2CCC(C(*)=O)CC2)cc1 ; *OCCOCCNc1cnc(N2CCC(C(*)=O)CC2)cn1 ; *OCCOCCNC(=O)c1ccc([C@H]2CC[C@H](C(*)=O)CC2)cn1 ; *OCCOCCNC(=O)c1ccc([C@H]2CC[C@H](C(*)=O)CC2)nc1 ; *OCCOCCNC(=O)c1ccc([C@H]2CC[C@H](C(*)=O)CC2)cc1 ; *CCCCOc1ccc(C2=CCC(C(*)=O)CC2)cc1]

In the preceding embodiment, it is understood that for linkers of Formula (L-j), the point of attachment indicated on the carbonyl group is the attachment point to Y¹.

In certain embodiments, L is selected from the group consisting of

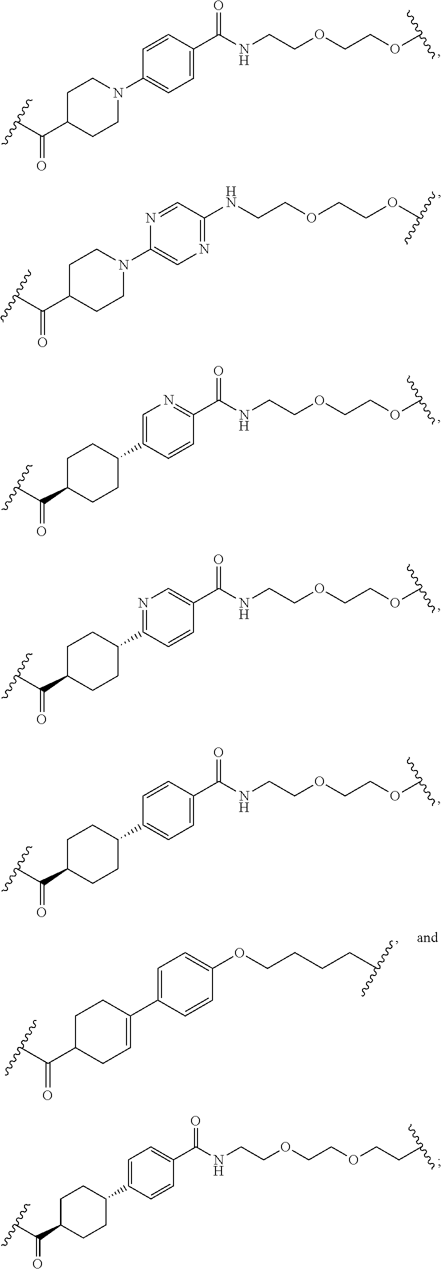

wherein the point of attachment indicated on the carbonyl group is the attachment point to Y¹.

In certain embodiments, L is a divalent linker of Formula (L-k):

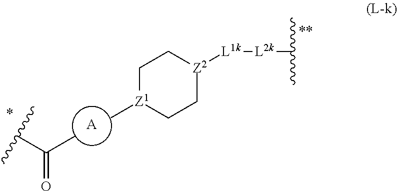

wherein:
Ring A is phenyl or a 5 or 6 membered heteroarylene having 1 or 2 nitrogen ring atoms;
each of Z¹ and Z² is independently CH or N;
L^{1k} is a bond, —C(O)—, —C(O)NH— or —NHC(O)—; and
L^{2k} is a C₃₋₈ straight chain alkylene or

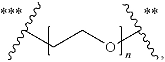

wherein n is 1, 2, or 3, and

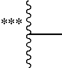

represents a covalent bond to L^{1k};
wherein

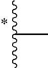

represents a covalent bond to Y¹, and

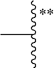

represents a covalent bond to X¹.

In certain embodiments, L is a divalent linker of Formula (L-k) selected from the group consisting of:

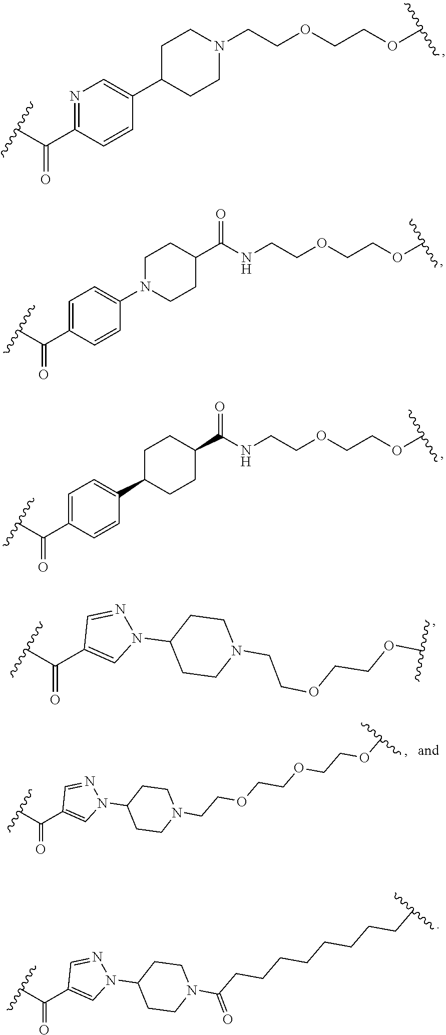 [SMILES: *OCCOCCN1CCC(c2ccc(C(*)=O)nc2)CC1 ; *OCCOCCNC(=O)C1CCN(c2ccc(C(*)=O)cc2)CC1 ; *OCCOCCNC(=O)[C@H]1CC[C@@H](c2ccc(C(*)=O)cc2)CC1 ; *OCCOCCN1CCC(n2cc(C(*)=O)cn2)CC1 ; *OCCOCCOCCN1CCC(n2cc(C(*)=O)cn2)CC1 ; *CCCCCCCCC(=O)N1CCC(n2cc(C(*)=O)cn2)CC1]

In the preceding embodiment, it is understood that for linkers of Formula (L-k), the point of attachment indicated on the carbonyl group is the attachment point to Y¹.

In certain embodiments, L is selected from the group consisting of:

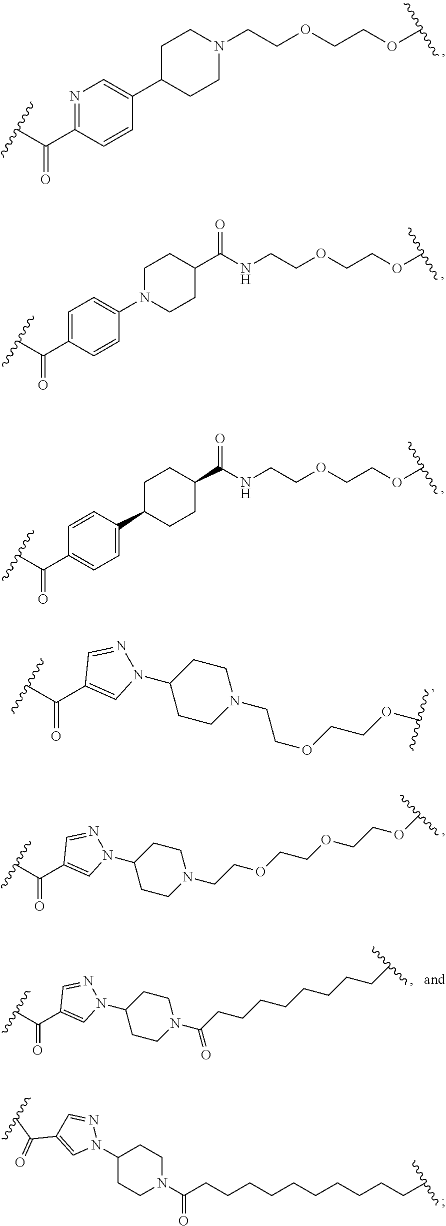

Wherein the point of attachment indicated on the carbonyl group is the attachment point to Y¹.

In certain embodiments, L is a divalent linker of Formula (L-m):

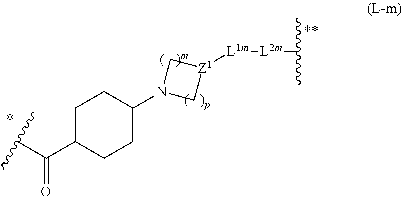

wherein:
Z¹ is CH or N;
m is 1 or 2;
p is 1 or 2;
0, 1, or 2 hydrogen atoms of

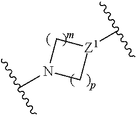

are replaced with F;
L^{1m} is a bond, —C(O)—, —C(O)NH—, —NHC(O)—, —S(O)₂NH— or —NHS(O)₂—; and
L^{2m} is C₃₋₆ linear alkylene, C₃₋₆ cycloalkylene, or

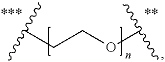

wherein n is 1 or 2, and

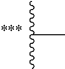

represents a covalent bond to L^{1m};
wherein

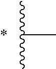

represents a covalent bond to Y¹, and

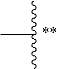

represents a covalent bond to X¹.

In certain embodiments, L is a divalent linker of Formula (L-m) selected from the group consisting of:

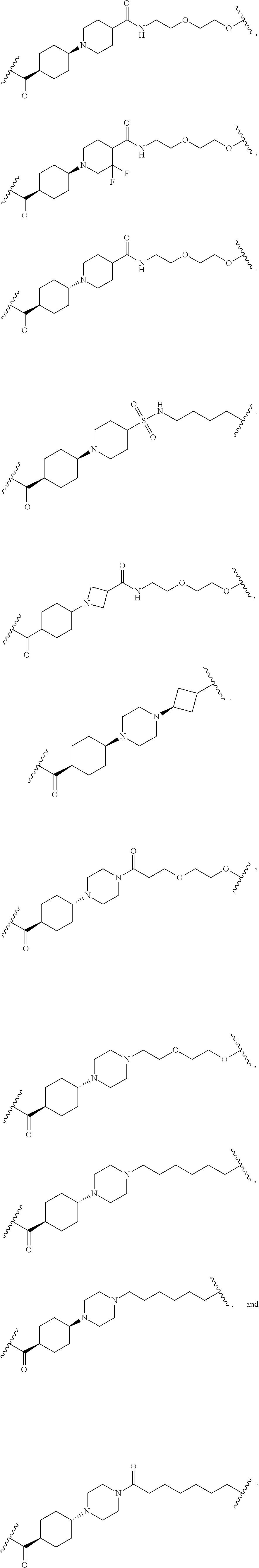

In the preceding embodiment, it is understood that for linkers of Formula (L-m), the point of attachment indicated on the carbonyl group is the attachment point to Y¹.

In certain embodiments, L is a divalent linker of Formula (L-n-i):

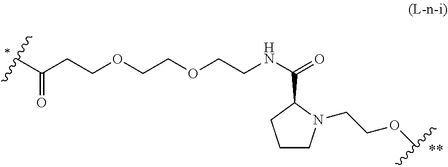 [SMILES: *OCCN1CCC[C@H]1C(=O)NCCOCCOCCC(*)=O]

wherein

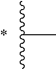

represents a covalent bond to Y¹, and

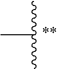

represents a covalent bond to X¹.

In certain embodiments, L is a divalent linker of Formula (L-n-ii):

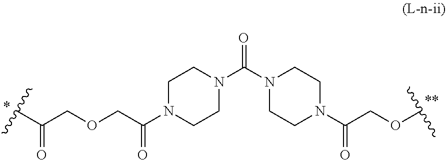 [SMILES: *OCC(=O)N1CCN(C(=O)N2CCN(C(=O)COCC(*)=O)CC2)CC1]

wherein

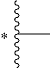

represents a covalent bond to Y¹, and

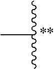

represents a covalent bond to X¹.

In certain embodiments, L is a divalent linker of Formula (L-n-iii):

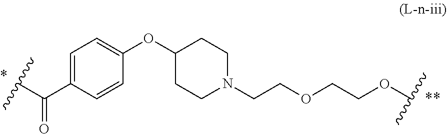 [SMILES: *OCCOCCN1CCC(Oc2ccc(C(*)=O)cc2)CC1]

wherein

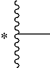

represents a covalent bond to Y¹, and

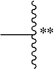

represents a covalent bond to X¹.

In certain embodiments, L is a divalent linker of Formula (L-n-iv):

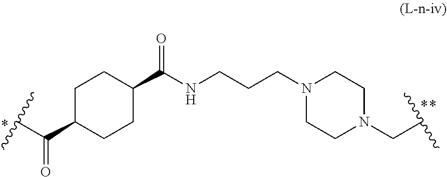 [SMILES: *CN1CCN(CCCNC(=O)[C@H]2CC[C@@H](C(*)=O)CC2)CC1]

wherein

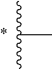

represents a covalent bond to Y¹, and

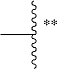

represents a covalent bond to X¹.

In certain embodiments, L is

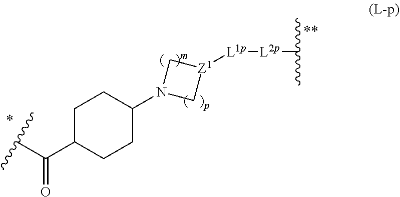

wherein Z¹ is CH or N; m is 1 or 2; p is 1 or 2; 0, 1, or 2 hydrogen atoms of

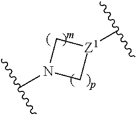

are replaced with F; L^{1p} is a bond, —C(O)—, —C(O)NH—, —NHC(O)—, —S(O)₂—, —S(O)₂NH—, or —NHS(O)₂—; and L^{2p} is -(4-6 membered saturated heterocyclylene containing 1 or 2 heteroatoms independently selected from nitrogen oxygen, and sulfur)-C(O))—; wherein

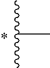

represents a covalent bond to Y¹, and

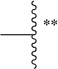

represents a covalent bond to X¹.

In certain embodiments, L is

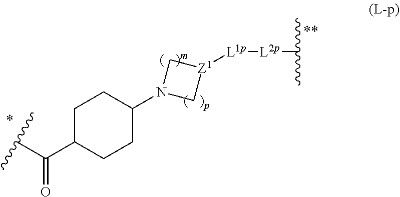

wherein Z¹ is CH or N; m is 1 or 2; p is 1 or 2; 0, 1, or 2 hydrogen atoms of

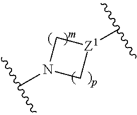

are replaced with F; L^{1p} is a bond, —C(O)—, —C(O)NH—, —NHC(O)—, —S(O)₂NH— or —NHS(O)₂—; and L^{2p} is -(4-6 membered saturated heterocyclylene containing 1 or 2 heteroatoms independently selected from nitrogen oxygen, and sulfur)-C(O))—.

In certain embodiments, L is

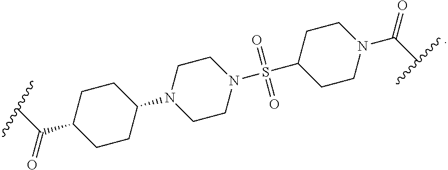 [SMILES: *C(=O)N1CCC(S(=O)(=O)N2CCN([C@H]3CC[C@@H](C(*)=O)CC3)CC2)CC1]

In certain embodiments, L is

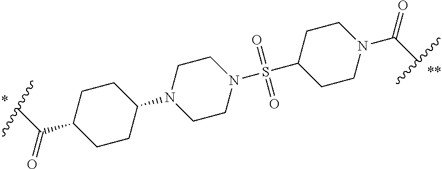 [SMILES: *C(=O)N1CCC(S(=O)(=O)N2CCN([C@H]3CC[C@@H](C(*)=O)CC3)CC2)CC1]

wherein

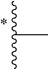

represents a covalent bond to Y¹, and

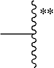

represents a covalent bond to X¹.

In certain embodiments, L is selected from the groups depicted in the compounds in Table 1, below. In certain embodiments, L is selected from the groups depicted in the compounds in Table 2, below.

### Additional Features

In certain embodiments,

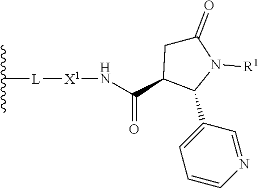

taken together are one of the following:

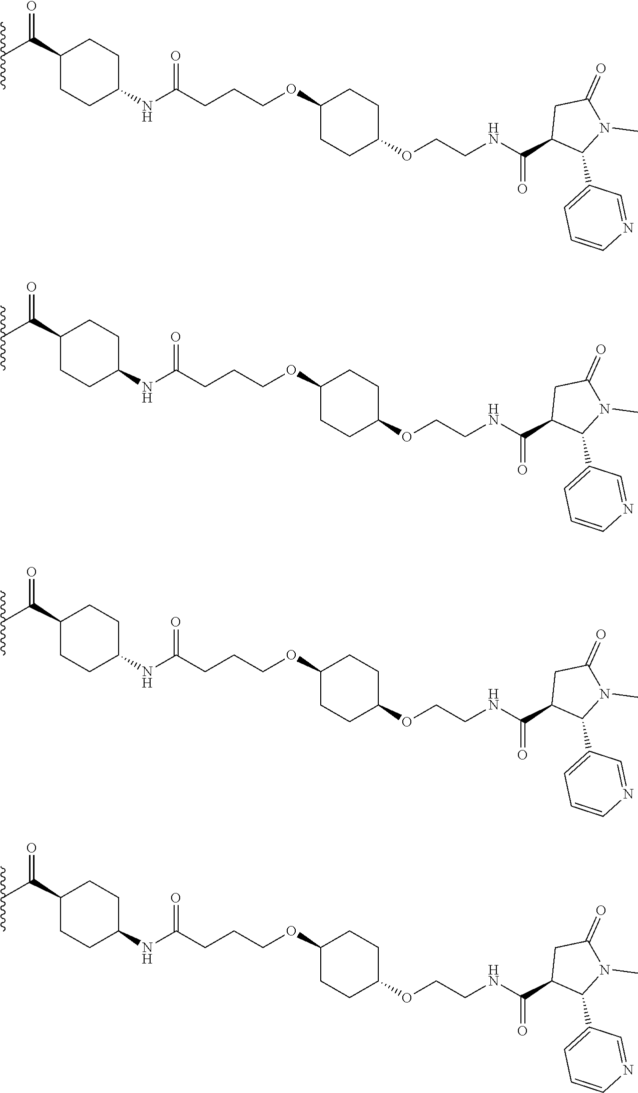 [SMILES: *C(=O)[C@H]1CC[C@H](NC(=O)CCCO[C@H]2CC[C@H](OCCNC(=O)[C@H]3CC(=O)N(C)[C@@H]3c3cccnc3)CC2)CC1 ; *C(=O)[C@H]1CC[C@@H](NC(=O)CCCO[C@H]2CC[C@@H](OCCNC(=O)[C@H]3CC(=O)N(C)[C@@H]3c3cccnc3)CC2)CC1 ; *C(=O)[C@H]1CC[C@H](NC(=O)CCCO[C@H]2CC[C@@H](OCCNC(=O)[C@H]3CC(=O)N(C)[C@@H]3c3cccnc3)CC2)CC1 ; *C(=O)[C@H]1CC[C@@H](NC(=O)CCCO[C@H]2CC[C@H](OCCNC(=O)[C@H]3CC(=O)N(C)[C@@H]3c3cccnc3)CC2)CC1]

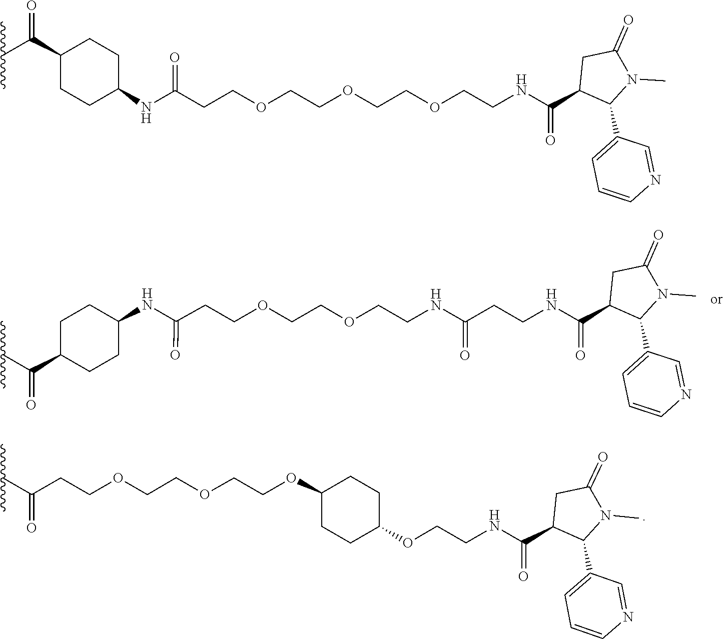

The description above describes multiple embodiments relating to compounds of Formula I. The patent application specifically contemplates all combinations of the embodiments.

Another aspect of the invention provides a compound represented by Formula II:

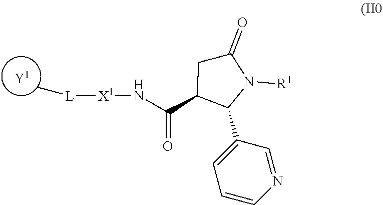

or a pharmaceutically acceptable salt thereof, wherein:
X¹ is C₁₋₅ alkylene or a covalent bond;
Y¹ is one of the following:

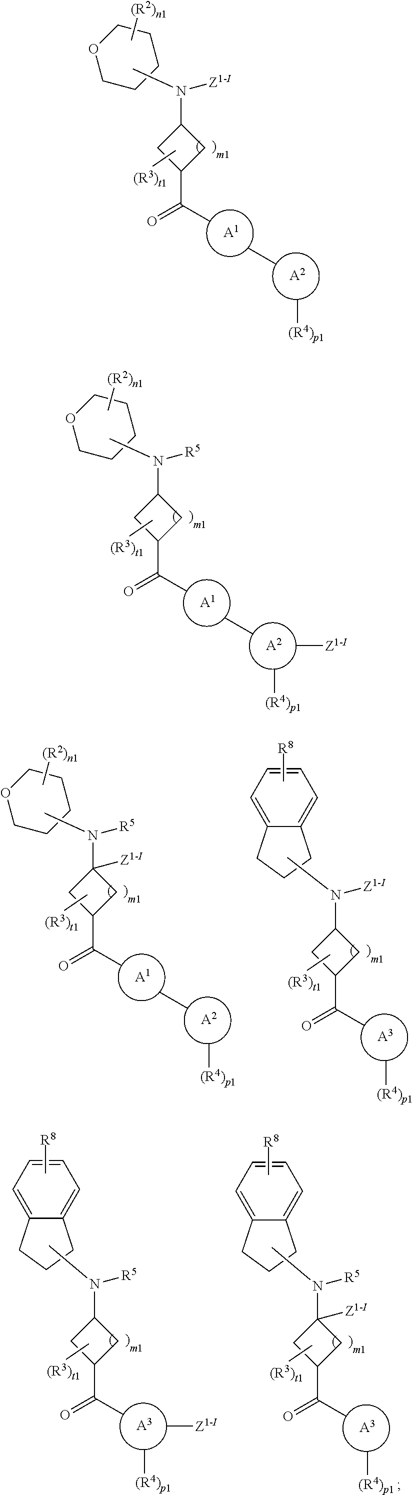

each of which is substituted by one occurrence of R⁷;
Z^{1-I} is C₁₋₆ alkylene-N(R⁶)₂ or —(C₁₋₆ alkylene)-(4-6 membered saturated heterocyclyl containing 1 or 2 heteroatoms selected from nitrogen);
R¹ is C₁₋₄ alkyl or C₃₋₆ cycloalkyl;
R² represents independently for each occurrence C₁₋₄ alkoxyl or C₁₋₄ alkyl;
R³ and R⁹ each represent independently for each occurrence C₁₋₄ alkyl;
R⁵ and R⁶ are independently hydrogen or C₁₋₄ alkyl;
R⁴ and R⁸ each represent independently for each occurrence C₁₋₄ haloalkyl, halo, or C₁₋₄ alkyl;
R⁷ is a bond to L;
A¹ is (i) a 5-7 membered saturated heterocyclylene containing 1 or 2 heteroatoms independently selected from nitrogen and oxygen or (ii) a 6-8 membered bridged bicyclic saturated heterocyclylene containing 1 or 2 heteroatoms independently selected from nitrogen and oxygen; wherein the heterocyclylene is substituted with w1 occurrences of R⁹;
A² is a 5-6 membered heteroarylene containing 1 or 2 heteroatoms independently selected from nitrogen, oxygen, or sulfur;
A³ is an 8-10 membered bicyclic partially unsaturated ring containing 1, 2, or 3 heteroatoms selected from nitrogen;
n1, p1, and t1 are independently 1, 2, or 3;
m1 is 1 or 2;
w1 is 0, 1, or 2;
L is a divalent linker selected from:
(i) a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, optionally substituted 3-10 membered heterocyclyl containing 1, 2, 3, or 4 heteroatoms independently selected from nitrogen, oxygen, and sulfur, or optionally substituted 5-6 membered heteroaryl containing 1, 2, or 3 heteroatoms independently selected from nitrogen, oxygen, and sulfur;
(ii)

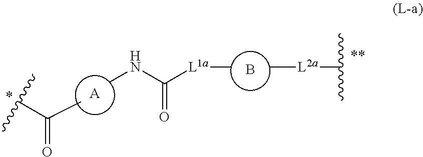

wherein Ring A and Ring B are each independently C₄₋₆ cycloalkylene; L^{1a} is C₃₋₅ linear alkylene, wherein 1 or 2 methylene units are replaced with —O— or —NR^{a}—; each R^{a} is independently hydrogen or C₁₋₃ alkyl; and L^{2a} is —O—, —NHC(O)—, or —CH₂—O—;
(iii)

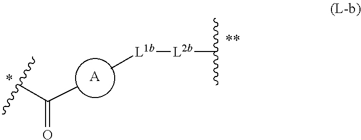

wherein Ring A is C₄₋₆ cycloalkylene or C₇₋₉ bridged bicyclic cycloalkylene; L^{1b} is —CH₂—NH—C(O)—, —NHC(O)—, or —C(O)NH—; L^{2b} is C₆-12 linear alkylene, wherein 1, 2, 3, or 4 methylene units are replaced with —O—, —NR^{1b}—, —C(O)NR^{1b}—, or —NR^{1b}C(O)—; or L²b is

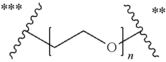

wherein n is 1, 2, 3, or 4, and

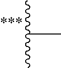

represents a covalent bond to L^{1b}; and each R^{1b} is independently hydrogen or C₁₋₃ alkyl;
(iv)

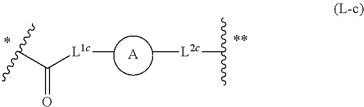

wherein L^{1c} is C₂₋₁₀ linear alkylene, wherein 1, 2, or 3 methylene units are replaced with —O—, —NH—, —NHC(O)—, or —C(O)NH—; Ring A is C₄₋₆ cycloalkylene or C₇₋₉ bridged bicyclic cycloalkylene; and L^{2c} is —O— or a saturated C₂₋₁₀ linear alkylene, wherein 1, 2, or 3 methylene units are replaced with —O—, —NH—, —NHC(O)—, or —C(O)NH—;
(v)

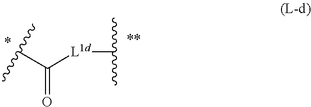

wherein L^{1d} is C₁₂₋₂₂ linear alkylene, wherein 1, 2, 3, 4, or 5 methylene units are replaced with —NH—, —O—, —C(O)NH—, —NHC(O)—, or —NHC(O)—NH—;
(vi)

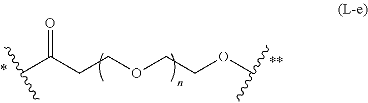

wherein n is an integer of 3 to 50;
(vii)

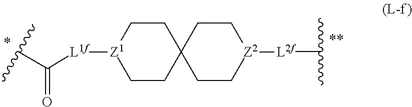

wherein L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—; L^{2f} is a bond, —NHC(O)—, —C(O)NH—, or a C₁₋₆ linear alkylene, wherein 0, 1, or 2 methylene units are replaced with —O—; and each of Z¹ and Z² is independently N or CH;
(viii)

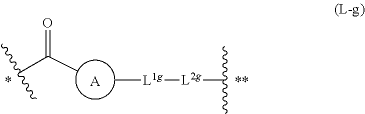

wherein Ring A is a 5 to 6 membered heteroarylene having 1 or 2 nitrogen ring atoms; L^{1g} is a bond, —CH₂—, —NH—, or —O—; and
L^{2g} is

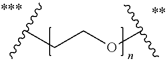

wherein n is 1, 2, 3, 4, or 5, and

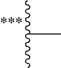

represents a covalent bond to L^{1g};
(ix)

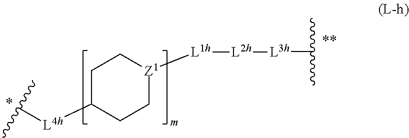

wherein each Z¹ is independently N or CH; L^{1h} is a bond, —C(O)—, —C(O)—NH—, or —NHC(O)—; L^{2h} is C₂₋₁₀ linear alkylene or

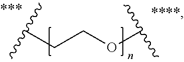

wherein n is 1, 2, 3, or 4, and

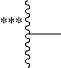

represents a covalent bond to L^{1h} and

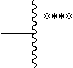

represents a covalent bond to L^{3h}; L^{3h} is a bond, —C(O)CH₂—, —O—(C₃₋₆ cycloalkylene)-O—, or —C(O)NH(CH₂)₃OCH₂—; L^{4h} is a bond, —C(O)—, —CH₂C(O)—, or —C(O)CH₂—; and m is 1, 2, or 3;
(x)

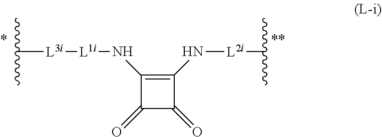

wherein L¹ⁱ is a bond, C₁₋₁₂ linear alkylene, or

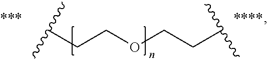

wherein n is 1, 2, 3, 4, or 5, and

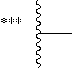

represents a covalent bond to L³ⁱ and

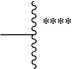

represents a covalent bond to NH; L²ⁱ is a bond, C₁₋₁₂ linear alkylene, or

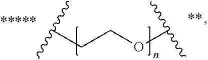

wherein n is 1, 2, 3, 4, or 5, and

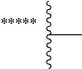

represents a covalent bond to HN; and L³ⁱ is a bond or —C(O)—;
(xi)

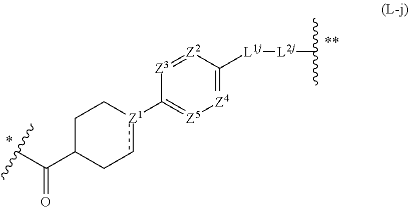

wherein Z¹ is C, CH, or N; each of Z², Z³, Z⁴ and Z⁵ is independently CH or N, provided that no more than two of Z², Z³, Z⁴ and Z⁵ are N; L^{1j} is —NH—, —C(O)NH—, —NHC(O)—, or —O—; L^{2j} is C₁₋₆ linear alkylene or

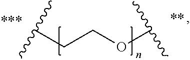

wherein n is 1 or 2, and

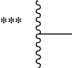

represents a covalent bond to L¹; and [symbol] represents a single bond or a double bond;
(xii)

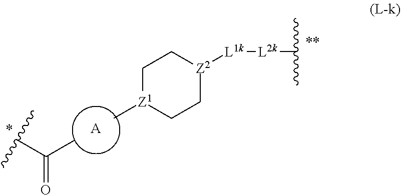

wherein Ring A is phenyl or a 5 or 6 membered heteroarylene having 1 or 2 nitrogen ring atoms; each of Z¹ and Z² is independently CH or N; L^{1k} is a bond, —C(O)—, —C(O)NH— or —NHC(O)—; and L^{2k} is a C₃₋₈ straight chain alkylene or

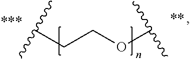

wherein n is 1, 2, or 3, and

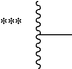

represents a covalent bond to L^{1k};
(xiii)

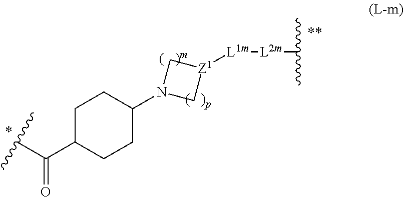

wherein Z¹ is CH or N; m is 1 or 2; p is 1 or 2; 0, 1, or 2 hydrogen atoms of

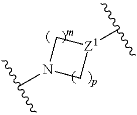

are replaced with F; L^{1m} is a bond, —C(O)—, —C(O)NH—, —NHC(O)—, —S(O)₂NH— or —NHS(O)₂—; and L^{2m} is C₃₋₆ linear alkylene, C₃₋₆ cycloalkylene, or

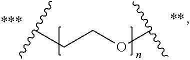

wherein n is 1 or 2, and

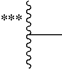

represents a covalent bond to L^{1m};
(xiv) one of

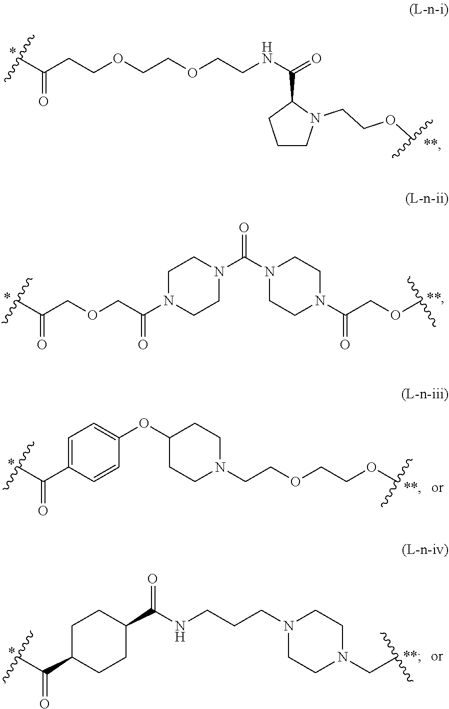

(xv)

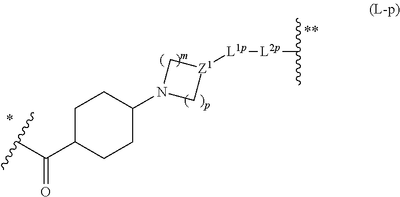

wherein Z¹ is CH or N; m is 1 or 2; p is 1 or 2; 0, 1, or 2 hydrogen atoms of

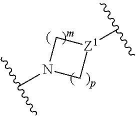

are replaced with F; L^{1p} is a bond, —C(O)—, —C(O)NH—, —NHC(O)—, —S(O)₂NH— or —NHS(O)₂—; and L^{2p} is -(4-6 membered saturated heterocyclylene containing 1 or 2 heteroatoms independently selected from nitrogen oxygen, and sulfur)-C(O))—;
wherein each

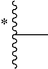

represents a covalent bond to Y¹, and each

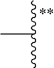

represents a covalent bond to X¹.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

In certain embodiments, Z^{1-I} is C₁₋₆ alkylene-N(R⁶)₂. In certain embodiments, Z^{1-I} is C₁₋₃ alkylene-N(R⁶)₂. In certain embodiments, Z^{1-I} is —CH₂—N(R⁶)₂. In certain embodiments, Z¹⁻¹ is —CH₂CH₂—N(R⁶)₂. In certain embodiments, Z^{1-I} is —CH₂CH₂CH₂—N(R⁶)₂.

In certain embodiments, Z^{1-I} is —(C₁₋₆ alkylene)-(4-6 membered saturated heterocyclyl containing 1 or 2 heteroatoms selected from nitrogen). In certain embodiments, Z^{1-I} is —(C₁₋₆ alkylene)-(6-membered saturated heterocyclyl containing 1 heteroatom selected from nitrogen). In certain embodiments, Z^{1-I} is —(C₁₋₆ alkylene)-(piperidinyl). In certain embodiments, Z^{1-I} is —(C₂₋₃ alkylene)-(piperidinyl). In certain embodiments, Z^{1-I} is -(ethylene)-(piperidinyl). In certain embodiments, Z^{1-I} is

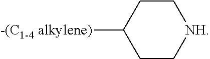

In certain embodiments, Z^{1-I} is —(CH₂CH₂) NH

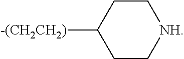

In certain embodiments, X¹, Y¹, R¹, R², R³, R⁴, R, R⁶, R⁷, R⁹, A¹, A², A³, n1, p1, and t1, m1, w1, and L are as defined above in connection with Formula I, or one of the embodiments described above in connection with Formula I.

### Exemplary Specific Compounds

In certain embodiments, the compound is a compound in Table 1 or a pharmaceutically acceptable salt thereof. In certain embodiments, the compound is a compound in Table 1. In certain embodiments, the compound is a compound in Table 2 or a pharmaceutically acceptable salt thereof. In certain embodiments, the compound is a compound in Table 2. In certain embodiments, the compound is a compound in Table 1 or 2, or a pharmaceutically acceptable salt thereof. In certain embodiments, the compound is a compound in Table 1 or 2.

TABLE 1
Exemplary Compounds

| Compound | Chemical |
|---|---|
| No. | Structure |
| I-1 | 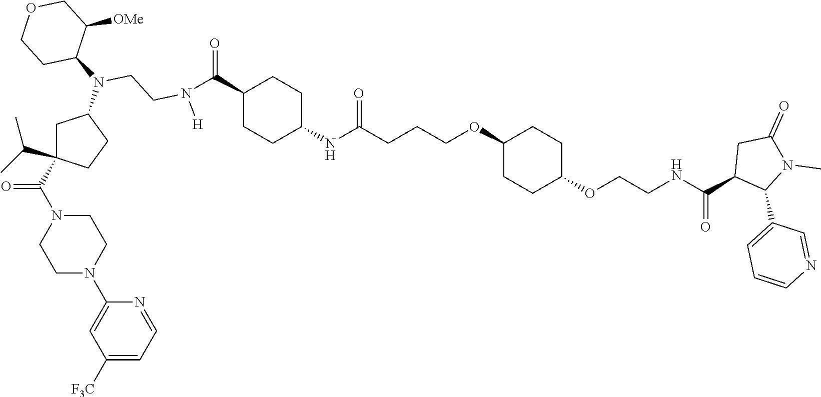 [SMILES: CO[C@@H]1COCC[C@@H]1N(CCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2CCN(c3cc(C(F)(F)F)ccn3)CC2)(C(C)C)C1] |
| I-2 | 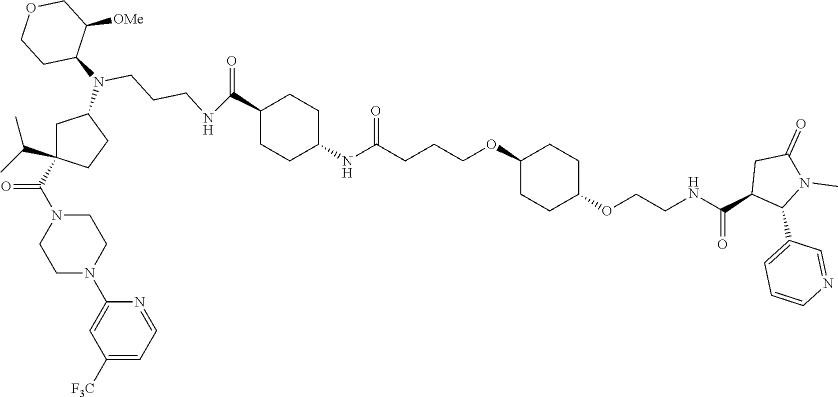 [SMILES: CO[C@@H]1COCC[C@@H]1N(CCCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2CCN(c3cc(C(F)(F)F)ccn3)CC2)(C(C)C)C1] |
| I-3 | 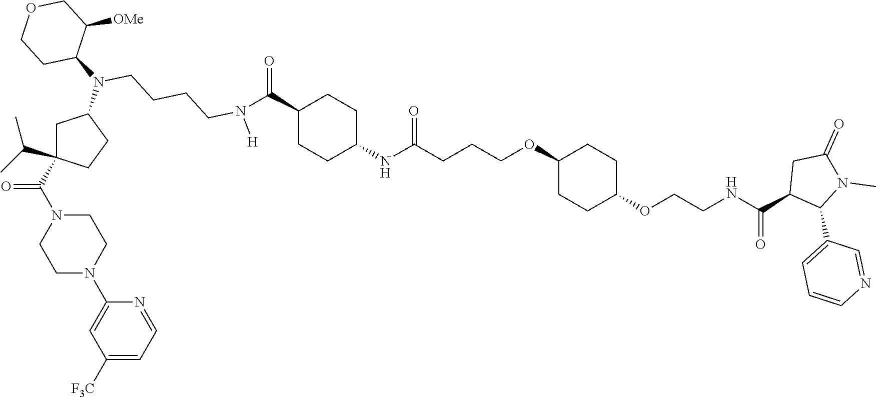 [SMILES: CO[C@@H]1COCC[C@@H]1N(CCCCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2CCN(c3cc(C(F)(F)F)ccn3)CC2)(C(C)C)C1] |
| I-4 | 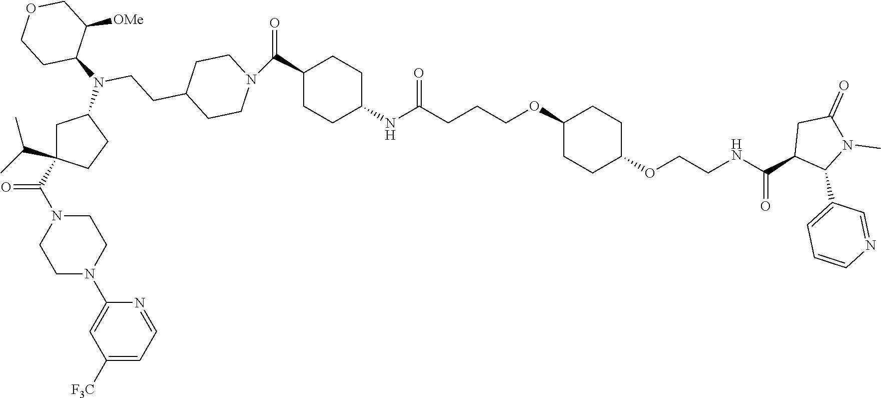 [SMILES: CO[C@@H]1COCC[C@@H]1N(CCC1CCN(C(=O)[C@H]2CC[C@H](NC(=O)CCCO[C@H]3CC[C@H](OCCNC(=O)[C@H]4CC(=O)N(C)[C@@H]4c4cccnc4)CC3)CC2)CC1)[C@@H]1CC[C@@](C(=O)N2CCN(c3cc(C(F)(F)F)ccn3)CC2)(C(C)C)C1] |
| I-5 | 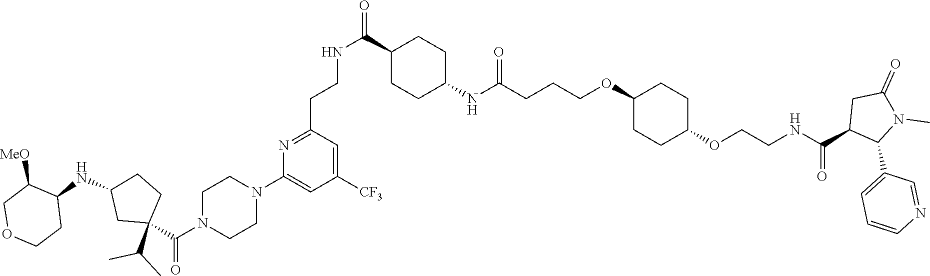 [SMILES: CO[C@@H]1COCC[C@@H]1N[C@@H]1CC[C@@](C(=O)N2CCN(c3cc(C(F)(F)F)cc(CCNC(=O)[C@H]4CC[C@H](NC(=O)CCCO[C@H]5CC[C@H](OCCNC(=O)[C@H]6CC(=O)N(C)[C@@H]6c6cccnc6)CC5)CC4)n3)CC2)(C(C)C)C1] |
| I-6 | 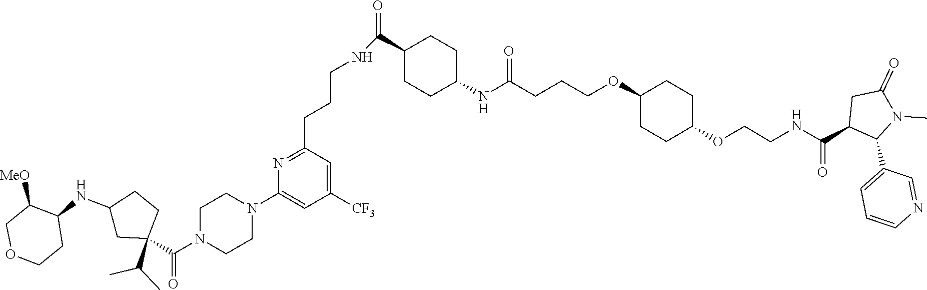 [SMILES: CO[C@@H]1COCC[C@@H]1NC1CC[C@@](C(=O)N2CCN(c3cc(C(F)(F)F)cc(CCCNC(=O)[C@H]4CC[C@H](NC(=O)CCCO[C@H]5CC[C@H](OCCNC(=O)[C@H]6CC(=O)N(C)[C@@H]6c6cccnc6)CC5)CC4)n3)CC2)(C(C)C)C1] |
| I-7 | 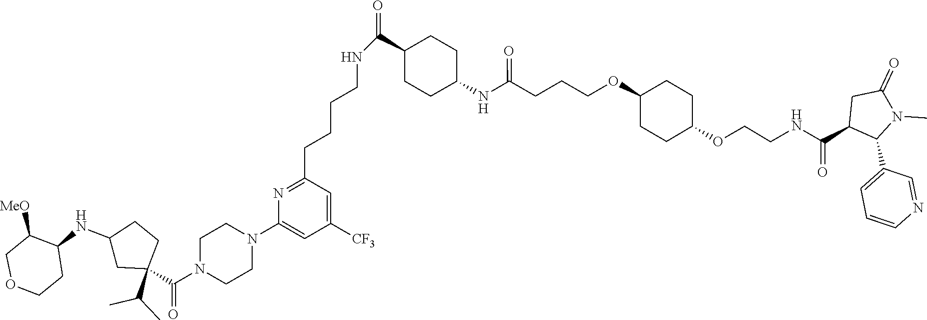 [SMILES: CO[C@@H]1COCC[C@@H]1NC1CC[C@@](C(=O)N2CCN(c3cc(C(F)(F)F)cc(CCCCNC(=O)[C@H]4CC[C@H](NC(=O)CCCO[C@H]5CC[C@H](OCCNC(=O)[C@H]6CC(=O)N(C)[C@@H]6c6cccnc6)CC5)CC4)n3)CC2)(C(C)C)C1] |
| I-8 | 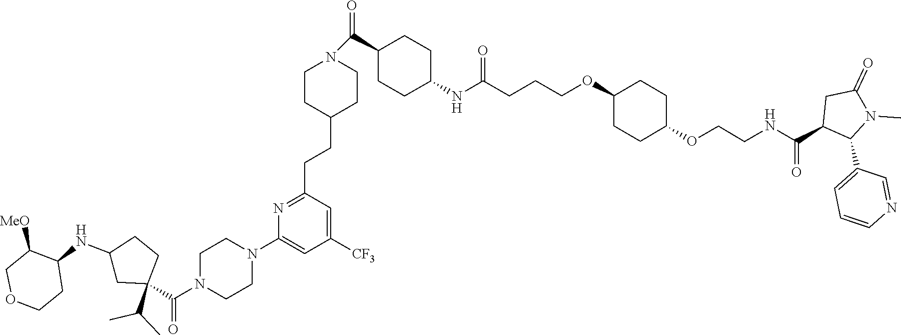 [SMILES: CO[C@@H]1COCC[C@@H]1NC1CC[C@@](C(=O)N2CCN(c3cc(C(F)(F)F)cc(CCC4CCN(C(=O)[C@H]5CC[C@H](NC(=O)CCCO[C@H]6CC[C@H](OCCNC(=O)[C@H]7CC(=O)N(C)[C@@H]7c7cccnc7)CC6)CC5)CC4)n3)CC2)(C(C)C)C1] |
| I-9 | 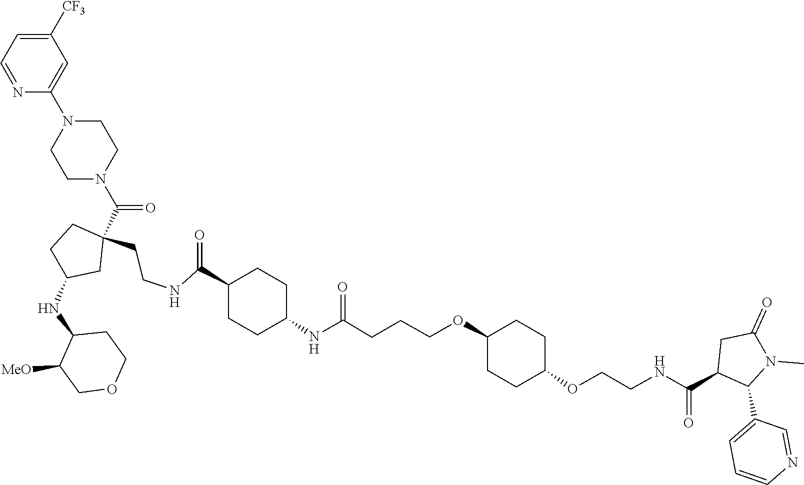 [SMILES: CO[C@@H]1COCC[C@@H]1N[C@@H]1CC[C@](CCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCN(c3cc(C(F)(F)F)ccn3)CC2)C1] |
| I-10 | 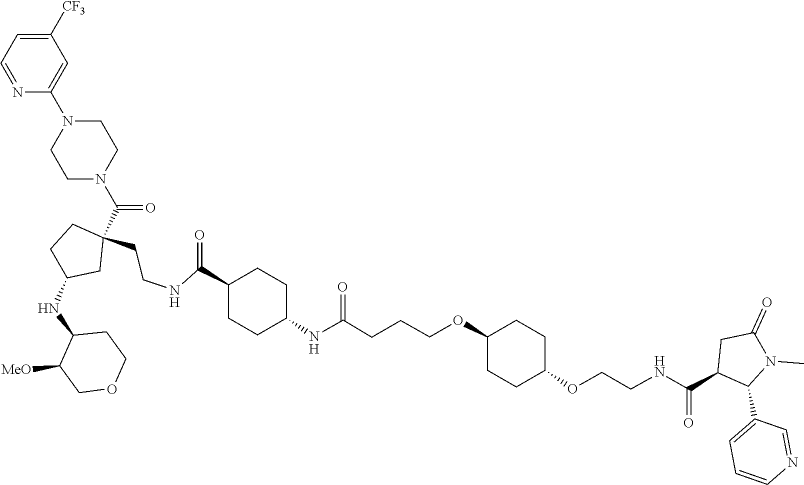 [SMILES: CO[C@@H]1COCC[C@@H]1N[C@@H]1CC[C@](CCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCN(c3cc(C(F)(F)F)ccn3)CC2)C1] |
| I-11 | 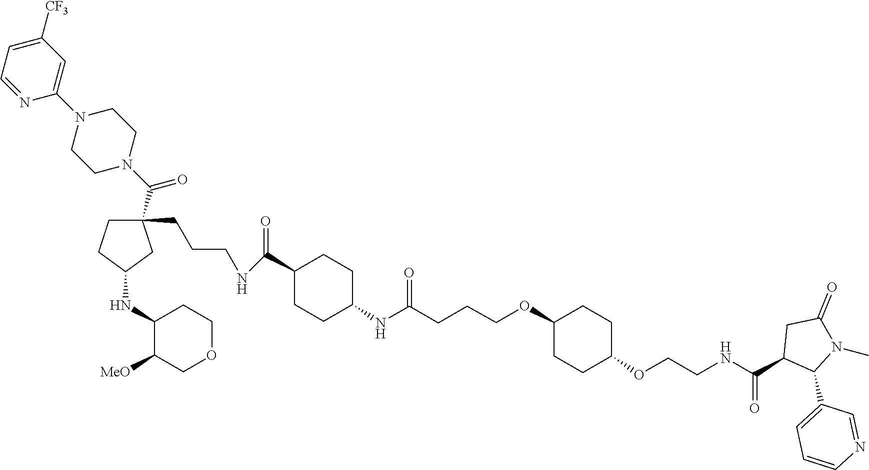 [SMILES: CO[C@@H]1COCC[C@@H]1N[C@@H]1CC[C@](CCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCN(c3cc(C(F)(F)F)ccn3)CC2)C1] |
| I-12 | 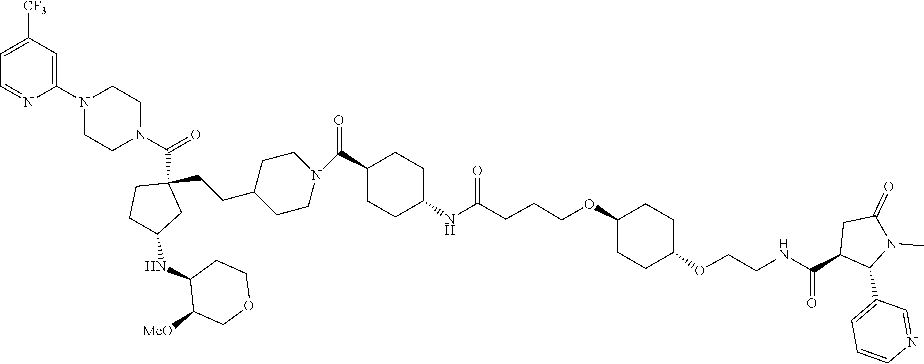 [SMILES: CO[C@@H]1COCC[C@@H]1N[C@@H]1CC[C@](CCC2CCN(C(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)(C(=O)N2CCN(c3cc(C(F)(F)F)ccn3)CC2)C1] |
| I-13 | 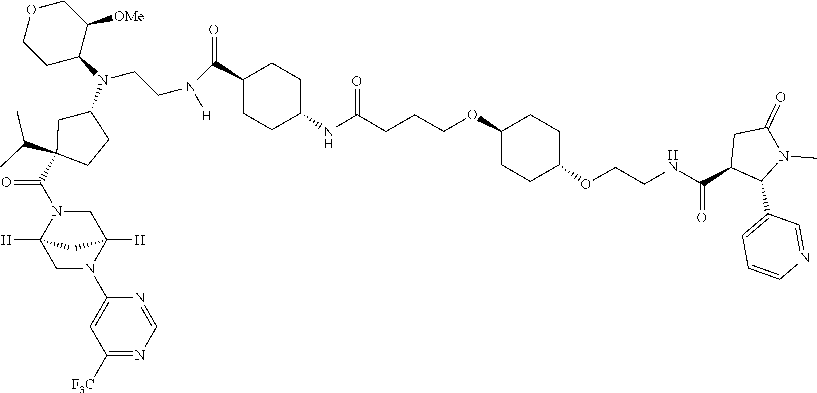 [SMILES: CO[C@@H]1COCC[C@@H]1N(CCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2C[C@@H]3C[C@H]2CN3c2cc(C(F)(F)F)ncn2)(C(C)C)C1] |
| I-14 | 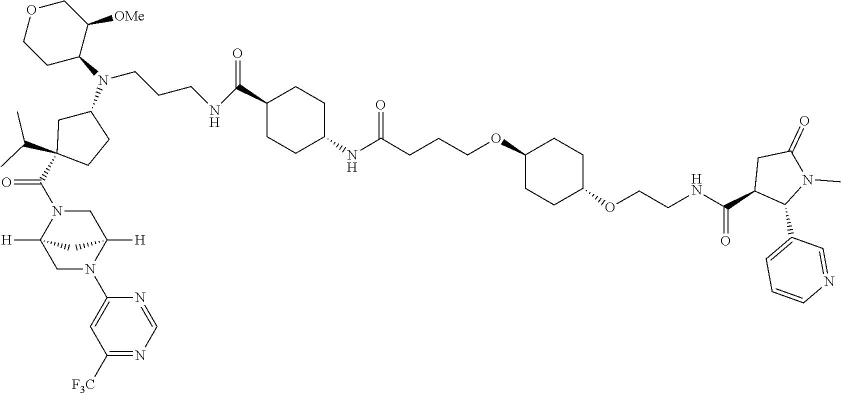 [SMILES: CO[C@@H]1COCC[C@@H]1N(CCCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2C[C@@H]3C[C@H]2CN3c2cc(C(F)(F)F)ncn2)(C(C)C)C1] |
| I-15 | 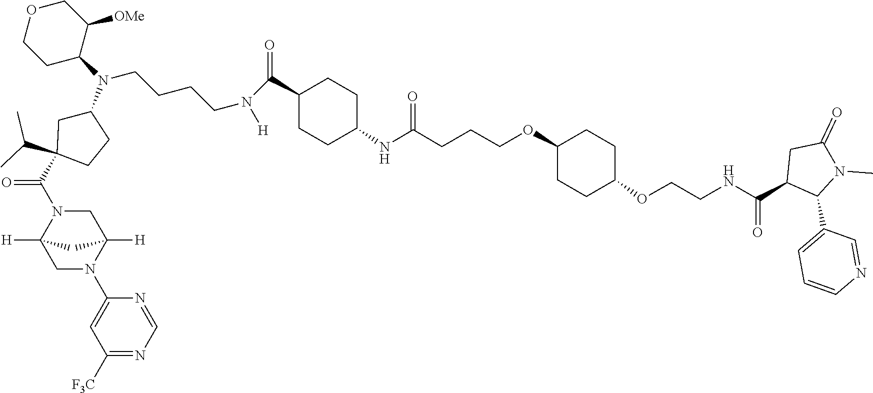 [SMILES: CO[C@@H]1COCC[C@@H]1N(CCCCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2C[C@@H]3C[C@H]2CN3c2cc(C(F)(F)F)ncn2)(C(C)C)C1] |
| I-16 | 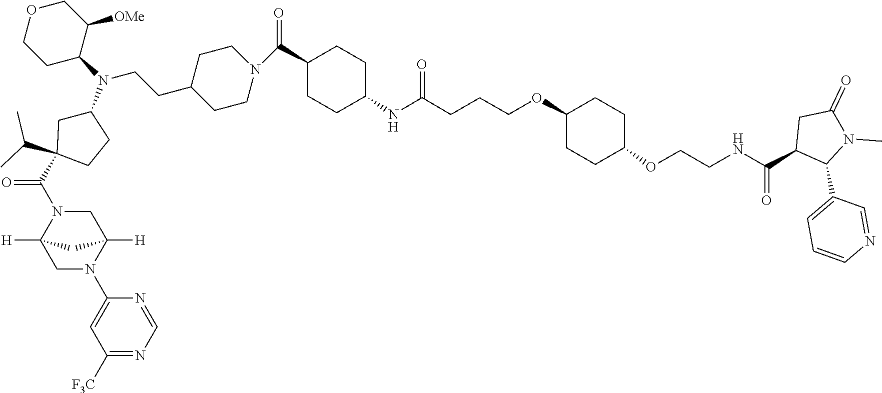 [SMILES: CO[C@@H]1COCC[C@@H]1N(CCC1CCN(C(=O)[C@H]2CC[C@H](NC(=O)CCCO[C@H]3CC[C@H](OCCNC(=O)[C@H]4CC(=O)N(C)[C@@H]4c4cccnc4)CC3)CC2)CC1)[C@@H]1CC[C@@](C(=O)N2C[C@@H]3C[C@H]2CN3c2cc(C(F)(F)F)ncn2)(C(C)C)C1] |
| I-17 | 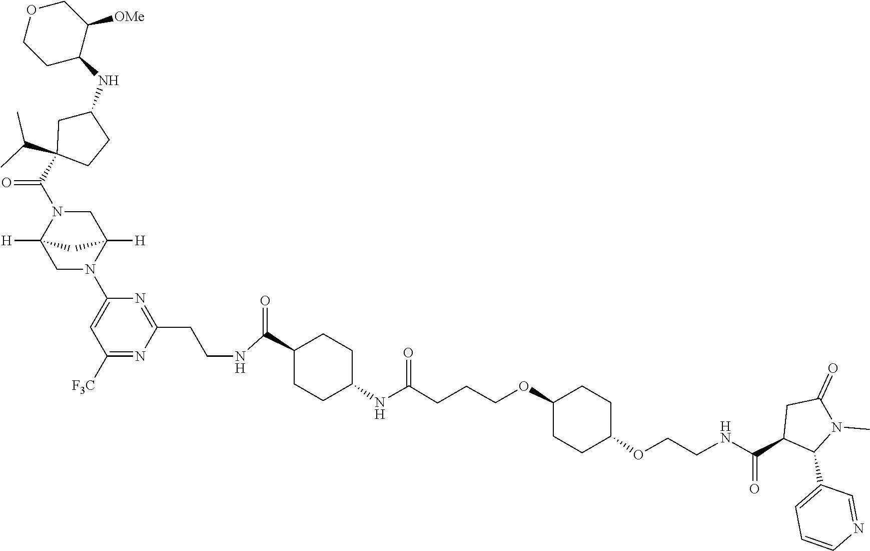 [SMILES: CO[C@@H]1COCC[C@@H]1N[C@@H]1CC[C@@](C(=O)N2C[C@@H]3C[C@H]2CN3c2cc(C(F)(F)F)nc(CCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)n2)(C(C)C)C1] |
| I-18 | 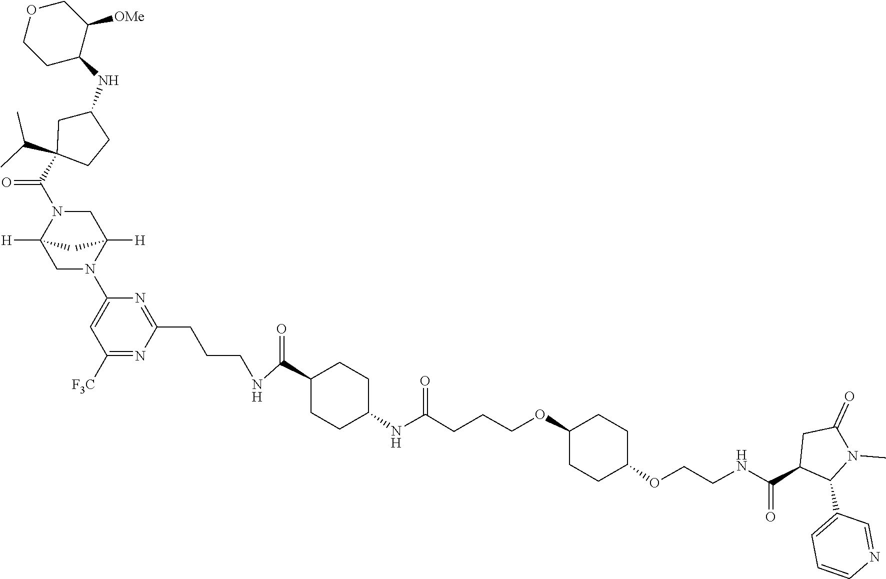 [SMILES: CO[C@@H]1COCC[C@@H]1N[C@@H]1CC[C@@](C(=O)N2C[C@@H]3C[C@H]2CN3c2cc(C(F)(F)F)nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)n2)(C(C)C)C1] |
| I-19 | 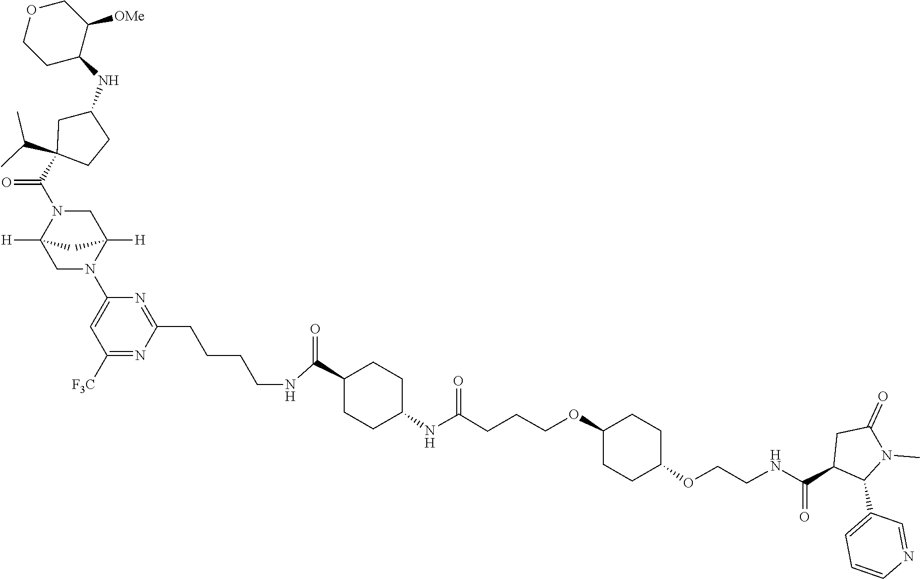 [SMILES: CO[C@@H]1COCC[C@@H]1N[C@@H]1CC[C@@](C(=O)N2C[C@@H]3C[C@H]2CN3c2cc(C(F)(F)F)nc(CCCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)n2)(C(C)C)C1] |
| I-20 | 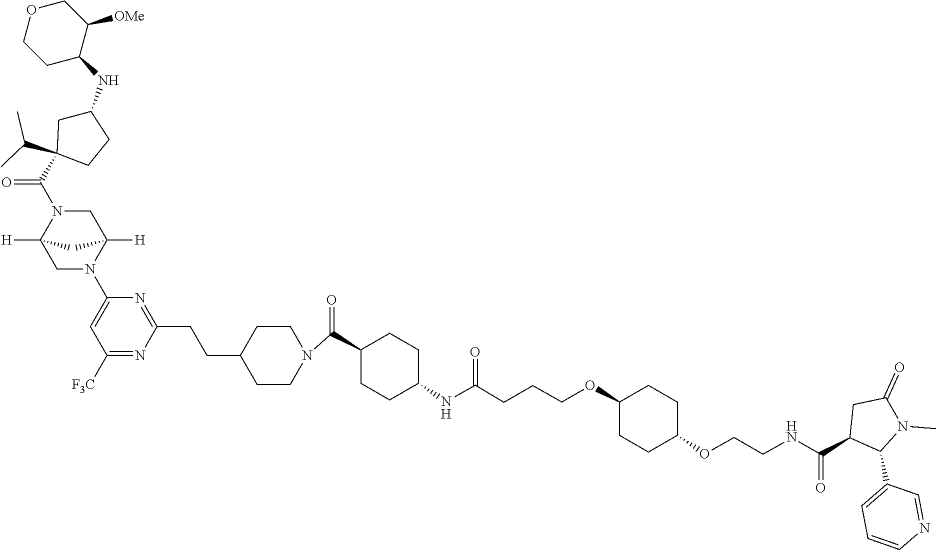 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)n2)(C(C)C)C1] |
| I-21 | 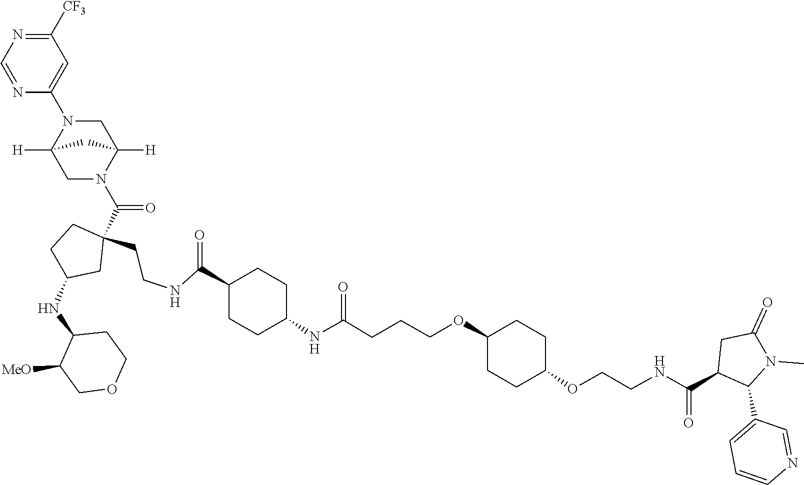 [SMILES: CO[C@@H]1COCC[C@@H]1N[C@@H]1CC[C@](CCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2C[C@@H]3C[C@H]2CN3c2cc(C(F)(F)F)ncn2)C1] |
| I-22 | 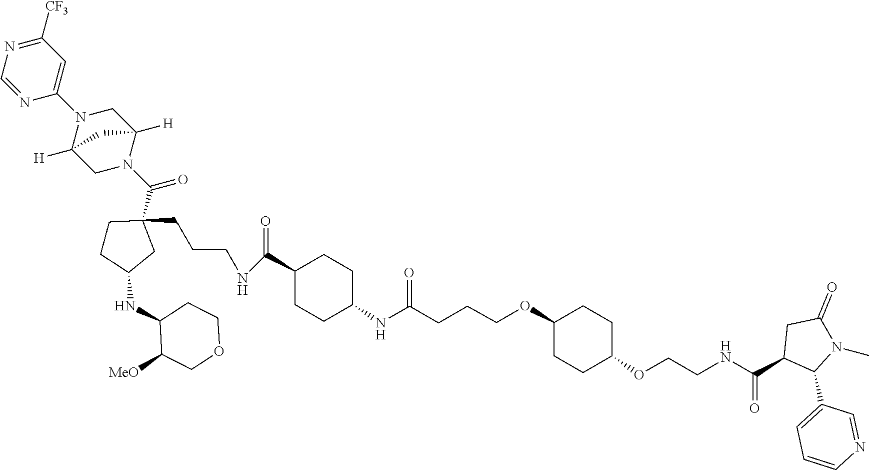 [SMILES: CO[C@@H]1COCC[C@@H]1N[C@@H]1CC[C@](CCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2C[C@@H]3C[C@H]2CN3c2cc(C(F)(F)F)ncn2)C1] |
| I-23 | 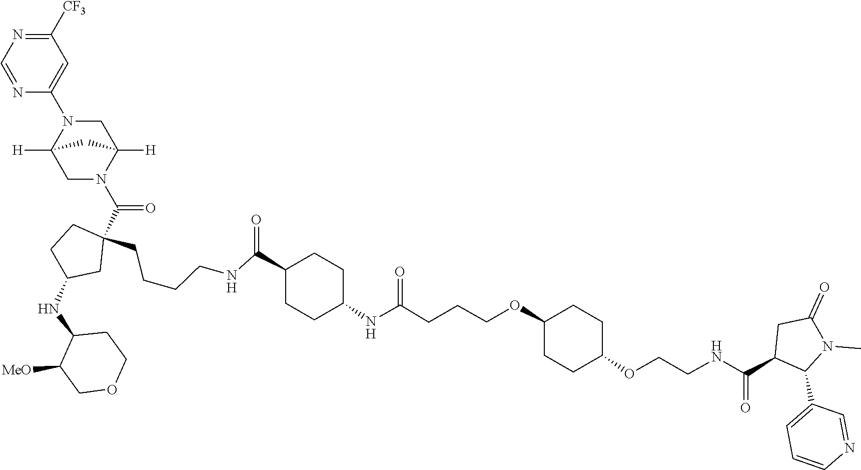 [SMILES: CO[C@@H]1COCC[C@@H]1N[C@@H]1CC[C@](CCCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2C[C@@H]3C[C@H]2CN3c2cc(C(F)(F)F)ncn2)C1] |
| I-24 | 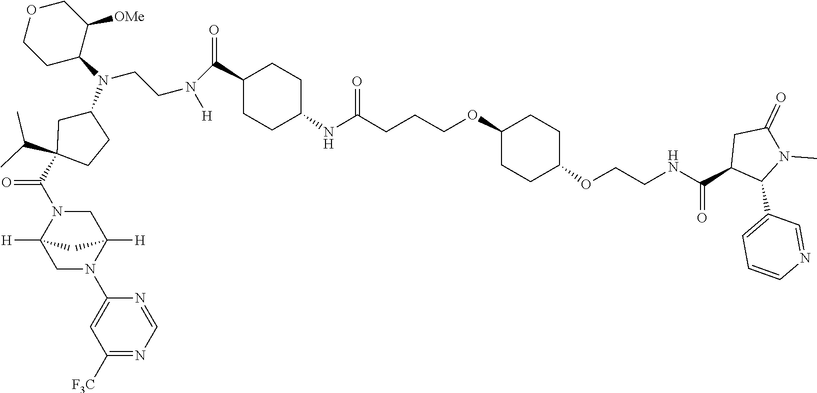 [SMILES: CO[C@@H]1COCC[C@@H]1N(CCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2C[C@@H]3C[C@H]2CN3c2cc(C(F)(F)F)ncn2)(C(C)C)C1] |
| I-25 | 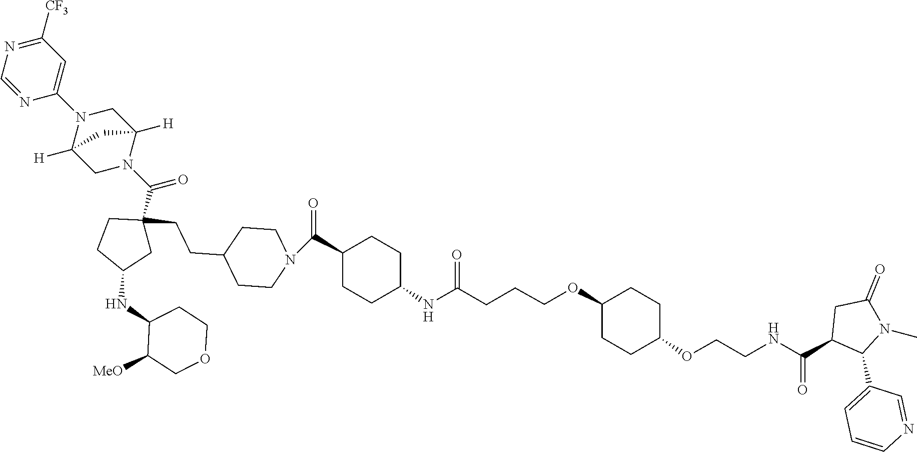 |
| I-26 | 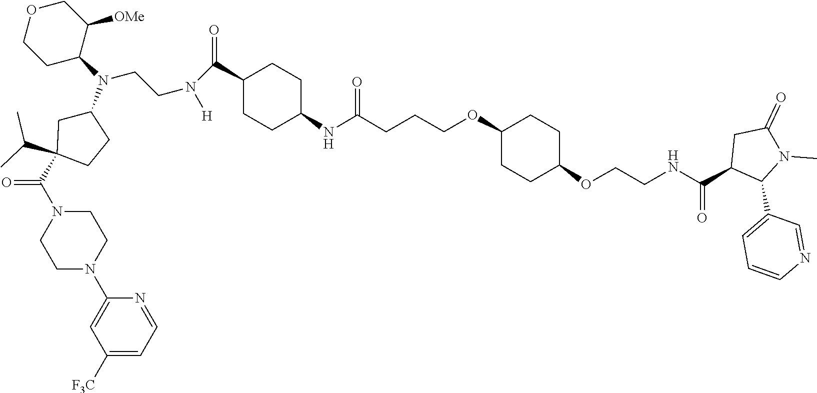 [SMILES: CO[C@@H]1COCC[C@@H]1N(CCNC(=O)[C@H]1CC[C@@H](NC(=O)CCCO[C@H]2CC[C@@H](OCCNC(=O)[C@H]3CC(=O)N(C)[C@@H]3c3cccnc3)CC2)CC1)[C@@H]1CC[C@@](C(=O)N2CCN(c3cc(C(F)(F)F)ccn3)CC2)(C(C)C)C1] |
| I-27 | 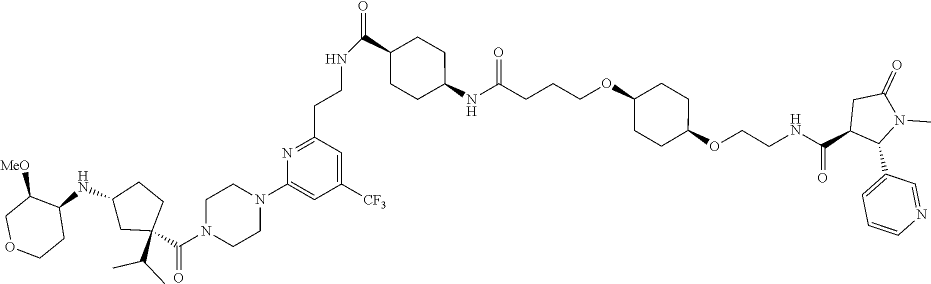 [SMILES: CO[C@@H]1COCC[C@@H]1N[C@@H]1CC[C@@](C(=O)N2CCN(c3cc(C(F)(F)F)cc(CCNC(=O)[C@H]4CC[C@@H](NC(=O)CCCO[C@H]5CC[C@@H](OCCNC(=O)[C@H]6CC(=O)N(C)[C@@H]6c6cccnc6)CC5)CC4)n3)CC2)(C(C)C)C1] |
| I-28 | 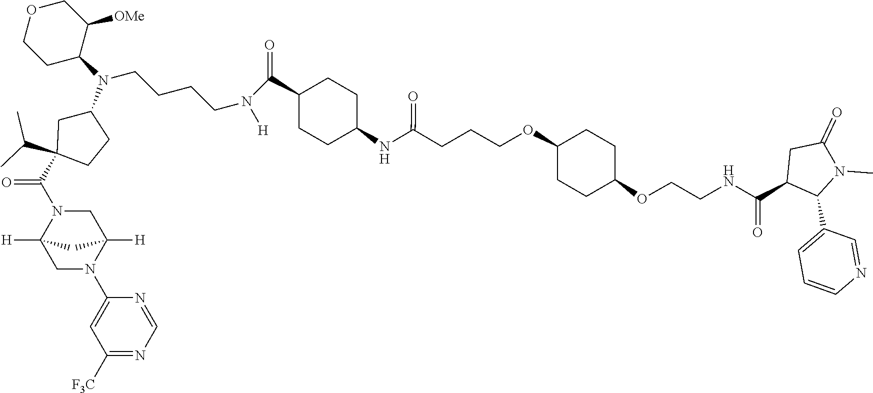 [SMILES: CO[C@@H]1COCC[C@@H]1N(CCCCNC(=O)[C@H]1CC[C@@H](NC(=O)CCCO[C@H]2CC[C@@H](OCCNC(=O)[C@H]3CC(=O)N(C)[C@@H]3c3cccnc3)CC2)CC1)[C@@H]1CC[C@@](C(=O)N2C[C@@H]3C[C@H]2CN3c2cc(C(F)(F)F)ncn2)(C(C)C)C1] |
| I-29 | 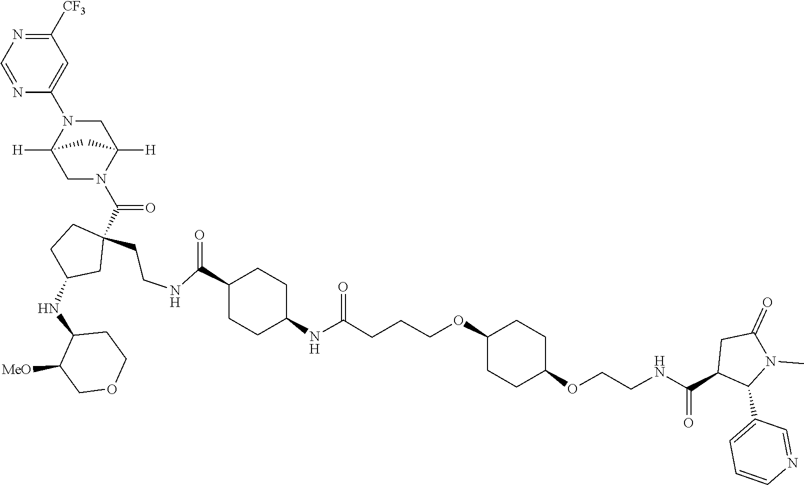 [SMILES: CO[C@@H]1COCC[C@@H]1N[C@@H]1CC[C@](CCNC(=O)[C@H]2CC[C@@H](NC(=O)CCCO[C@H]3CC[C@@H](OCCNC(=O)[C@H]4CC(=O)N(C)[C@@H]4c4cccnc4)CC3)CC2)(C(=O)N2C[C@@H]3C[C@H]2CN3c2cc(C(F)(F)F)ncn2)C1] |
| I-30 | 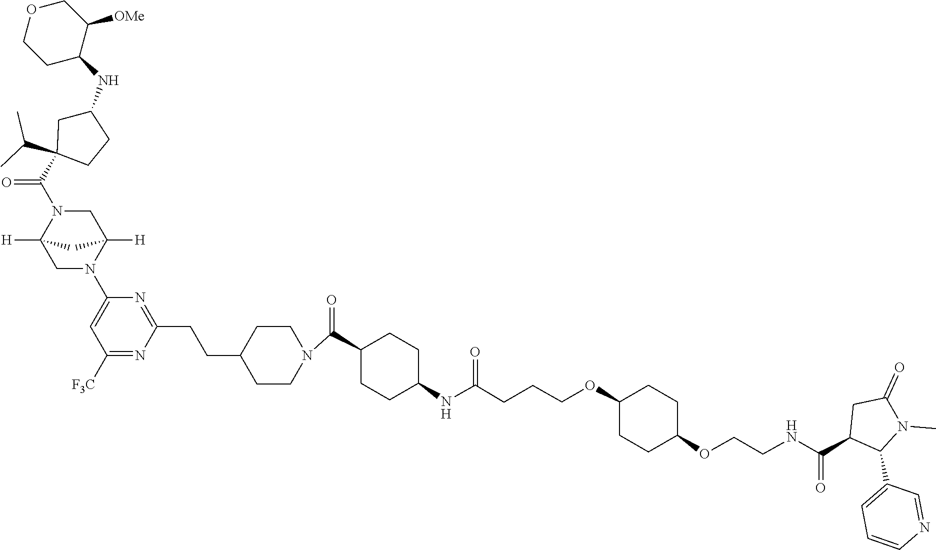 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4CC[C@@H](NC(=O)CCCO[C@H]5CC[C@@H](OCCNC(=O)[C@H]6CC(=O)N(C)[C@@H]6c6cccnc6)CC5)CC4)CC3)n2)(C(C)C)C1] |
| I-31 | 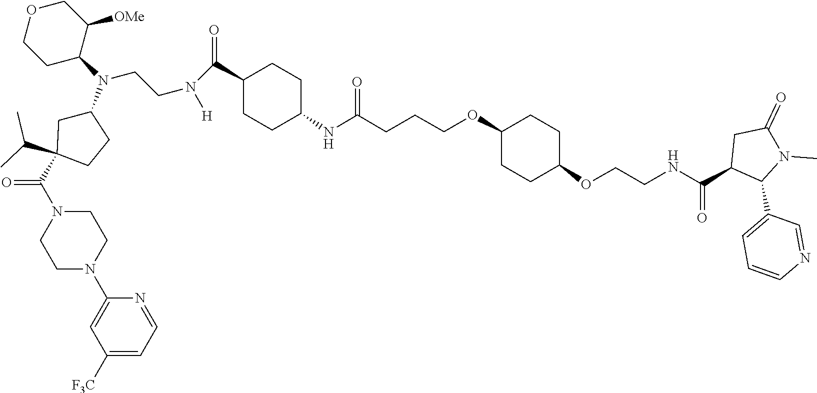 [SMILES: CO[C@@H]1COCC[C@@H]1N(CCNC(=O)[C@H]1CC[C@H](NC(=O)CCCO[C@H]2CC[C@@H](OCCNC(=O)[C@H]3CC(=O)N(C)[C@@H]3c3cccnc3)CC2)CC1)[C@@H]1CC[C@@](C(=O)N2CCN(c3cc(C(F)(F)F)ccn3)CC2)(C(C)C)C1] |
| I-32 | 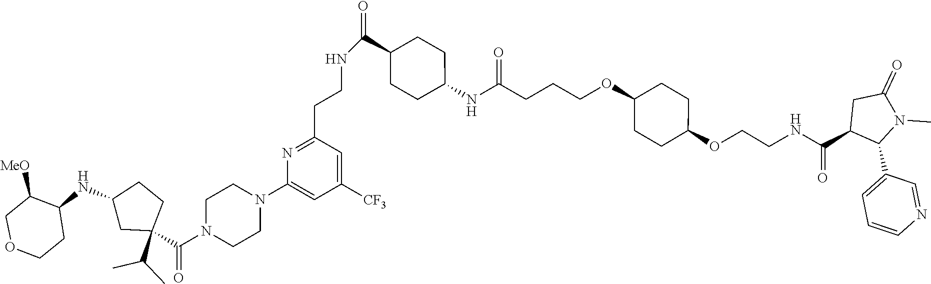 [SMILES: CO[C@@H]1COCC[C@@H]1N[C@@H]1CC[C@@](C(=O)N2CCN(c3cc(C(F)(F)F)cc(CCNC(=O)[C@H]4CC[C@H](NC(=O)CCCO[C@H]5CC[C@@H](OCCNC(=O)[C@H]6CC(=O)N(C)[C@@H]6c6cccnc6)CC5)CC4)n3)CC2)(C(C)C)C1] |
| I-33 | 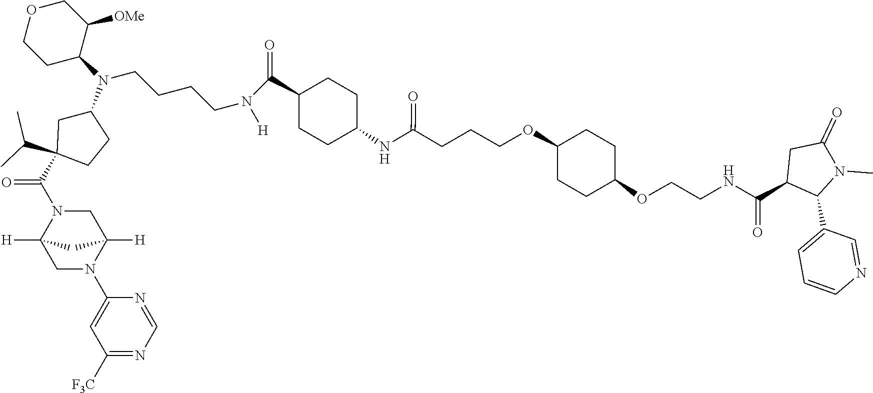 [SMILES: CO[C@@H]1COCC[C@@H]1N(CCCCNC(=O)[C@H]1CC[C@H](NC(=O)CCCO[C@H]2CC[C@@H](OCCNC(=O)[C@H]3CC(=O)N(C)[C@@H]3c3cccnc3)CC2)CC1)[C@@H]1CC[C@@](C(=O)N2C[C@@H]3C[C@H]2CN3c2cc(C(F)(F)F)ncn2)(C(C)C)C1] |
| I-34 | 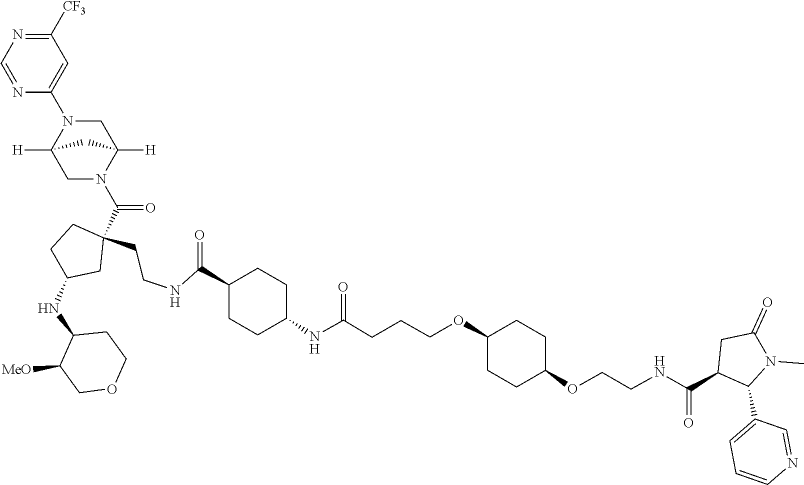 [SMILES: CO[C@@H]1COCC[C@@H]1N[C@@H]1CC[C@](CCNC(=O)[C@H]2CC[C@H](NC(=O)CCCO[C@H]3CC[C@@H](OCCNC(=O)[C@H]4CC(=O)N(C)[C@@H]4c4cccnc4)CC3)CC2)(C(=O)N2C[C@@H]3C[C@H]2CN3c2cc(C(F)(F)F)ncn2)C1] |
| I-35 | 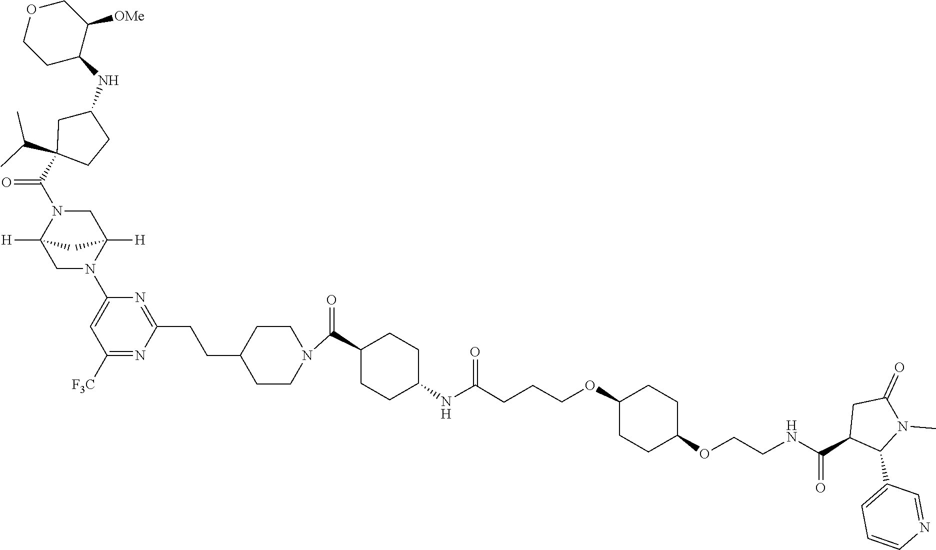 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4CC[C@H](NC(=O)CCCO[C@H]5CC[C@@H](OCCNC(=O)[C@H]6CC(=O)N(C)[C@@H]6c6cccnc6)CC5)CC4)CC3)n2)(C(C)C)C1] |
| I-36 | 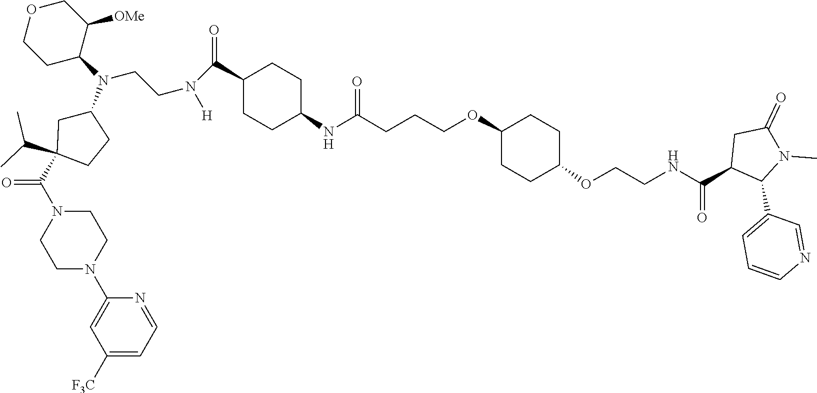 [SMILES: CO[C@@H]1COCC[C@@H]1N(CCNC(=O)[C@H]1CC[C@@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2CCN(c3cc(C(F)(F)F)ccn3)CC2)(C(C)C)C1] |
| I-37 | 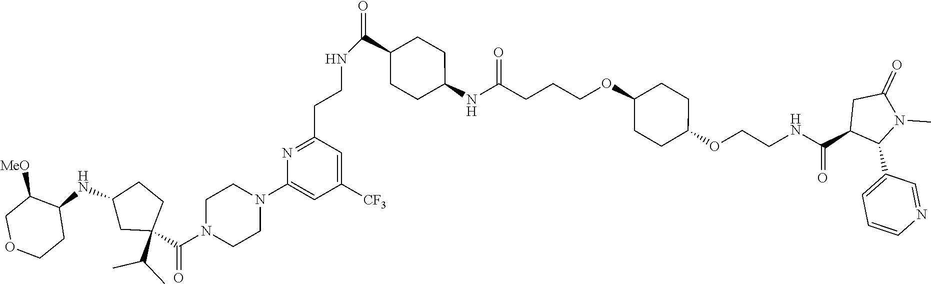 [SMILES: CO[C@@H]1COCC[C@@H]1N[C@@H]1CC[C@@](C(=O)N2CCN(c3cc(C(F)(F)F)cc(CCNC(=O)[C@H]4CC[C@@H](NC(=O)CCCO[C@H]5CC[C@H](OCCNC(=O)[C@H]6CC(=O)N(C)[C@@H]6c6cccnc6)CC5)CC4)n3)CC2)(C(C)C)C1] |
| I-38 | 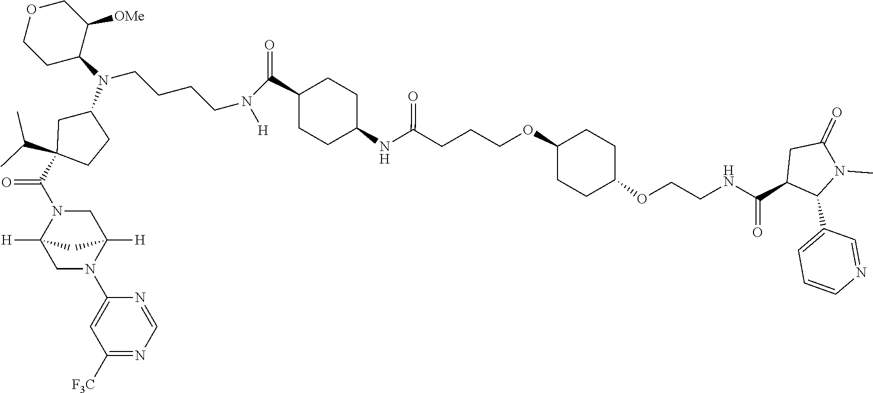 [SMILES: CO[C@@H]1COCC[C@@H]1N(CCCCNC(=O)[C@H]1CC[C@@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2C[C@@H]3C[C@H]2CN3c2cc(C(F)(F)F)ncn2)(C(C)C)C1] |
| I-39 | 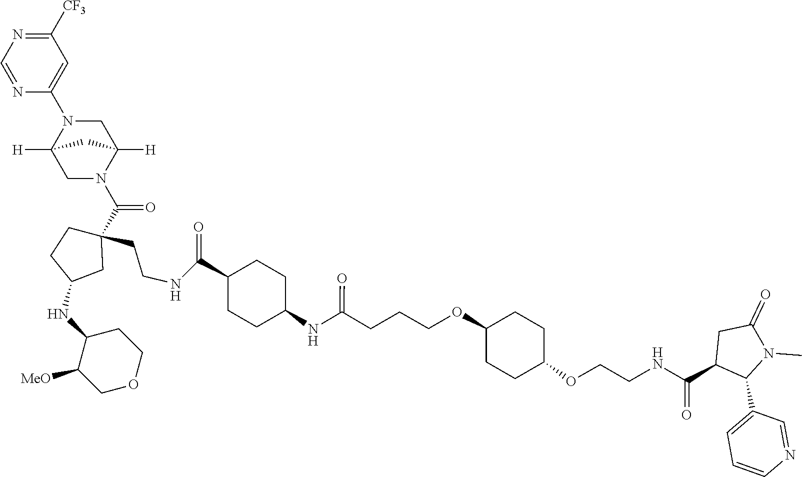 [SMILES: CO[C@@H]1COCC[C@@H]1N[C@@H]1CC[C@](CCNC(=O)[C@H]2CC[C@@H](NC(=O)CCCO[C@H]3CC[C@H](OCCNC(=O)[C@H]4CC(=O)N(C)[C@@H]4c4cccnc4)CC3)CC2)(C(=O)N2C[C@@H]3C[C@H]2CN3c2cc(C(F)(F)F)ncn2)C1] |
| I-40 | 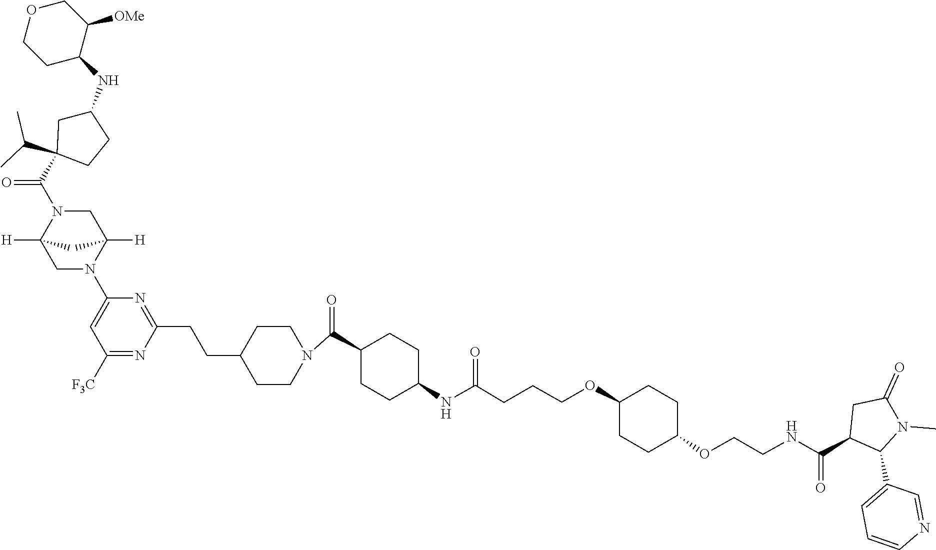 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4CC[C@@H](NC(=O)CCCO[C@H]5CC[C@H](OCCNC(=O)[C@H]6CC(=O)N(C)[C@@H]6c6cccnc6)CC5)CC4)CC3)n2)(C(C)C)C1] |
| I-41 | 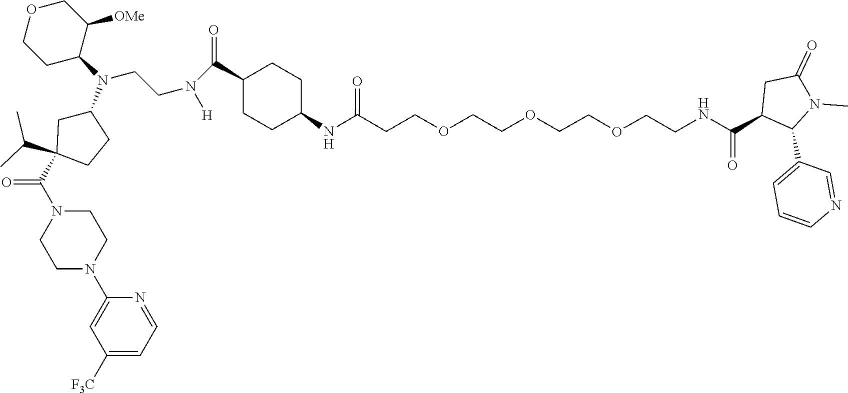 [SMILES: CO[C@@H]1COCC[C@@H]1N(CCNC(=O)[C@H]1CC[C@@H](NC(=O)CCOCCOCCOCCNC(=O)[C@H]2CC(=O)N(C)[C@@H]2c2cccnc2)CC1)[C@@H]1CC[C@@](C(=O)N2CCN(c3cc(C(F)(F)F)ccn3)CC2)(C(C)C)C1] |
| I-42 | 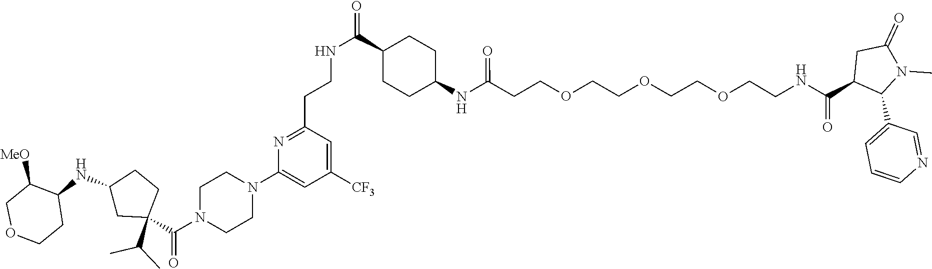 [SMILES: CO[C@@H]1COCC[C@@H]1N[C@@H]1CC[C@@](C(=O)N2CCN(c3cc(C(F)(F)F)cc(CCNC(=O)[C@H]4CC[C@@H](NC(=O)CCOCCOCCOCCNC(=O)[C@H]5CC(=O)N(C)[C@@H]5c5cccnc5)CC4)n3)CC2)(C(C)C)C1] |
| I-43 | 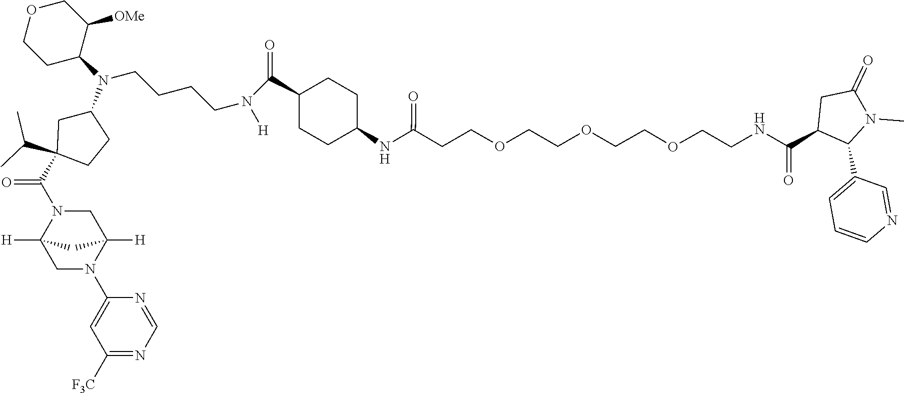 [SMILES: CO[C@@H]1COCC[C@@H]1N(CCCCNC(=O)[C@H]1CC[C@@H](NC(=O)CCOCCOCCOCCNC(=O)[C@H]2CC(=O)N(C)[C@@H]2c2cccnc2)CC1)[C@@H]1CC[C@@](C(=O)N2C[C@@H]3C[C@H]2CN3c2cc(C(F)(F)F)ncn2)(C(C)C)C1] |
| I-44 | 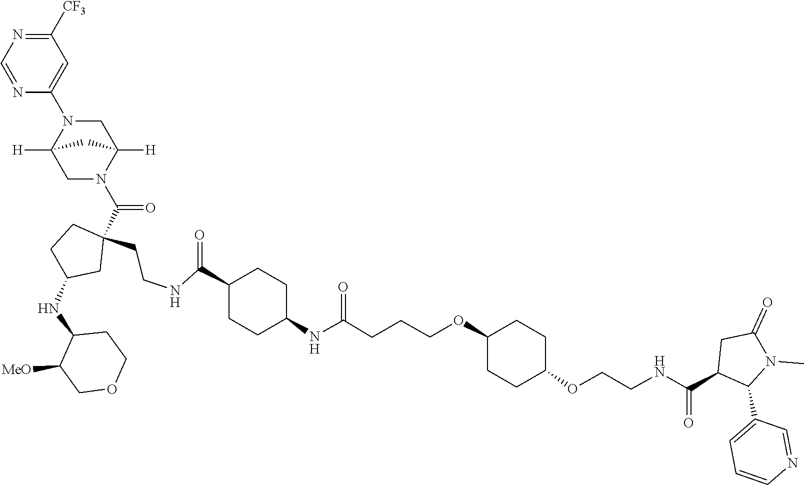 [SMILES: CO[C@@H]1COCC[C@@H]1N[C@@H]1CC[C@](CCNC(=O)[C@H]2CC[C@@H](NC(=O)CCCO[C@H]3CC[C@H](OCCNC(=O)[C@H]4CC(=O)N(C)[C@@H]4c4cccnc4)CC3)CC2)(C(=O)N2C[C@@H]3C[C@H]2CN3c2cc(C(F)(F)F)ncn2)C1] |
| I-45 | 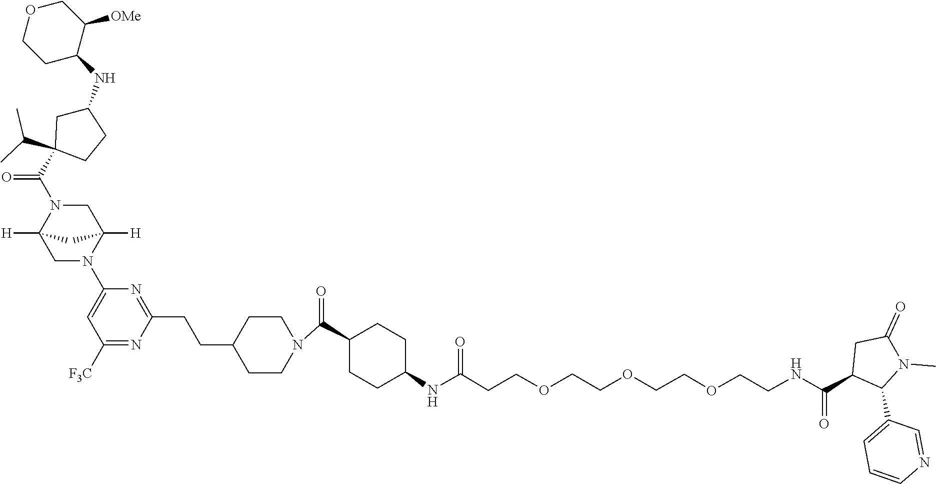 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4CC[C@@H](NC(=O)CCOCCOCCOCCNC(=O)[C@H]5CC(=O)N(C)[C@@H]5c5cccnc5)CC4)CC3)n2)(C(C)C)C1] |
| I-46 | 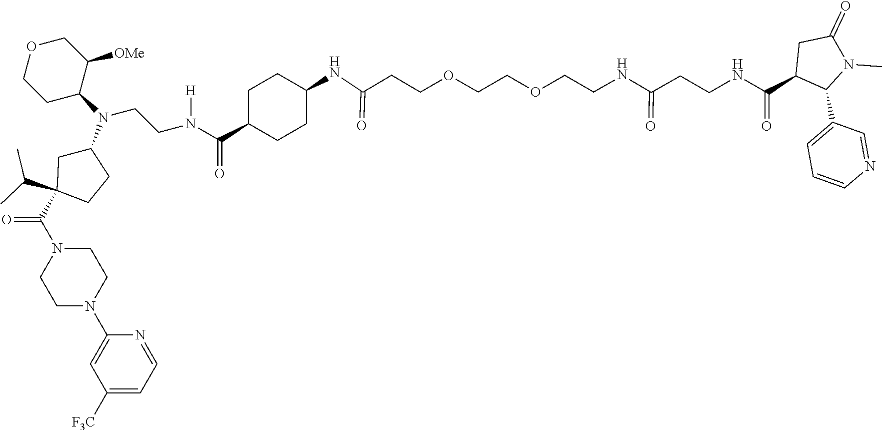 [SMILES: CO[C@@H]1COCC[C@@H]1N(CCNC(=O)[C@H]1CC[C@@H](NC(=O)CCOCCOCCNC(=O)CCNC(=O)[C@H]2CC(=O)N(C)[C@@H]2c2cccnc2)CC1)[C@@H]1CC[C@@](C(=O)N2CCN(c3cc(C(F)(F)F)ccn3)CC2)(C(C)C)C1] |
| I-47 | 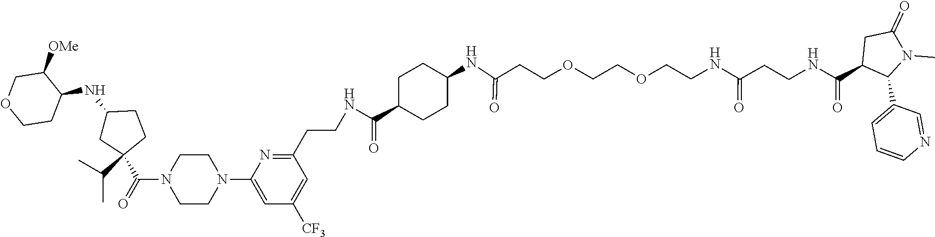 [SMILES: CO[C@@H]1COCC[C@@H]1N[C@@H]1CC[C@@](C(=O)N2CCN(c3cc(C(F)(F)F)cc(CCNC(=O)[C@H]4CC[C@@H](NC(=O)CCOCCOCCNC(=O)CCNC(=O)[C@H]5CC(=O)N(C)[C@@H]5c5cccnc5)CC4)n3)CC2)(C(C)C)C1] |
| I-48 | 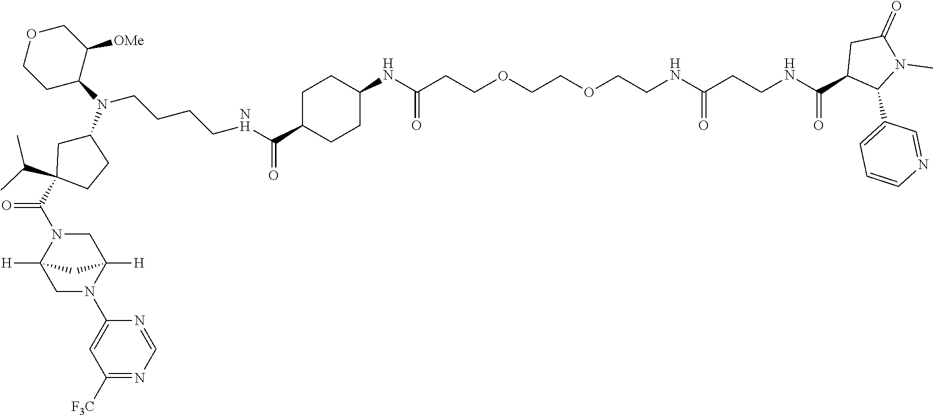 [SMILES: CO[C@@H]1COCC[C@@H]1N(CCCCNC(=O)[C@H]1CC[C@@H](NC(=O)CCOCCOCCNC(=O)CCNC(=O)[C@H]2CC(=O)N(C)[C@@H]2c2cccnc2)CC1)[C@@H]1CC[C@@](C(=O)N2C[C@@H]3C[C@H]2CN3c2cc(C(F)(F)F)ncn2)(C(C)C)C1] |
| I-49 | 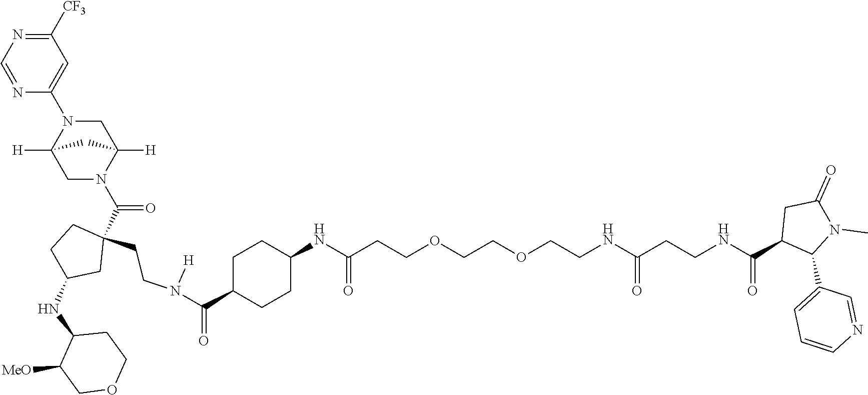 [SMILES: CO[C@@H]1COCC[C@@H]1N[C@@H]1CC[C@](CCNC(=O)[C@H]2CC[C@@H](NC(=O)CCOCCOCCNC(=O)CCNC(=O)[C@H]3CC(=O)N(C)[C@@H]3c3cccnc3)CC2)(C(=O)N2C[C@@H]3C[C@H]2CN3c2cc(C(F)(F)F)ncn2)C1] |
| I-50 | 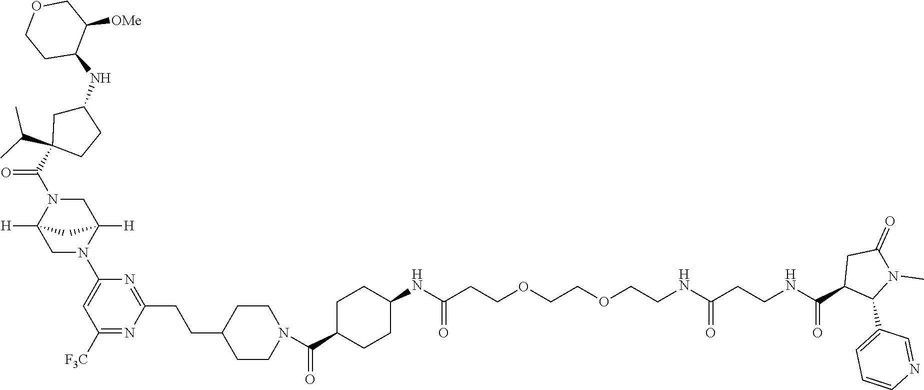 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4CC[C@@H](NC(=O)CCOCCOCCNC(=O)CCNC(=O)[C@H]5CC(=O)N(C)[C@@H]5c5cccnc5)CC4)CC3)n2)(C(C)C)C1] |
| I-51 | 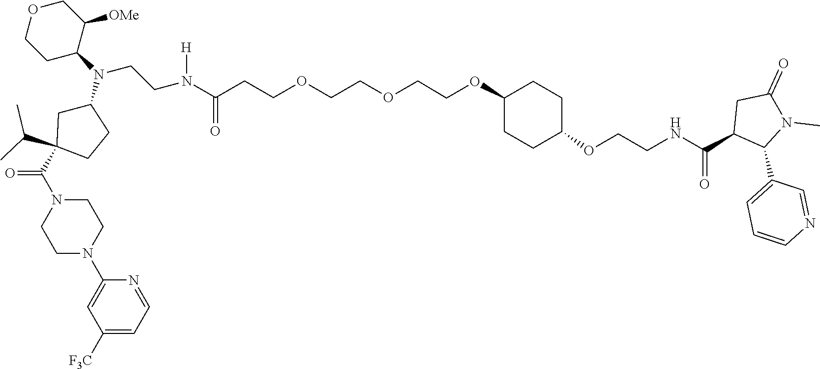 [SMILES: CO[C@@H]1COCC[C@@H]1N(CCNC(=O)CCOCCOCCO[C@H]1CC[C@H](OCCNC(=O)[C@H]2CC(=O)N(C)[C@@H]2c2cccnc2)CC1)[C@@H]1CC[C@@](C(=O)N2CCN(c3cc(C(F)(F)F)ccn3)CC2)(C(C)C)C1] |
| I-52 | 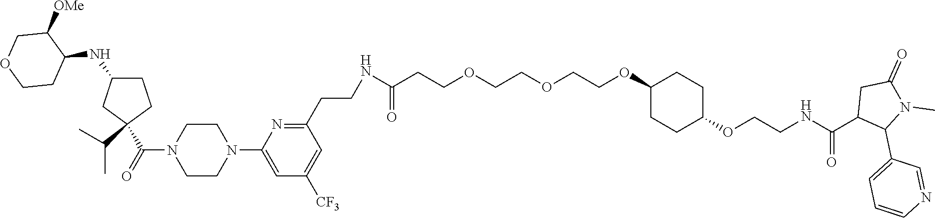 [SMILES: CO[C@@H]1COCC[C@@H]1N[C@@H]1CC[C@@](C(=O)N2CCN(c3cc(C(F)(F)F)cc(CCNC(=O)CCOCCOCCO[C@H]4CC[C@H](OCCNC(=O)C5CC(=O)N(C)C5c5cccnc5)CC4)n3)CC2)(C(C)C)C1] |
| I-53 | 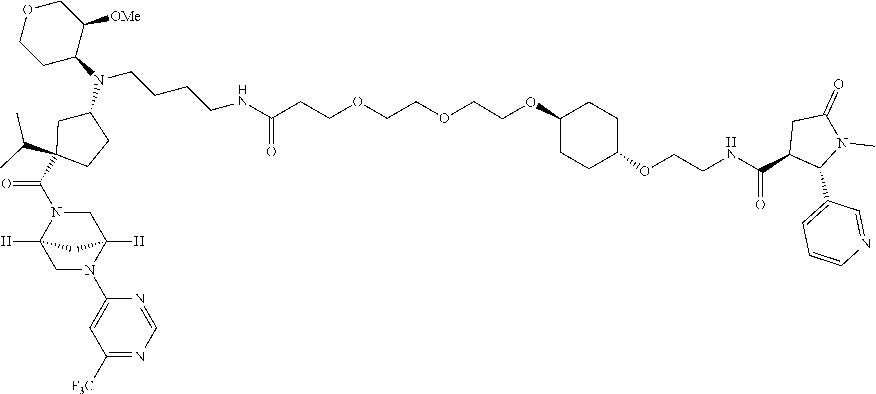 |
| I-54 | 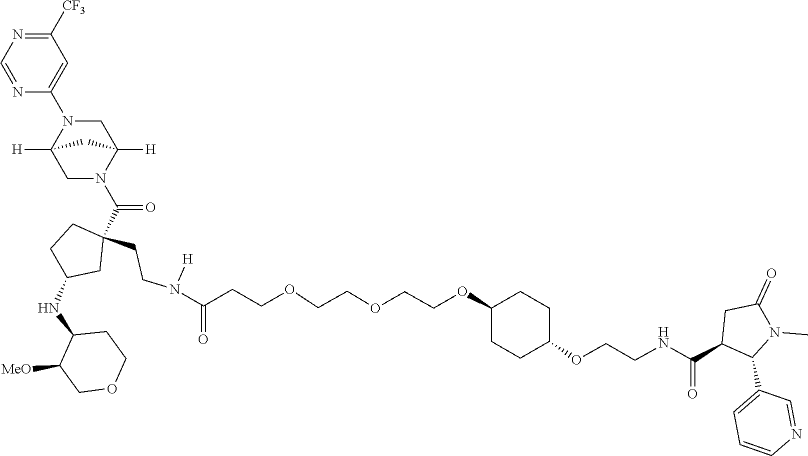 [SMILES: CO[C@@H]1COCC[C@@H]1N[C@@H]1CC[C@](CCNC(=O)CCOCCOCCO[C@H]2CC[C@H](OCCNC(=O)[C@H]3CC(=O)N(C)[C@@H]3c3cccnc3)CC2)(C(=O)N2C[C@@H]3C[C@H]2CN3c2cc(C(F)(F)F)ncn2)C1] |
| I-55 | 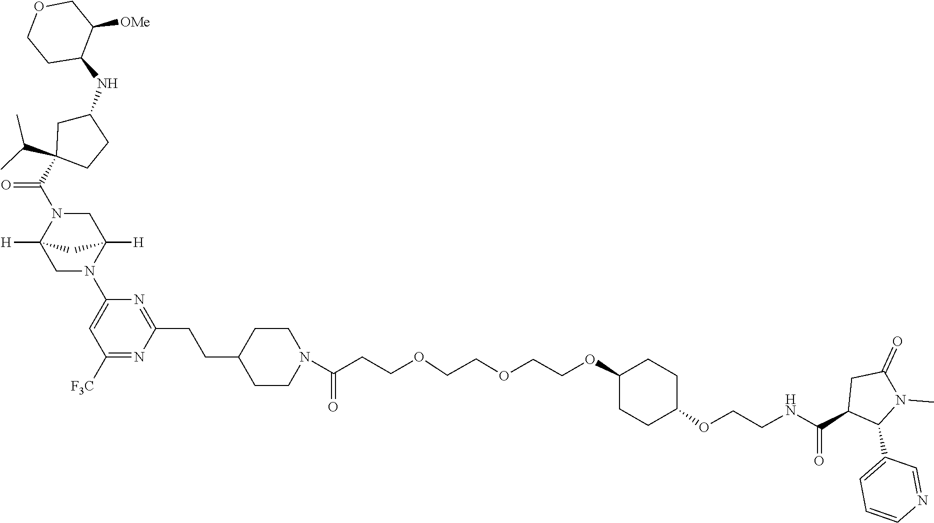 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)CCOCCOCCO[C@H]4CC[C@H](OCCNC(=O)[C@H]5CC(=O)N(C)[C@@H]5c5cccnc5)CC4)CC3)n2)(C(C)C)C1] |
| I-56 | 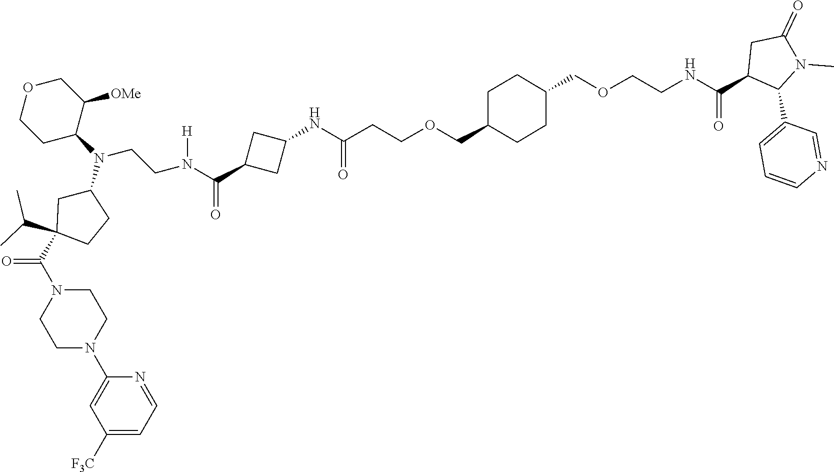 [SMILES: CO[C@@H]1COCC[C@@H]1N(CCNC(=O)[C@H]1C[C@H](NC(=O)CCOC[C@H]2CC[C@H](COCCNC(=O)[C@H]3CC(=O)N(C)[C@@H]3c3cccnc3)CC2)C1)[C@@H]1CC[C@@](C(=O)N2CCN(c3cc(C(F)(F)F)ccn3)CC2)(C(C)C)C1] |
| I-57 | 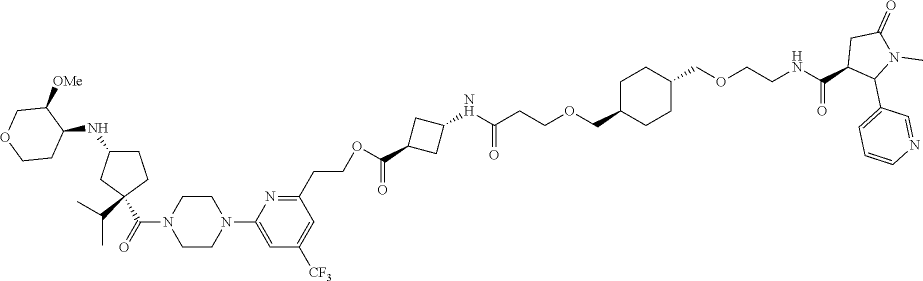 [SMILES: CO[C@@H]1COCC[C@@H]1N[C@@H]1CC[C@@](C(=O)N2CCN(c3cc(C(F)(F)F)cc(CCOC(=O)[C@H]4C[C@H](NC(=O)CCOC[C@H]5CC[C@H](COCCNC(=O)[C@H]6CC(=O)N(C)C6c6cccnc6)CC5)C4)n3)CC2)(C(C)C)C1] |
| I-58 | 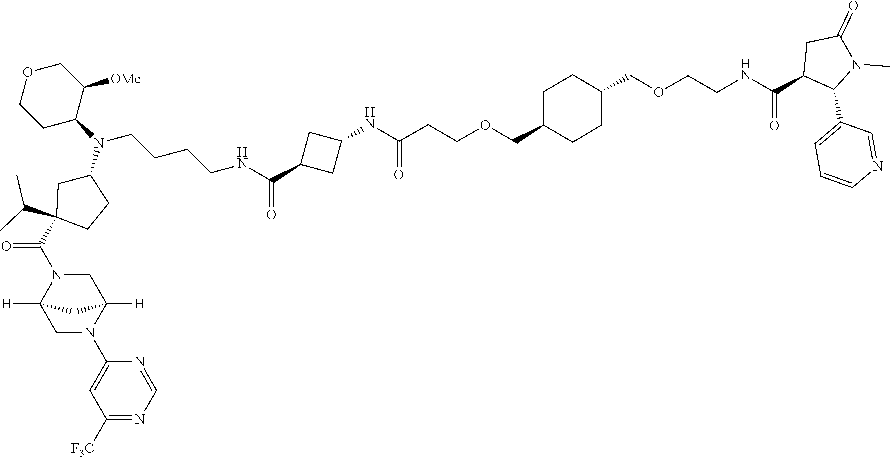 [SMILES: CO[C@@H]1COCC[C@@H]1N(CCCCNC(=O)[C@H]1C[C@H](NC(=O)CCOC[C@H]2CC[C@H](COCCNC(=O)[C@H]3CC(=O)N(C)[C@@H]3c3cccnc3)CC2)C1)[C@@H]1CC[C@@](C(=O)N2C[C@@H]3C[C@H]2CN3c2cc(C(F)(F)F)ncn2)(C(C)C)C1] |
| I-59 | 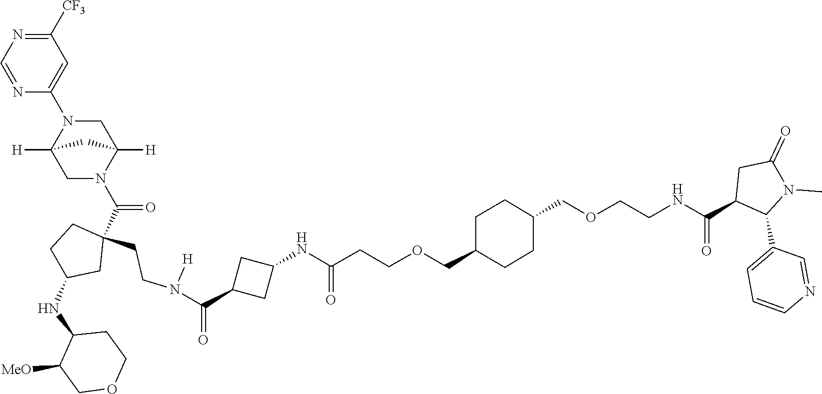 [SMILES: CO[C@@H]1COCC[C@@H]1N[C@@H]1CC[C@](CCNC(=O)[C@H]2C[C@H](NC(=O)CCOC[C@H]3CC[C@H](COCCNC(=O)[C@H]4CC(=O)N(C)[C@@H]4c4cccnc4)CC3)C2)(C(=O)N2C[C@@H]3C[C@H]2CN3c2cc(C(F)(F)F)ncn2)C1] |
| I-60 | 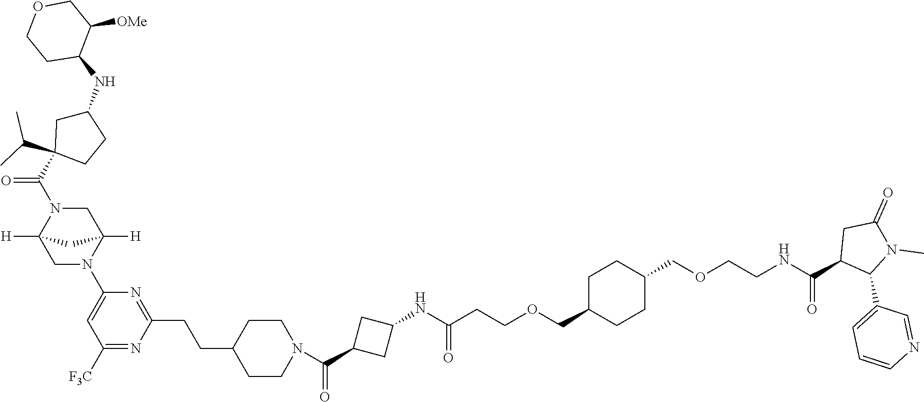 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4C[C@H](NC(=O)CCOC[C@H]5CC[C@H](COCCNC(=O)[C@H]6CC(=O)N(C)[C@@H]6c6cccnc6)CC5)C4)CC3)n2)(C(C)C)C1] |
| I-61 | 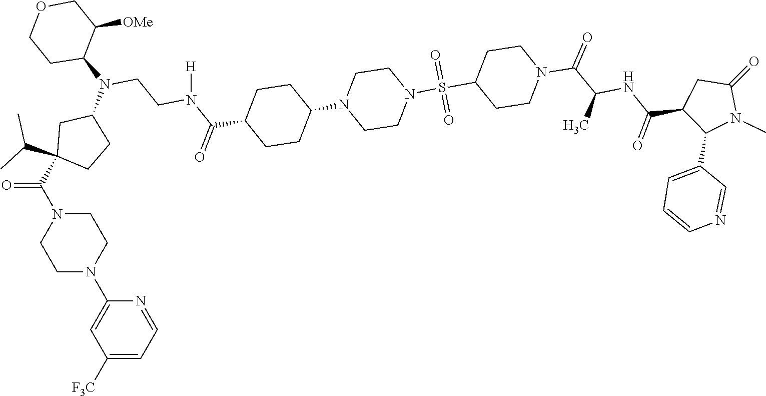 [SMILES: CO[C@@H]1COCC[C@@H]1N(CCNC(=O)[C@H]1CC[C@@H](N2CCN(S(=O)(=O)C3CCN(C(=O)[C@H](C)NC(=O)[C@H]4CC(=O)N(C)[C@@H]4c4cccnc4)CC3)CC2)CC1)[C@@H]1CC[C@@](C(=O)N2CCN(c3cc(C(F)(F)F)ccn3)CC2)(C(C)C)C1] |
| I-62 | 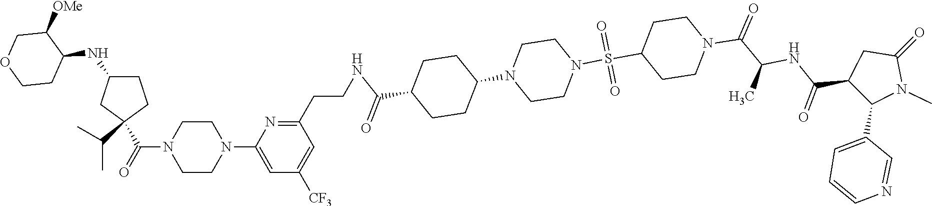 [SMILES: CO[C@@H]1COCC[C@@H]1N[C@@H]1CC[C@@](C(=O)N2CCN(c3cc(C(F)(F)F)cc(CCNC(=O)[C@H]4CC[C@@H](N5CCN(S(=O)(=O)C6CCN(C(=O)[C@H](C)NC(=O)[C@H]7CC(=O)N(C)[C@@H]7c7cccnc7)CC6)CC5)CC4)n3)CC2)(C(C)C)C1] |
| I-63 | 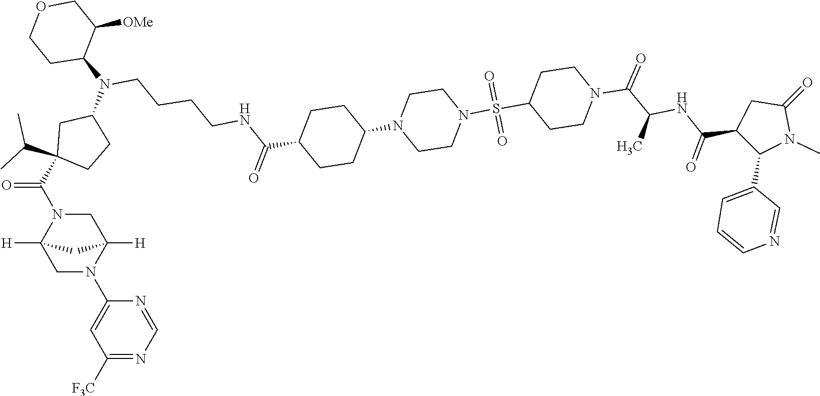 [SMILES: CO[C@@H]1COCC[C@@H]1N(CCCCNC(=O)[C@H]1CC[C@@H](N2CCN(S(=O)(=O)C3CCN(C(=O)[C@H](C)NC(=O)[C@H]4CC(=O)N(C)[C@@H]4c4cccnc4)CC3)CC2)CC1)[C@@H]1CC[C@@](C(=O)N2C[C@@H]3C[C@H]2CN3c2cc(C(F)(F)F)ncn2)(C(C)C)C1] |
| I-64 | 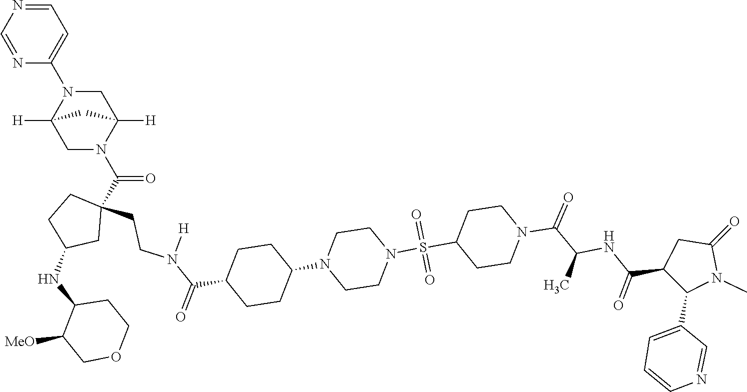 [SMILES: CO[C@@H]1COCC[C@@H]1N[C@@H]1CC[C@](CCNC(=O)[C@H]2CC[C@@H](N3CCN(S(=O)(=O)C4CCN(C(=O)[C@H](C)NC(=O)[C@H]5CC(=O)N(C)[C@@H]5c5cccnc5)CC4)CC3)CC2)(C(=O)N2C[C@@H]3C[C@H]2CN3c2ccncn2)C1] |
| I-65 | 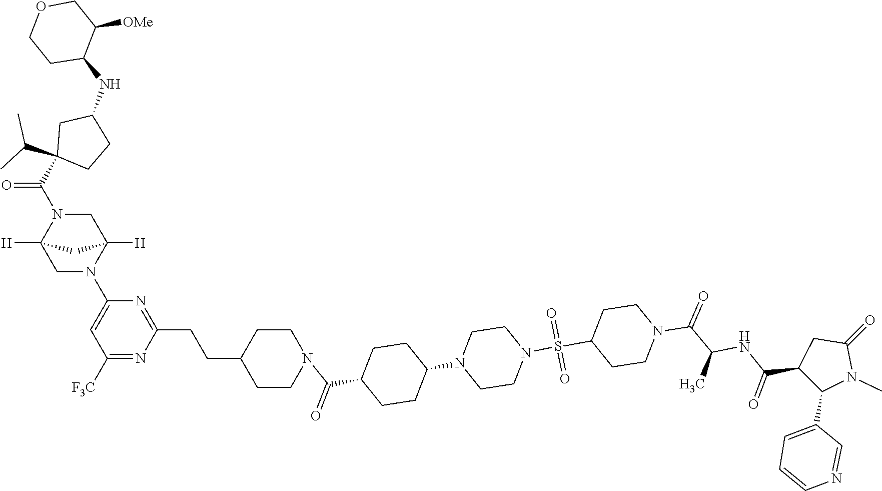 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4CC[C@@H](N5CCN(S(=O)(=O)C6CCN(C(=O)[C@H](C)NC(=O)[C@H]7CC(=O)N(C)[C@@H]7c7cccnc7)CC6)CC5)CC4)CC3)n2)(C(C)C)C1] |
| I-66 | 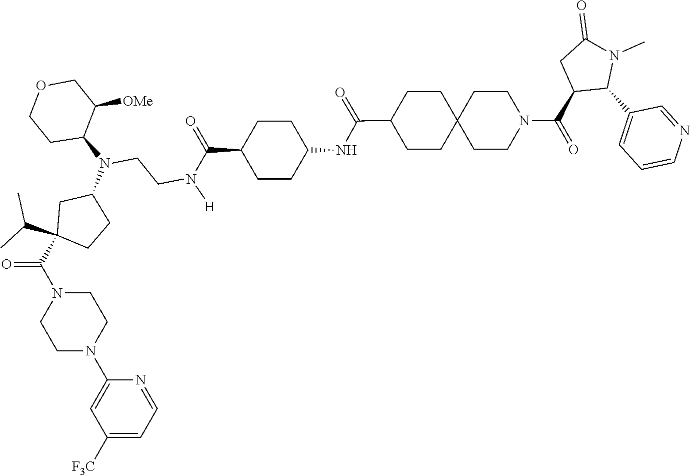 |
| I-67 | 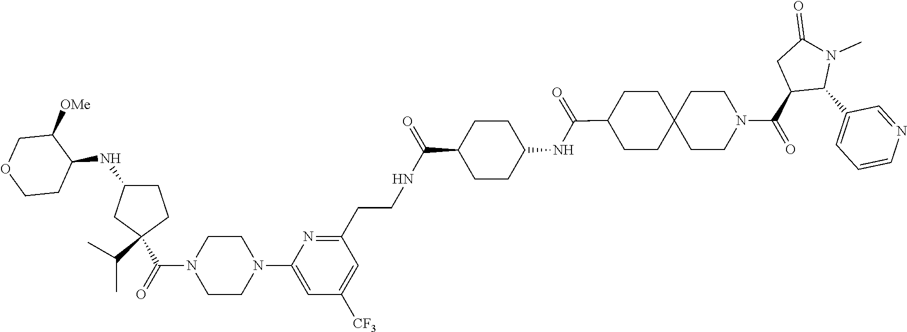 |
| I-68 | 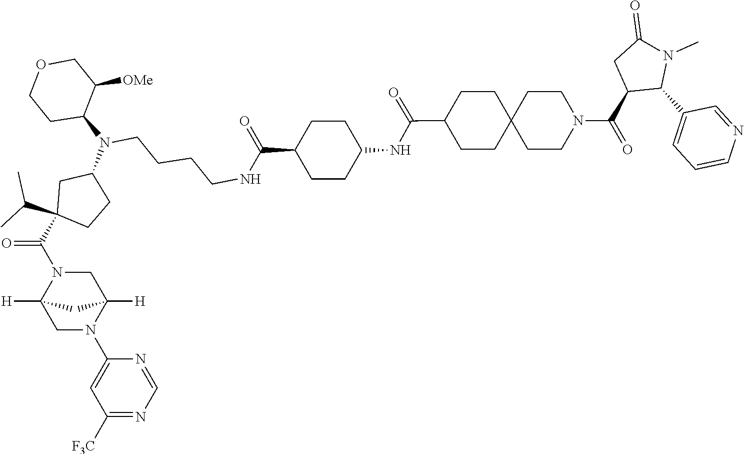 |
| I-69 | 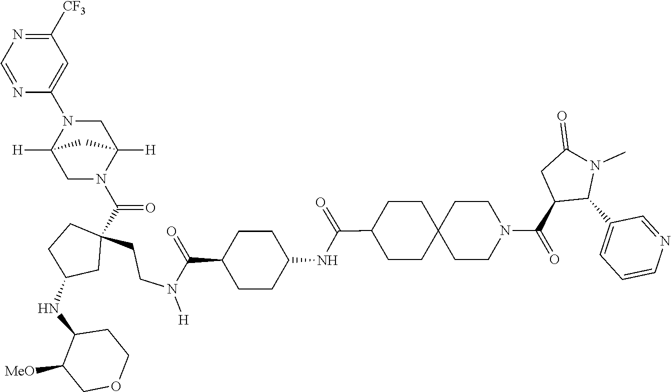 |
| I-70 | 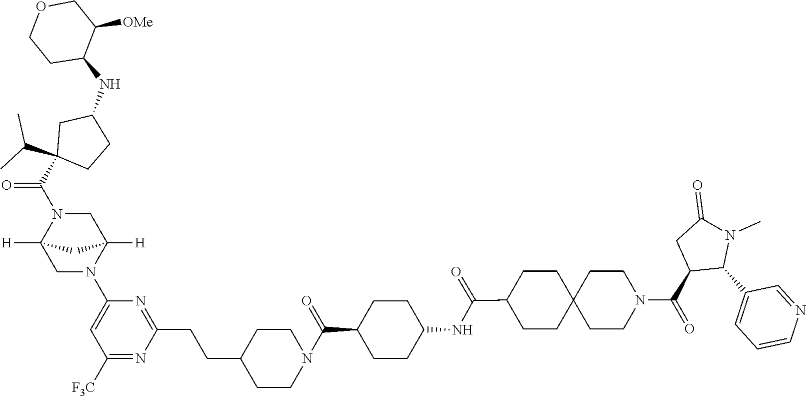 |
| I-71 | 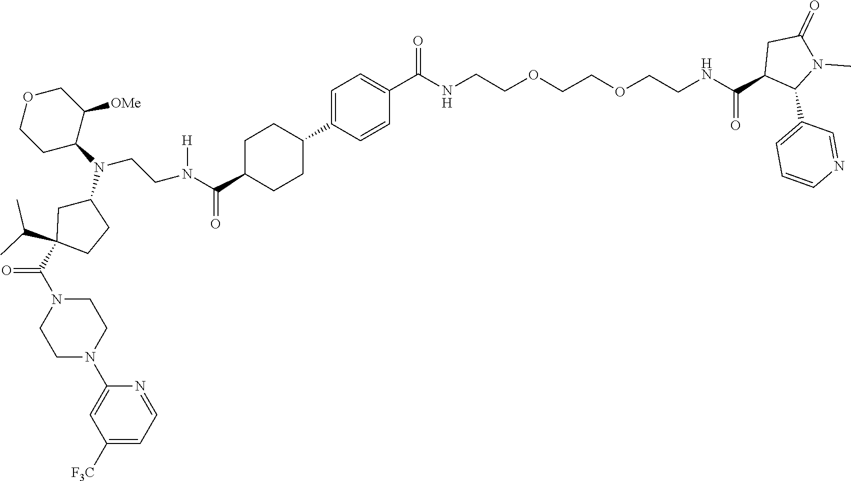 [SMILES: CO[C@@H]1COCC[C@@H]1N(CCNC(=O)[C@H]1CC[C@H](c2ccc(C(=O)NCCOCCOCCNC(=O)[C@H]3CC(=O)N(C)[C@@H]3c3cccnc3)cc2)CC1)[C@@H]1CC[C@@](C(=O)N2CCN(c3cc(C(F)(F)F)ccn3)CC2)(C(C)C)C1] |
| I-72 | 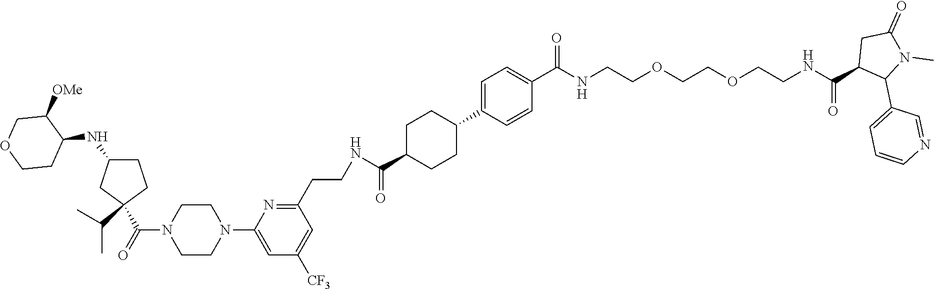 [SMILES: CO[C@@H]1COCC[C@@H]1N[C@@H]1CC[C@@](C(=O)N2CCN(c3cc(C(F)(F)F)cc(CCNC(=O)[C@H]4CC[C@H](c5ccc(C(=O)NCCOCCOCCNC(=O)[C@H]6CC(=O)N(C)C6c6cccnc6)cc5)CC4)n3)CC2)(C(C)C)C1] |
| I-73 | 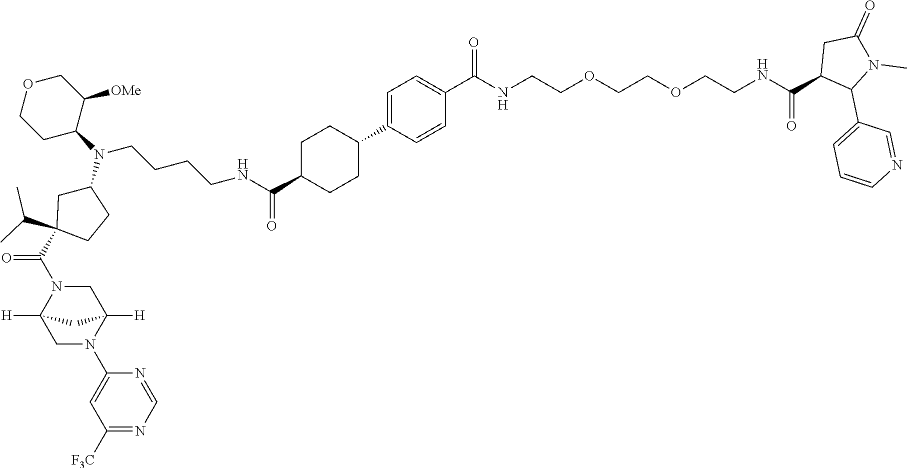 [SMILES: CO[C@@H]1COCC[C@@H]1N(CCCCNC(=O)[C@H]1CC[C@H](c2ccc(C(=O)NCCOCCOCCNC(=O)[C@H]3CC(=O)N(C)C3c3cccnc3)cc2)CC1)[C@@H]1CC[C@@](C(=O)N2C[C@@H]3C[C@H]2CN3c2cc(C(F)(F)F)ncn2)(C(C)C)C1] |
| I-74 | 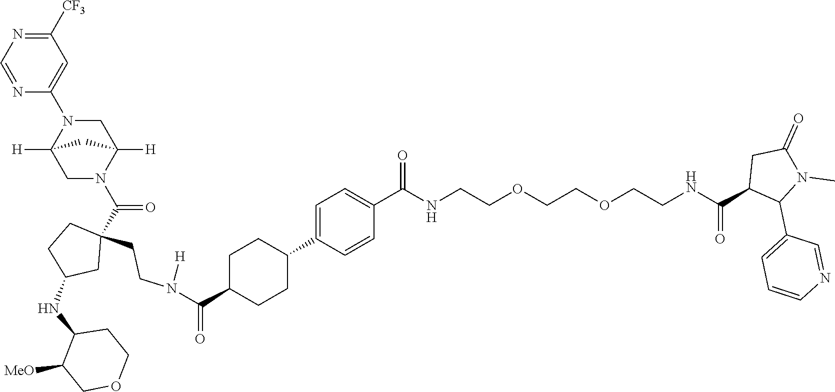 [SMILES: CO[C@@H]1COCC[C@@H]1N[C@@H]1CC[C@](CCNC(=O)[C@H]2CC[C@H](c3ccc(C(=O)NCCOCCOCCNC(=O)[C@H]4CC(=O)N(C)C4c4cccnc4)cc3)CC2)(C(=O)N2C[C@@H]3C[C@H]2CN3c2cc(C(F)(F)F)ncn2)C1] |
| I-75 | 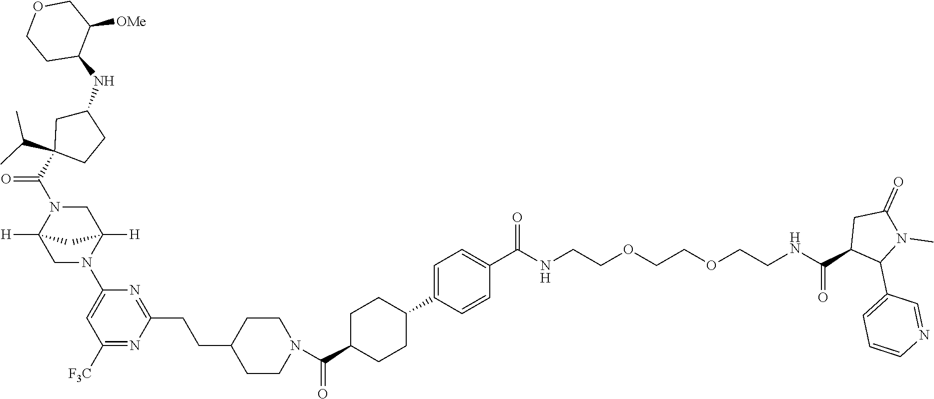 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)[C@H]4CC[C@H](c5ccc(C(=O)NCCOCCOCCNC(=O)[C@H]6CC(=O)N(C)C6c6cccnc6)cc5)CC4)CC3)n2)(C(C)C)C1] |
| I-76 | 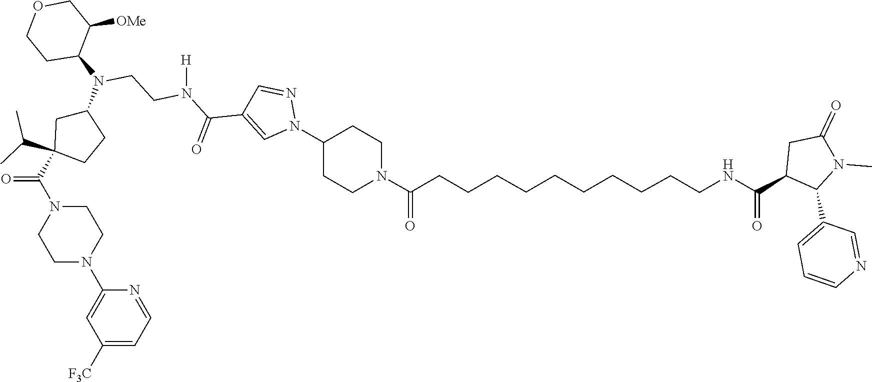 [SMILES: CO[C@@H]1COCC[C@@H]1N(CCNC(=O)c1cnn(C2CCN(C(=O)CCCCCCCCCCNC(=O)[C@H]3CC(=O)N(C)[C@@H]3c3cccnc3)CC2)c1)[C@@H]1CC[C@@](C(=O)N2CCN(c3cc(C(F)(F)F)ccn3)CC2)(C(C)C)C1] |
| I-77 | 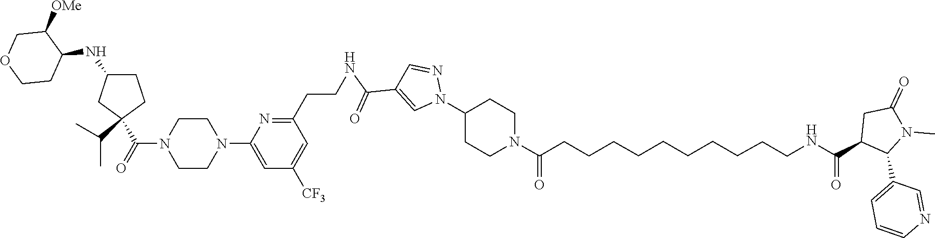 [SMILES: CO[C@@H]1COCC[C@@H]1N[C@@H]1CC[C@@](C(=O)N2CCN(c3cc(C(F)(F)F)cc(CCNC(=O)c4cnn(C5CCN(C(=O)CCCCCCCCCCNC(=O)[C@H]6CC(=O)N(C)[C@@H]6c6cccnc6)CC5)c4)n3)CC2)(C(C)C)C1] |
| I-78 | 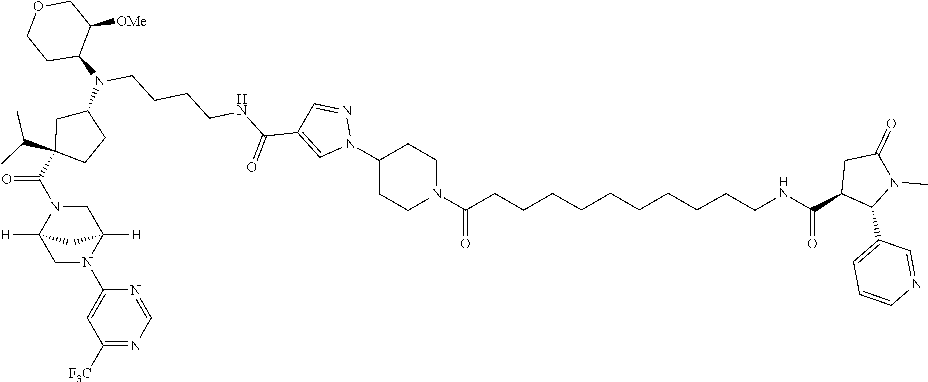 [SMILES: CO[C@@H]1COCC[C@@H]1N(CCCCNC(=O)c1cnn(C2CCN(C(=O)CCCCCCCCCCNC(=O)[C@H]3CC(=O)N(C)[C@@H]3c3cccnc3)CC2)c1)[C@@H]1CC[C@@](C(=O)N2C[C@@H]3C[C@H]2CN3c2cc(C(F)(F)F)ncn2)(C(C)C)C1] |
| I-79 | 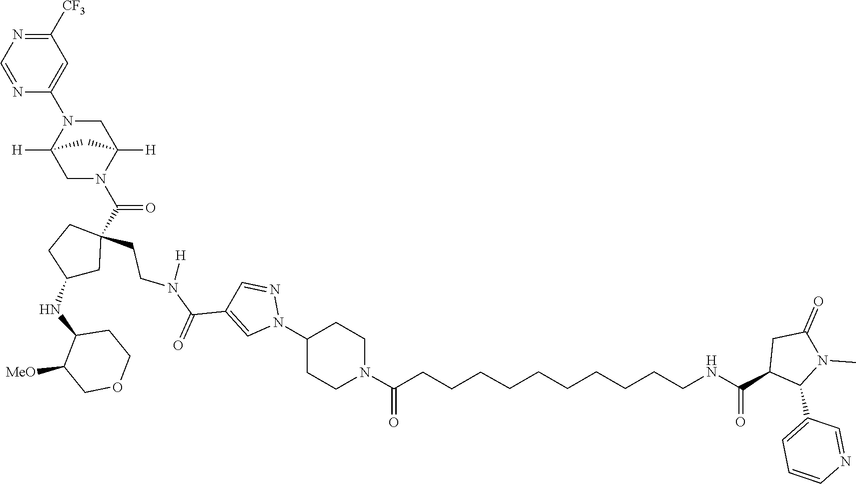 [SMILES: CO[C@@H]1COCC[C@@H]1N[C@@H]1CC[C@](CCNC(=O)c2cnn(C3CCN(C(=O)CCCCCCCCCCNC(=O)[C@H]4CC(=O)N(C)[C@@H]4c4cccnc4)CC3)c2)(C(=O)N2C[C@@H]3C[C@H]2CN3c2cc(C(F)(F)F)ncn2)C1] |
| I-80 | 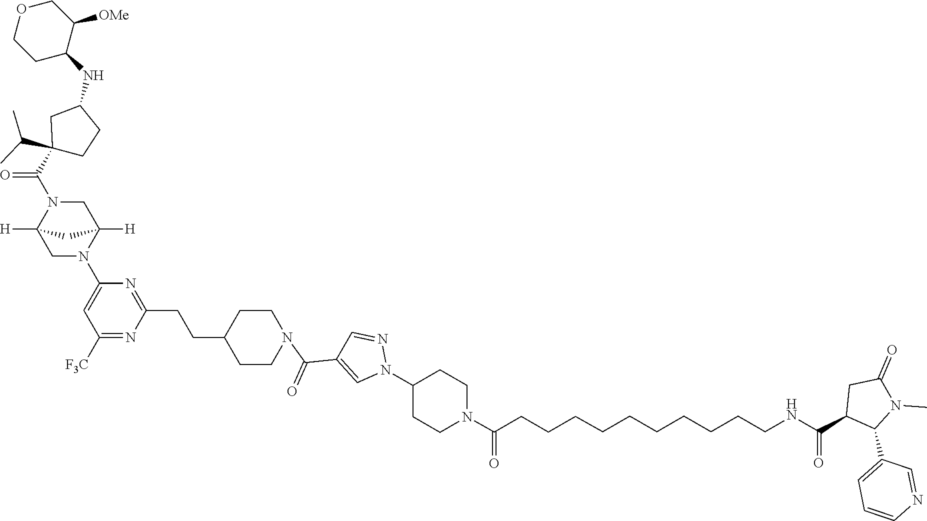 [SMILES: CO[C@@H]1COCC[C@@H]1N[C@@H]1CC[C@@](C(=O)N2C[C@@H]3C[C@H]2CN3c2cc(C(F)(F)F)nc(CCC3CCN(C(=O)c4cnn(C5CCN(C(=O)CCCCCCCCCCNC(=O)[C@H]6CC(=O)N(C)[C@@H]6c6cccnc6)CC5)c4)CC3)n2)(C(C)C)C1] |

TABLE 2
Exemplary Compounds

| Compound | |
|---|---|
| No. | Chemical Structure |
| II-1 | 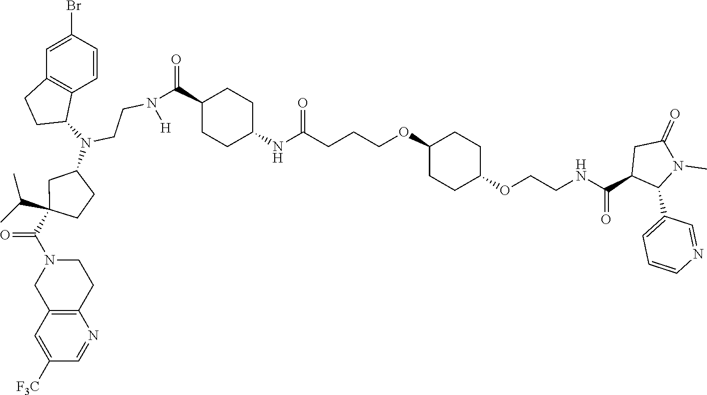 [SMILES: CC(C)[C@]1(C(=O)N2CCc3ncc(C(F)(F)F)cc3C2)CC[C@@H](N(CCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)[C@@H]2CCc3cc(Br)ccc32)C1] |
| II-2 | 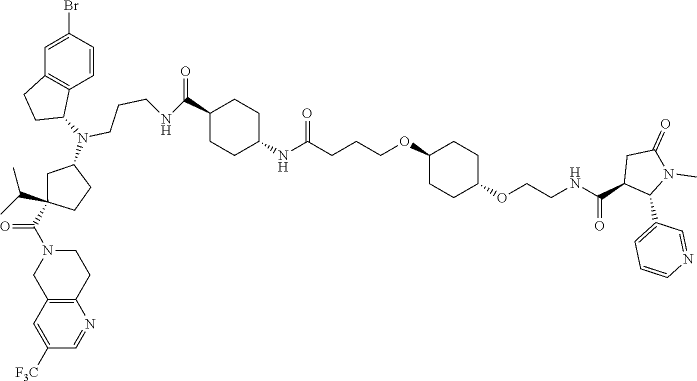 [SMILES: CC(C)[C@]1(C(=O)N2CCc3ncc(C(F)(F)F)cc3C2)CC[C@@H](N(CCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)[C@@H]2CCc3cc(Br)ccc32)C1] |
| II-3 | 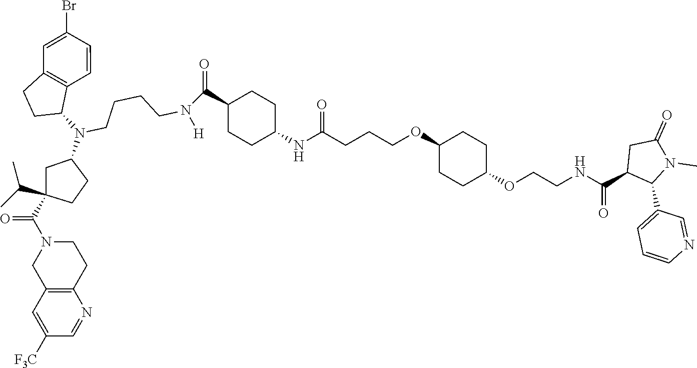 [SMILES: CC(C)[C@]1(C(=O)N2CCc3ncc(C(F)(F)F)cc3C2)CC[C@@H](N(CCCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)[C@@H]2CCc3cc(Br)ccc32)C1] |
| II-4 | 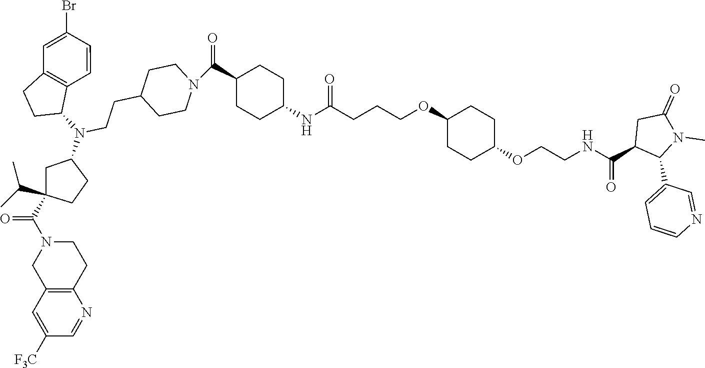 [SMILES: CC(C)[C@]1(C(=O)N2CCc3ncc(C(F)(F)F)cc3C2)CC[C@@H](N(CCC2CCN(C(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)[C@@H]2CCc3cc(Br)ccc32)C1] |
| II-5 | 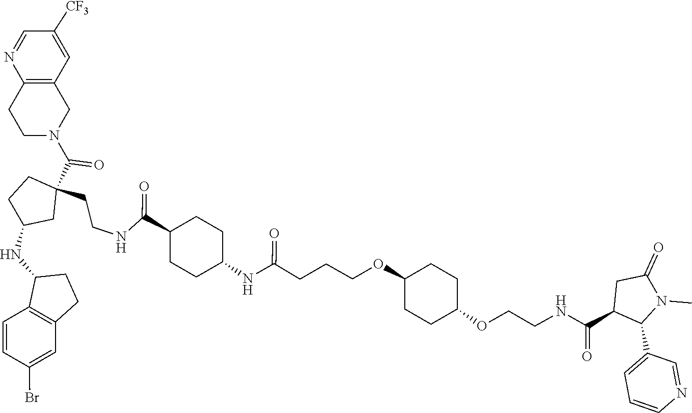 [SMILES: CN1C(=O)C[C@H](C(=O)NCCO[C@H]2CC[C@H](OCCCC(=O)N[C@H]3CC[C@H](C(=O)NCC[C@]4(C(=O)N5CCc6ncc(C(F)(F)F)cc6C5)CC[C@@H](N[C@@H]5CCc6cc(Br)ccc65)C4)CC3)CC2)[C@H]1c1cccnc1] |
| II-6 | 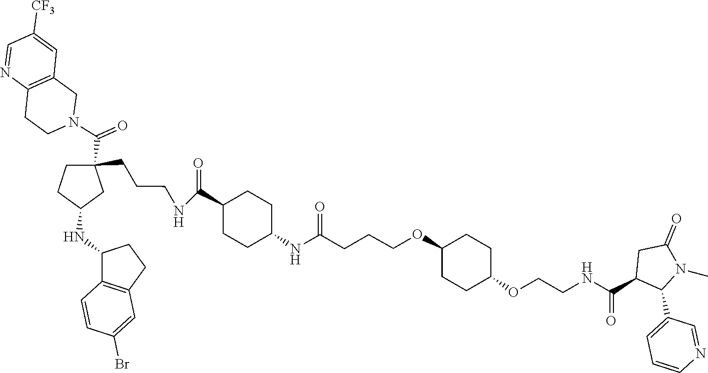 [SMILES: CN1C(=O)C[C@H](C(=O)NCCO[C@H]2CC[C@H](OCCCC(=O)N[C@H]3CC[C@H](C(=O)NCCC[C@]4(C(=O)N5CCc6ncc(C(F)(F)F)cc6C5)CC[C@@H](N[C@@H]5CCc6cc(Br)ccc65)C4)CC3)CC2)[C@H]1c1cccnc1] |
| II-7 | 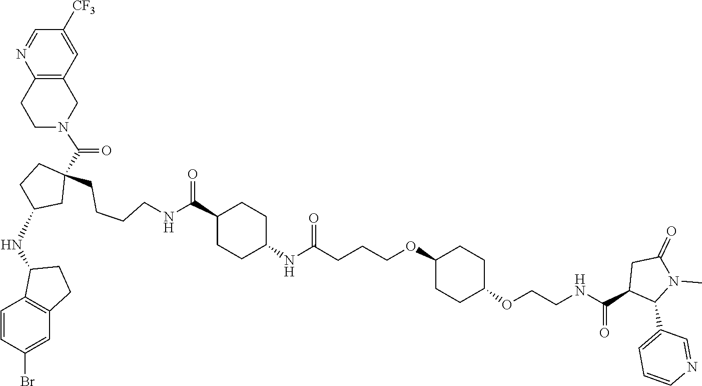 [SMILES: CN1C(=O)C[C@H](C(=O)NCCO[C@H]2CC[C@H](OCCCC(=O)N[C@H]3CC[C@H](C(=O)NCCCC[C@]4(C(=O)N5CCc6ncc(C(F)(F)F)cc6C5)CC[C@@H](N[C@@H]5CCc6cc(Br)ccc65)C4)CC3)CC2)[C@H]1c1cccnc1] |
| II-8 | 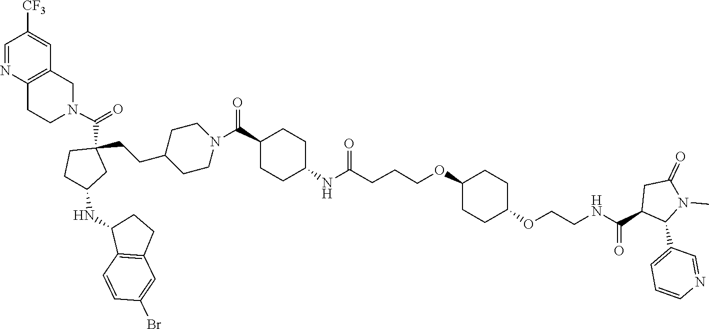 [SMILES: CN1C(=O)C[C@H](C(=O)NCCO[C@H]2CC[C@H](OCCCC(=O)N[C@H]3CC[C@H](C(=O)N4CCC(CC[C@]5(C(=O)N6CCc7ncc(C(F)(F)F)cc7C6)CC[C@@H](N[C@@H]6CCc7cc(Br)ccc76)C5)CC4)CC3)CC2)[C@H]1c1cccnc1] |
| II-9 | 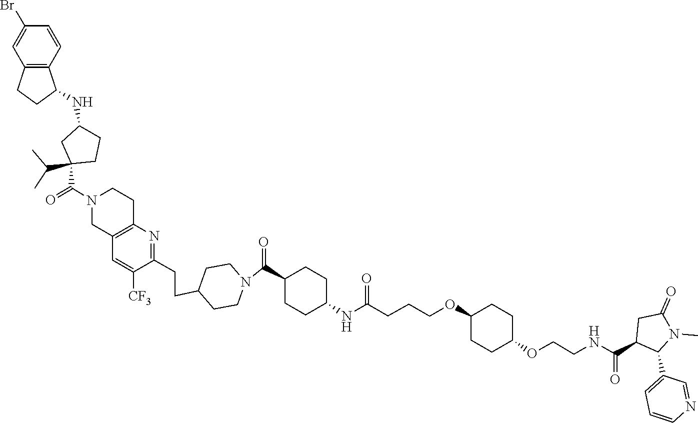 [SMILES: CC(C)[C@]1(C(=O)N2CCc3nc(CCC4CCN(C(=O)[C@H]5CC[C@H](NC(=O)CCCO[C@H]6CC[C@H](OCCNC(=O)[C@H]7CC(=O)N(C)[C@@H]7c7cccnc7)CC6)CC5)CC4)c(C(F)(F)F)cc3C2)CC[C@@H](N[C@@H]2CCc3cc(Br)ccc32)C1] |
| II-10 | 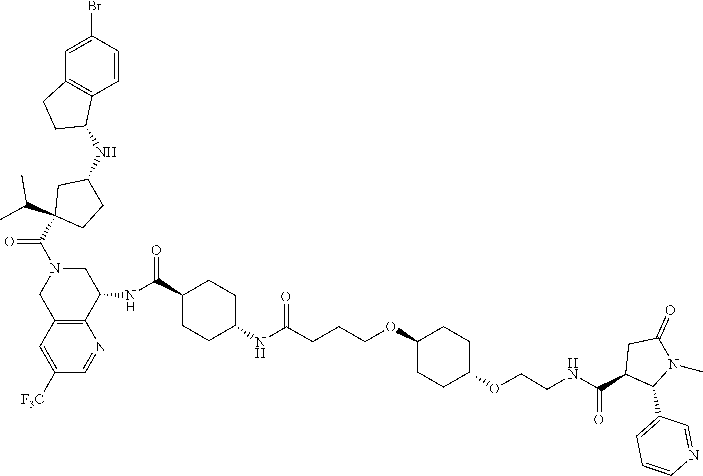 [SMILES: CC(C)[C@]1(C(=O)N2Cc3cc(C(F)(F)F)cnc3[C@@H](NC(=O)[C@H]3CC[C@H](NC(=O)CCCO[C@H]4CC[C@H](OCCNC(=O)[C@H]5CC(=O)N(C)[C@@H]5c5cccnc5)CC4)CC3)C2)CC[C@@H](N[C@@H]2CCc3cc(Br)ccc32)C1] |
| II-11 | 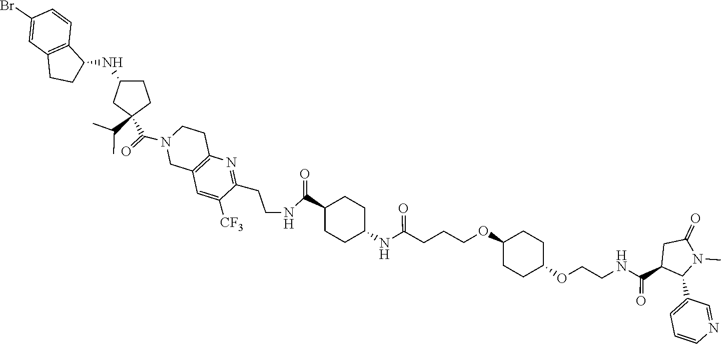 [SMILES: CC(C)[C@]1(C(=O)N2CCc3nc(CCNC(=O)[C@H]4CC[C@H](NC(=O)CCCO[C@H]5CC[C@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-12 | 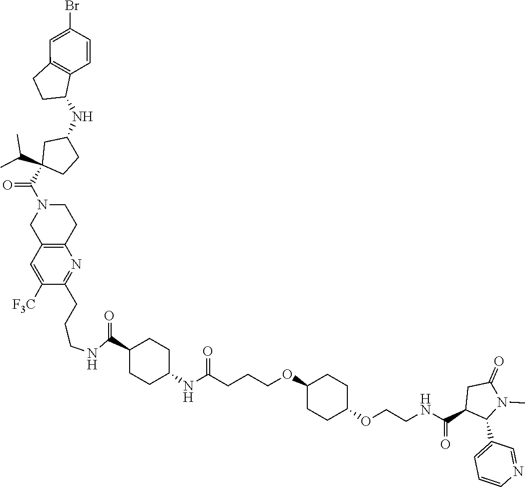 [SMILES: CC(C)[C@]1(C(=O)N2CCc3nc(CCCNC(=O)[C@H]4CC[C@H](NC(=O)CCCO[C@H]5CC[C@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-13 | 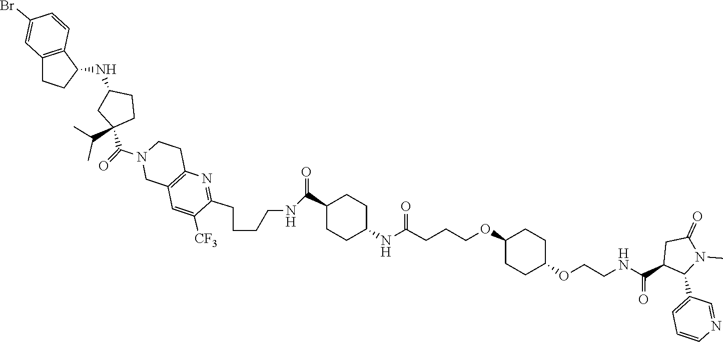 [SMILES: CC(C)[C@]1(C(=O)N2CCc3nc(CCCCNC(=O)[C@H]4CC[C@H](NC(=O)CCCO[C@H]5CC[C@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-14 | 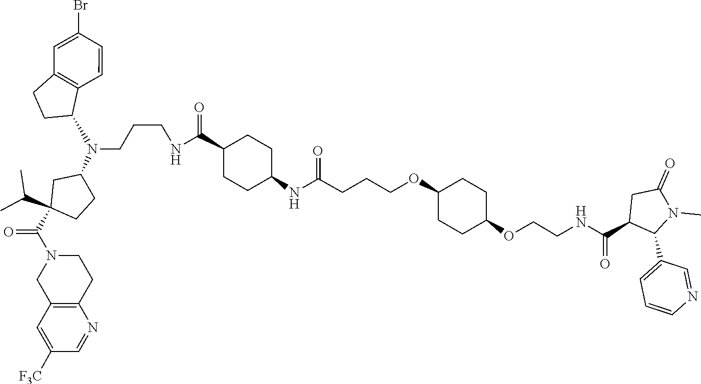 [SMILES: CC(C)[C@]1(C(=O)N2CCc3ncc(C(F)(F)F)cc3C2)CC[C@@H](N(CCCNC(=O)[C@H]2CC[C@@H](NC(=O)CCCO[C@H]3CC[C@@H](OCCNC(=O)[C@H]4CC(=O)N(C)[C@@H]4c4cccnc4)CC3)CC2)[C@@H]2CCc3cc(Br)ccc32)C1] |
| II-15 | 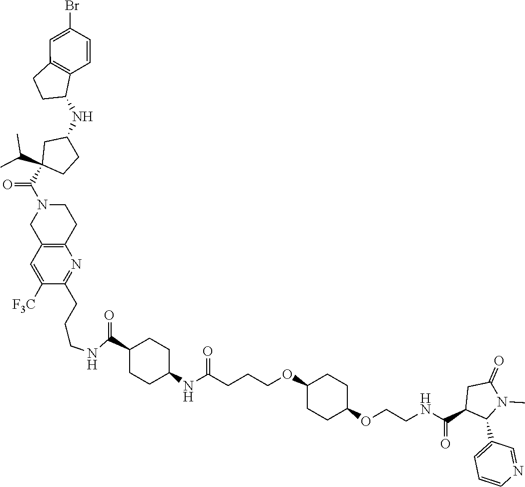 [SMILES: CC(C)[C@]1(C(=O)N2CCc3nc(CCCNC(=O)[C@H]4CC[C@@H](NC(=O)CCCO[C@H]5CC[C@@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-16 | 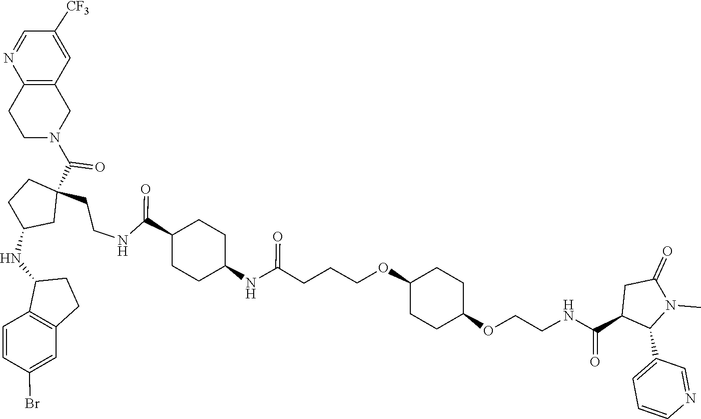 [SMILES: CN1C(=O)C[C@H](C(=O)NCCO[C@H]2CC[C@@H](OCCCC(=O)N[C@H]3CC[C@@H](C(=O)NCC[C@]4(C(=O)N5CCc6ncc(C(F)(F)F)cc6C5)CC[C@@H](N[C@@H]5CCc6cc(Br)ccc65)C4)CC3)CC2)[C@H]1c1cccnc1] |
| II-17 | 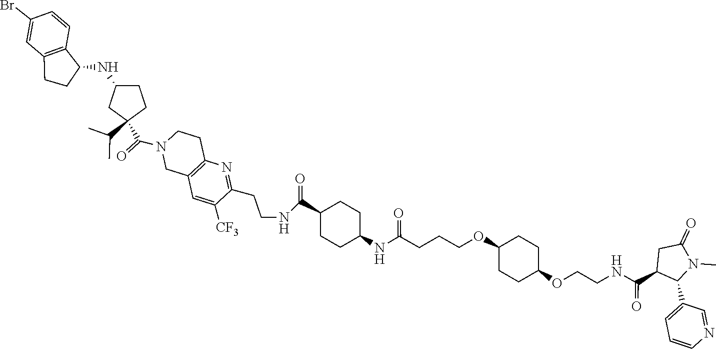 [SMILES: CC(C)[C@]1(C(=O)N2CCc3nc(CCNC(=O)[C@H]4CC[C@@H](NC(=O)CCCO[C@H]5CC[C@@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-18 | 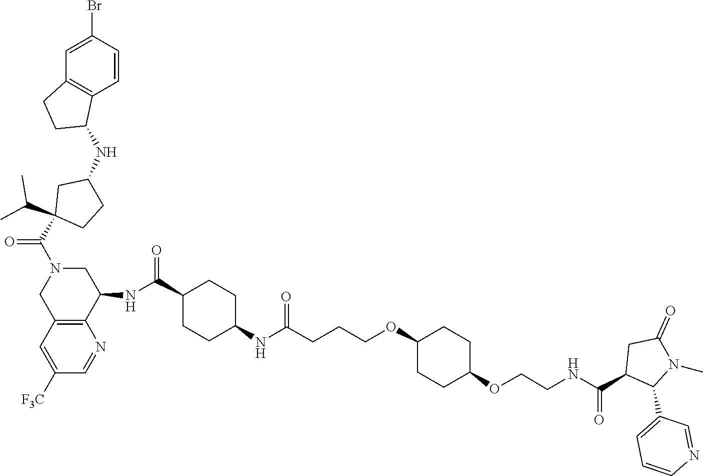 [SMILES: CC(C)[C@]1(C(=O)N2Cc3cc(C(F)(F)F)cnc3[C@H](NC(=O)[C@H]3CC[C@@H](NC(=O)CCCO[C@H]4CC[C@@H](OCCNC(=O)[C@H]5CC(=O)N(C)[C@@H]5c5cccnc5)CC4)CC3)C2)CC[C@@H](N[C@@H]2CCc3cc(Br)ccc32)C1] |
| II-19 | 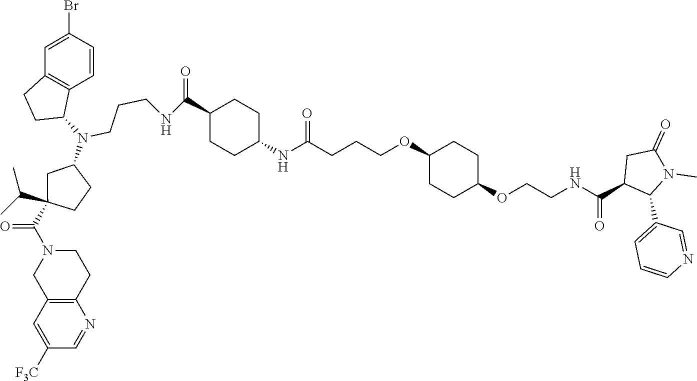 [SMILES: CC(C)[C@]1(C(=O)N2CCc3ncc(C(F)(F)F)cc3C2)CC[C@@H](N(CCCNC(=O)[C@H]2CC[C@H](NC(=O)CCCO[C@H]3CC[C@@H](OCCNC(=O)[C@H]4CC(=O)N(C)[C@@H]4c4cccnc4)CC3)CC2)[C@@H]2CCc3cc(Br)ccc32)C1] |
| II-20 | 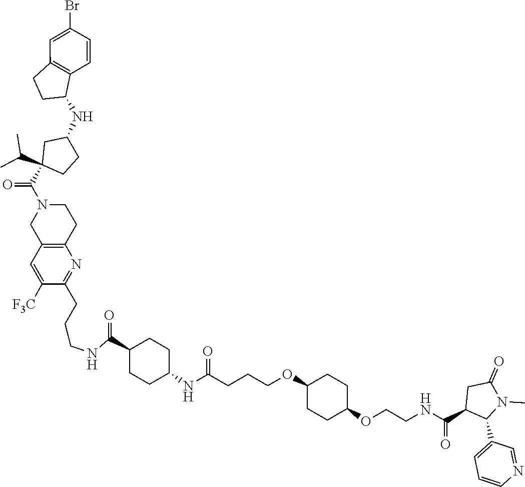 [SMILES: CC(C)[C@]1(C(=O)N2CCc3nc(CCCNC(=O)[C@H]4CC[C@H](NC(=O)CCCO[C@H]5CC[C@@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-21 | 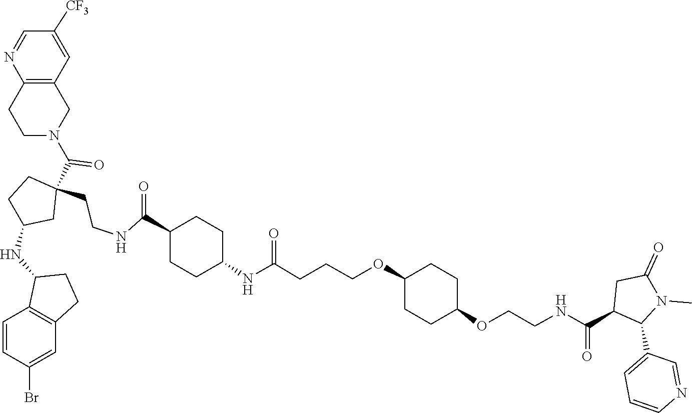 [SMILES: CN1C(=O)C[C@H](C(=O)NCCO[C@H]2CC[C@@H](OCCCC(=O)N[C@H]3CC[C@H](C(=O)NCC[C@]4(C(=O)N5CCc6ncc(C(F)(F)F)cc6C5)CC[C@@H](N[C@@H]5CCc6cc(Br)ccc65)C4)CC3)CC2)[C@H]1c1cccnc1] |
| II-22 | 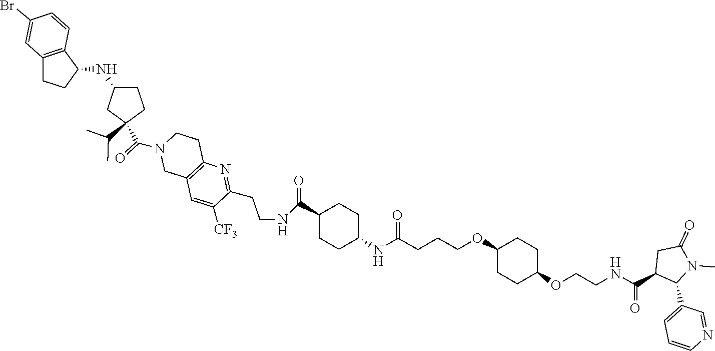 [SMILES: CC(C)[C@]1(C(=O)N2CCc3nc(CCNC(=O)[C@H]4CC[C@H](NC(=O)CCCO[C@H]5CC[C@@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-23 | 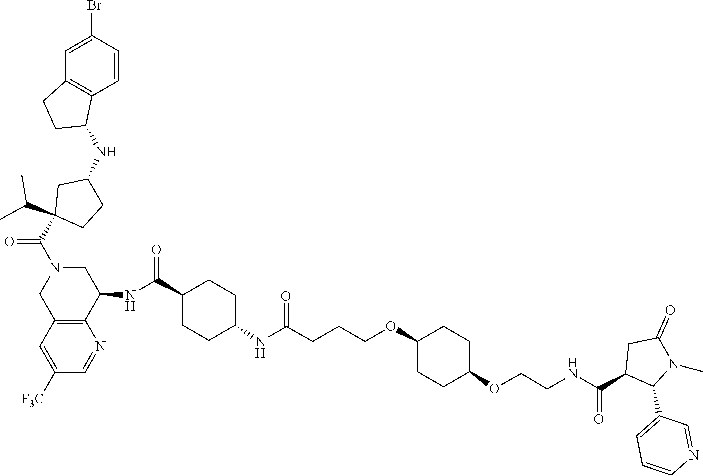 [SMILES: CC(C)[C@]1(C(=O)N2Cc3cc(C(F)(F)F)cnc3[C@H](NC(=O)[C@H]3CC[C@H](NC(=O)CCCO[C@H]4CC[C@@H](OCCNC(=O)[C@H]5CC(=O)N(C)[C@@H]5c5cccnc5)CC4)CC3)C2)CC[C@@H](N[C@@H]2CCc3cc(Br)ccc32)C1] |
| II-24 | 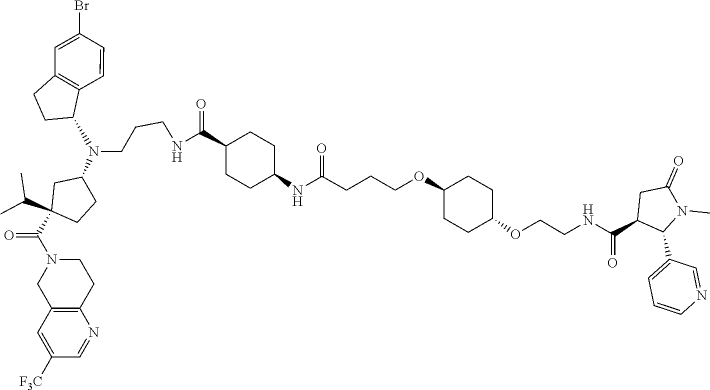 [SMILES: CC(C)[C@]1(C(=O)N2CCc3ncc(C(F)(F)F)cc3C2)CC[C@@H](N(CCCNC(=O)[C@H]2CC[C@@H](NC(=O)CCCO[C@H]3CC[C@H](OCCNC(=O)[C@H]4CC(=O)N(C)[C@@H]4c4cccnc4)CC3)CC2)[C@@H]2CCc3cc(Br)ccc32)C1] |
| II-25 | 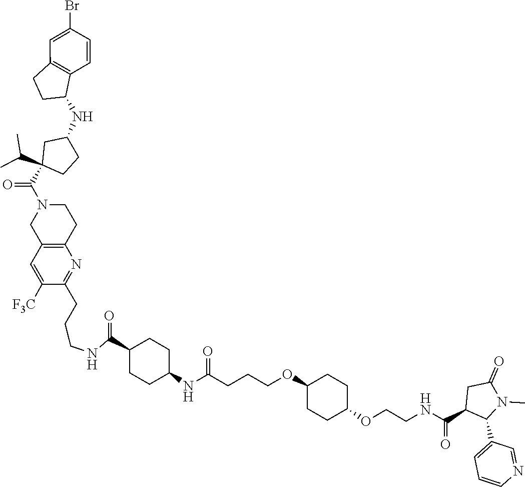 [SMILES: CC(C)[C@]1(C(=O)N2CCc3nc(CCCNC(=O)[C@H]4CC[C@@H](NC(=O)CCCO[C@H]5CC[C@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-26 | 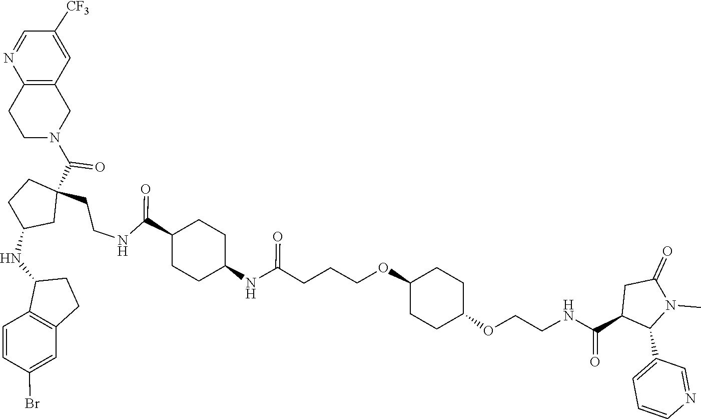 [SMILES: CN1C(=O)C[C@H](C(=O)NCCO[C@H]2CC[C@H](OCCCC(=O)N[C@H]3CC[C@@H](C(=O)NCC[C@]4(C(=O)N5CCc6ncc(C(F)(F)F)cc6C5)CC[C@@H](N[C@@H]5CCc6cc(Br)ccc65)C4)CC3)CC2)[C@H]1c1cccnc1] |
| II-27 | 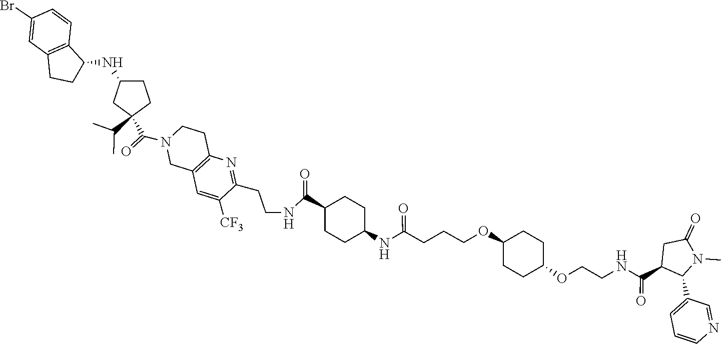 [SMILES: CC(C)[C@]1(C(=O)N2CCc3nc(CCNC(=O)[C@H]4CC[C@@H](NC(=O)CCCO[C@H]5CC[C@H](OCCNC(=O)[C@H]6CC(=O)N(C)[C@@H]6c6cccnc6)CC5)CC4)c(C(F)(F)F)cc3C2)CC[C@@H](N[C@@H]2CCc3cc(Br)ccc32)C1] |
| II-28 | 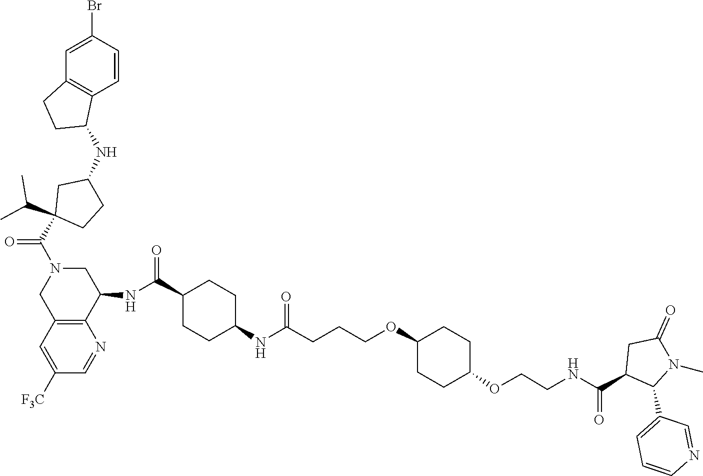 [SMILES: CC(C)[C@]1(C(=O)N2Cc3cc(C(F)(F)F)cnc3[C@H](NC(=O)[C@H]3CC[C@@H](NC(=O)CCCO[C@H]4CC[C@H](OCCNC(=O)[C@H]5CC(=O)N(C)[C@@H]5c5cccnc5)CC4)CC3)C2)CC[C@@H](N[C@@H]2CCc3cc(Br)ccc32)C1] |
| II-29 | 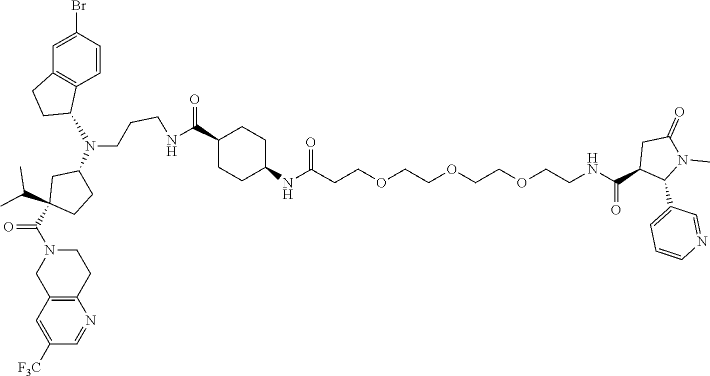 [SMILES: CC(C)[C@]1(C(=O)N2CCc3ncc(C(F)(F)F)cc3C2)CC[C@@H](N(CCCNC(=O)[C@H]2CC[C@@H](NC(=O)CCOCCOCCOCCNC(=O)[C@H]3CC(=O)N(C)[C@@H]3c3cccnc3)CC2)[C@@H]2CCc3cc(Br)ccc32)C1] |
| II-30 | 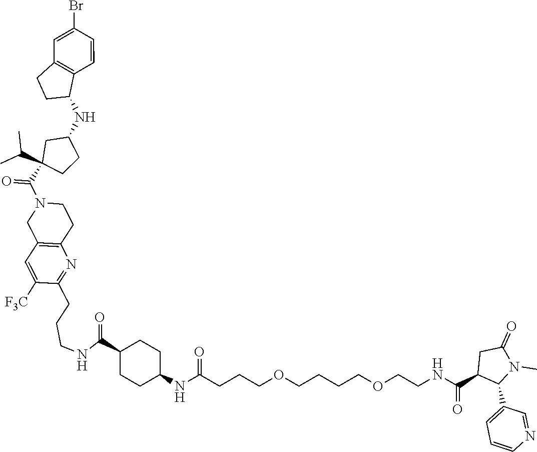 [SMILES: CC(C)[C@]1(C(=O)N2CCc3nc(CCCNC(=O)[C@H]4CC[C@@H](NC(=O)CCCOCCCCOCCNC(=O)[C@H]5CC(=O)N(C)[C@@H]5c5cccnc5)CC4)c(C(F)(F)F)cc3C2)CC[C@@H](N[C@@H]2CCc3cc(Br)ccc32)C1] |
| II-31 | 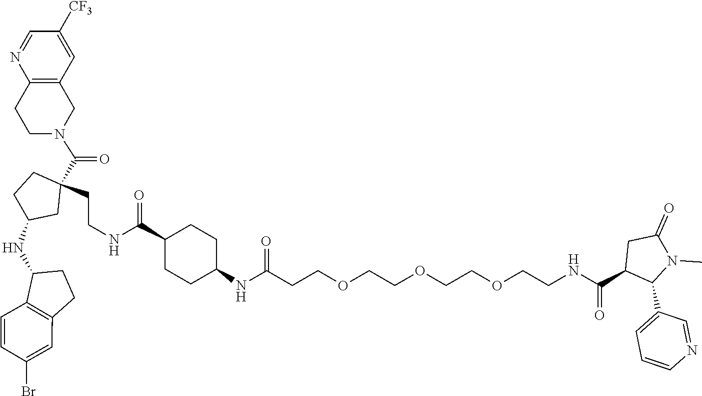 [SMILES: CN1C(=O)C[C@H](C(=O)NCCOCCOCCOCCC(=O)N[C@H]2CC[C@@H](C(=O)NCC[C@]3(C(=O)N4CCc5ncc(C(F)(F)F)cc5C4)CC[C@@H](N[C@@H]4CCc5cc(Br)ccc54)C3)CC2)[C@H]1c1cccnc1] |
| II-32 | 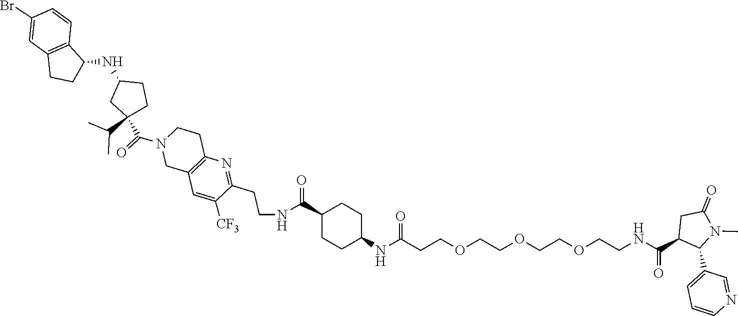 [SMILES: CC(C)[C@]1(C(=O)N2CCc3nc(CCNC(=O)[C@H]4CC[C@@H](NC(=O)CCOCCOCCOCCNC(=O)[C@H]5CC(=O)N(C)[C@@H]5c5cccnc5)CC4)c(C(F)(F)F)cc3C2)CC[C@@H](N[C@@H]2CCc3cc(Br)ccc32)C1] |
| II-33 | 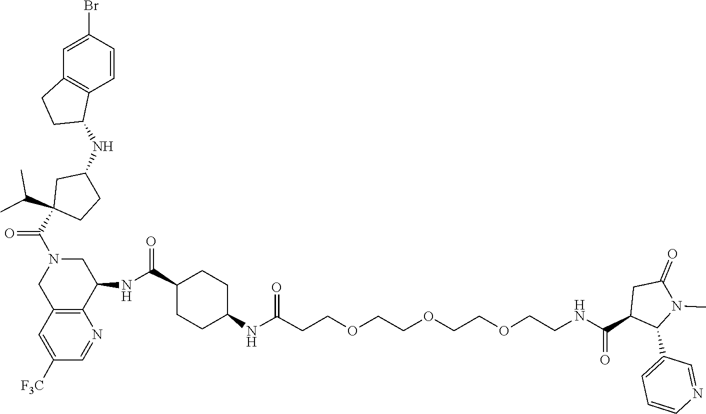 [SMILES: CC(C)[C@]1(C(=O)N2Cc3cc(C(F)(F)F)cnc3[C@H](NC(=O)[C@H]3CC[C@@H](NC(=O)CCOCCOCCOCCNC(=O)[C@H]4CC(=O)N(C)[C@@H]4c4cccnc4)CC3)C2)CC[C@@H](N[C@@H]2CCc3cc(Br)ccc32)C1] |
| II-34 | 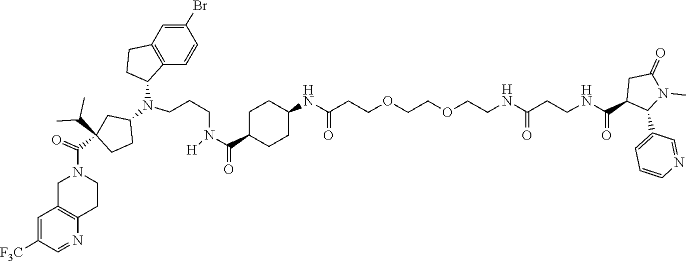 [SMILES: CC(C)[C@]1(C(=O)N2CCc3ncc(C(F)(F)F)cc3C2)CC[C@@H](N(CCCNC(=O)[C@H]2CC[C@@H](NC(=O)CCOCCOCCNC(=O)CCNC(=O)[C@H]3CC(=O)N(C)[C@@H]3c3cccnc3)CC2)[C@@H]2CCc3cc(Br)ccc32)C1] |
| II-35 | 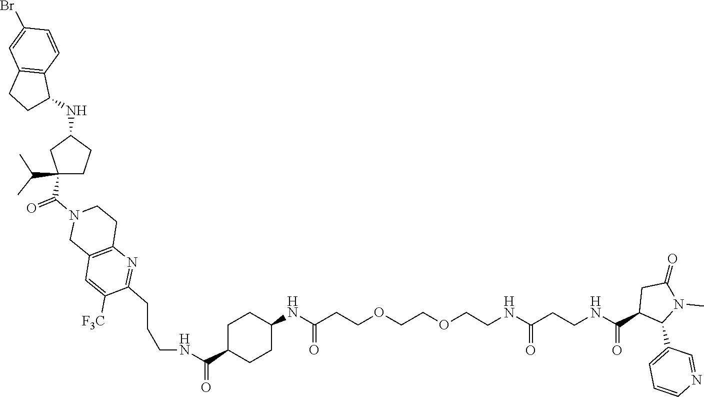 [SMILES: CC(C)[C@]1(C(=O)N2CCc3nc(CCCNC(=O)[C@H]4CC[C@@H](NC(=O)CCOCCOCCNC(=O)CCNC(=O)[C@H]5CC(=O)N(C)[C@@H]5c5cccnc5)CC4)c(C(F)(F)F)cc3C2)CC[C@@H](N[C@@H]2CCc3cc(Br)ccc32)C1] |
| II-36 | 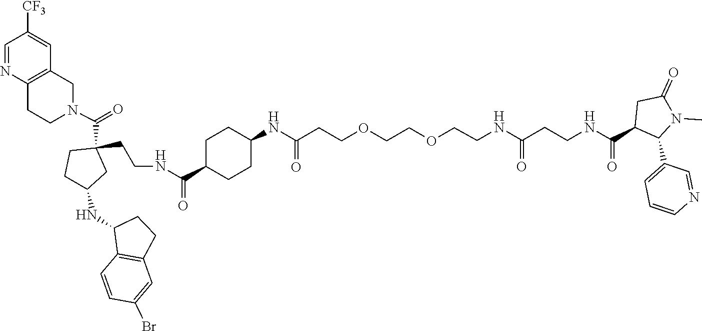 [SMILES: CN1C(=O)C[C@H](C(=O)NCCC(=O)NCCOCCOCCC(=O)N[C@H]2CC[C@@H](C(=O)NCC[C@]3(C(=O)N4CCc5ncc(C(F)(F)F)cc5C4)CC[C@@H](N[C@@H]4CCc5cc(Br)ccc54)C3)CC2)[C@H]1c1cccnc1] |
| II-37 | 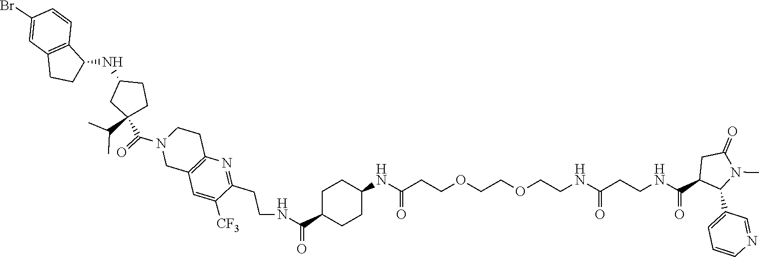 [SMILES: CC(C)[C@]1(C(=O)N2CCc3nc(CCNC(=O)[C@H]4CC[C@@H](NC(=O)CCOCCOCCNC(=O)CCNC(=O)[C@H]5CC(=O)N(C)[C@@H]5c5cccnc5)CC4)c(C(F)(F)F)cc3C2)CC[C@@H](N[C@@H]2CCc3cc(Br)ccc32)C1] |
| II-38 | 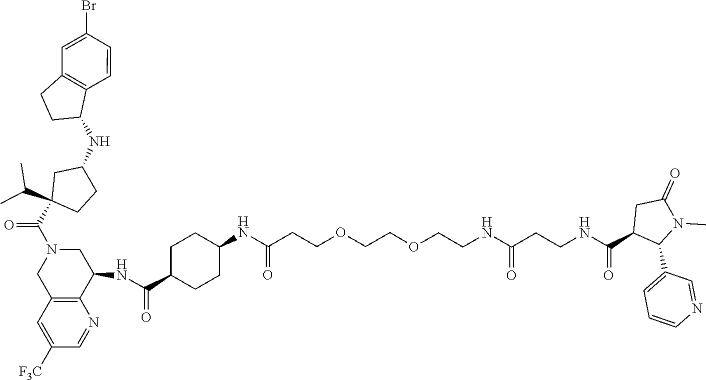 [SMILES: CC(C)[C@]1(C(=O)N2Cc3cc(C(F)(F)F)cnc3[C@H](NC(=O)[C@H]3CC[C@@H](NC(=O)CCOCCOCCNC(=O)CCNC(=O)[C@H]4CC(=O)N(C)[C@@H]4c4cccnc4)CC3)C2)CC[C@@H](N[C@@H]2CCc3cc(Br)ccc32)C1] |
| II-39 | 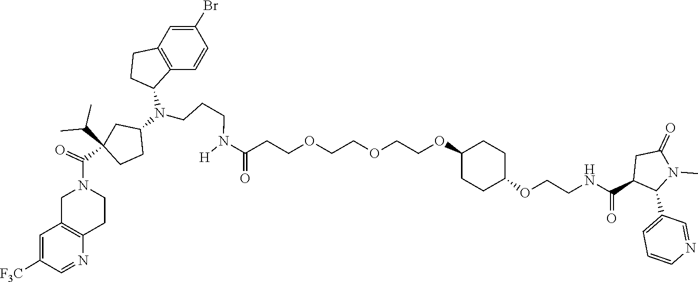 [SMILES: CC(C)[C@]1(C(=O)N2CCc3ncc(C(F)(F)F)cc3C2)CC[C@@H](N(CCCNC(=O)CCOCCOCCO[C@H]2CC[C@H](OCCNC(=O)[C@H]3CC(=O)N(C)[C@@H]3c3cccnc3)CC2)[C@@H]2CCc3cc(Br)ccc32)C1] |
| II-40 | 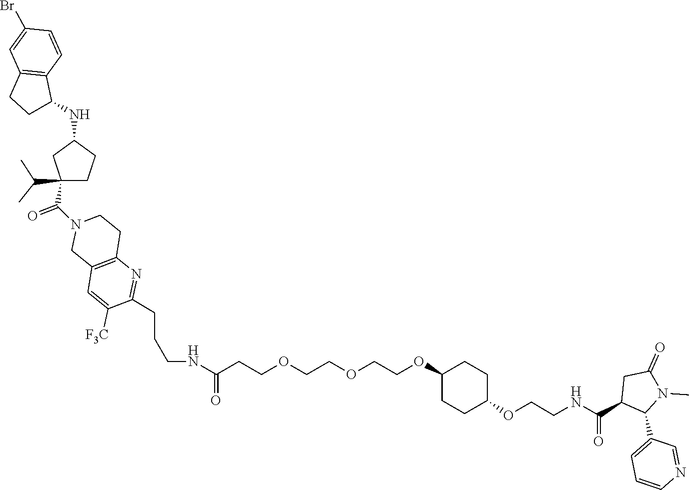 [SMILES: CC(C)[C@]1(C(=O)N2CCc3nc(CCCNC(=O)CCOCCOCCO[C@H]4CC[C@H](OCCNC(=O)[C@H]5CC(=O)N(C)[C@@H]5c5cccnc5)CC4)c(C(F)(F)F)cc3C2)CC[C@@H](N[C@@H]2CCc3cc(Br)ccc32)C1] |
| II-41 | 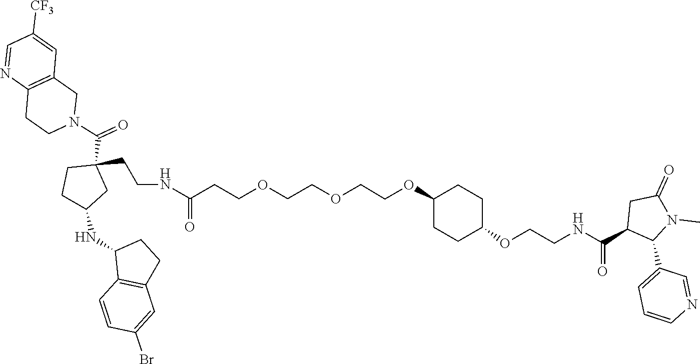 [SMILES: CN1C(=O)C[C@H](C(=O)NCCO[C@H]2CC[C@H](OCCOCCOCCC(=O)NCC[C@]3(C(=O)N4CCc5ncc(C(F)(F)F)cc5C4)CC[C@@H](N[C@@H]4CCc5cc(Br)ccc54)C3)CC2)[C@H]1c1cccnc1] |
| II-42 | 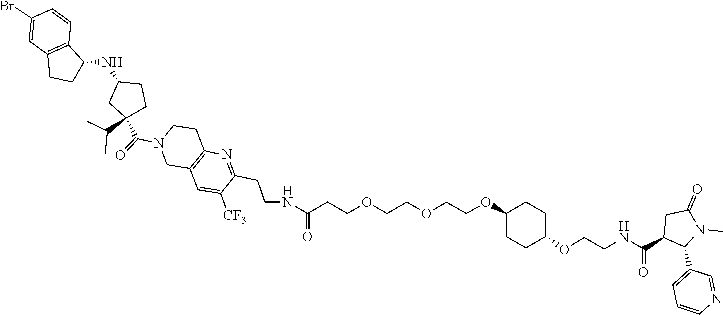 [SMILES: CC(C)[C@]1(C(=O)N2CCc3nc(CCNC(=O)CCOCCOCCO[C@H]4CC[C@H](OCCNC(=O)[C@H]5CC(=O)N(C)[C@@H]5c5cccnc5)CC4)c(C(F)(F)F)cc3C2)CC[C@@H](N[C@@H]2CCc3cc(Br)ccc32)C1] |
| II-43 | 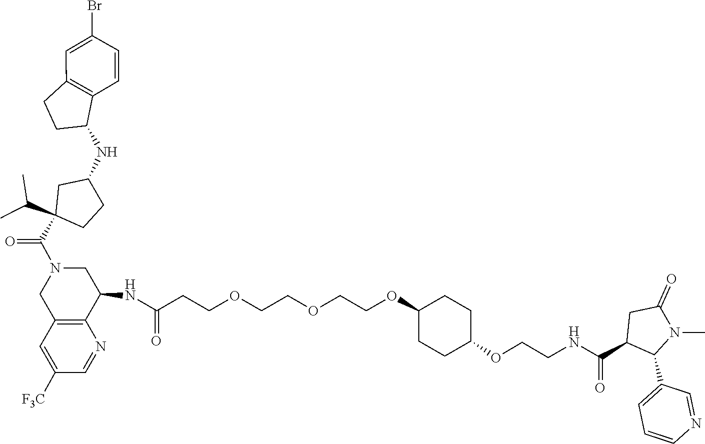 [SMILES: CC(C)[C@]1(C(=O)N2Cc3cc(C(F)(F)F)cnc3[C@H](NC(=O)CCOCCOCCO[C@H]3CC[C@H](OCCNC(=O)[C@H]4CC(=O)N(C)[C@@H]4c4cccnc4)CC3)C2)CC[C@@H](N[C@@H]2CCc3cc(Br)ccc32)C1] |
| II-44 | 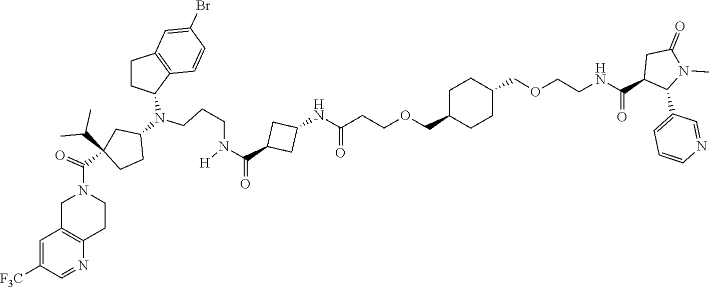 [SMILES: CC(C)[C@]1(C(=O)N2CCc3ncc(C(F)(F)F)cc3C2)CC[C@@H](N(CCCNC(=O)[C@H]2C[C@H](NC(=O)CCOC[C@H]3CC[C@H](COCCNC(=O)[C@H]4CC(=O)N(C)[C@@H]4c4cccnc4)CC3)C2)[C@@H]2CCc3cc(Br)ccc32)C1] |
| II-45 | 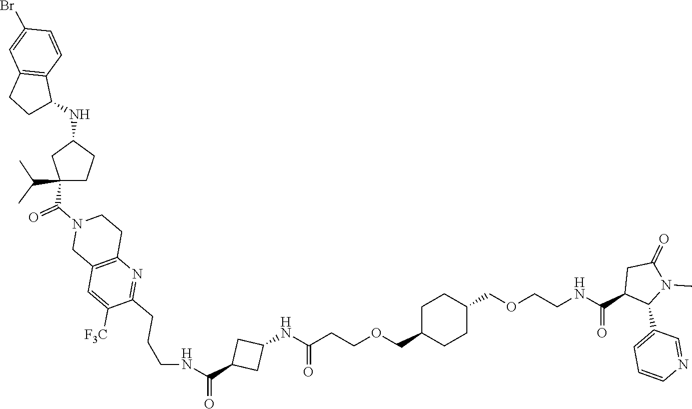 [SMILES: CC(C)[C@]1(C(=O)N2CCc3nc(CCCNC(=O)[C@H]4C[C@H](NC(=O)CCOC[C@H]5CC[C@H](COCCNC(=O)[C@H]6CC(=O)N(C)[C@@H]6c6cccnc6)CC5)C4)c(C(F)(F)F)cc3C2)CC[C@@H](N[C@@H]2CCc3cc(Br)ccc32)C1] |
| II-46 | 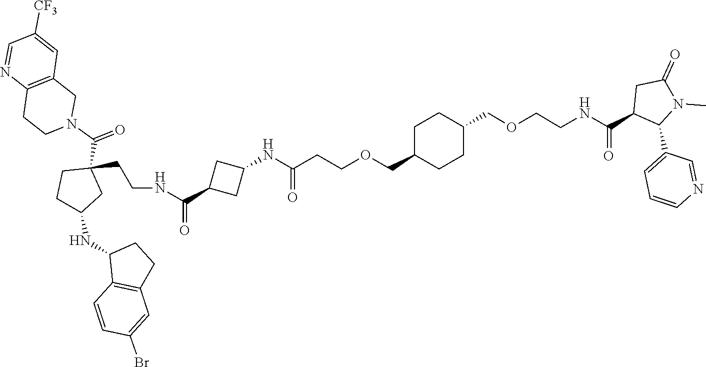 [SMILES: CN1C(=O)C[C@H](C(=O)NCCOC[C@H]2CC[C@H](COCCC(=O)N[C@H]3C[C@H](C(=O)NCC[C@]4(C(=O)N5CCc6ncc(C(F)(F)F)cc6C5)CC[C@@H](N[C@@H]5CCc6cc(Br)ccc65)C4)C3)CC2)[C@H]1c1cccnc1] |
| II-47 | 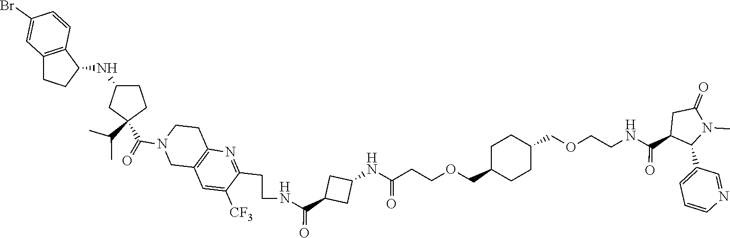 [SMILES: CC(C)[C@]1(C(=O)N2CCc3nc(CCNC(=O)[C@H]4C[C@H](NC(=O)CCOC[C@H]5CC[C@H](COCCNC(=O)[C@H]6CC(=O)N(C)[C@@H]6c6cccnc6)CC5)C4)c(C(F)(F)F)cc3C2)CC[C@@H](N[C@@H]2CCc3cc(Br)ccc32)C1] |
| II-48 | 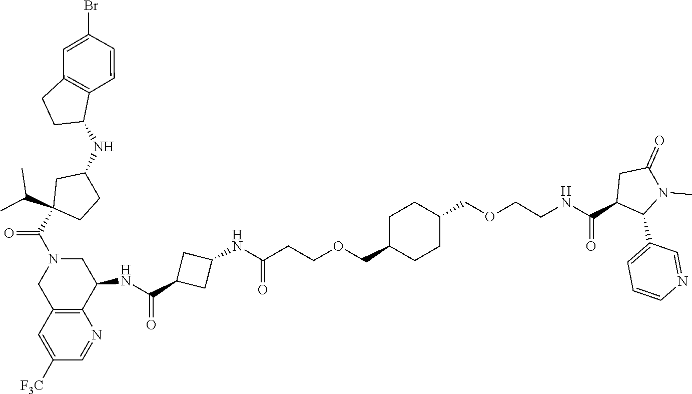 [SMILES: CC(C)[C@]1(C(=O)N2Cc3cc(C(F)(F)F)cnc3[C@H](NC(=O)[C@H]3C[C@H](NC(=O)CCOC[C@H]4CC[C@H](COCCNC(=O)[C@H]5CC(=O)N(C)[C@@H]5c5cccnc5)CC4)C3)C2)CC[C@@H](N[C@@H]2CCc3cc(Br)ccc32)C1] |
| II-49 | 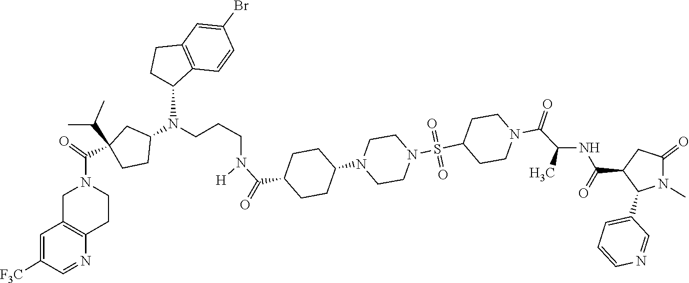 [SMILES: CC(C)[C@]1(C(=O)N2CCc3ncc(C(F)(F)F)cc3C2)CC[C@@H](N(CCCNC(=O)[C@H]2CC[C@@H](N3CCN(S(=O)(=O)C4CCN(C(=O)[C@H](C)NC(=O)[C@H]5CC(=O)N(C)[C@@H]5c5cccnc5)CC4)CC3)CC2)[C@@H]2CCc3cc(Br)ccc32)C1] |
| II-50 | 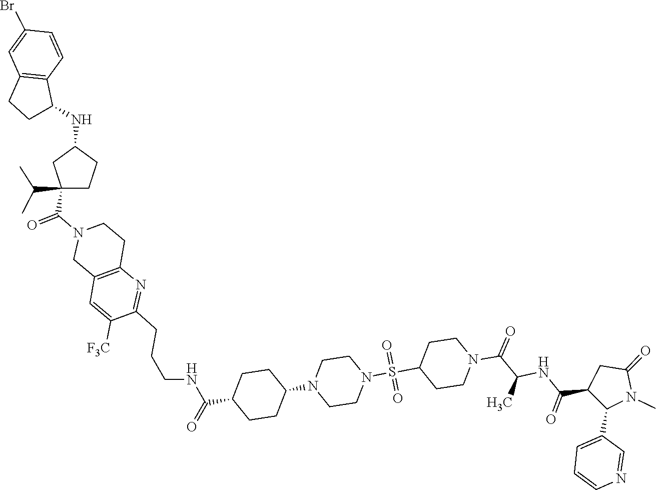 [SMILES: CC(C)[C@]1(C(=O)N2CCc3nc(CCCNC(=O)[C@H]4CC[C@@H](N5CCN(S(=O)(=O)C6CCN(C(=O)[C@H](C)NC(=O)[C@H]7CC(=O)N(C)[C@@H]7c7cccnc7)CC6)CC5)CC4)c(C(F)(F)F)cc3C2)CC[C@@H](N[C@@H]2CCc3cc(Br)ccc32)C1] |
| II-51 | 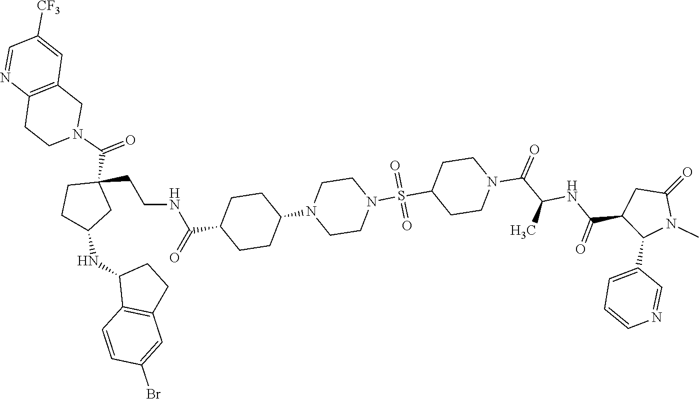 [SMILES: C[C@H](NC(=O)[C@H]1CC(=O)N(C)[C@@H]1c1cccnc1)C(=O)N1CCC(S(=O)(=O)N2CCN([C@H]3CC[C@@H](C(=O)NCC[C@]4(C(=O)N5CCc6ncc(C(F)(F)F)cc6C5)CC[C@@H](N[C@@H]5CCc6cc(Br)ccc65)C4)CC3)CC2)CC1] |
| II-52 | 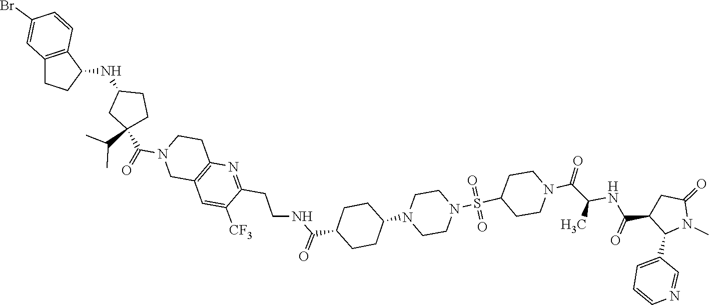 [SMILES: CC(C)[C@]1(C(=O)N2CCc3nc(CCNC(=O)[C@H]4CC[C@@H](N5CCN(S(=O)(=O)C6CCN(C(=O)[C@H](C)NC(=O)[C@H]7CC(=O)N(C)[C@@H]7c7cccnc7)CC6)CC5)CC4)c(C(F)(F)F)cc3C2)CC[C@@H](N[C@@H]2CCc3cc(Br)ccc32)C1] |
| II-53 | 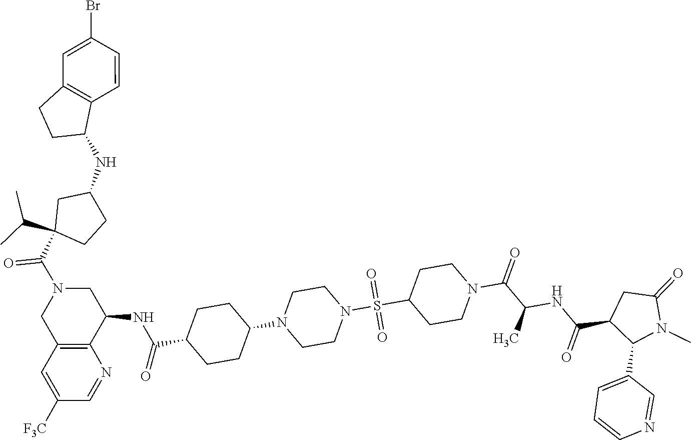 [SMILES: CC(C)[C@]1(C(=O)N2Cc3cc(C(F)(F)F)cnc3[C@H](NC(=O)[C@H]3CC[C@@H](N4CCN(S(=O)(=O)C5CCN(C(=O)[C@H](C)NC(=O)[C@H]6CC(=O)N(C)[C@@H]6c6cccnc6)CC5)CC4)CC3)C2)CC[C@@H](N[C@@H]2CCc3cc(Br)ccc32)C1] |
| II-54 | 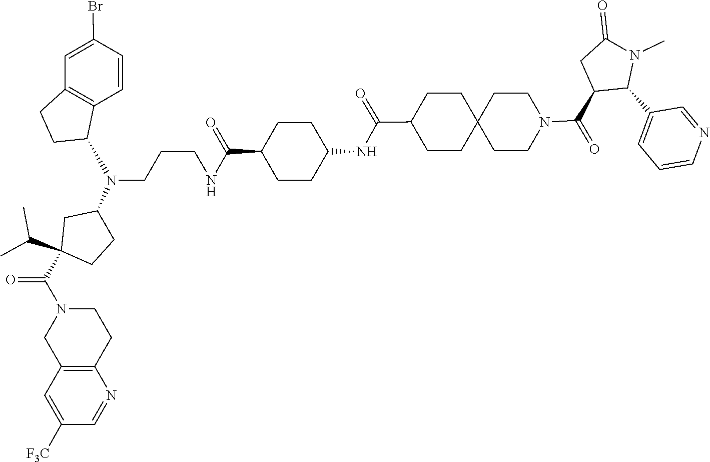 |
| II-55 | 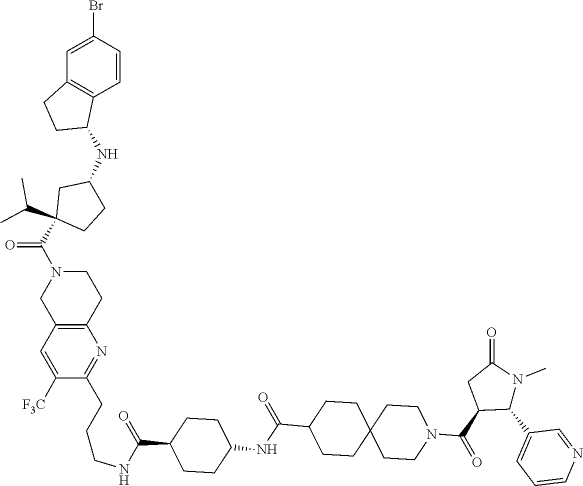 |
| II-56 | 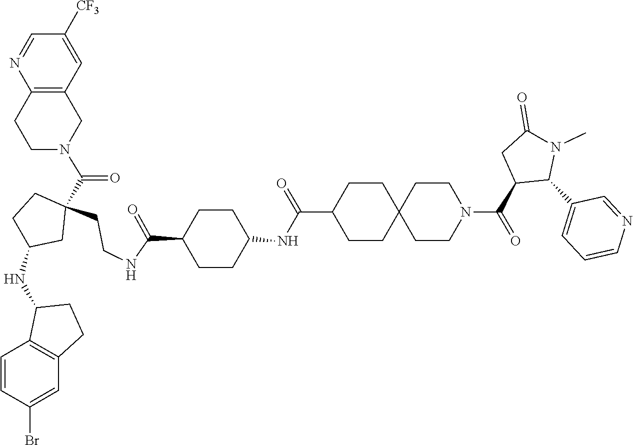 |
| II-57 | 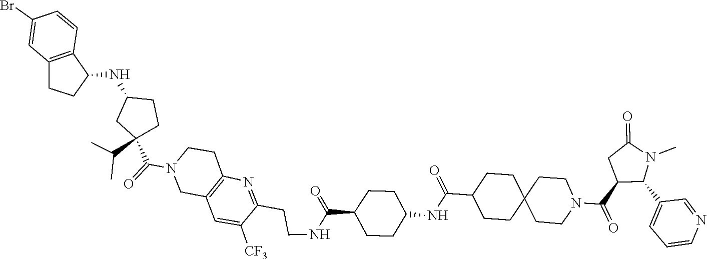 |
| II-58 | 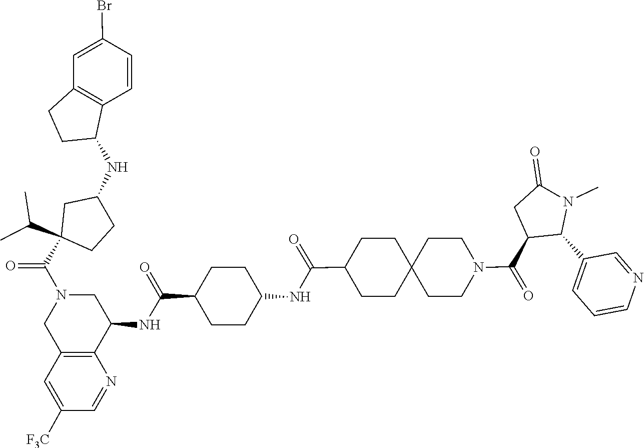 |
| II-59 | 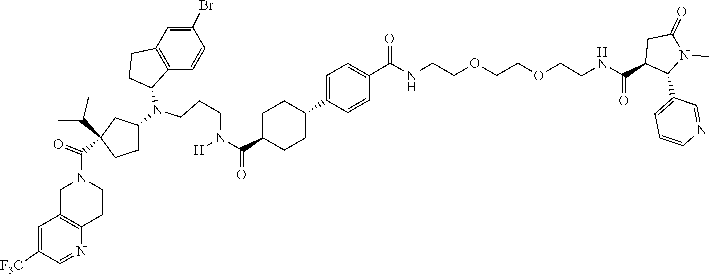 [SMILES: CC(C)[C@]1(C(=O)N2CCc3ncc(C(F)(F)F)cc3C2)CC[C@@H](N(CCCNC(=O)[C@H]2CC[C@H](c3ccc(C(=O)NCCOCCOCCNC(=O)[C@H]4CC(=O)N(C)[C@@H]4c4cccnc4)cc3)CC2)[C@@H]2CCc3cc(Br)ccc32)C1] |
| II-60 | 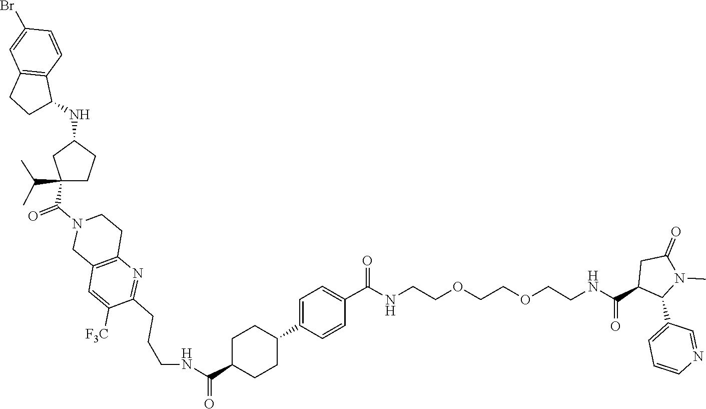 [SMILES: CC(C)[C@]1(C(=O)N2CCc3nc(CCCNC(=O)[C@H]4CC[C@H](c5ccc(C(=O)NCCOCCOCCNC(=O)[C@H]6CC(=O)N(C)[C@@H]6c6cccnc6)cc5)CC4)c(C(F)(F)F)cc3C2)CC[C@@H](N[C@@H]2CCc3cc(Br)ccc32)C1] |
| II-61 | 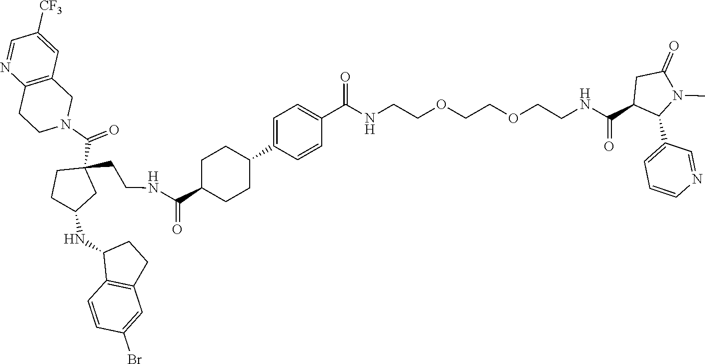 [SMILES: CN1C(=O)C[C@H](C(=O)NCCOCCOCCNC(=O)c2ccc([C@H]3CC[C@H](C(=O)NCC[C@]4(C(=O)N5CCc6ncc(C(F)(F)F)cc6C5)CC[C@@H](N[C@@H]5CCc6cc(Br)ccc65)C4)CC3)cc2)[C@H]1c1cccnc1] |
| II-62 | 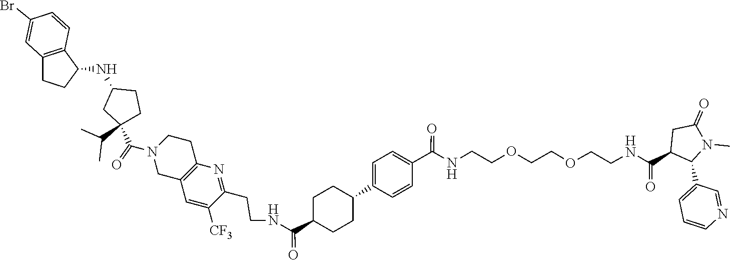 [SMILES: CC(C)[C@]1(C(=O)N2CCc3nc(CCNC(=O)[C@H]4CC[C@H](c5ccc(C(=O)NCCOCCOCCNC(=O)[C@H]6CC(=O)N(C)[C@@H]6c6cccnc6)cc5)CC4)c(C(F)(F)F)cc3C2)CC[C@@H](N[C@@H]2CCc3cc(Br)ccc32)C1] |
| II-63 | 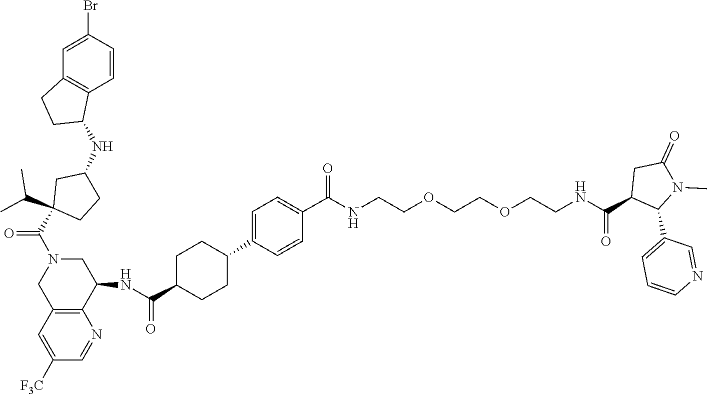 [SMILES: CC(C)[C@]1(C(=O)N2Cc3cc(C(F)(F)F)cnc3[C@H](NC(=O)[C@H]3CC[C@H](c4ccc(C(=O)NCCOCCOCCNC(=O)[C@H]5CC(=O)N(C)[C@@H]5c5cccnc5)cc4)CC3)C2)CC[C@@H](N[C@@H]2CCc3cc(Br)ccc32)C1] |
| II-64 | 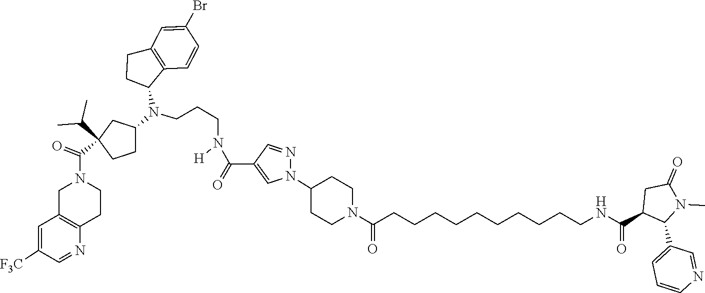 [SMILES: CC(C)[C@]1(C(=O)N2CCc3ncc(C(F)(F)F)cc3C2)CC[C@@H](N(CCCNC(=O)c2cnn(C3CCN(C(=O)CCCCCCCCCCNC(=O)[C@H]4CC(=O)N(C)[C@@H]4c4cccnc4)CC3)c2)[C@@H]2CCc3cc(Br)ccc32)C1] |
| II-65 | 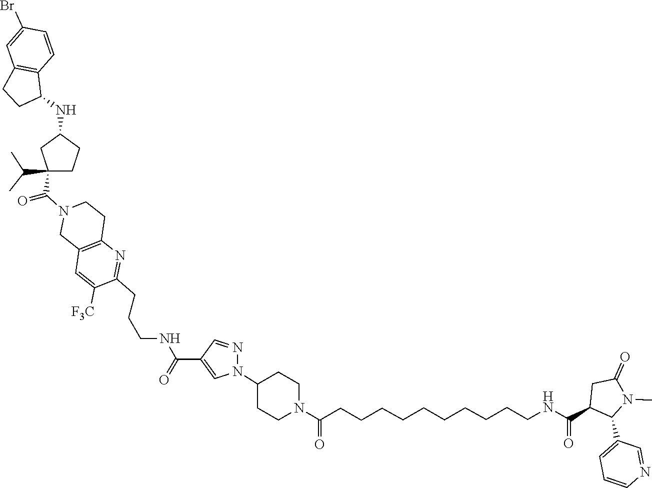 [SMILES: CC(C)[C@]1(C(=O)N2CCc3nc(CCCNC(=O)c4cnn(C5CCN(C(=O)CCCCCCCCCCNC(=O)[C@H]6CC(=O)N(C)[C@@H]6c6cccnc6)CC5)c4)c(C(F)(F)F)cc3C2)CC[C@@H](N[C@@H]2CCc3cc(Br)ccc32)C1] |
| II-66 | 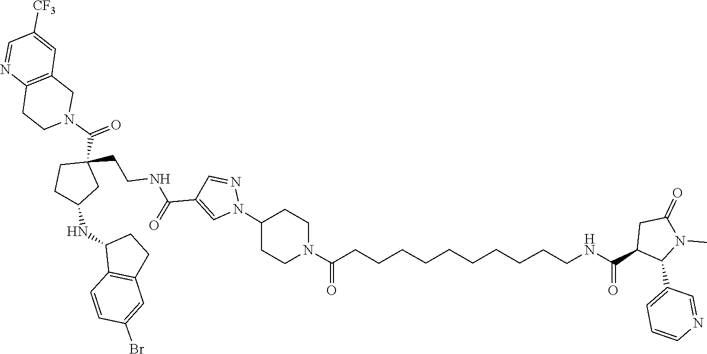 [SMILES: CN1C(=O)C[C@H](C(=O)NCCCCCCCCCCC(=O)N2CCC(n3cc(C(=O)NCC[C@]4(C(=O)N5CCc6ncc(C(F)(F)F)cc6C5)CC[C@@H](N[C@@H]5CCc6cc(Br)ccc65)C4)cn3)CC2)[C@H]1c1cccnc1] |
| II-67 | 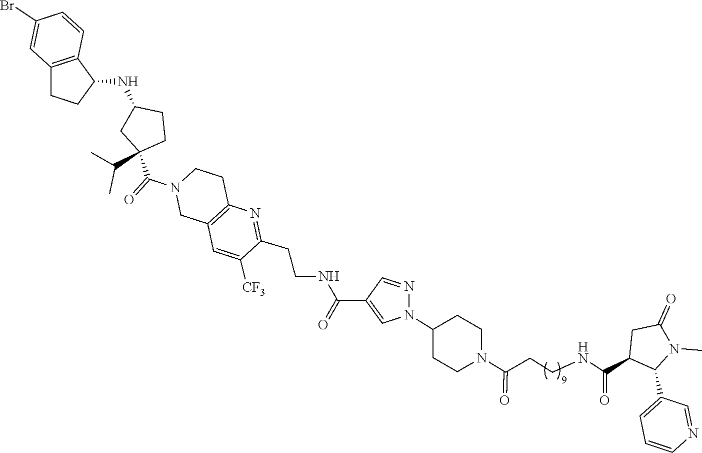 |
| II-68 | 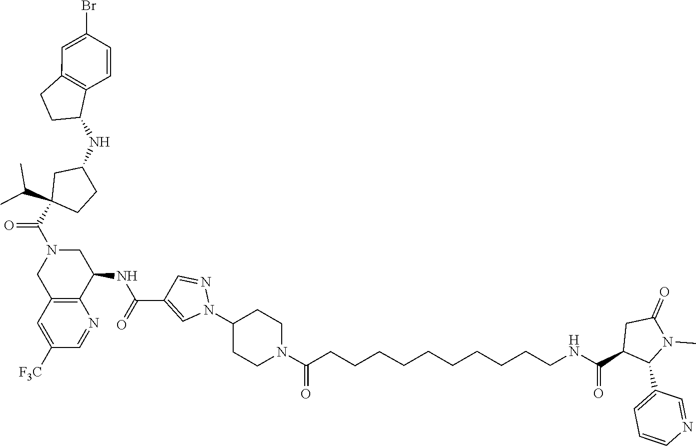 [SMILES: CC(C)[C@]1(C(=O)N2Cc3cc(C(F)(F)F)cnc3[C@H](NC(=O)c3cnn(C4CCN(C(=O)CCCCCCCCCCNC(=O)[C@H]5CC(=O)N(C)[C@@H]5c5cccnc5)CC4)c3)C2)CC[C@@H](N[C@@H]2CCc3cc(Br)ccc32)C1] |

### Synthetic Methods

Methods for preparing compounds described herein are illustrated in the following synthetic Schemes. The Schemes are given for the purpose of illustrating the invention, and not intended to limit the scope or spirit of the invention. Starting materials shown in the Schemes can be obtained from commercial sources or can be prepared based on procedures described in the literature.

Scheme 1 is an illustrative general method for preparing compound E, an intermediate in the synthesis of compounds of Formula I. Reaction of BOC-protected amine A with methyl 4-(2,5-dimethyl-1H-pyrrol-1-yl)cyclopent-2-ene-1-carboxylate in the presence of base (for example LiHMDS), followed by base-mediated hydrolysis, provides carboxylic acid B. Peptide coupling of carboxylic acid B (for example with HATU/DIPEA) with 1-(4-(trifluoromethyl) pyridin-2-yl)piperazine, followed by deprotection of the 2,5-dimethylpyrrole group (for example with hydroxylamine HCl) affords amine C. Reductive amination of amine C (for example with sodium triacetoxyborohydride) with 3-methoxytetrahydro-4H-pyran-4-one affords compound D. Starting with compound D, reduction of the olefin (for example with Pd/C under hydrogen) and treatment with HCl provides amine intermediate E.

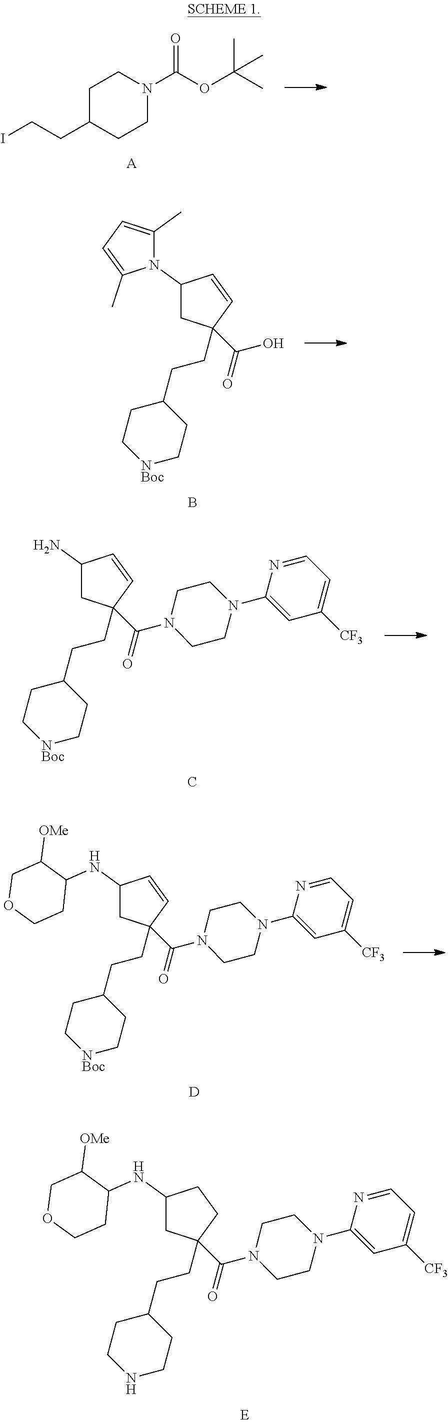

Scheme 2 illustrates a general method for preparing a compound of Formula I. Amine intermediate A is coupled (for example with HATU/DIPEA) with carboxylic acid B, where the carboxylic linker may, for instance, be a linker “L” described herein, to provide compound C.

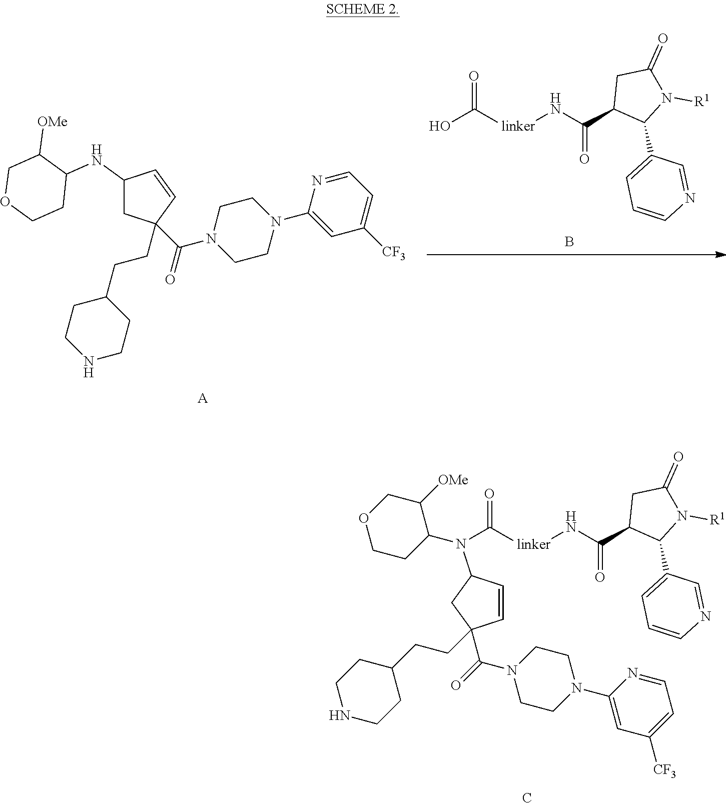

Scheme 3 illustrates a general method for preparing E, a compound of Formula I. Amine A is prepared by analogy to the procedure depicted in Scheme 1. Amine A is reductively aminated with aldehyde B. Benzoyl deprotection (for example via hydrolysis) provides compound C. Peptide coupling of C with carboxylic acid D, as described for Scheme 2, provides E, a compound of Formula I.

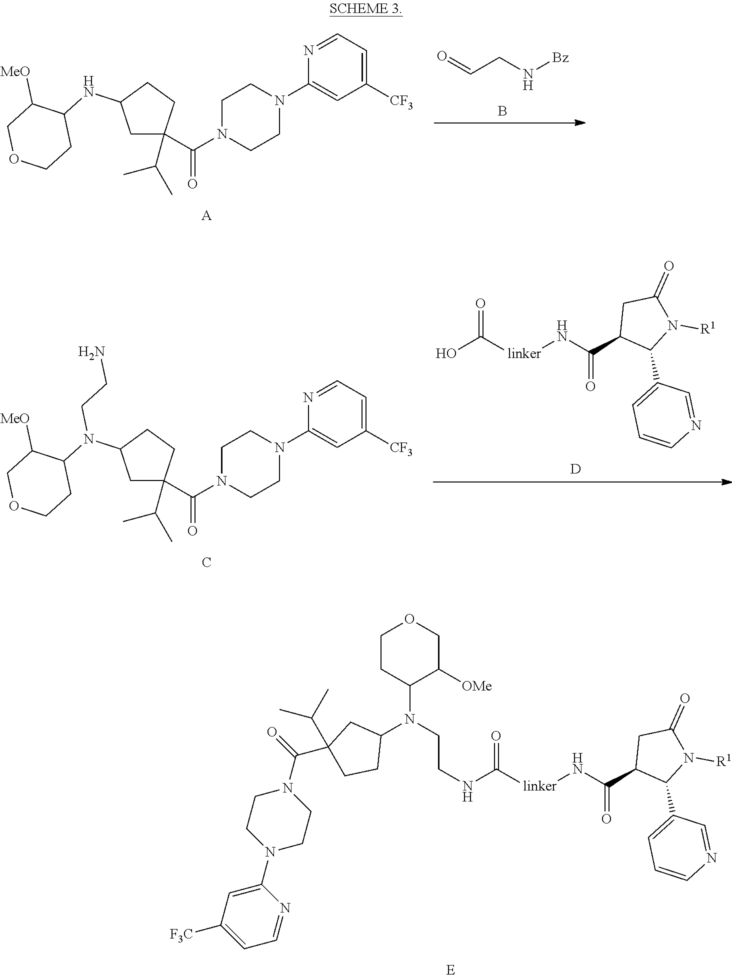

Scheme 4 illustrates a general method for preparing compound E, a compound of Formula I. Amine A is prepared by analogy to the procedure depicted in Scheme 1. Amine A is coupled to piperazine B (for example via HATU/DIPEA), and the Boc group is removed (for example using HCl) to provide primary amine intermediate C. Peptide coupling of C with carboxylic acid D, as described for Scheme 2, provides compound E, a compound of Formula I.

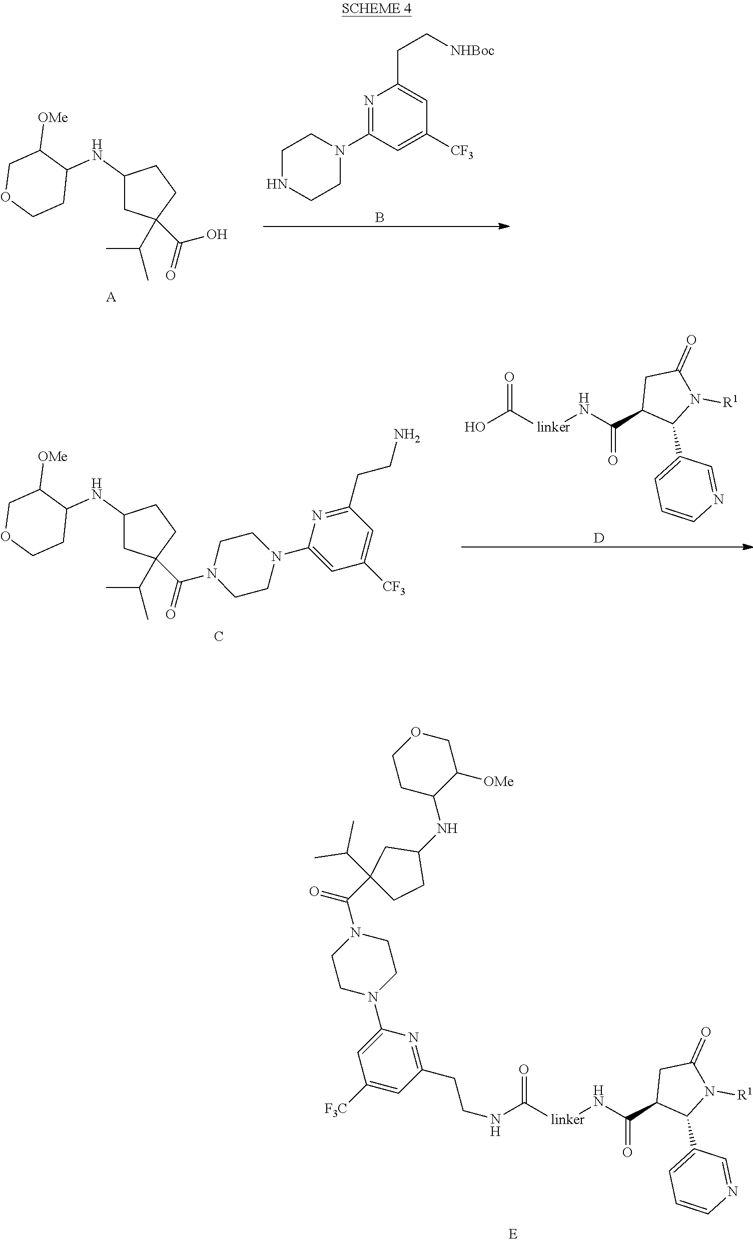

Scheme 5 illustrates a general method for preparing compound C, a compound of Formula I. Amine intermediate A (compound E from Scheme 1) is coupled (for example with HATU/DIPEA) with carboxylic acid B, where the carboxylic linker may, for instance, be a linker “L” described herein, to provide compound C.

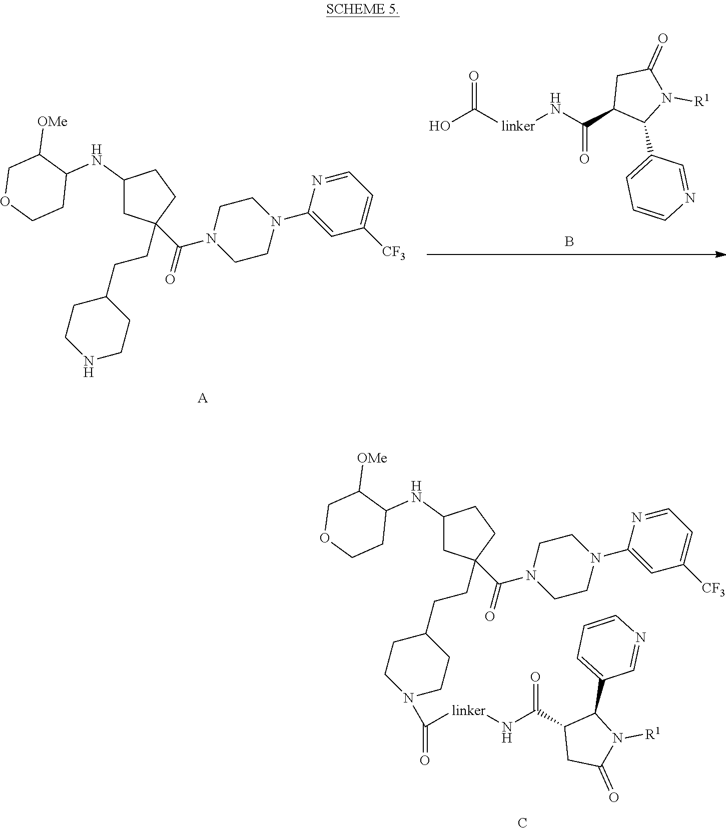

In the Schemes disclosed herein, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference. The modular synthetic route illustrated in the Schemes can also be readily modified by one of skill in the art to provide additional compounds, by conducting functional group transformations on the intermediate and final compounds. Such functional group transformations are well known in the art, as described in, for example, “Comprehensive Organic Synthesis” (B. M. Trost & I. Fleming, eds., 1991-1992).

### II. Anti-Cotinine Antibody

Also provided is an antibody, or antigen-binding fragment thereof, that binds to a cotinine moiety. As used herein, the term “anti-cotinine antibody or antigen-binding fragment thereof” refers to an antibody, or antigen binding fragment thereof that binds to a cotinine moiety. Cotinine has the following structure:

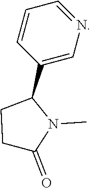

As used herein, the term “cotinine moiety” refers to cotinine or an analog of cotinine. Compounds of Formula (I) described herein comprise a cotinine moiety linked via a linker to a CCR2-binding moiety. In certain embodiments, the cotinine moiety has the following structure:

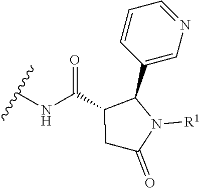

wherein R¹ is C₁₋₄ alkyl or C₃₋₆ cycloalkyl. In certain embodiments, R¹ is methyl, ethyl, 1-propyl, 2-propyl, 1-butyl, 2-butyl, or t-butyl. In certain embodiments, R¹ is methyl. In certain embodiments, R¹ is cyclopropyl, cyclobutyl, cyclopentyl, or cyclohexyl.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, 23(9): 1126-1136).

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or X light chain. The majority of antibodies in the serum belong to the IgG class, there are four isotypes of human IgG (IgG1, IgG2, IgG3 and IgG4), the sequences of which differ mainly in their hinge region.

“CDRs” are defined as the complementarity determining region amino acid sequences of an antibody or antigen binding fragment thereof. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al., Nature, 1989, 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 3 below represents one definition using each numbering convention for each CDR or binding unit. It should be noted that some of the CDR definitions may vary depending on the individual publication used.

TABLE 3

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

In a further embodiment, the anti-cotinine antibody is humanized. In a further embodiment, the Fc region of the anti-cotinine antibody is modified to increase ADCC activity, ADCP activity, and/or CDC activity, suitable modifications of which are provided below. In a further embodiment, the Fc region of the anti-cotinine antibody is modified to increase ADCC activity.

Fc engineering methods can be applied to modify the functional or pharmacokinetics properties of an antibody. Effector function may be altered by making mutations in the Fc region that increase or decrease binding to C1q or Fcγ receptors and modify CDC or ADCC activity respectively. Modifications to the glycosylation pattern of an antibody can also be made to change the effector function. The in vivo half-life of an antibody can be altered by making mutations that affect binding of the Fc to the FcRn (neonatal Fc receptor).

The term “effector function” as used herein refers to one or more of antibody-mediated effects including antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP).

The interaction between the Fc region of an antigen binding protein or antibody and various Fc receptors (FcR), including FcγRI (CD64), FcγRII (CD32), FcγRIII (CD16), FcRn, C1q, and type II Fc receptors is believed to mediate the effector functions of the antigen binding protein or antibody. Significant biological effects can be a consequence of effector functionality. Usually, the ability to mediate effector function requires binding of the antigen binding protein or antibody to an antigen and not all antigen binding proteins or antibodies will mediate every effector function.

Effector function can be assessed in a number of ways including, for example, evaluating ADCC effector function of antibody coated to target cells mediated by Natural Killer (NK) cells via FcγRIII, or monocytes/macrophages via FcγRI, or evaluating CDC effector function of antibody coated to target cells mediated by complement cascade via C1q. For example, an antibody, or antigen binding fragment thereof, of the present invention can be assessed for ADCC effector function in a Natural Killer cell assay. Examples of such assays can be found in Shields et al., The Journal of Biological Chemistry, 2001, 276: 6591-6604; Chappel et al., The Journal of Biological Chemistry, 1993, 268: 25124-25131; Lazar et al., PNAS, 2006, 103: 4005-4010.

Examples of assays to determine CDC function include those described in J Imm Meth, 1995, 184: 29-38.

The effects of mutations on effector functions (e.g., FcRn binding, FcγRs and C1q binding, CDC, ADCML, ADCC, ADCP) can be assessed, e.g., as described in Grevys et al., J Immunol., 2015,194(11): 5497-5508; Tam et al., Antibodies, 2017, 6(3): 12; or Monnet et al., mAbs, 2014, 6(2): 422-436.

Throughout this specification, amino acid residues in Fc regions, in antibody sequences or full-length antigen binding protein sequences, are numbered according to the EU index numbering convention.

Human IgG1 constant regions containing specific mutations have been shown to enhance binding to Fc receptors. In some cases these mutations have also been shown to enhance effector functions, such as ADCC and CDC, as described below. Antibodies, or antigen binding fragments thereof, of the present invention may include any of the following mutations.

Enhanced CDC: Fc engineering can be used to enhance complement-based effector function. For example (with reference to IgG1), K326W/E333S; S267E/H268F/S324T; and IgG1/IgG3 cross subclass can increase C1q binding; E345R (Diebolder et al., Science, 2014, 343: 1260-1293) and E345R/E430G/S440Y results in preformed IgG hexamers (Wang et al., Protein Cell, 2018, 9(1): 63-73).

Enhanced ADCC: Fc engineering can be used to enhance ADCC. For example (with reference to IgG1), F243L/R292P/Y300L/V305I/P396L; S239D/I332E; and S298A/E333A/K334A increase FcγRIIIa binding; S239D/I332E/A330L increases FcγRIIIa binding and decreases FcγRIIb binding; G236A/S239D/I332E improves binding to FcγRIIa, improves the FcγRIIa/FcγRIIb binding ratio (activating/inhibitory ratio), and enhances phagocytosis of antibody-coated target cells by macrophages. An asymmetric Fc in which one heavy chain contains L234Y/L235Q/G236W/S239M/H268D/D270E/S298A mutations and D270E/K326D/A330M/K334E in the opposing heavy chain, increases affinity for FcγRIIIa F158 (a lower-affinity allele) and FcγRIIIa V158 (a higher-affinity allele) with no increased binding affinity to inhibitory FcγRIIb (Mimoto et al., mAbs, 2013, 5(2): 229-236).

Enhanced ADCP: Fc engineering can be used to enhance ADCP. For example (with reference to IgG1), G236A/S239D/I332E increases FcγRIIa binding and increases FcγRIIIa binding (Richards, J. et al., Mol. Cancer Ther., 2008, 7: 2517-2527).

Increased co-engagement: Fc engineering can be used to increase co-engagement with FcRs. For example (with reference to IgG1), S267E/L328F increases FcγRIIb binding; N325S/L328F increases FcγRIIa binding and decreases FcγRIIIa binding Wang et al., Protein Cell, 2018, 9(1): 63-73).

In a further embodiment, an antibody, or antigen binding fragment thereof, of the present invention may comprise a heavy chain constant region with an altered glycosylation profile, such that the antibody, or antigen binding fragment thereof, has an enhanced effector function, e.g., enhanced ADCC, enhanced CDC, or both enhanced ADCC and CDC. Examples of suitable methodologies to produce an antibody, or antigen binding fragment thereof, with an altered glycosylation profile are described in WO 2003/011878, WO 2006/014679 and EP1229125.

The absence of the α1,6 innermost fucose residues on the Fc glycan moiety on N297 of IgG1 antibodies enhances affinity for FcγRIIIA. As such, afucosylated or low fucosylated monoclonal antibodies may have increased therapeutic efficacy (Shields et al., J Biol Chem., 2002, 277(30): 26733-40 and Monnet et al., mAbs, 2014, 6(2): 422-436).

In certain embodiments there is provided an antibody, or antigen binding fragment thereof, comprising a chimeric heavy chain constant region. In an embodiment, the antibody, or antigen binding fragment thereof, comprises an IgG1/IgG3 chimeric heavy chain constant region, such that the antibody, or antigen binding fragment thereof, has an enhanced effector function, for example enhanced ADCC or enhanced CDC, or enhanced ADCC and CDC functions. For example, a chimeric antibody, or antigen binding fragment thereof, of the invention may comprise at least one CH2 domain from IgG3. In one such embodiment, the antibody, or antigen binding fragment thereof, comprises one CH2 domain from IgG3 or both CH2 domains may be from IgG3. In a further embodiment, the chimeric antibody, or antigen binding fragment thereof, comprises an IgG1 CH1 domain, an IgG3 CH2 domain, and an IgG3 CH3 domain. In a further embodiment, the chimeric antibody, or antigen binding fragment thereof, comprises an IgG1 CH1 domain, an IgG3 CH2 domain, and an IgG3 CH3 domain except for position 435 that is histidine.

In a further embodiment, the chimeric antibody, or antigen binding fragment thereof, comprises an IgG1 CH1 domain and at least one CH2 domain from IgG3. In an embodiment, the chimeric antibody, or antigen binding fragment thereof, comprises an IgG1 CH1 domain and the following residues, which correspond to IgG3 residues, in a CH2 domain: 274Q, 276K, 296F, 300F and 339T. In an embodiment, the chimeric antibody, or antigen binding fragment thereof, also comprises 356E, which corresponds to an IgG3 residue, within a CH3 domain. In an embodiment, the antibody, or antigen binding fragment thereof, also comprises one or more of the following residues, which correspond to IgG3 residues within a CH3 domain: 358M, 3845, 392N, 397M, 4221, 435R, and 436F.

Also provided is a method of producing an antibody, or antigen binding fragment thereof, according to the invention comprising the steps of:
a) culturing a recombinant host cell comprising an expression vector comprising a nucleic acid sequence encoding a chimeric Fc region having both IgG1 and IgG3 Fc region amino acid residues (e.g. as described above); and
b) recovering the antibody, or antigen binding fragment thereof.

Such methods for the production of antibody, or antigen binding fragment thereof, with chimeric heavy chain constant regions can be performed, for example, using the COMPLEGENT technology system available from BioWa, Inc. (Princeton, NJ) and Kyowa Hakko Kirin Co., Ltd. The COMPLEGENT system comprises a recombinant host cell comprising an expression vector in which a nucleic acid sequence encoding a chimeric Fc region having both IgG1 and IgG3 Fc region amino acid residues is expressed to produce an antibody, or antigen binding fragment thereof, having enhanced CDC activity, i.e. CDC activity is increased relative to an otherwise identical antibody, or antigen binding fragment thereof, lacking such a chimeric Fc region, as described in WO 2007/011041 and US 2007/0148165, each of which are incorporated herein by reference. In an alternative embodiment, CDC activity may be increased by introducing sequence specific mutations into the Fc region of an IgG chain. Those of ordinary skill in the art will also recognize other appropriate systems.

Also provided is a method of producing an antibody, or antigen binding fragment thereof, according to the invention comprising the steps of:
a) culturing a recombinant host cell comprising an expression vector comprising a nucleic acid encoding the antibody, or antigen binding fragment thereof, optionally wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the POTELLIGENT technology system available from BioWa, Inc. (Princeton, NJ) in which CHOK1SV cells lacking a functional copy of the FUT8 gene produce monoclonal antibodies having enhanced ADCC activity that is increased relative to an identical monoclonal antibody produced in a cell with a functional FUT8 gene as described in U.S. Pat. Nos. 7,214,775, 6,946,292, WO 00/61739 and WO 02/31240, all of which are incorporated herein by reference. Those of ordinary skill in the art will also recognize other appropriate systems.

In certain embodiments, the antibody, or antigen binding fragment thereof, is produced in a host cell in which the FUT8 gene has been inactivated. In a further embodiment, the antibody, or antigen binding fragment thereof, is produced in a −/− FUT8 host cell. In a further embodiment, the antibody, or antigen binding fragment thereof, is afucosylated at Asn297 (IgG1).

It will be apparent to those skilled in the art that such modifications may not only be used alone but may be used in combination with each other in order to further enhance effector function.

In one such embodiment, there is provided an antibody, or antigen binding fragment thereof, comprising a heavy chain constant region that comprises a both a mutated and chimeric heavy chain constant region, individually described above. For example, an antibody, or antigen binding fragment thereof, comprising at least one CH2 domain from IgG3 and one CH2 domain from IgG1, and wherein the IgG1 CH2 domain has one or more mutations at positions selected from 239, 332 and 330 (for example the mutations may be selected from S239D, 1332E and A330L), such that the antibody, or antigen binding fragment thereof, has enhanced effector function, e.g. enhanced ADCC or enhanced CDC, or enhanced ADCC and enhanced CDC in comparison to an equivalent antibody, or antigen binding fragment thereof, with an IgG1 heavy chain constant region lacking said mutations. In certain embodiments, the IgG1 CH2 domain has the mutations S239D and 1332E. In certain embodiments, the IgG1 CH2 domain has the mutations S239D, A330L, and 1332E.

In an alternative embodiment, there is provided an antibody, or antigen binding fragment thereof, comprising both a chimeric heavy chain constant region and an altered glycosylation profile, as individually described above. In an embodiment, the antibody, or antigen binding fragment thereof, comprises an altered glycosylation profile such that the ratio of fucose to mannose is 0.8:3 or less. In one such embodiment, the heavy chain constant region comprises at least one CH2 domain from IgG3 and one CH2 domain from IgG1 and has an altered glycosylation profile such that the ratio of fucose to mannose is 0.8:3 or less, for example wherein the antibody, or antigen binding fragment thereof, is defucosylated. Said antibody, or antigen binding fragment thereof, has an enhanced effector function, e.g. enhanced ADCC or enhanced CDC, or enhanced ADCC and enhanced CDC, in comparison to an equivalent antibody, or antigen binding fragment thereof, with an IgG1 heavy chain constant region lacking said glycosylation profile.

In an alternative embodiment, the antibody, or antigen binding fragment thereof, has at least one IgG3 heavy chain CH2 domain and at least one heavy chain constant domain from IgG1 wherein both IgG CH2 domains are mutated in accordance with the limitations described herein.

In one aspect, there is provided a method of producing an antibody, or antigen binding fragment thereof, according to the invention described herein comprising the steps of:
a) culturing a recombinant host cell containing an expression vector comprising a nucleic acid sequence encoding a chimeric Fc domain having both IgG1 and IgG3 Fc domain amino acid residues (e.g. as described above); and wherein the FUT8 gene encoding alpha-1,6-fucosyltransferase has been inactivated in the recombinant host cell; and
b) recovering the antibody, or antigen binding fragment thereof.

Such methods for the production of an antibody, or antigen binding fragment thereof, can be performed, for example, using the ACCRETAMAB technology system available from BioWa, Inc. (Princeton, NJ) that combines the POTELLIGENT and COMPLEGENT technology systems to produce an antibody, or antigen binding fragment thereof, having both enhanced ADCC and CDC activity relative to an otherwise identical monoclonal antibody that lacks a chimeric Fc domain and that is fucosylated.

In certain embodiments, there is provided an antibody, or antigen binding fragment thereof, comprising a mutated and chimeric heavy chain constant region wherein said antibody, or antigen binding fragment thereof, has an altered glycosylation profile such that the antibody, or antigen binding fragment thereof, has enhanced effector function, e.g. enhanced ADCC or enhanced CDC, or both enhanced ADCC and CDC. In certain embodiments the mutations are selected from positions 239, 332 and 330, e.g. S239D, 1332E and A330L. In a further embodiment the heavy chain constant region comprises at least one CH2 domain from IgG3 and one CH1 domain from IgG1. In certain embodiments the heavy chain constant region has an altered glycosylation profile such that the ratio of fucose to mannose is 0.8:3 or less, e.g. the antibody, or antigen binding fragment thereof, is defucosylated, such that said antibody, or antigen binding fragment thereof, has an enhanced effector function in comparison with an equivalent non-chimeric antibody, or antigen binding fragment thereof, lacking said mutations and lacking said altered glycosylation profile.

In a further embodiment, the anti-cotinine antibody, or antigen binding fragment thereof, comprises a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6. In a further embodiment, the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/I332E or S239D/I332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/I332E, wherein residue numbering is according to the EU Index.

In a further embodiment, the anti-cotinine antibody, or antigen binding fragment thereof, comprises a heavy chain variable region (VH) having SEQ ID NO: 7, a light chain variable region (VL) having SEQ ID NO: 8. In a further embodiment, the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8. In a further embodiment, the anti-cotinine antibody is of IgG1 isotype. In a further embodiment, the anti-cotinine antibody is of IgG1 isotype comprising a substitution in an Fc region to increase or enhance ADCC activity. In a further embodiment, the anti-cotinine antibody is of IgG1 isotype comprising a substitution in an Fc region to increase or enhance ADCC activity, wherein the substitution is S239D/I332E or S239D/I332E/A330L, wherein residue numbering is according to the EU Index. In a further embodiment, the anti-cotinine antibody is of IgG1 isotype comprising a substitution in an Fc region to increase or enhance ADCC activity, wherein the substitution is S239D/I332E, wherein residue numbering is according to the EU Index.

In a further embodiment, the anti-cotinine antibody has a heavy chain comprising SEQ ID NO: 9 and a light chain comprising SEQ ID NO: 10.

Also provided is a pharmaceutical composition comprising an anti-cotinine antibody, or antigen binding fragment thereof as disclosed herein, and a pharmaceutically acceptable excipient, carrier, or diluent.

Also provided is a combination comprising a heterobifunctional compound described herein (e.g., a compound of Formula (I)), and an anti-cotinine antibody, or antigen-binding fragment thereof as disclosed herein. The heterobifunctional compound and anti-cotinine antibody, or antigen binding fragment thereof can be present in the same composition or in separate compositions. In certain embodiments, a combination comprises a pharmaceutical composition comprising a heterobifunctional compound (e.g., a compound of Formula (I)) and an anti-cotinine antibody, or antigen binding fragment thereof as disclosed herein, and a pharmaceutically acceptable carrier, diluent, or excipient. In certain embodiments, a combination comprises a first pharmaceutical composition comprising a heterobifunctional compound (e.g., a compound of Formula (I)) and a pharmaceutically acceptable carrier, diluent, or excipient; and a second pharmaceutical composition comprising an anti-cotinine antibody or antigen binding fragment thereof as disclosed herein, and a pharmaceutically acceptable carrier, excipient, or diluent.

The heterobifunctional compounds (e.g., a compound of Formula (I)) and pharmaceutically acceptable salts thereof are capable of simultaneously binding a cell surface-expressed CCR2 and an anti-cotinine antibody, or antigen binding fragment thereof to form a ternary complex for the treatment and/or prevention of diseases or disorders associated with CCR2-expressing cells.

Also provided is a method of treating and/or preventing a disease or disorder in a patient in need thereof comprising administering to the patient a therapeutically effective amount of a heterobifunctional compound (e.g., a compound of Formula (I), or a pharmaceutically acceptable salt thereof) and an anti-cotinine antibody, or antigen-binding fragment thereof, wherein the disease or disorder is selected from a cancer, an inflammatory disease, an autoimmune disease, a viral infection, or a bacterial infection.

In a further embodiment, the compound and the antibody, or antigen-binding fragment thereof, are administered simultaneously. In a further embodiment, the compound and the antibody, or antigen-binding fragment thereof, are administered simultaneously from a single composition, including as a fixed-dose composition or by pre-mixing the compound and the antibody, or antigen-binding fragment thereof, prior to administration. For example, the compound and the antibody, or antigen-binding fragment thereof, can be pre-mixed about 2 seconds to about 30 seconds, about 30 seconds to about 2 minutes, about 2 minutes to about 10 minutes, about 10 minutes to about 30 minutes, or about 30 minutes to about 2 hours prior to administration. In a further embodiment, the compound and the antibody, or antigen-binding fragment thereof, are administered simultaneously from two separate compositions.

In a further embodiment, the compound and the antibody, or antigen-binding fragment thereof, are administered sequentially.

In certain embodiments, the compound and the antibody, or antigen-binding fragment thereof, whether administered simultaneously or sequentially, may be administered by the same route or may be administered by different routes. In certain embodiments, the compound and the antibody, or antigen-binding fragment thereof, are both administered intravenously or subcutaneously, in the same composition or in separate compositions. In certain embodiments, the compound is administered orally and the antibody or antigen-binding fragment thereof is administered intravenously or subcutaneously.

In a further embodiment, the compound and the antibody, or antigen-binding fragment thereof, are administered in a molar ratio of compound to antibody, or antigen-binding fragment thereof, of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody, or antigen-binding fragment thereof, are present as a combination in a molar ratio of compound to antibody, or antigen-binding fragment thereof, of about 2:1, about 1.8:1, about 1.6:1, about 1.5:1, about 1.4:1, about 1.3:1, about 1.2:1, about 1:1, about 1:1.2, about 1:1.3, about 1:1.4, about 1:1.5, about 1:1.6, about 1:1.8, about 1:2, about 2:1 to about 1.5:1, about 1.5:1 to about 1.2:1, about 1.2:1 to about 1:1, about 1:1 to about 1:1.2, about 1:1.2 to about 1:1.5, or about 1:1.5 to about 1:2.

In a further embodiment, the compound and the antibody, or antigen-binding fragment thereof, are administered at a dosage of compound of 0.0001 mg/kg to 1 mg/kg and antibody of 0.01 mg/kg to 100 mg/kg. For example, in a further embodiment, the compound is administered at a dosage of about 0.0001 mg/kg to about 0.0002 mg/kg, about 0.0002 mg/kg to about 0.0003 mg/kg, about 0.0003 mg/kg to about 0.0004 mg/kg, about 0.0004 mg/kg to about 0.0005 mg/kg, about 0.0005 mg/kg to about 0.001 mg/kg, about 0.001 mg/kg to about 0.002 mg/kg, about 0.002 mg/kg to about 0.003 mg/kg, about 0.003 mg/kg to about 0.004 mg/kg, about 0.004 mg/kg to about 0.005 mg/kg, about 0.005 mg/kg to about 0.01 mg/kg, about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, and/or about 0.5 mg/kg to about 1 mg/kg, and the antibody, or antigen-binding fragment thereof, is administered at a dosage of about 0.01 mg/kg to about 0.02 mg/kg, about 0.02 mg/kg to about 0.03 mg/kg, about 0.03 mg/kg to about 0.04 mg/kg, about 0.04 mg/kg to about 0.05 mg/kg, about 0.05 mg/kg to about 0.1 mg/kg, about 0.1 mg/kg to about 0.2 mg/kg, about 0.2 mg/kg to about 0.3 mg/kg, about 0.3 mg/kg to about 0.4 mg/kg, about 0.4 mg/kg to about 0.5 mg/kg, about 0.5 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, about 45 mg/kg to about 50 mg/kg, about 50 mg/kg to about 60 mg/kg, about 60 mg/kg to about 70 mg/kg, about 70 mg/kg to about 80 mg/kg, about 80 mg/kg to about 90 mg/kg, and/or about 90 mg/kg to about 100 mg/kg.

In a further embodiment, the compound and the antibody, or antigen-binding fragment thereof, are administered at a dosage of compound of 0.007 mg to 70 mg and antibody of 0.7 mg to 7000 mg. For example, in a further embodiment, the compound is administered at a dosage of about 0.007 mg to about 0.01 mg, about 0.01 mg to about 0.02 mg, about 0.02 mg to about 0.03 mg, about 0.03 mg to about 0.04 mg, about 0.04 mg to about 0.05 mg, about 0.05 mg to about 0.1 mg, about 0.1 mg to about 0.2 mg, about 0.2 mg to about 0.3 mg, about 0.3 mg to about 0.4 mg, about 0.4 mg to about 0.5 mg, about 0.5 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 60 mg, and/or about 60 mg to about 70 mg, and the antibody, or antigen-binding fragment thereof, is administered at a dosage of about 0.7 mg to about 1 mg, about 1 mg to about 2 mg, about 2 mg to about 3 mg, about 3 mg to about 4 mg, about 4 mg to about 5 mg, about 5 mg to about 10 mg, about 10 mg to about 20 mg, about 20 mg to about 30 mg, about 30 mg to about 40 mg, about 40 mg to about 50 mg, about 50 mg to about 100 mg, about 100 mg to about 500 mg, about 500 mg to about 1000 mg, about 1000 mg to about 1500 mg, about 1500 mg to about 2000 mg, about 2000 mg to about 2500 mg, about 2500 mg to about 3000 mg, about 3000 mg to about 3500 mg, about 3500 mg to about 4000 mg, about 4000 mg to about 4500 mg, about 4500 mg to about 5000 mg, about 5000 mg to about 5500 mg, about 5500 mg to about 6000 mg, about 6000 mg to about 6500 mg, and/or about 6500 mg to about 7000 mg.

In a further embodiment, the compound and the antibody, or antigen-binding fragment thereof, are administered in a molar ratio and/or dosage as described herein once every week, once every two weeks, once every three weeks, once every four weeks, once every five weeks, or once every six weeks for a period of one week to one year, such as a period of one week, one month, two months, three months, four months, five months, six months, seven months, eight months, nine months, ten months, eleven months, or twelve months.

### III. Therapeutic Applications

One aspect of the invention provides a method of treating or preventing a disease or disorder in a patient in need thereof, wherein the method comprises administering to the patient a therapeutically effective amount of a heterobifunctional compound described herein (e.g., a compound of Formula I or a pharmaceutically acceptable salt thereof) and an anti-cotinine antibody, or antigen-binding fragment thereof.

In certain embodiments, the invention provides a method of treating or preventing a disease or disorder in a patient in need thereof, wherein the method comprises administering to the patient a therapeutically effective amount of a heterobifunctional compound described herein (e.g., a compound of Formula I or a pharmaceutically acceptable salt thereof) and an anti-cotinine antibody, or antigen-binding fragment thereof, wherein the disease or disorder is selected from a cancer, an inflammatory disease, an autoimmune disease, a viral infection, or a bacterial infection.

In certain embodiments, the method is to treating the disease or disorder. In certain embodiments, the method is to preventing the disease or disorder.

In certain embodiments, the disease or disorder is mediated by C—C motif chemokine receptor 2 (CCR2) and/or is associated with CCR2-positive pathogenic cells.

In certain embodiments, the disease is a cancer that is a solid tumor. In certain embodiments, the cancer is selected from non-small cell lung cancer (NSCLC), hepatocellular carcinoma (HCC), colorectal cancer (CRC), cervical squamous cell carcinoma (CESC), head and neck squamous cell carcinoma (HNSC), pancreatic cancer, metastatic castration-resistant prostate cancer (mCRPC), ovarian cancer, bladder cancer, or breast cancer. In certain embodiments, the disease is a cancer selected from non-small cell lung cancer (NSCLC), hepatocellular carcinoma (HCC), colorectal cancer (CRC), cervical squamous cell carcinoma (CESC), head and neck squamous cell carcinoma (HNSC), pancreatic cancer, metastatic castration-resistant prostate cancer (mCRPC), ovarian cancer, bladder cancer, or breast cancer.

In certain embodiments, the compound and the antibody, or antigen-binding fragment thereof, are administered simultaneously. In certain embodiments, the compound and the antibody, or antigen-binding fragment thereof, are administered sequentially.

Another aspect of the invention provides a method of increasing antibody-dependent cell cytotoxicity (ADCC) of C—C motif chemokine receptor 2 (CCR2)-expressing cells, wherein the method comprises contacting the cells with an effective amount of a heterobifunctional compound described herein (e.g., a compound of Formula I or a pharmaceutically acceptable salt thereof) and an anti-cotinine antibody, or antigen-binding fragment thereof, wherein the CCR2-binding moiety of the compound binds the CCR2 expressed on the cells.

Another aspect of the invention provides a method of depleting C—C motif chemokine receptor 2 (CCR2)-expressing cells, wherein the method comprising contacting the cells with an effective amount of a heterobifunctional compound described herein (e.g., a compound of Formula I or a pharmaceutically acceptable salt thereof) and an anti-cotinine antibody, or antigen-binding fragment thereof, wherein the CCR2-binding moiety of the compound binds the CCR2 expressed on the cells.

In certain embodiments, the CCR2-expressing cells are myeloid-derived suppressor cells (MDSCs), T regulatory cells (Tregs), neutrophils, macrophages, B regulatory cells (Bregs), CD8 regulatory cells, (CD8regs), exhausted T cells, or cancer-associated fibroblasts (CAFs).

In certain embodiments, the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6. In certain embodiments, the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8.

In certain embodiments, the anti-cotinine antibody is of IgG1 isotype comprising a substitution in an Fc region to increase ADCC activity. In certain embodiments, the substitution in the Fc region is S239D/I332E, wherein residue numbering is according to the EU Index.

In certain embodiments, the anti-cotinine antibody has a heavy chain comprising SEQ ID NO: 9 and a light chain comprising SEQ ID NO: 10.

Another aspect of the invention provides a combination comprising a heterobifunctional compound described herein (e.g., a compound of Formula I or a pharmaceutically acceptable salt thereof) and an anti-cotinine antibody, or antigen-binding fragment thereof.

In certain embodiments, the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6. In certain embodiments, the anti-cotinine antibody has a heavy chain and a light chain, the heavy chain comprising a heavy chain variable region (VH) having SEQ ID NO: 7, and the light chain comprising a light chain variable region (VL) having SEQ ID NO: 8.

In certain embodiments, the anti-cotinine antibody is of IgG1 isotype comprising a substitution in an Fc region to increase ADCC activity. In certain embodiments, the substitution in the Fc region is S239D/I332E, wherein residue numbering is according to the EU Index.

In certain embodiments, the anti-cotinine antibody has a heavy chain comprising SEQ ID NO: 9 and a light chain comprising SEQ ID NO: 10.

Another aspect of the invention provides a method of treating cancer, wherein the method comprises administering to a subject in need thereof a therapeutically effective amount of a heterobifunctional compound described herein, such as a compound of Formula I, to treat the cancer. In certain embodiments, the particular compound of Formula I is a compound defined by one of the embodiments described in Section I, above. In certain embodiments, the method further comprises administering to the subject an effective amount of an anti-cotinine antibody, or antigen-binding fragment thereof.

Methods described herein may be further defined according to additional features, such as the identity of the cancer and/or the subject.

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, leukemia, urothelial cancer, colorectal cancer, or glioblastoma multiforme.

In certain embodiments, the cancer is a breast invasive carcinoma, colon adenocarcinoma, head and neck cancer, lung adenocarcinoma, rectal adenocarcinoma, acute myeloid leukemia, glioblastoma multiforme, brain lower grade glioma, colorectal cancer, or metastatic melanoma. In certain embodiments, the cancer is a melanoma.

In certain embodiments, the disorder is a cancer selected from the group consisting of ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, and leukemia.

In certain embodiments, the cancer is a solid tumor. In certain embodiments, the cancer is a sarcoma or carcinoma. In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is prostate cancer, breast cancer, lung cancer, liver cancer, bladder cancer, urinary tract cancer, or eye cancer. In certain embodiments, the cancer is prostate cancer. In certain embodiments, the cancer is breast cancer. In certain embodiments, the cancer is lung cancer. In certain embodiments, the cancer is liver cancer. In certain embodiments, the cancer is bladder cancer. In certain embodiments, the cancer is urinary tract cancer. In certain embodiments, the cancer is eye cancer.

In certain embodiments, the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas (e.g., Burkitt's lymphoma and Non-Hodgkin's lymphoma); benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

In certain embodiments, the cancer is a neuroblastoma, craniopharyngioma, glioma, glioblastoma, schwannoma, astrocytoma, oligodendroglioma, medulloblastoma, pinealoma, hemangioblastoma, retinoblastoma, ependymoma, chordoma, meningioma, medullary carcinoma, small cell lung carcinoma, papillary adenocarcinoma, papillary carcinoma, mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct and gallbladder cancers, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

In certain embodiments, the cancer is a lymphoma. In certain embodiments, the cancer is Burkitt's lymphoma, diffuse large B-cell lymphoma (DLBCL), follicular lymphoma, non-Hodgkin's lymphoma, lymphoid malignancies of T-cell or B-cell origin, peripheral T-cell lymphoma, adult T-cell leukemia-lymphoma, or Waldenstrom's macroglobulinemia.

In certain embodiments, the cancer is a leukemia. In certain embodiments, the cancer is acute leukemia, lymphoblastic leukemia, acute lymphoblastic leukemia, myelogenous leukemia, acute myelogenous leukemia, acute T-cell leukemia, chronic leukemia, chronic lymphocytic leukemia, chronic myelocytic leukemia, chronic myelogenous leukemia, polycythemia vera, multiple myeloma, or erythroleukemia.

In certain embodiments, the cancer is a myelodysplastic and/or myeloproliferative syndrome. In certain embodiments, the cancer is a myelodysplastic syndrome. In certain embodiments, the cancer is a myeloproliferative syndrome.

In certain embodiments, the cancer is a cancer or related myeloproliferative disorder selected from histiocytosis, essential thrombocythemia, myelofibrosis, heavy chain disease, and other malignancies and hyperproliferative disorders of the bladder, breast, colon, lung, ovaries, pancreas, prostate, skin and uterus.

In certain embodiments, the cancer is a B-cell non-Hodgkin's lymphoma, advanced solid tumor, soft tissue sarcoma, INI1-deficient cancer, BAP1-deficient cancer, follicular lymphoma, relapsed/refractory follicular lymphoma, diffuse large B-cell lymphoma, relapsed/refractory diffuse large B-cell lymphoma, non-Hodgkin's lymphoma, pediatric non-Hodgkin's lymphoma, pediatric non-Hodgkin's lymphoma with EZH2, SMARCB1, or SMARCA4 mutation, histiocytic disorder, pediatric histiocytic disorder, pediatric histiocytic disorder with EZH2, SMARCB1, or SMARCA4 mutation, solid tumor with EZH2, SMARCB1, or SMARCA4 mutation, resistant prostate cancer, relapsed/refractory small-cell lung carcinoma, B-cell lymphoma, relapsed/refractory B-cell lymphoma, adult T-cell leukemia-lymphoma, or advanced diffuse large B-cell lymphoma.

In certain embodiments, the cancer is a malignant rhabdoid tumor, atypical teratoid rhabdoid tumor, epithelioid sarcoma, renal medullary carcinoma, pancreatic undifferentiated rhabdoid carcinoma, schwannoma, epithelioid malignant peripheral nerve sheath tumor, or diffuse intrinsic glioma.

In certain embodiments, the cancer is retinoblastoma multiforme, metastatic castration-resistant prostate cancer, prostate small cell neuroendocrine carcinoma, small-cell lung cancer, triple-negative breast cancer, hepatocellular carcinoma, bladder cancer, or urinary tract cancer.

In certain embodiments, the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, and hemangioblastoma. In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is a metastatic cancer. In certain embodiments, the cancer is a relapsed and/or refractory cancer.

In certain embodiments, the cancer is ovarian cancer, uterine cancer, gestational trophoblastic disease, endometrial cancer, cervical cancer, embryonal carcinoma, choriocarcinoma, prostate cancer (including hormone insensitive and castrate resistant prostate cancers), testicular tumors (including germ cell testicular cancer/seminoma), cystadenocarcinoma, breast cancer (including estrogen-receptor positive breast cancer), brain tumors (including neuroblastoma, craniopharyngioma, glioma, glioblastoma, schwannoma, astrocytoma, oligodendroglioma, medulloblastoma, and pinealoma), hemangioblastoma, retinoblastoma, ependymoma, chordoma, meningioma, medullary carcinoma, lung cancer (including small cell lung carcinoma, papillary adenocarcinomas, and papillary carcinoma), mesothelioma, nasopharyngeal carcinoma, acoustic neuroma, oral cancer, esophageal cancer, head and neck cancer, stomach cancer, colon cancer, rectal cancer, skin cancer, melanoma, sweat gland carcinoma, sebaceous gland carcinoma, squamous cell carcinoma, basal cell carcinoma, bile duct and gallbladder cancers, liver cancer, hepatocellular carcinoma, pancreatic cancer, bladder carcinoma, renal cell carcinoma, kidney cancer, Wilms' tumor, thyroid cancer, parathyroid tumor, synovioma, soft tissue sarcoma (e.g., rhabdomyosarcoma (RMS)), Kaposi sarcoma, synovial sarcoma, osteosarcoma, Ewing's sarcoma, malignant rhabdoid tumor, leiomyosarcoma, liposarcoma, lymphangioendothelio-sarcoma, lymphangiosarcoma, myxosarcoma, osteogenic sarcoma, fibrosarcoma, chondrosarcoma, or endotheliosarcoma.

### Subjects

In certain embodiments, the subject is a human. In certain embodiments, the subject is an adult human. In certain embodiments, the subject is a pediatric human.

### Medical Uses

Another aspect of the invention provides for the use of a heterobifunctional compound described herein (such as a compound of Formula I, or other compounds in Section I) in the manufacture of a medicament. In certain embodiments, the medicament is for treating a disorder described herein, such as cancer.

Another aspect of the invention provides for the use of a heterobifunctional compound described herein (such as a compound of Formula I, or other compounds in Section I) for treating a medical disorder, such as a medical disorder described herein, such as cancer.

Another aspect of the invention provides a heterobifunctional compound described herein (such as a compound of Formula I, or other compounds in Section I) for use in treating a medical disorder, such as a medical disorder described herein, such as cancer.

### IV. Biological Assays for Evaluating Compound Activity

Heterobifunctional compounds may be evaluated for biological activity using one or more of the assays described below.

### Assay 1: Antibody Dependent Cellular Cytotoxicity Reporter Assay

An antibody dependent cellular cytotoxicity reporter assay is conducted using the following four assay components: (i) ARM compound of Formula (I) targeting CCR2 (concentrations ranging from 1 pM to 10 μM) (ii) anti-cotinine antibody having a heavy chain sequence of SEQ ID NO: 11 and a light chain sequence of SEQ ID NO: 12 (rabbit variable region with human IgG1 Fc domain containing a DE mutation (S239D/I332E)) (concentrations ranging from 0.01 μg/mL to 200 μg/mL); (iii) target cells: CHOK1 cells engineered to overexpress either human CCR2 (typically 1000-20,000 cells per well) and (iv) reporter cells: Jurkat cells engineered to express FcγRIIIa with the reporter gene luciferase under the control of the NFAT promoter (typically 3000-75,000 cells per well). Reagents are combined in a final volume of 20 μL in a 384-well tissue culture treated plate. All four assay components are incubated together for about 12-18 hours. Thereafter, BioGlo Detection reagent (Promega) is added to the wells to lyse the cells and provide a substrate for the luciferase reporter protein. Luminescence signal is measured on a microplate reader and signal:background is calculated by dividing the signal of a test well by the signal obtained when no heterobivalent compound of Formula (I) was added. EC50 calculations are done using Graphpad Prism Software, specifically a nonlinear regression curve fit (Y=Bottom+(Top−Bottom)/(1+10\hat{ }((Log EC50−X)*HillSlope))).

### Assay 2: PBMC Ex Vivo Depletion Assay

A human PBMC (peripheral blood mononuclear cells) monocyte depletion flow cytometry assay can be used to screen compounds in a 384-well plate format. This assay incorporates endogenous natural killer cells to deplete endogenous target monocytes.

To screen compounds in the human PBMC monocyte depletion flow cytometry assay, 11-point dose-response curves are made from 1 mM stocks in neat DMSO by serially diluting an ARM compound of Formula (I) four-fold into a 384-well plate row-wise. These serially diluted compounds are then stamped into a flat bottom polypropylene 384-well plate (Greiner Bio-One) at 100 nL/well. Fifty microliters of 30 μg/mL anti-cotinine antibody having a heavy chain sequence of SEQ ID NO: 11 and a light chain sequence of SEQ ID NO: 12 (rabbit variable region with human IgG1 Fc domain containing a DE mutation (S239D/I332E)) diluted in assay medium (RPMI 1640 (Gibco) containing 10% fetal bovine serum (Gibco), 1% Glutamax (Gibco) and 1% sodium pyruvate (Corning) is added to the wells of the plate. The plate is centrifuged for 1 min at 500 rpm (revolutions per minute) and then shaken for 5 min at room temperature. The plate is incubated for 15 minutes at 37° C./5% CO₂.

Frozen PBMC cells (HemaCare BioResearch) are thawed and centrifuged at 1,200 rpm for 4 minutes. The cells are diluted to a cell density of 2×10⁶ cells/mL in assay medium. Fifty microliters of the diluted cells are added to the wells of the plate. The plate is centrifuged for 1 min at 500 rpm and incubated for 18-24 hours at 37° C./5% CO₂.

After 18-24 hours incubation, 2 μL of 6.25 μg human FC block (BD Pharmingen) diluted in assay medium is added to the wells of the plate. The plate is centrifuged for 1 minute at 500 rpm and then incubated at room temperature for 15 minutes. Five microliters of 750 nM Helix NP NIR (Biolegend) dilute in assay medium is added to the wells of the plate. Nine microliters of antibody cocktail containing 1/5 diluted fluorescein isothiocyanate (FITC) labeled anti-CD45 antibody (Beckman Coulter), PC7 labeled anti-CD14 antibody (Beckman Coulter) and phyocoerythrin (PE) anti-CD4 antibody (Beckman Coulter) is then added to the wells of the plate. The plate is centrifuged for 1 min at 500 rpm and incubated for 30 minutes at 4-8° C. Samples from the plate ae run on BD FACS Canto II flow cytometer and data is analyzed using FlowJo software.

### Assay 3: In Vivo Depletion Assay

Human CCR2 (hCCR2) knock-in mice (C57 background) are dosed intravenously with a PBS solution containing a compound of Formula (I) to measure depletion of CCR2 expressing cells in peripheral blood. Peripheral blood from IV dosed mice is also analyzed to determine PK properties of the ARM compounds of Formula (I).

Stock solution preparation: Stock solutions of compounds of Formula (I) are prepared at 20 mg/nL and 100 mg/mL in DMSO for PK/PD and dose tolerability studies respectively. Stock solutions are stored at −20° C. until further usage.

Formulations preparation: On the day of experiment, the stock solution of the compound of Formula (I) is removed from storage at −20° C. and thawed at room temperature. Anti-cotinine antibody having a heavy chain sequence of SEQ ID NO: 13 and a light chain sequence of SEQ ID NO: 14 (rabbit variable region sequence with mouse IgG2a Fc domain), if required is removed from storage at −80° C. and thawed at room temperature. Antibody vials are immediately transferred into wet ice after thawing. Compounds of Formula (I) are further diluted in DMSO as per experimental requirements.

Formulation composition: The formulation composition may be Saline: DMSO: PBS. Saline is added based on the quantity required and then stock solution of the compound of Formula (I) prepared in DMSO, followed by addition of antibody in PBS. Formulations are incubated at room temperature for 30 minutes before administration to the mouse. DMSO is used at 1 to 2% (v/v) in the final formulation.

Administration to Animal: Solution formulation of antibody and compound of Formula (I) is injected (bolus injection) to the restrained mouse in the right/left lateral tail vein. Animals are dosed with 0.1 milligram per kilogram (mpk) of the compound of Formula (I) and 10 mpk of the anti-cotinine antibody.

Collection of Blood for PK: Blood is collected at time points 0.25 hour, 2 hours, and 4 hours following administration (50 μL/time point) through retro-orbital bleeding under mild isoflurane anesthesia.

Terminal bleeding at end of experiment (48 hr): Approximately 250 μL of blood in K₂EDTA tube and approximately 250 μL of blood in SST (serum separation tube) is collected from each mouse through retro-orbital bleeding under deep isoflurane anesthesia. After bleeding, each mouse is sacrificed by cervical dislocation. The blood distribution at termination may be determined as follows:

For PK: 50 μL of K₂EDTA blood is transferred to another tube for PK

Flow cytometry: Remaining blood (˜200 μL) is used for flow cytometry analysis.

For serum collection: serum separator tubes (BD) are centrifuged at 4° C., 5000 rpm for 15 minutes. Approximately 100 μL serum is separated and stored at −80° C. for further usage.

Blood drug concentration: Samples collected at the various timepoints (0.25 hr, 2 hr, 24 and 48 hr) are analyzed to determine blood drug concentration.

Flow cytometry analysis: CCR2 depletion in peripheral blood may be determined by flow cytometry. Briefly, peripheral blood is collected from all animals at the end of study termination and processed. Blood samples are lysed using 1×RBC Lysis buffer and the resulting cell pellet is washed twice in FACS buffer (HBSS containing 5% FBS). Cells are subjected to Fc blocking using mouse Fc block from Biolegend (Trustain, anti-mouse CD16/32, Cat: 101319) to block non-specific binding followed by surface staining for immune markers (CD45, CD3, CD11b, Ly6C, human CCR2). Surface staining is done for 30 minutes in ice and cells are washed twice to remove unbound antibodies. Cells are stained with 7AAD to determine viability and acquired on BD FACSVerse flow cytometer. Appropriate FMO controls and single-color controls are included in the experiment for gating and compensation respectively.

CCR2+monocytes are gated as CD11b+Ly6C+Hi, medium and Low populations and treatment induced reduction in CD11b+Ly6c+Hi cells (i.e., cells with high CCR2 expression) is evaluated. Percent depletion of CD11b+Ly6c+Hi cells and CCR2 expression for the different treatment groups can be determined.

Blood drug concentration analysis: Drug concentration in blood samples may be determined by an LC-MS/MS-based bioanalytical method. Samples are analyzed on Q-Trap, API-5500 LC-MS/MS system coupled with Exion UHPLC system from SCIEX, USA operated in multiple reaction monitoring mode employing electrospray ionization technique in positive polarity. Analyte and internal standard peaks are resolved on Synergi Polar, 75×2.0 mm, 4μ column using mobile phase 10 mM Ammonium acetate in Milli-Q water as phase A and 0.1% Formic acid in acetonitrile as Phase B. Gradient elution is performed with initial composition 95% Phase A at 0.0 min, holding it for 0.2 minutes, ramping to 5% by 1.0 minute, keeping the same for next 0.5 minutes and coming back to 95% by 1.6 minutes. The total run time is 2 minutes.

Working dilutions for calibration curve and quality control standards are prepared by serially diluting 20 mg/mL stock solution with DMSO. Spiked concentrations for calibration curve in the whole blood may range from 1 ng/mL to 1000 ng/mL. The working solution of internal standard (Verapamil, 25 ng/mL) is prepared in acetonitrile. 10 μL of the study sample and calibration curve, quality control, and blank whole blood samples are aliquoted in 96 deep well plates for processing. 10 μL of Milli-Q water is added to all the samples and briefly vortexed to initiate complete hemolysis. 10 μL of 20 mM dithiothreitol (DTT) is added to all the samples and incubated for 30 minutes at 37° C. The addition of DTT enhanced the recovery of ARM compounds of Formula (I) from the biological matrix. 300 μL of working internal standard solution is added to all samples except total blank and wash samples, where 300 μL of acetonitrile is added. All the samples are vortex mixed for 5 minutes, followed by centrifugation at 4000 rpm for 10 minutes at 4° C. Supernatants are transferred to the loading plate and injected 3 μL to LC-MS/MS system for analysis.

### Assay 4: Binding of Anti-Cotinine Antibody to ARM Compounds of Formula (I) Measured by Surface Plasmon Resonance (SPR)

Anti-cotinine antibodies having a heavy chain sequence of SEQ ID NO: 11 and a light chain sequence of SEQ ID NO: 12 (rabbit variable region with human IgG1 Fc domain containing a DE mutation (S239D/I332E)) can be captured on one or more flow cells of a protein A sensor chip (Cytiva) using a Biacore T200 while reserving flow cell 1 as a reference. Following capture, a 3000 second wait step is included to reduce drift during compound analysis. ARM compounds of Formula (I) are then injected at 100 μL/min with 200 and 2000 second association and dissociation times. The entire experiment may be run at 37° C. with running buffer containing 10 mM HEPES pH 7.4, 150 mM NaCl, 0.005% P20 and 1% DMSO. ARM compounds of Formula (I) are titrated with a top concentration of 200 nM using a 3-fold 5-point dilution series and a corresponding 5-injection buffer cycle is run for blank subtraction. Data are double referenced by subtracting the response of the reference flow cell from that of the antibody-containing flow cell and subsequently subtracting the referenced blank sensorgrams. Following compound analysis, the surface is regenerated using a 30 second injection of pH 1.5 glycine at a flow rate of 30 μL/min after which antibody is re-captured. The experiment may be run using Biacore T200 control software and evaluated using Biacore T200 Evaluation software. Curves are fit with a 1:1 kinetic binding model to obtain kₒₙ (1/Ms), k_{off} (1/s), K_{d} (M) determined as k_{off}/kₒₙ, and residence time determined as 1/k_{off}. (s).

Anti-cotinine antibodies having a heavy chain sequence of SEQ ID NO: 9 and a light chain sequence of SEQ ID NO: 10 (humanized version) or a heavy chain sequence of SEQ ID NO: 11 and a light chain sequence of SEQ ID NO: 12 (rabbit version) may be captured via the Fc domain to a protein A or A/G surface on flow cell 2 of a CM5 sensor chip using the Biacore 8k. Both antibodies may have a human IgG1 Fc domain containing a DE mutation (S239D/I332E).

After antibody capture, a 1500 second wait step is included to reduce drift in the binding step. ARM compounds of Formula (I) are then flowed over the captured antibodies at varying top concentrations ranging from 250 nM to 4 μM. The top concentration is diluted 4-fold over 5 dilutions (with a 0 nM compound cycle included to blank subtract the data). Association and dissociation times of the ARMs compounds of Formula (I) may vary between experiments as follows: reference 1—association for 600 seconds and dissociation for 1200 seconds at 20 μl/min; reference 2—association for 240 seconds and dissociation for 300 seconds at 30 μl/min; reference 3—association for 360 seconds and dissociation for 600 seconds at 30 μl/min. Anti-cotinine antibody bound to the protein A or A/G surface is regenerated using 50 mM NaOH. Experiments are run at 25° C. at pH 7.4 using HBS-EP+ buffer using Biacore 8K control software and evaluated using Biacore Insight Evaluation software. Curves are fit with the 1:1 kinetic fit inherent to the software, utilising a local Rmax and local drift setting.

### Assay 5: Nicotinic Acetylcholine Receptor Antagonism Assay

Cells expressing human alpha1 nicotinic acetylcholine (nACh) receptor (nAChR) are washed and dissociated using Trypsin containing buffer. Cells are then washed and seeded at a density of 3-5 million cells/mL and allowed to grow for 3 days. Then, 60-80 μL of cells are added to plates and incubated with dose response of an ARM compound of Formula (I) with a top concentration of either 10 μM or 100 μM. The receptor is then stimulated via addition of acetylcholine (ACh) and activation of the receptor is monitored via the IONFlux automated patch clamp system. IonFlux Data Analyzer software is utilized to perform leak subtraction and measure (min) peak current amplitude. These data are then exported to Excel.

Data processing: (1) Current values recorded for vehicle only applications in individual traps/patterns are subtracted from corresponding current amplitude values for control, test article and control+test article. (2) Control response is calculated as % of the average response of the 3rd out of total 3 ACh (10 mM) applications alone. If the 3rd ACh response is obscured due to technical artifacts, the 2nd and/or 1st ACh (10 mM) application is considered as control. The peak current amplitude generated in the presence of each test article concentration is then used to calculate the % inhibition as compared to control response (10 mM ACh alone). These estimates are further corrected for vehicle response and run down corrected by normalizing with time matched control responses (10 mM ACh alone). The corrected percentages are then fit with a non-linear function in GraphPad Prism 6 (or later versions) to determine the IC₅₀.

Exclusion criteria: (i) any n's with poor recording quality, (ii) control current amplitude <500 pA or (iii) significant outliers, as determined by the Grubbs' test.

ARMs compounds of Formula (I) of the invention are tested for antagonism of the nAChR in the above assay in or more experimental runs. Antagonistic activity of the compounds of Formula (I) of the invention is reported as an IC₅₀ value (half maximal inhibitory concentration) and the maximum percentage antagonism observed at the highest concentration of compound of Formula (I) tested. For compounds tested in more than one experimental run, the IC₅₀ value is reported as an average.

### Assay 6: Dosing and Analysis of Tumor Bearing Animals

MC38 cells are injected subcutaneously into the flank of human CCR2 knock-in C57 mice. Tumor volume is measured using a digital Vernier caliper, and the length (1) and width (w) of the tumor is measured. Tumor volume is calculated using the following formula: Tumor Volume (mm³)=L×W2/2, Where, L=Length (mm); W=Width (mm). Anti-cotinine antibody (rabbit variable region sequence with mouse IgG2a Fc domain having a heavy chain sequence of SEQ ID NO: 13 and a light chain sequence of SEQ ID NO: 14), and ARM compound of Formula (I) are diluted in saline and administered intravenously to give the final concentrations tested. Antibody is tested at concentrations of 10 milligrams per kilogram (mpk), 5 mpk, 2.5 mpk, or 1.25 mpk; and compound of Formula (I) was tested at 0.2 mpk, 0.1 mpk, 0.05 mpk, and 0.025 mpk.

Flow cytometry of peripheral blood collected from the tumor bearing mice and blood drug concentration analysis are conducted essentially as described above. For flow cytometry analysis of tumors, tumor isolation procedures are performed in laminar flow hood following sterile techniques. All dissection tools used during the procedure are sterilized. The tools are kept in 70% ethanol between usage. Mice bearing subcutaneous tumor are humanely euthanized using carbon dioxide asphyxiation, sprayed down with 70% ethanol and then wiped with Betadine to remove loose fur. The skin of the tumor site is lifted up using forceps and cut down with a dissection scissor to expose the subcutaneous tumor. The exposed tumor is cut and removed using a small dissection scissor. The tumor sample is placed in a 50 mL falcon tube adequately filled with RPMI 1640 serum free media and analyzed by flow cytometry. Antibody staining for flow cytometry analysis is conducted in the same manner as for the peripheral blood samples. Survival analysis-Animals are humanely euthanized when tumors reached >1500 mm³ or ulcerations of the tumor were visible.

### Assay 7: Ex Vivo Depletion and T-Cell Expansion Assay

Freshly collected human blood from healthy donors is provided by the GSK blood collection unit. Lymphoprep (15 mL) is added to Accuspin tube and centrifuged at 800 g for 1 minute. Room temperature blood (20 mL) is diluted to a final volume of 35 mL by addition of room temperature phosphate buffered saline (PBS) and this is layered on top of the 15 mL of centrifuged Lymphoprep solution. Tubes are centrifuged at 800 g for 20 minutes. The top layer (down to the PBMC interface) is aspirated. The PBMC interface is decanted into 50 mL Falcon tubes. PBS is added to a final volume of 50 mL and samples are centrifuged at 250 g for 10 minutes at room temperature. Supernatant is decanted and pellets are resuspended in PBS and centrifuged at 300 g for 6 minutes at room temperature. Supernatant is decanted, and cell pellet was resuspended to a final concentration of 1 million cells/mL in PBS.

Cells are labeled with CFSE by addition of 1 μL of 0.25 mM Cell Trace CFSE dye per 1 mL of cells and incubated at 37° C. for 20 minutes. The cells are washed with 10 mL of RPMI+10% low IgG FBS. Cells are resuspended to 4 million cells/mL in RPMI+10% low IgG FBS.

Depletion of target cells is conducted in 100 μL final volume in 384-well plates. Cells are added to a final concentration of 1×10⁶ cells/mL. Anti-cotinine antibody (rabbit variable region with human IgG1 Fc domain containing a DE mutation (S239D/I332E) having heavy chain sequence of SEQ ID NO: 11 and a light chain sequence of SEQ ID NO: 12) is added to a final concentration of 2.5 μg/mL and the compound is added to a final concentration of approximately 16 nM. CD3/CD28 beads are added at a concentration of 0.25×10⁶ beads/mL. Samples are incubated at 37° C. overnight (about 16 hours).

Following the depletion step, samples are subjected to staining for flow cytometry. Samples are transferred to a 96-well round bottom polypropylene plate. Then, 100 μL of diluted (1:1000) Live/Dead dye is added to each sample. The plate is incubated in the dark for 15 minutes at room temperature. Then, 100 μL of HANK's buffer+1% BSA is added. The plate is centrifuged at 12000 rpm for 3 minutes and the supernatant is discarded. Fc Blocker (BD) was added with Hanks Buffer+1% BSA and antibody solution. Panel 1: (PerCP-Cy5.5-CD4-cat #560650, CD14-PC7-cat #A22331, CD45-BV421-cat #563879) Panel 2: (PerCP-Cy5.5-CD4-cat #560650, CD45-BV421-cat #563879, PE-CD8-cat #555635). Staining is conducted on ice for 20 minutes. Cells are washed with 200 μL of HANK's buffer+1% BSA and resuspended in 100 μL of Hank's buffer+1% BSA. Samples are analyzed on BD FACS Fortessa. Percent divided of the CD8+ T cell population was calculated by analyzing CFSE staining in the CD45+/CD8+ cell population.

### Assay 8: Alternative In Vivo Dosing Strategies in Mice

Experiments are conducted in human CCR2 knock-in mice (C57 background) and anti-cotinine antibody having a heavy chain sequence of SEQ ID NO: 13 and a light chain sequence of SEQ ID NO: 14 (rabbit variable region sequence with mouse IgG2a Fc domain).

In some instances, a dosing strategy in which the anti-cotinine antibody is administered intravenously as described in the in vivo experiment above and ARM compound of Formula (I) (Example 1) is administered subcutaneously following a 2-hour delay from antibody dosing is used. Subcutaneous administrations of the ARM compound are made into the loose skin of mice over the interscapular. BD syringe (1 mL) with 26 G needle is used for the subcutaneous administration. The needle is inserted under the skin of the interscapular area tented by the thumb and forefinger and ARM compound is injected. Various molar ratios of small molecule ARM compound to anti-cotinine antibody are tested according to this dosing strategy (1:1, 2:1, 4:1, 8:1, 8:0). Blood concentration of the small molecule ARM compound at various timepoints following dosing is determined according to the methods described above.

In other instances, anti-cotinine antibody is administered intravenously (10 mpk) and the small molecule ARM compound is administered at various concentrations (2 mpk, 10 mpk, 50 mpk) via oral gavage after a brief delay (<30 minutes following antibody administration). Oral dosing utilizes a ball tip 22G stainless steel needle and the ARM compound is administered slowly. Blood concentration of the small molecule ARM compound at various timepoints following dosing is measured and percent depletion of CCR2+ cells is determined by flow cytometry according to the methods described above.

In other instances, anti-cotinine antibody is administered intravenously and the small molecule ARM compound was administered intravenously after a delay of 30 minutes, 2 hours or 24 hours. Antibody is administered at a concentration of 10 mpk and ARM compound is administered at a concentration of 0.2 mpk. Blood concentration of the small molecule ARM compound at various timepoints following dosing is measured as described above. Flow cytometry is used in some cases to determine the effect of treatment on the levels of CD11B+/Ly6cHi cells in the peripheral.

### Assay 9: Cynomolgus Monkey Dosing Study—Analysis of ARM Compound PK and Depletion of Target Expressing Cells

Anti-cotinine antibody having a heavy chain sequence of SEQ ID NO: 9 and a light chain sequence of SEQ ID NO: 10 is supplied as a 125.86 mg/mL stock solution prepared in and diluted with in 20 mM Histidine, 180 mM Trehalose, 40 mM arginine, 8 mM Methionine, 0.05 mM EDTA, 0.02% PS80 [pH 5.89](pH range of diluted test formulations 5.77 to 5.91) and administered to monkeys at a dose volume of 2 mL/kg. The ARM compound is formulated as a solution (pH range of test formulations 7.26 to 7.43) in 2% Cavitron in 0.01 M phosphate buffered saline (PBS) [pH 7.37] and administered to monkeys at a dose volume of 2 mL/kg. Treatment groups and dosing schedule are shown below in Table 4 and are hereinafter referred to as Groups 1, 2, 3, and 4.

TABLE 4
Dosing Regimen for Cynomolgus Monkey Study.

| | | Anti-Cotinine | | | |
|---|---|---|---|---|---|
| | | Antibody | Example 1 | Animal Number | |
| Group # | Days of Dosing | (mg/kg/dose) | (mg/kg/dose) | Male | Female |
| 1 | Day 1 | 0 | 0 | 100 | 150 |
| 2 | Days 1 & 8 | 2 | 0.028 | 200 | 250 |
| 3 | Day 1 | 10 | 0.14 | 300 | 350 |
| 4 | Day 1 | 50 | 0.7 | 400 | 450 |

On Day 1, all monkeys in Groups 2 through 4 are dosed with small molecule ARM compound by slow IV bolus (approximately 2 minutes via tail vein and/or saphenous vein using a temporary IV catheter) followed by dosing with anti-cotinine antibody approximately 5 minutes after. Group 1 (Control) monkeys receive the respective vehicles for anti-cotinine antibody and ARM compound according to the same dosing regimen. The catheters are flushed with ˜2 mL of sterile saline following administration of each formulation. On Day 8, only monkeys in Group 2 are dosed using the same regimen as Day 1 to determine if adding a second dose resulted in extended PD response.

Monkey plasma samples are analyzed for anti-cotinine antibody using an analytical method based on immunocapture/protein digestion followed by LC-MS/MS analysis.

Cynomolgus monkey blood/water (50/50, v/v) samples are analyzed for total amount of compound using an analytical method based on protein precipitation followed by UHPLC/MS/MS analysis.

Peripheral blood samples are collected in potassium EDTA anti-coagulant prior to dosing on Day 1, and at 2 hours, 24 hours and 48 hours post-dose on Day 1, and Days 8, 15, and 22 from male and female monkeys in Groups 1, 3, and 4. Peripheral blood samples are collected in potassium EDTA anti-coagulant prior to dosing on Days 1 and 8, and at 2 hours, 24 hours, and 48 hours post-dose on Days 1 and 8, and Days 15 and 22 from male and female monkeys in Group 2. An additional sample is collected for immunophenotyping only from all monkeys prior to randomization at Screen (Day −6).

Whole blood is transferred to appropriate tubes for immunophenotyping analysis. Plasma is separated from remaining whole blood by centrifugation, aliquoted, and then stored in a freezer set to maintain −70° C. until cytokine analysis. Red blood cells are lysed using Lysis Buffer (eBioscience, San Diego, CA). After a wash, non-specific cell surface binding is blocked by pre-incubating cells (minimum of 5 minutes on wet ice) with human FC block (BD Biosciences, San Jose, CA), mouse IgG (Millipore Sigma, St. Louis, MO) and purified isotype-matched controls (BD Biosciences) at a minimum 2-fold excess. Monoclonal antibodies specific for human cell surface antigens (cross-reactive with cynomolgus monkey) are added to cells as a single panel and incubated for ˜30 minutes on wet ice with protection from light:

Cells are washed and brought up in Stain Buffer (BD Biosciences), then analyzed the same day using the FACSymphony A3 flow cytometer with FACSDiva.

Lymphocytes and monocytes are gated from live, singlet cells based on side scatter light properties and CD45. Data are expressed as percent parent and/or grandparent and absolute numbers of lymphocyte and monocyte subsets, as calculated by multiplying the flow cytometric percentages by the absolute lymphocyte and monocyte counts determined by hematology measurements.

### Assay 10: Cotinine Off-Switch Study

Human CCR2 Knock-in C₅₇ mice are dosed intravenously with anti-cotinine antibody having a heavy chain sequence of SEQ ID NO: 13 and a light chain sequence of SEQ ID NO: 14 (rabbit variable region sequence with mouse IgG2a Fc domain) and ARM compound (10 mpk antibody premixed with 0.1 mpk compound) intravenously. An additional three animals receive only saline vehicle (vehicle control animals). After 48 hours, the vehicle control animals and 3 animals from the treated group are sacrificed and blood is collected by cardiac puncture for flow cytometry analysis. The remaining animals are split into two groups: Group 2 receive 50 mpk (S)-cotinine at 48 hours and again at 60 hours while Group 1 receives no additional treatment. Blood is collected at 2, 24, 48, 48.25, 50, 60, 60.25, 62, 72, 96, 120, 144, and 168 hours post dosing of the anti-cotinine antibody and ARM compound. The blood is analyzed for concentration of the ARM compound as described above. At 72, 96, 120 and 168 hours, 3 animals are sacrificed from Groups 1 and 2 and blood is collected via cardiac puncture. The blood is processed and analyzed via flow cytometry as described above for the levels of CD45+/CD3−/CD11b+/Ly6Chi cells.

### Assay 11: CD4/CD8 T Cell Depletion Study

MC38 tumors are initiated by subcutaneous injection of 1.5×10⁶ MC38 cells in the flank of human CCR2 knock-in mice. After 5 days, 200 μg of Anti-CD8 (clone 2.43) and 200 μg of Anti-CD4 (clone GK1.5) are injected intraperitoneally (the control groups received isotype control antibodies). After 2 additional days, animals are randomized and anti-cotinine antibody having a heavy chain sequence of SEQ ID NO: 13 and a light chain sequence of SEQ ID NO: 14 (rabbit variable region sequence with mouse IgG2a Fc domain) and ARM compound are dosed intravenously as a pre-mixed solution every 6 days. At the start of dosing and at 120 hours post-dosing flow cytometry is conducted on blood samples to confirm CD4/CD8 cell depletion (data not shown). Tumor volume is measured as described above.

Assay 12: Mass Cytometry (CyTOF) Analysis of PBMCs from Healthy Donor Vs. Cancer Patient

Mass cytometry (CyTOF) is used to analyze depletion of CCR2 expressing cells in PBMCs isolated from healthy donor and cancer patient to determine whether a combination of anti-cotinine antibody and ARM compound of Formula (I) selectively depletes CCR2 expressing cells.

CyTOF Antibodies: Metal tagged antibodies are either purchased directly from Fluidigm or were prepared in house using MaxPar® antibody conjugation kits (Fluidigm). For in-house conjugation reactions, purified antibodies, in BSA-free buffer are coupled to isotopically purified metals following the manufacturer's instructions (Fluidigm) (Lou X, Angew Chem Int Ed Engl 2007; 46(32):6111-4). Following conjugation, the protein concentration of the metal labeled antibodies are measured on a NanoDrop ND-1000 (Thermo Fisher) at an absorbance of 280 nm and diluted to a final concentration of 0.5 mg/mL in Candor PBS Antibody Stabilization solution (Candor Bioscience). All antibodies, antibodies with associated metal conjugated kits and/or metal-tagged secondary antibodies utilized in the CyTOF experiments, are listed in Table 5:

TABLE 5
Mass Cytometry Staining Panel.

| Metal | Ab | | | | Metal | | Catalog |
|---|---|---|---|---|---|---|---|
| Tag | Target | Clone | Vendor | Catalog # | kit | Vendor | # |
| 89Y | CD45 | HI30 | Fluidigm | 3089003B | | | |
| 111Cd | CD3 | UCHT1 | BioLegend | 300443 | 111Cd | Fluidigm | 201111A |
| 112Cd | HLA-DR | L243 | BioLegend | 307651 | 112Cd | Fluidigm | 201112A |
| 113Cd | CD4 | RPA-T4 | BioLegend | 300541 | 113Cd | Fluidigm | 201113A |
| 114Cd | CD11b | ICRF44 | BioLegend | 301337 | 114Cd | Fluidigm | 201114A |
| 116Cd | CD8a | RPA-T8 | BioLegend | 301053 | 116Cd | Fluidigm | 201116A |
| 141Pr | EpCAM | 9C4 | Fluidigm | 3141006B | | | |
| 142Nd | CD57 | HNK-1 | Fluidigm | 3142007B | | | |
| 143Nd | CD127 | A019D5 | Fluidigm | 3143012B | | | |
| 144Nd | CD15 | W6D3 | Fluidigm | 3144019B | | | |
| 145Nd | TCF1 | 7F11A10 | BioLegend | 655202 | 145Nd | Fluidigm | 201145A |
| 146Nd | CD11c | 3.9 | Fluidigm | 3146014B | | | |
| 147Nd | CD182 | CXCR2 | Fluidigm | 3147010B | | | |
| 148Nd | PD-L1 | 29E.2A3 | Fluidigm | 3148017B | | | |
| 149Sm | CCR1 | 53504.0 | R&D | MAB145- | 149Sm | Fluidigm | 201149A |
| | | | Systems | 100 | | | |
| 150Nd | Biotin | 1D4C5 | Fluidigm | 3150008B | | | |
| | IL-10 | JES3- | BioLegend | 501502 | | | |
| | (biotin) | 12G8 | | | | | |
| 151Eu | CD14 | M5E2 | Fluidigm | 3151009B | | | |
| 152Sm | FoxP3 | PCH101 | eBioscience | 14-4776- | 152Sm | Fluidigm | 201152A |
| | | | | 82 | | | |
| 153Eu | CD192 | KO36C2 | Fluidigm | 3153023B | | | |
| | (CCR2) | | | | | | |
| 154Sm | TIM-3 | F38-2E2 | Fluidigm | 3154010B | | | |
| 155Gd | CD56 | B159 | Fluidigm | 3155008B | | | |
| 156Gd | IFNg | 4S.B4 | eBioscience | 14-7319- | 1526Gd | Fluidigm | 201156A |
| | | | | 81 | | | |
| 159Tb | CCR7 | G043H7 | Fluidigm | 3159003A | | | |
| 160Gd | FITC | FIT22 | Fluidigm | 3160011B | | | |
| | IL-35 | FIT22 | LS Bio | LS- | | | |
| | (FITC) | | | C693362- | | | |
| | | | | 500 | | | |
| 161Gd | Tbet | 4B10 | Fluidigm | 3161014B | | | |
| 162Dy | CD27 | L128 | Fluidigm | 3162009B | | | |
| 163Dy | CCR8 | L263G8 | BioLegend | 360602 | 163Dy | Fluidigm | 201163A |
| 164Dy | ARG-1 | 14D2C43 | Fluidigm | 3164030B | | | |
| 165Ho | CD19 | HIB 19 | Fluidigm | 3165025B | | | |
| 166Er | CD314 | ON72 | Fluidigm | 3166016B | | | |
| 167Er | CD38 | HIT2 | Fluidigm | 3167001B | | | |
| 168Er | CD206 | 15-2 | Fluidigm | 3168008B | | | |
| 169Tm | CD25 | 2A3 | Fluidigm | 3169003B | | | |
| 170Er | CD45RA | HI100 | Fluidigm | 3170010B | | | |
| 171Yb | CD195 | NP-6G4 | Fluidigm | 3171017A | | | |
| | (CCR5) | | | | | | |
| 172Yb | CX3CR1 | 2A9-1 | Fluidigm | 3172017B | | | |
| 173Yb | Granzyme | GB11 | Fluidigm | 3173006B | | | |
| | B | | | | | | |
| 174Yb | CD49d | 9F10 | Fluidigm | 3174018B | | | |
| 175Lu | PD-1 | EH12.2H7 | Fluidigm | 3175008B | | | |
| 176Yb | ICOS | C398.4A | BioLegend | 313502l | 176Yb | Fluidigm | 201176A |
| 209Bi | CD16 | 3G8 | Fluidigm | 3209002B | | | |

### Mass Cytometry (CyTOF)

A compound treatment plate for PBMC incubation is prepared using an HIP Dispenser (HP). First, a 10 M stock of ARM compound is obtained. Then, using the HIP Dispenser, 200 nL of the compound is added to the wells of a 96-well plate. An equal volume of DMSO is added to control wells. Second, anti-cotinine antibody (heavy chain of SEQ TD NO: 11 and light chain of SEQ TD NO: 12) is diluted in AIM-V media (Gibco) and 2 μL of antibody is added to treatment wells or 2 μL of AIM-V media is added to control wells. Once the treatment plate is prepared, then PBMC samples are thawed.

Cryopreserved PBMC samples, from either a healthy donor (Hemacare) or a cancer patient (Discovery Life Sciences) are thawed in a 37° C. water bath. PBMC samples are then added to 8 mL warm AIM-V media (Gibco), pelleted by centrifugation and resuspended in 1 mL AIM-V media. Cells are counted on the ViCell (Beckman Coulter). Equal numbers of PBMCs are transferred to each treatment well in the previously prepared 96-well plate and incubated at 37° C. for 24 hours.

Following a 24 hour incubation period, Protein Transport Inhibitor Cocktail (500X) (Invitrogen/eBioscience) is added to all wells and incubated at 37° C. for 2 hours. Samples are then centrifuged and washed twice with MaxPar phosphate-buffered saline (PBS) (Fluidigm). Cells are then incubated with a viability reagent (200 nM cisplatin-198, Fluidigm) diluted in MaxPar PBS for 5 min at room temperature, then washed twice in MaxPar Cell Staining Buffer (Fluidigm). The resultant cell pellets are resuspended in MaxPar Cell Staining Buffer. Samples are then blocked for 10 minutes at room temperature in Human TruStain FcX (BioLegend). Immediately following, cells are stained with all cell surface markers, included in Table 9, for 20 minutes at room temperature. Following cell surface staining, samples are washed twice with MaxPar Cell Staining Buffer. Cells are then permeabilized using eBioscience FoxP3 fix/perm buffer (Thermo Fisher) for 45 min at 4° C. Samples are pelleted by centrifugation and washed twice in the fix/perm buffer from the eBioscience FoxP3 fix/perm kit (Thermo Fisher). Samples are resuspended in the same FoxP3 fix/perm buffer. All intracellular antibodies, listed in Table 9, are then added and incubated at room temperature for 30 minutes. Following intracellular staining, cells are then pelleted by centrifugation and washed twice in the fix/perm buffer from the eBioscience FoxP3 fix/perm kit (Thermo Fisher). Cells are again resuspended in the FoxP3 fix/perm buffer and then incubated with secondary antibodies, anti-biotin and anti-FITC, for 25 minutes at room temperature. Samples are pelleted by centrifugation and washed twice with MaxPar Cell Staining Buffer. Cells are then resuspended in MaxPar Barcode Perm Buffer (Fluidigm) and then washed twice with the MaxPar Barcode Perm Buffer. To enable cell identification during CyTOF2 acquisition, each sample is then resuspended in Cell ID DNA Intercalator Ir (Fluidigm) diluted in MaxPar Fix/Perm Buffer (Fluidigm). Finally, to enable sample pooling, each sample is given a unique palladium barcode from the Cell-ID 20-Plex Pd Barcoding Kit (Fluidigm). Samples are incubated with the DNA Intercalator and palladium barcode at 4° C. minimally overnight or until time of CyTOF2 acquisition.

On the day of CyTOF2 acquisition, all samples are washed twice in Cell Staining Buffer. Following the second wash, and resuspension in residual amount of Cell Staining Buffer, all samples are pooled into a single sample and washed twice with 2 mL MilliQ Water (18.2 mΩ). The final sample is then resuspended in 1 mL MilliQ Water (18.2 mΩ) and strained through a 40 μm porosity Flowmi Strainer (Bel-Art). The pooled sample is then counted on a ViCell and diluted to 400,000 cells/mL in MilliQ Water (18.2 mΩ). Finally, a 1:10 ratio of EQ Beads (Fluidigm) is added to the sample and was able to proceed with CyTOF2 (Fluidigm) acquisition using the Supersampler (Victorian Airship & Scientific Apparatus). All samples are acquired with CyTOF software.

CyTOF data analysis: Raw FCS files are normalized, concatenated and debarcoded with standalone software CyTOF. Debarcoded FCS files areuploaded onto GSK Cytobank Enterprise (cytobank.org). EQ reference bead signal is removed from all samples. Viable cells are then identified using a combination Cisplatin Pt198 and Ir131 DNA Intercalator gating, with viable cells being defined as Cisplatin Pt198 low and Ir131 high. Viable cells are then further gated into viable singles, with a combination of the event length channel and CD45-89Y staining. Viable singlets are defined as events lower than 60 with a CD45 metal intensity greater than 30. Unsupervised clustering of the viable singlets is then completed with the viSNE application embedded within Cytobank.

### V. Combination Therapy

Another aspect of the invention provides for combination therapy. Heterobifunctional compounds described herein (such as a compound of Formula I, or other compounds in Section I) or their pharmaceutically acceptable salts may be used in combination with additional therapeutic agents to treat medical disorders, such as cancer.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method further comprises administering an anti-cotinine antibody, or antigen-binding fragment thereof. In some embodiments, the method includes co-administering one additional therapeutic agent. In some embodiments, the method includes co-administering two additional therapeutic agents. In some embodiments, the method includes co-administering three additional therapeutic agents. In some embodiments, the method includes co-administering four additional therapeutic agents. In some embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen more than 24 hours apart.

### Anti-Cancer Agents

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

Radiation therapy may also be used as part of a combination therapy.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. The CTLA4 inhibitor ipilumumab has been approved by the United States Food and Drug Administration for treating melanoma. In certain embodiments, the immune checkpoint inhibitor comprises pembrolizumab.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Accordingly, another aspect of the invention provides a method of treating cancer in a patient, where the method comprises administering to the patient in need thereof (i) a therapeutically effective amount of a compound described herein and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor;
a cytokine selected from IL-12, IL-15, GM-CSF, and G-CSF;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

### Additional Considerations

The doses and dosage regimen of the active ingredients used in the combination therapy may be determined by an attending clinician. In certain embodiments, the compound described herein (such as a compound of Formula I, or other compounds in Section I) and the additional therapeutic agent(s) are administered in doses commonly employed when such agents are used as monotherapy for treating the disorder. In other embodiments, the compound described herein (such as a compound of Formula I, or other compounds in Section I) and the additional therapeutic agent(s) are administered in doses lower than the doses commonly employed when such agents are used as monotherapy for treating the disorder. In certain embodiments, the compound described herein (such as a compound of Formula I, or other compounds in Section I) and the additional therapeutic agent(s) are present in the same composition, which is suitable for oral administration.

In certain embodiments, the compound described herein (such as a compound of Formula I, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the compound described herein (such as a compound of Formula I, or other compounds in Section I), a pharmaceutically acceptable carrier, vehicle or diluent, and optionally at least one additional therapeutic agent listed above. In some embodiments, the kit further comprises an anti-cotinine antibody, or antigen-binding fragment thereof.

### VI. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (such as a compound of Formula I, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

VII. SEQUENCE LISTINGS

Heavy chain CDR1 amino acid sequence (SEQ ID NO: 1):
NYWMS
Heavy chain CDR2 amino acid sequence (SEQ ID NO: 2):
DIHGNRGFNYHASWAKG
Heavy chain CDR3 amino acid sequence (SEQ ID NO: 3):
ADDSGSHDI
Light chain CDR1 amino acid sequence (SEQ ID NO: 4):
QSSQSVYSAKLS
Light chain CDR2 amino acid sequence (SEQ ID NO: 5):
YGSTLAS
Light chain CDR3 amino acid sequence (SEQ ID NO: 6):
QGTFYGPDWYFA
Variable heavy chain amino acid sequence (SEQ ID NO: 7):
EVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIW
GQGTLVTVSS
Variable light chain amino acid sequence (SEQ ID NO: 8):
DIQMTQSPSSLSASVGDRVTITCQSSQSVYSAKLSWYQQKPGKAPKLLIYYGSTLASGVP
SRFSGSGSGTQFTLTISSLQPEDFATYYCQGTFYGPDWYFAFGGGTKVEIK
Heavy chain amino acid sequence (SEQ ID NO: 9):
EVQLVESGGGLVQPGGSLRLSCAASGFTFSNYWMSWVRQAPGKGLEWVGDIHGNRGF
NYHASWAKGRFTVSRSKNTLYLQMNSLRAEDTAVYYCAKADDSGSHDIWGQGTLVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPELL
GGPDVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPEEKTISKAKGQPREPQVYTL
PPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK
Light chain amino acid sequence (SEQ ID NO: 10):
DIQMTQSPSSLSASVGDRVTITCQSSQSVYSAKLSWYQQKPGKAPKLLIYYGST
LASGVPSRFSGSGSGTQFTLTISSLQPEDFATYYCQGTFYGPDWYFAFGGGTKV
EIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
Heavy chain amino acid sequence (SEQ ID NO: 11):
QQQLVESGGR
LVTPGGSLTL TCTASGFSLN NYWMSWVRQA PGKGLEWIGD IHGNRGFNYH
ASWAKGRFTV SRTSTTVDLR MTSLTTEDTA IYFCARADDS GSHDIWGPGT
LVTVSSASTK GPSVFPLAPS SKSTSGGTAA LGCLVKDYFP EPVTVSWNSG
ALTSGVHTFP AVLQSSGLYS LSSVVTVPSS SLGTQTYICN VNHKPSNTKV
DKKVEPKSCD KTHTCPPCPA PELLGGPDVF LFPPKPKDTL MISRTPEVTC
VVVDVSHEDP EVKFNWYVDG VEVHNAKTKP REEQYNSTYR VVSVLTVLHQ
DWLNGKEYKC KVSNKALPAP EEKTISKAKG QPREPQVYTL PPSRDELTKN
QVSLTCLVKG FYPSDIAVEW ESNGQPENNY KTTPPVLDSD GSFFLYSKLT
VDKSRWQQGN VFSCSVMHEA LHNHYTQKSL SLSPGK
Light chain amino acid sequence (SEQ ID NO: 12):
ELDLTQTPSPVSAAVGDTVTINCQSSQSVYSAKLSWYQQKPGQPPKLLIYYGSTLASGV
PSRFKGSGSGTQFSLTISDVQCADAATYYCQGTYYGPDWYFAFGGGTEVVVKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Heavy chain amino acid sequence (SEQ ID NO: 13):
QQQLVESGGRLVTPGGSLTLTCTASGFSLNNYWMSWVRQAPGKGLEWIGDIHGNRGF
NYHASWAKGRFTVSRTSTTVDLRMTSLTTEDTAIYFCARADDSGSHDIWGPGTLVTVS
SAKTTAPSVYPLAPVCGDTTGSSVTLGCLVKGYFPEPVTLTWNSGSLSSGVHTFPAVLQS
DLYTLSSSVTVTSSTWPSQSITCNVAHPASSTKVDKKIEPRGPTIKPCPPCKCPAPNLLGG
PSVFIFPPKIKDVLMISLSPMVTCVVVDVSEDDPDVQISWFVNNVEVLTAQTQTHREDYN
STLRVVSALPIQHQDWMSGKEFKCKVNNKALPAPIERTISKPKGSVRAPQVYVLPPPEEE
MTKKQVTLTCMVTDFMPEDIYVEWTNNGKTELNYKNTEPVLDSDGSYFMYSKLRVEK
KNWVERNSYSCSVVHEGLHNHHTTKSFSRTPGK
Light chain amino acid sequence (SEQ ID NO: 14):
ELDLTQTPSPVSAAVGDTVTINCQSSQSVYSAKLSWYQQKPGQPPKLLIYYGSTLASGV
PSRFKGSGSGTQFSLTISDVQCADAATYYCQGTYYGPDWYFAFGGGTEVVVKRADAA
PTVSIFPPSSEQLTSGGASVVCFLNNFYPKDINVKWKIDGSERQNGVLNSWTDQDSKDST
YSMSSTLTLTKDEYERHNSYTCEATHKTSTSPIVKSFNRNEC

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula (I): 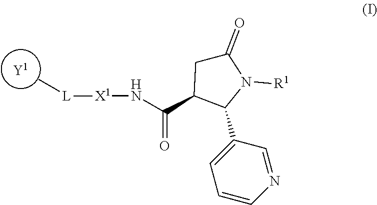
or a pharmaceutically acceptable salt thereof, wherein:
X¹ is C₁₋₅ alkylene or a covalent bond;
Y¹ is one of the following: 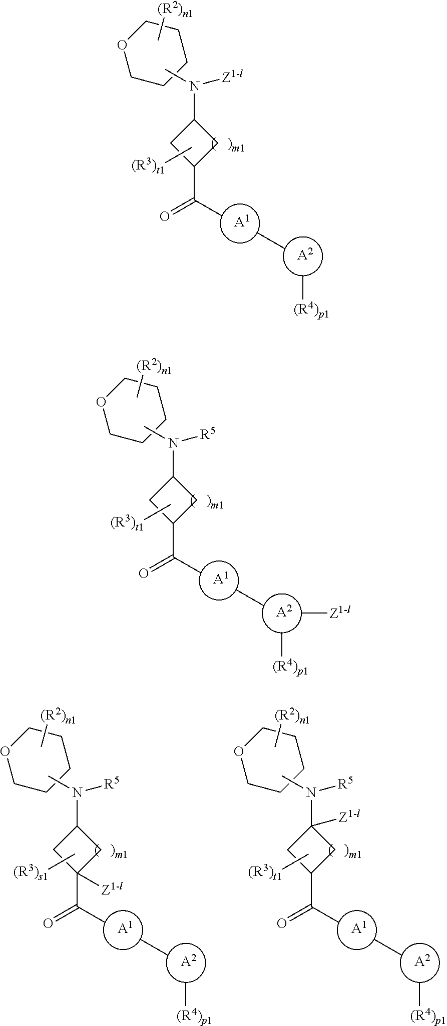 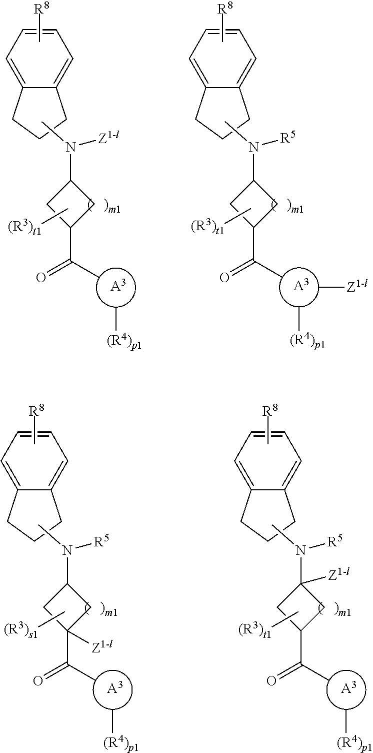
Z^{1-I} is —(C₀₋₆ alkylene)-N(R⁶)—R⁷, —(C₁₋₆ alkylene)-(4-6 membered saturated heterocyclylene containing 1 or 2 heteroatoms selected from nitrogen)-R⁷, or —(C₀₋₆ alkylene)-O—R⁷;
R¹ is C₁₋₄ alkyl or C₃₋₆ cycloalkyl;
R² represents independently for each occurrence C₁₋₄ alkoxyl or C₁₋₄ alkyl;
R³ and R⁹ each represent independently for each occurrence C₁₋₄ alkyl;
R⁵ and R⁶ are independently hydrogen or C₁₋₄ alkyl;
R⁴ and R⁸ each represent independently for each occurrence C₁₋₄ haloalkyl, halo, or C₁₋₄ alkyl;
R⁷ is a bond to L;
A¹ is (i) a 5-7 membered saturated heterocyclylene containing 1 or 2 heteroatoms independently selected from nitrogen and oxygen or (ii) a 6-8 membered bridged bicyclic saturated heterocyclylene containing 1 or 2 heteroatoms independently selected from nitrogen and oxygen; wherein the heterocyclylene is substituted with w1 occurrences of R⁹;
A² is a 5-6 membered heteroarylene containing 1 or 2 heteroatoms independently selected from nitrogen, oxygen, or sulfur;
A³ is an 8-10 membered bicyclic heteroarylene containing 1, 2, or 3 heteroatoms selected from nitrogen;
n1, p1, and t1 are independently 1, 2, or 3;
m1 is 1 or 2;
w1 and s1 are independently 0, 1, or 2; and
L is a divalent linker selected from:
(i) a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, optionally substituted 3-10 membered heterocyclyl containing 1, 2, 3, or 4 heteroatoms independently selected from nitrogen, oxygen, and sulfur, or optionally substituted 5-6 membered heteroaryl containing 1, 2, or 3 heteroatoms independently selected from nitrogen, oxygen, and sulfur;
(ii) 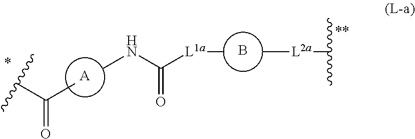
wherein Ring A and Ring B are each independently C₄₋₆ cycloalkylene; L^{1a} is C₃₋₅ linear alkylene, wherein 1 or 2 methylene units are replaced with —O— or —NR^{a}—; each R^{a} is independently hydrogen or C₁₋₃ alkyl; and
L^{2a} is —O—, —NHC(O)—, or —CH₂—O—;
(iii) 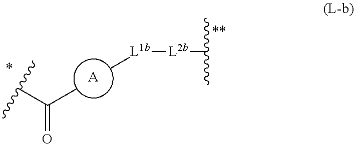
wherein Ring A is C₄₋₆ cycloalkylene or C₇₋₉ bridged bicyclic cycloalkylene; L^{1b} is —CH₂—NH—C(O)—, —NHC(O)—, or —C(O)NH—; L^{2b} is C₆₋₁₂ linear alkylene, wherein 1, 2, 3, or 4 methylene units are replaced with —O—, —NR^{1b}—, —C(O)NR^{b}—, or —NR^{1b}C(O)—; or L^{2b} is 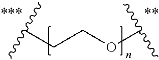
wherein n is 1, 2, 3, or 4, and 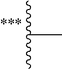
represents a covalent bond to L^{1b}; and each R^{1b} is independently hydrogen or C₁₋₃ alkyl;
(iv) 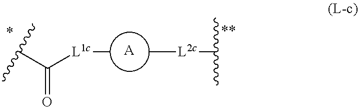
wherein L^{1c} is C₂₋₁₀ linear alkylene, wherein 1, 2, or 3 methylene units are replaced with —O—, —NH—, —NHC(O)—, or —C(O)NH—; Ring A is C₄₋₆ cycloalkylene or C₇₋₉ bridged bicyclic cycloalkylene; and L^{2c} is —O— or a saturated C₂₋₁₀ linear alkylene, wherein 1, 2, or 3 methylene units are replaced with —O—, —NH—, —NHC(O)—, or —C(O)NH—;
(v) 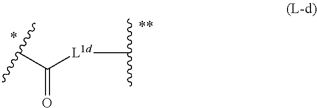
wherein L^{1d} is C₁₂₋₂₂ linear alkylene, wherein 1, 2, 3, 4, or 5 methylene units are replaced with —NH—, —O—, —C(O)NH—, —NHC(O)—, or —NHC(O)—NH—;
(vi) 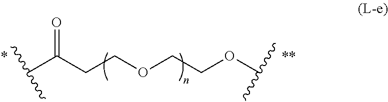
wherein n is an integer of 3 to 50;
(vii) 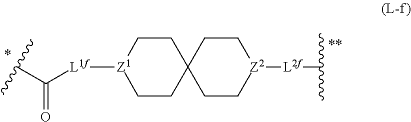
wherein L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—; L^{2f} is a bond, —NHC(O)—, —C(O)NH—, or a C₁₋₆ linear alkylene, wherein 0, 1, or 2 methylene units are replaced with —O—; and each of Z¹ and Z² is independently N or CH;
(viii) 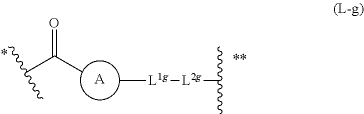
wherein Ring A is a 5 to 6 membered heteroarylene having 1 or 2 nitrogen ring atoms; L^{1g} is a bond, —CH₂—, —NH—, or —O—; and L^{2g} is 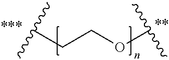
wherein n is 1, 2, 3, 4, or 5, and 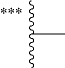
represents a covalent bond to L^{1g};
(ix) 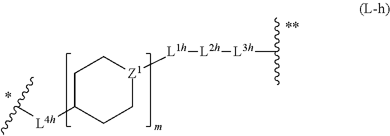
wherein each Z¹ is independently N or CH; L^{1h} is a bond, —C(O)—, —C(O)—NH—, or —NHC(O)-; L^{2h} is C₂₋₁₀ linear alkylene or 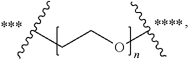
wherein n is 1, 2, 3, or 4, and 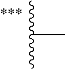
represents a covalent bond to L^{1h} and 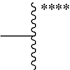
represents a covalent bond to L^{3h}; L^{3h} is a bond, —C(O)CH₂—, —O—(C₃₋₆ cycloalkylene)-O—, or —C(O)NH(CH₂)₃OCH₂—; L^{4h} is a bond, —C(O)—, —CH₂C(O)—, or —C(O)CH₂—; and m is 1, 2, or 3;
(x) 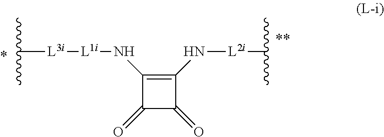
wherein L¹ⁱ is a bond, C₁₋₁₂ linear alkylene, or 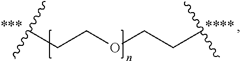
wherein n is 1, 2, 3, 4, or 5, and 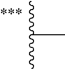
represents a covalent bond to L³ⁱ and 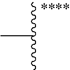
represents a covalent bond to NH; L²ⁱ is a bond, C₁₋₁₂ linear alkylene, or 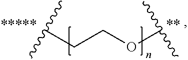
wherein n is 1, 2, 3, 4, or 5, and 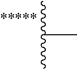
represents a covalent bond to HN; and L³ⁱ is a bond or —C(O)—;
(xi) 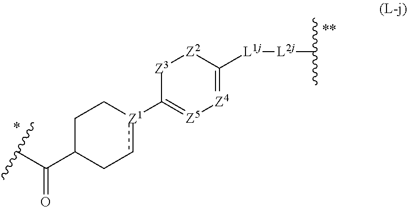
wherein Z¹ is C, CH, or N; each of Z², Z³, Z⁴ and Z⁵ is independently CH or N, provided that no more than two of Z², Z³, Z⁴ and Z⁵ are N; L^{1j} is —NH—, —C(O)NH—, —NHC(O)—, or —O—; L^{2j} is C₁₋₆ linear alkylene or 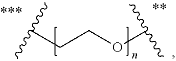
wherein n is 1 or 2, and 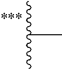
represents a covalent bond to L^{1j}; and [symbol] represents a single bond or a double bond;
(xii) 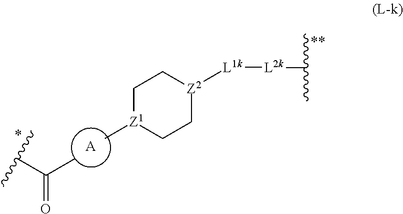
wherein Ring A is phenyl or a 5 or 6 membered heteroarylene having 1 or 2 nitrogen ring atoms; each of Z¹ and Z² is independently CH or N; L^{1k} is a bond, —C(O)—, —C(O)NH— or —NHC(O)—; and L^{2k} is a C₃₋₈ straight chain alkylene or 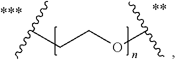
wherein n is 1, 2, or 3, and 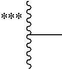
represents a covalent bond to L^{k};
(xiii) 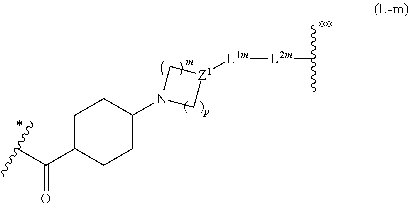
wherein Z¹ is CH or N; m is 1 or 2; p is 1 or 2; 0, 1, or 2 hydrogen atoms of 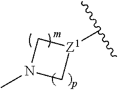
are replaced with F; L^{1m} is a bond, —C(O)—, —C(O)NH—, —NHC(O)—, —S(O)₂NH— or —NHS(O)₂—; and L^{2m} is C₃₋₆ linear alkylene, C₃₋₆ cycloalkylene, or 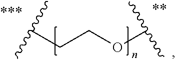
wherein n is 1 or 2, and 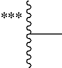
represents a covalent bond to L^{1m};
(xiv) one of 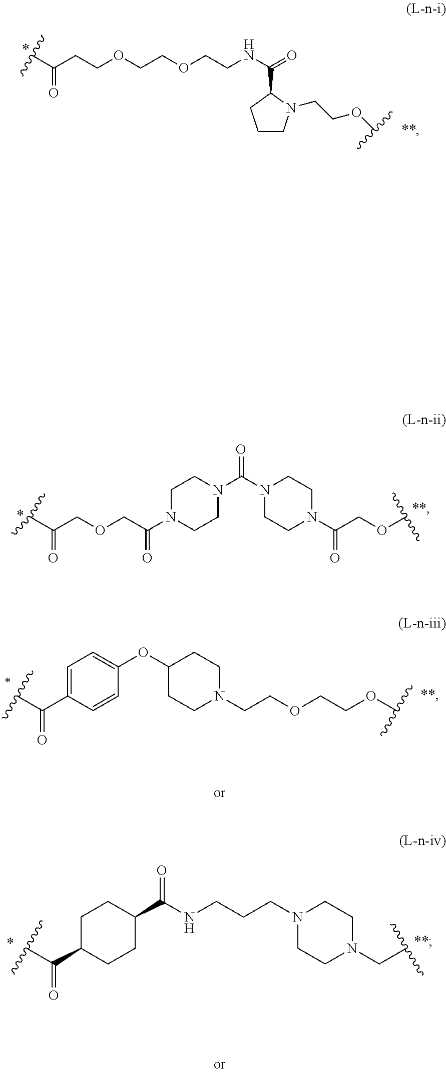
(xv) 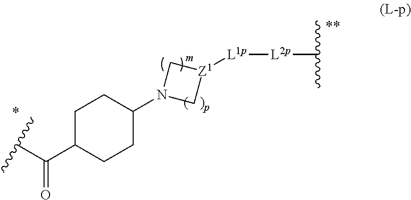
wherein Z¹ is CH or N; m is 1 or 2; p is 1 or 2; 0, 1, or 2 hydrogen atoms of 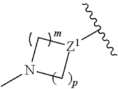
are replaced with F; L^{1P} is a bond, —C(O)—, —C(O)NH—, —NHC(O)—, —S(O)₂—, —S(O)₂NH—, or —NHS(O)₂—; and L^{2p} is -(4-6 membered saturated heterocyclylene containing 1 or 2 heteroatoms independently selected from nitrogen, oxygen, and sulfur)-C(O))—;
wherein each 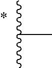
represents a covalent bond to Y¹, and each 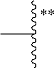
represents a covalent bond to X¹.

2. The compound of claim 1, wherein the compound is a compound of Formula I.

3. The compound of claim 1, wherein R¹ is —CH₃.

4. The compound of claim 1, wherein X¹ is C₁₋₅ alkylene.

5-6. (canceled)

7. The compound of claim 1, wherein Y¹ is

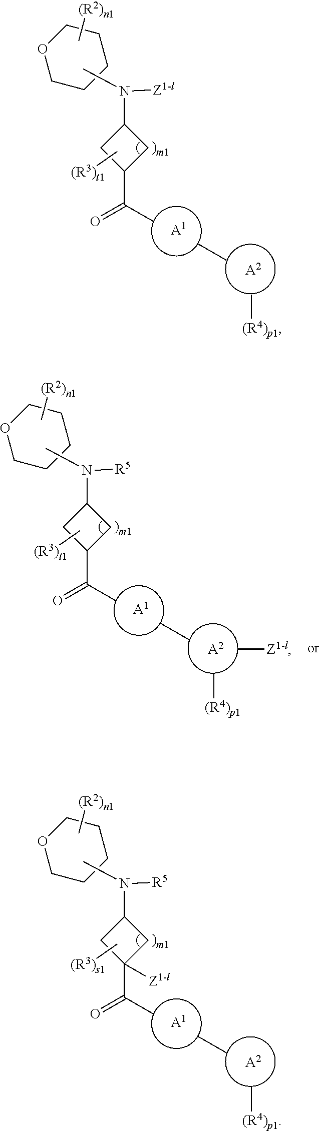

8. The compound of claim 1, wherein Y¹ is represented by

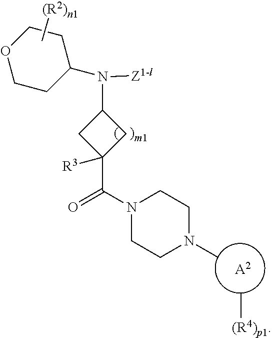

9-10. (canceled)

11. The compound of claim 1, wherein Y¹ is represented by

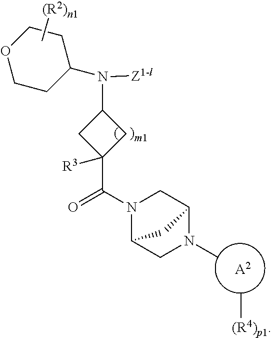

12-25. (canceled)

26. The compound of claim 1, wherein Y¹ is one of the following:

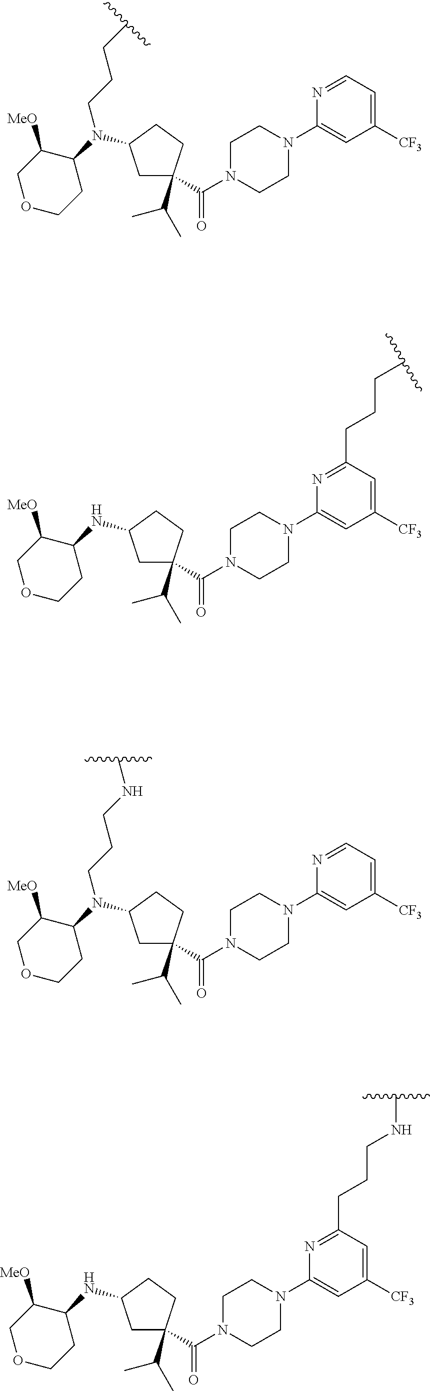 [SMILES: *CCCN([C@@H]1CC[C@@](C(=O)N2CCN(c3cc(C(F)(F)F)ccn3)CC2)(C(C)C)C1)[C@H]1CCOC[C@H]1OC ; *CCCc1cc(C(F)(F)F)cc(N2CCN(C(=O)[C@@]3(C(C)C)CC[C@@H](N[C@H]4CCOC[C@H]4OC)C3)CC2)n1 ; *NCCCN([C@@H]1CC[C@@](C(=O)N2CCN(c3cc(C(F)(F)F)ccn3)CC2)(C(C)C)C1)[C@H]1CCOC[C@H]1OC ; *NCCCc1cc(C(F)(F)F)cc(N2CCN(C(=O)[C@@]3(C(C)C)CC[C@@H](N[C@H]4CCOC[C@H]4OC)C3)CC2)n1]

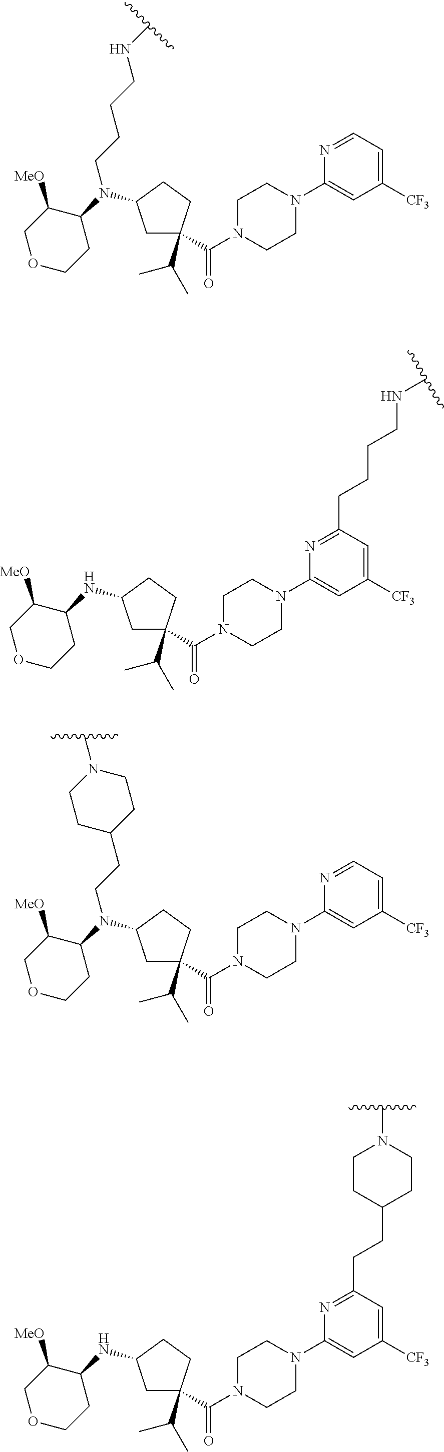 [SMILES: *NCCCCN([C@@H]1CC[C@@](C(=O)N2CCN(c3cc(C(F)(F)F)ccn3)CC2)(C(C)C)C1)[C@H]1CCOC[C@H]1OC ; *NCCCCc1cc(C(F)(F)F)cc(N2CCN(C(=O)[C@@]3(C(C)C)CC[C@@H](N[C@H]4CCOC[C@H]4OC)C3)CC2)n1 ; *N1CCC(CCN([C@@H]2CC[C@@](C(=O)N3CCN(c4cc(C(F)(F)F)ccn4)CC3)(C(C)C)C2)[C@H]2CCOC[C@H]2OC)CC1 ; *N1CCC(CCc2cc(C(F)(F)F)cc(N3CCN(C(=O)[C@@]4(C(C)C)CC[C@@H](N[C@H]5CCOC[C@H]5OC)C4)CC3)n2)CC1]

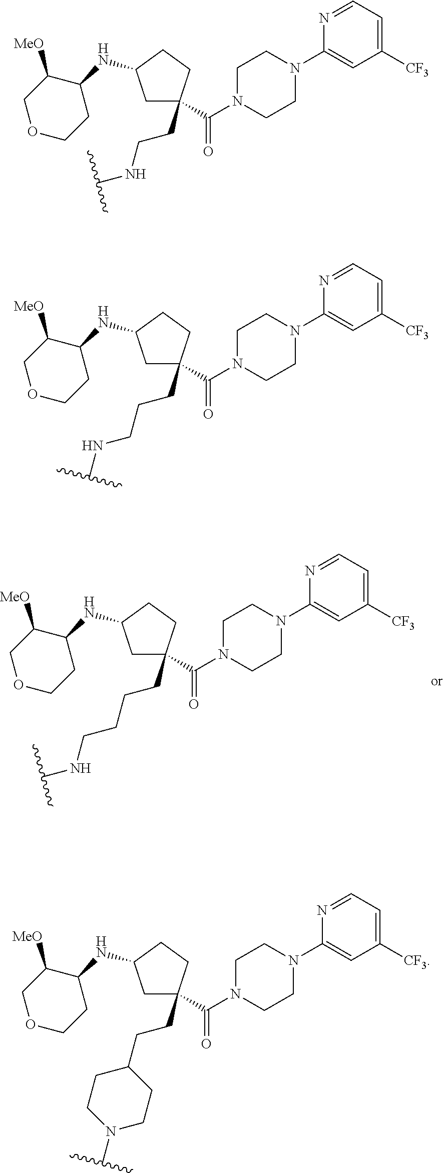 [SMILES: *NCC[C@]1(C(=O)N2CCN(c3cc(C(F)(F)F)ccn3)CC2)CC[C@@H](N[C@H]2CCOC[C@H]2OC)C1 ; *NCCC[C@]1(C(=O)N2CCN(c3cc(C(F)(F)F)ccn3)CC2)CC[C@@H](N[C@H]2CCOC[C@H]2OC)C1 ; *NCCCC[C@]1(C(=O)N2CCN(c3cc(C(F)(F)F)ccn3)CC2)CC[C@@H](N[C@H]2CCOC[C@H]2OC)C1 ; *N1CCC(CC[C@]2(C(=O)N3CCN(c4cc(C(F)(F)F)ccn4)CC3)CC[C@@H](N[C@H]3CCOC[C@H]3OC)C2)CC1]

27. The compound of claim 1, wherein Y¹ is one of the following:

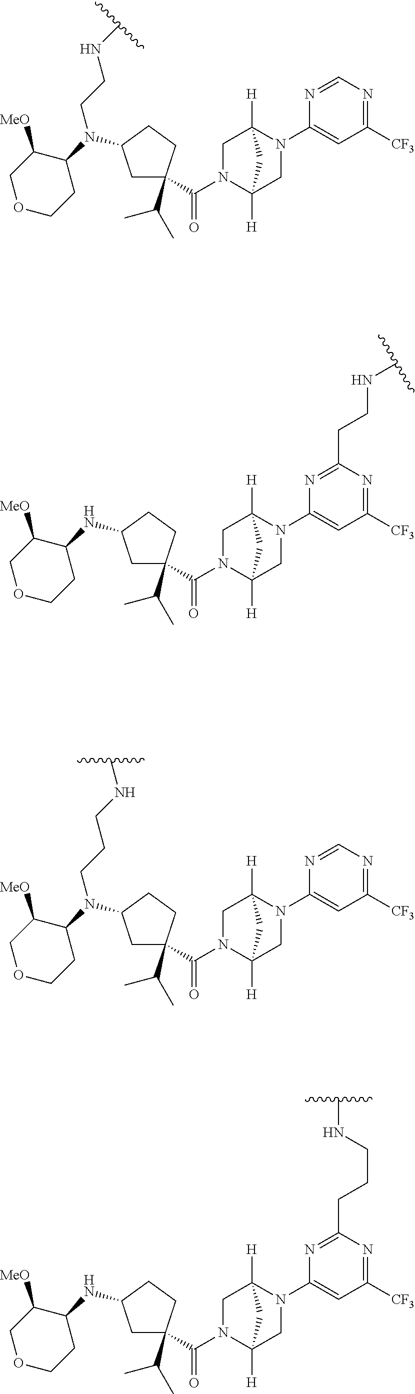 [SMILES: *NCCN([C@@H]1CC[C@@](C(=O)N2C[C@@H]3C[C@H]2CN3c2cc(C(F)(F)F)ncn2)(C(C)C)C1)[C@H]1CCOC[C@H]1OC ; *NCCc1nc(N2C[C@@H]3C[C@H]2CN3C(=O)[C@@]2(C(C)C)CC[C@@H](N[C@H]3CCOC[C@H]3OC)C2)cc(C(F)(F)F)n1 ; *NCCCN([C@@H]1CC[C@@](C(=O)N2C[C@@H]3C[C@H]2CN3c2cc(C(F)(F)F)ncn2)(C(C)C)C1)[C@H]1CCOC[C@H]1OC ; *NCCCc1nc(N2C[C@@H]3C[C@H]2CN3C(=O)[C@@]2(C(C)C)CC[C@@H](N[C@H]3CCOC[C@H]3OC)C2)cc(C(F)(F)F)n1]

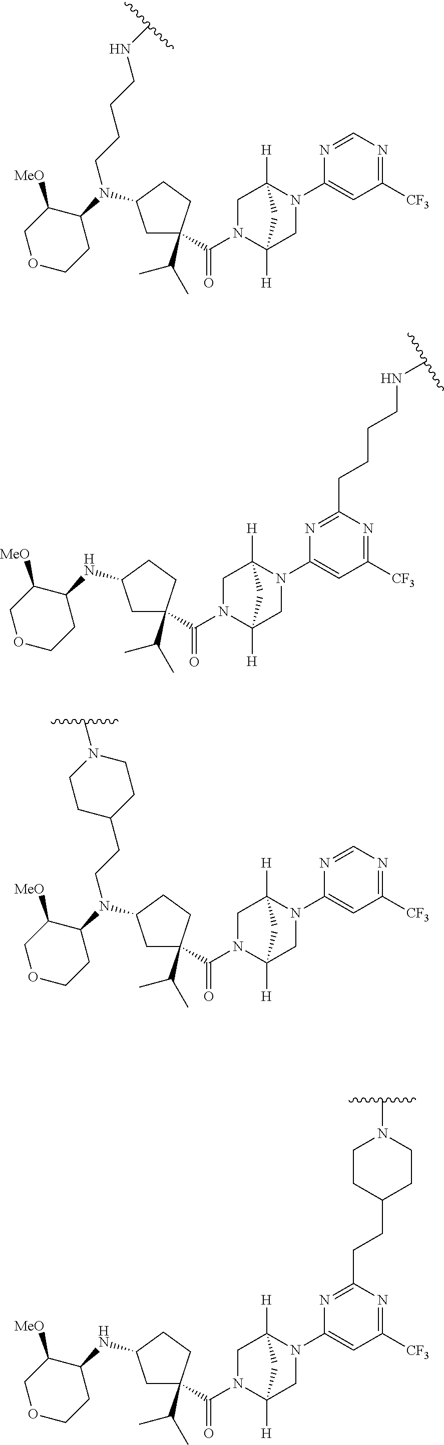 [SMILES: *NCCCCN([C@@H]1CC[C@@](C(=O)N2C[C@@H]3C[C@H]2CN3c2cc(C(F)(F)F)ncn2)(C(C)C)C1)[C@H]1CCOC[C@H]1OC ; *NCCCCc1nc(N2C[C@@H]3C[C@H]2CN3C(=O)[C@@]2(C(C)C)CC[C@@H](N[C@H]3CCOC[C@H]3OC)C2)cc(C(F)(F)F)n1 ; *N1CCC(CCN([C@@H]2CC[C@@](C(=O)N3C[C@@H]4C[C@H]3CN4c3cc(C(F)(F)F)ncn3)(C(C)C)C2)[C@H]2CCOC[C@H]2OC)CC1 ; *N1CCC(CCc2nc(N3C[C@@H]4C[C@H]3CN4C(=O)[C@@]3(C(C)C)CC[C@@H](N[C@H]4CCOC[C@H]4OC)C3)cc(C(F)(F)F)n2)CC1]

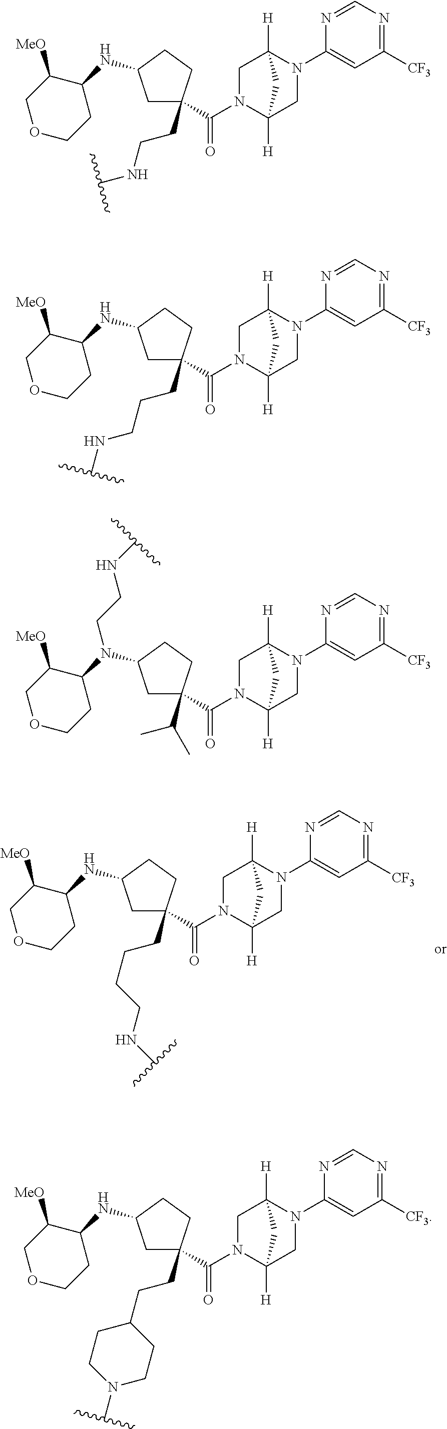 [SMILES: *NCC[C@]1(C(=O)N2C[C@@H]3C[C@H]2CN3c2cc(C(F)(F)F)ncn2)CC[C@@H](N[C@H]2CCOC[C@H]2OC)C1 ; *NCCC[C@]1(C(=O)N2C[C@@H]3C[C@H]2CN3c2cc(C(F)(F)F)ncn2)CC[C@@H](N[C@H]2CCOC[C@H]2OC)C1 ; *NCCN([C@@H]1CC[C@@](C(=O)N2C[C@@H]3C[C@H]2CN3c2cc(C(F)(F)F)ncn2)(C(C)C)C1)[C@H]1CCOC[C@H]1OC ; *NCCCC[C@]1(C(=O)N2C[C@@H]3C[C@H]2CN3c2cc(C(F)(F)F)ncn2)CC[C@@H](N[C@H]2CCOC[C@H]2OC)C1 ; *N1CCC(CC[C@]2(C(=O)N3C[C@@H]4C[C@H]3CN4c3cc(C(F)(F)F)ncn3)CC[C@@H](N[C@H]3CCOC[C@H]3OC)C2)CC1]

28. The compound of claim 1, wherein Y¹ is

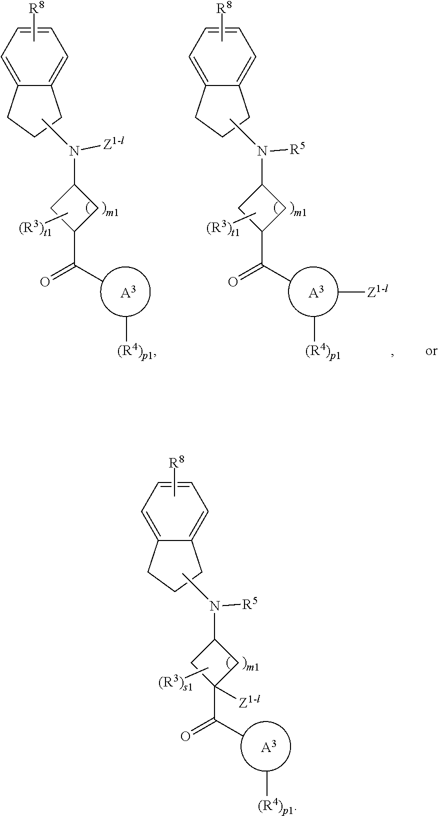

29-34. (canceled)

35. The compound of claim 1, wherein R⁸ is halo.

36. The compound of or claim 1, wherein R⁸ is bromo.

37. The compound of or claim 1, wherein A³ is a 5,6,7,8-tetrahydro-1,6-naphthyridinylene.

38-43. (canceled)

44. The compound of claim 1, wherein Y¹ is one of the following:

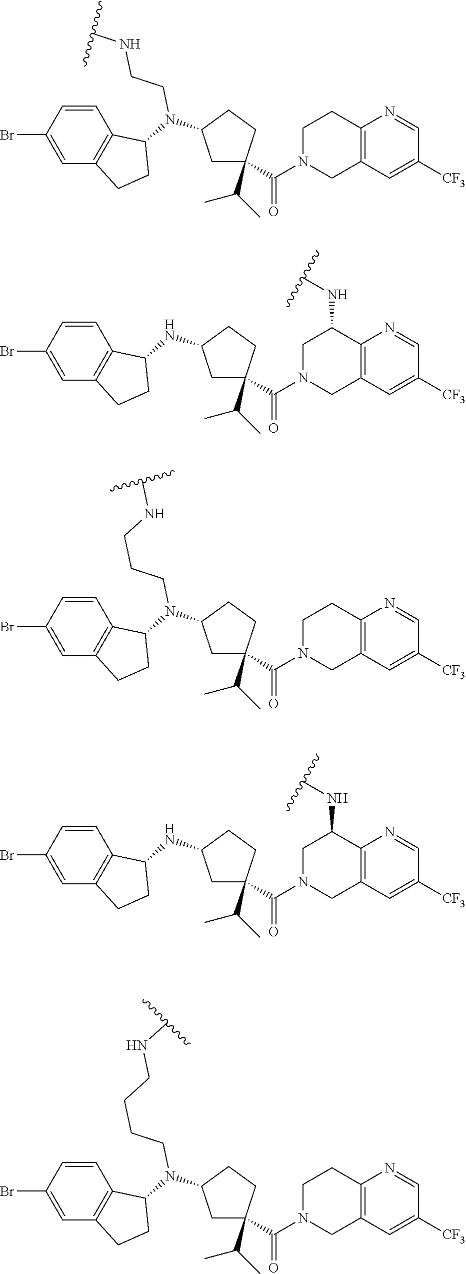 [SMILES: *NCCN([C@@H]1CC[C@@](C(=O)N2CCc3ncc(C(F)(F)F)cc3C2)(C(C)C)C1)[C@@H]1CCc2cc(Br)ccc21 ; *N[C@H]1CN(C(=O)[C@@]2(C(C)C)CC[C@@H](N[C@@H]3CCc4cc(Br)ccc43)C2)Cc2cc(C(F)(F)F)cnc21 ; *NCCCN([C@@H]1CC[C@@](C(=O)N2CCc3ncc(C(F)(F)F)cc3C2)(C(C)C)C1)[C@@H]1CCc2cc(Br)ccc21 ; *N[C@@H]1CN(C(=O)[C@@]2(C(C)C)CC[C@@H](N[C@@H]3CCc4cc(Br)ccc43)C2)Cc2cc(C(F)(F)F)cnc21 ; *NCCCCN([C@@H]1CC[C@@](C(=O)N2CCc3ncc(C(F)(F)F)cc3C2)(C(C)C)C1)[C@@H]1CCc2cc(Br)ccc21]

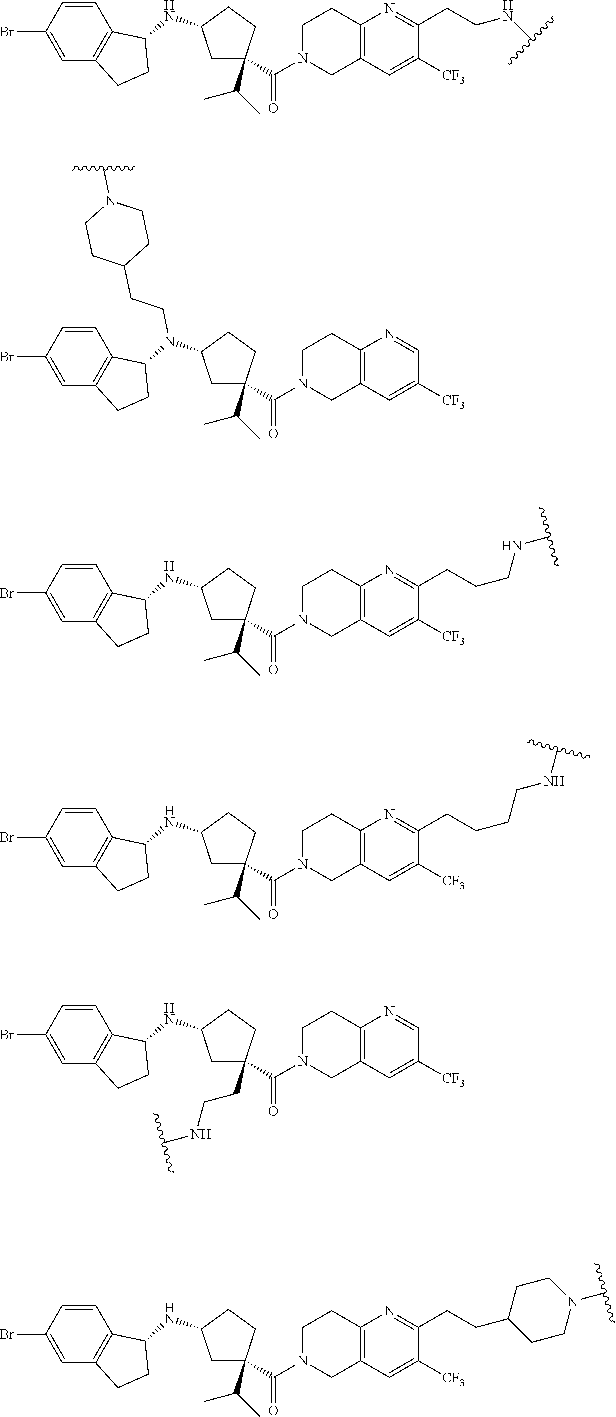

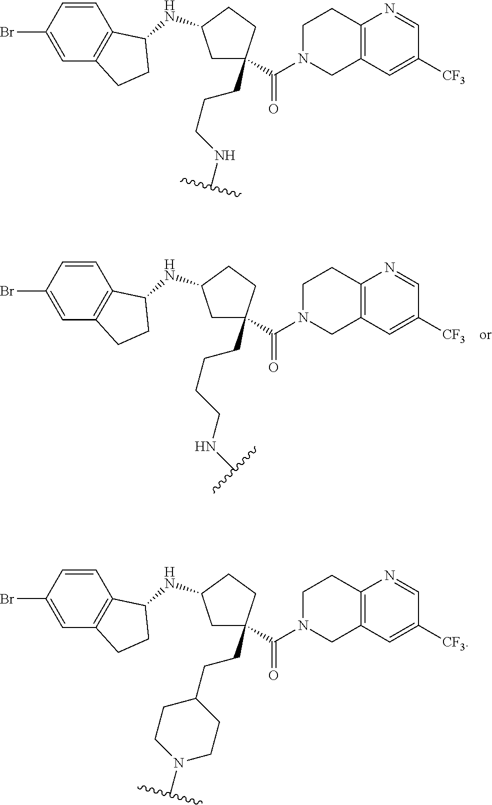 [SMILES: *NCCC[C@]1(C(=O)N2CCc3ncc(C(F)(F)F)cc3C2)CC[C@@H](N[C@@H]2CCc3cc(Br)ccc32)C1 ; *NCCCC[C@]1(C(=O)N2CCc3ncc(C(F)(F)F)cc3C2)CC[C@@H](N[C@@H]2CCc3cc(Br)ccc32)C1 ; *N1CCC(CC[C@]2(C(=O)N3CCc4ncc(C(F)(F)F)cc4C3)CC[C@@H](N[C@@H]3CCc4cc(Br)ccc43)C2)CC1]

45. The compound of claim 1, wherein L is a divalent linker of Formula (L-a-i): 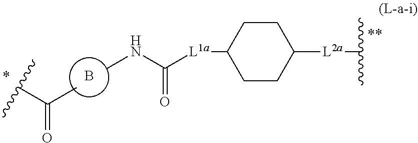
wherein Ring B, L^{1a}, L^{2a}, 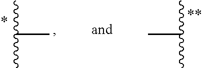
are as defined for Formula (L-a).

46-47. (canceled)

48. The compound of claim 1, wherein L is selected from the group consisting of: 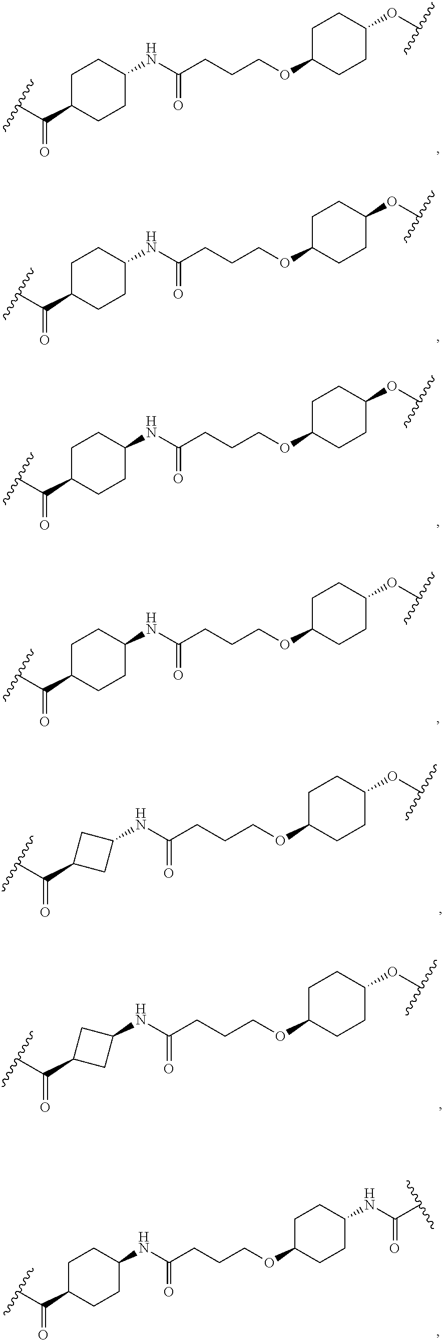 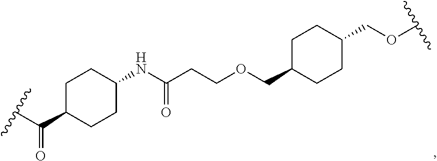 [SMILES: *OC[C@H]1CC[C@H](COCCC(=O)N[C@H]2CC[C@H](C(*)=O)CC2)CC1] 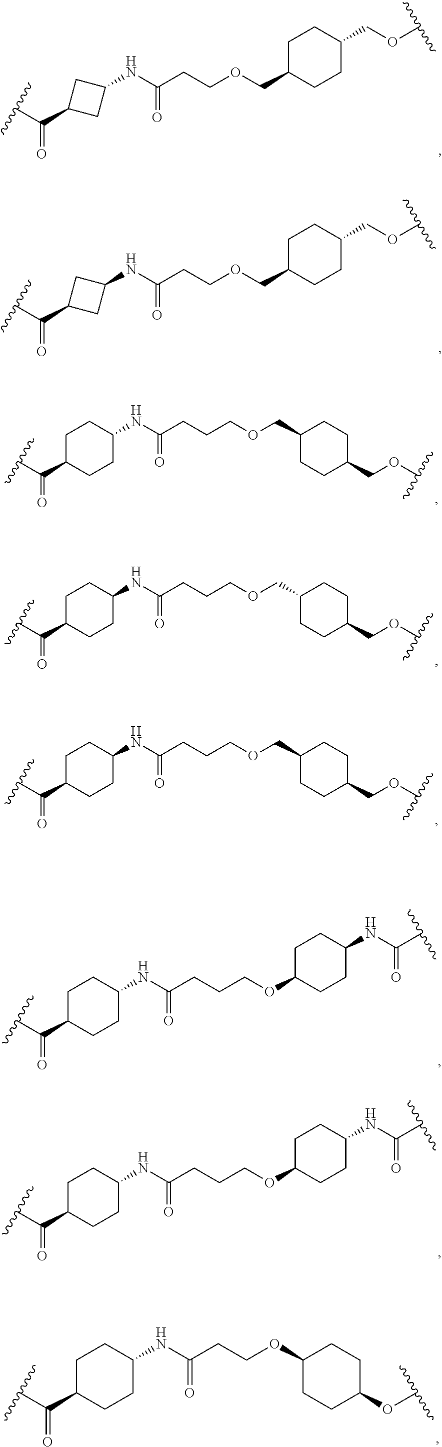 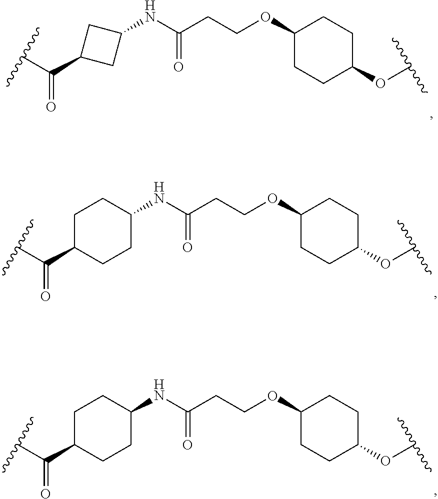 [SMILES: *O[C@H]1CC[C@@H](OCCC(=O)N[C@H]2C[C@H](C(*)=O)C2)CC1 ; *O[C@H]1CC[C@H](OCCC(=O)N[C@H]2CC[C@H](C(*)=O)CC2)CC1 ; *O[C@H]1CC[C@H](OCCC(=O)N[C@H]2CC[C@@H](C(*)=O)CC2)CC1] 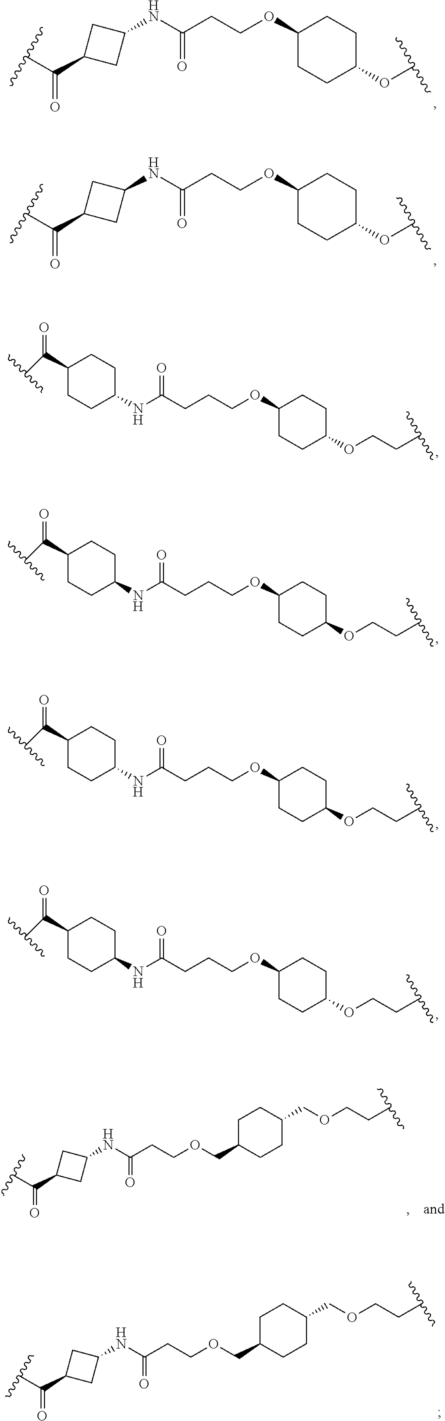
wherein the point of attachment indicated on the cycloalkyl-bound carbonyl group is the attachment point to Y¹.

49. The compound of claim 1, wherein L is a divalent linker of Formula (L-b-i): 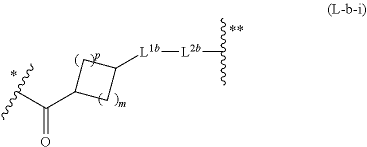
wherein L^{1b}, L^{2b}, 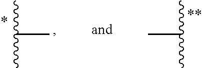
are as defined for Formula (L-b); p is 1 or 2; and m is 1 or 2.

50. (canceled)

51. The compound of claim 1, wherein L is a divalent linker of Formula (L-c-i): 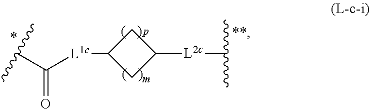
wherein L^{1c}, L^{2c}, 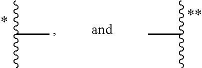
are as defined for Formula (L-c); p is 1 or 2; and m is 1 or 2.

52-54. (canceled)

55. The compound of claim 1, wherein L is a divalent linker of Formula (L-g-i): 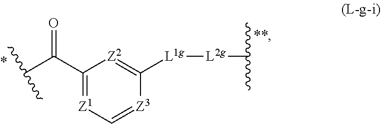
wherein L^{1g}, L^{2g} 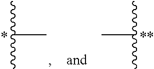
are as defined for Formula (L-g); Z¹, Z², and Z³ are each independently selected from N or CH, provided that one or two of Z¹, Z², and Z³ is N.

56-60. (canceled)

61. The compound of claim 1, wherein L is 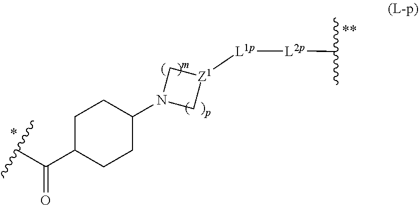
wherein Z¹ is CH or N; m is 1 or 2; p is 1 or 2; 0, 1, or 2 hydrogen atoms of 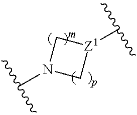
are replaced with F; L^{1p} is a bond, —C(O)—, —C(O)NH—, —NHC(O)—, —S(O)₂—, —S(O)₂NH—, or —NHS(O)₂—; and L^{2p} is -(4-6 membered saturated heterocyclylene containing 1 or 2 heteroatoms independently selected from nitrogen, oxygen, and sulfur)-C(O))—.

62. (canceled)

63. The compound of claim 1, wherein 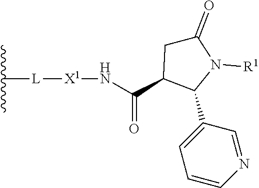
taken together are one of the following:

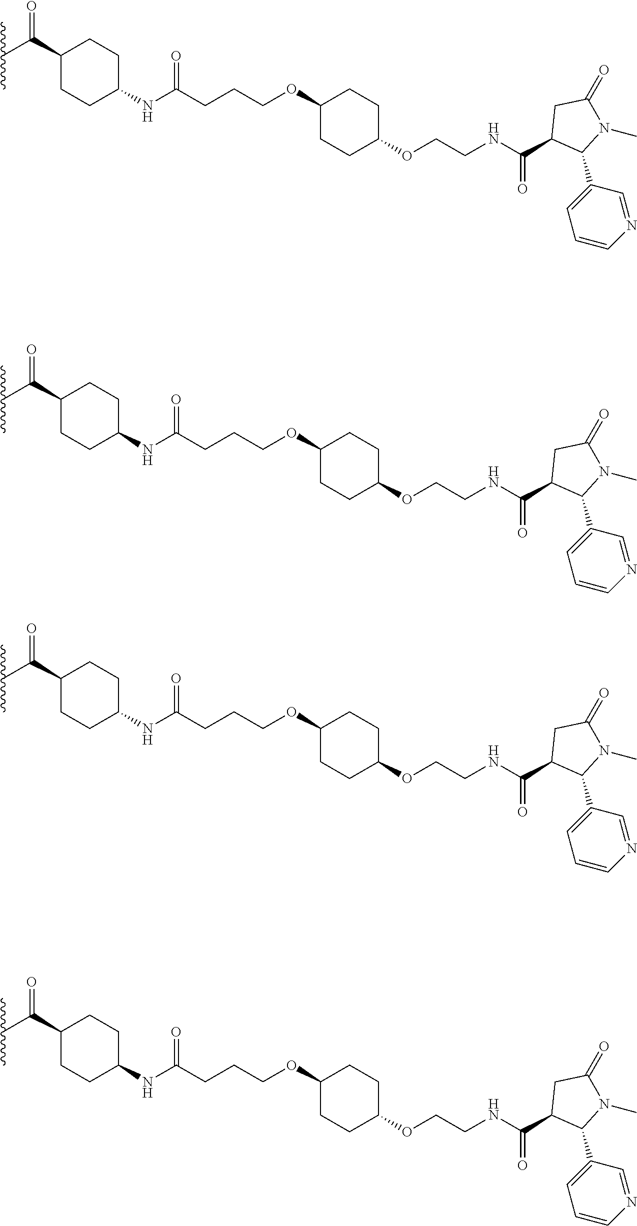 [SMILES: *C(=O)[C@H]1CC[C@H](NC(=O)CCCO[C@H]2CC[C@H](OCCNC(=O)[C@H]3CC(=O)N(C)[C@@H]3c3cccnc3)CC2)CC1 ; *C(=O)[C@H]1CC[C@@H](NC(=O)CCCO[C@H]2CC[C@@H](OCCNC(=O)[C@H]3CC(=O)N(C)[C@@H]3c3cccnc3)CC2)CC1 ; *C(=O)[C@H]1CC[C@H](NC(=O)CCCO[C@H]2CC[C@@H](OCCNC(=O)[C@H]3CC(=O)N(C)[C@@H]3c3cccnc3)CC2)CC1 ; *C(=O)[C@H]1CC[C@@H](NC(=O)CCCO[C@H]2CC[C@H](OCCNC(=O)[C@H]3CC(=O)N(C)[C@@H]3c3cccnc3)CC2)CC1]

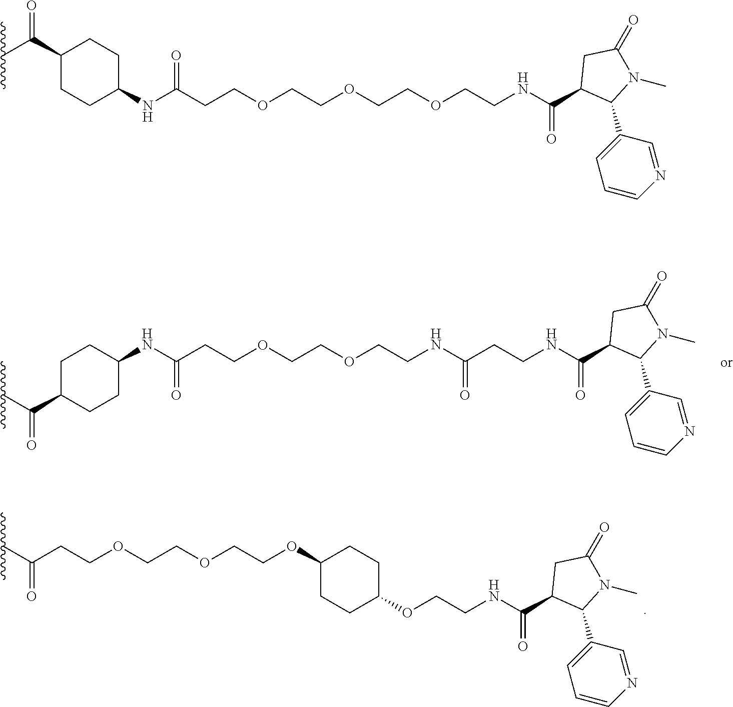 [SMILES: *C(=O)[C@H]1CC[C@@H](NC(=O)CCOCCOCCOCCNC(=O)[C@H]2CC(=O)N(C)[C@@H]2c2cccnc2)CC1 ; *C(=O)[C@H]1CC[C@@H](NC(=O)CCOCCOCCNC(=O)CCNC(=O)[C@H]2CC(=O)N(C)[C@@H]2c2cccnc2)CC1 ; *C(=O)CCOCCOCCO[C@H]1CC[C@H](OCCNC(=O)[C@H]2CC(=O)N(C)[C@@H]2c2cccnc2)CC1]

64-68. (canceled)

69. A compound in Table 1 or 2, or a pharmaceutically acceptable salt thereof.

70. A pharmaceutical composition comprising the compound of claim 1 and a pharmaceutically acceptable carrier.

71. A method of treating or preventing a disease or disorder in a patient in need thereof, the method comprising administering to the patient a therapeutically effective amount of a compound of claim 1 and an anti-cotinine antibody, or antigen-binding fragment thereof, wherein the disease or disorder is selected from a cancer, an inflammatory disease, an autoimmune disease, a viral infection, or a bacterial infection.

72-77. (canceled)

78. A method of increasing antibody-dependent cell cytotoxicity (ADCC) of C—C motif chemokine receptor 2 (CCR2)-expressing cells, the method comprising contacting the cells with an effective amount of a compound of claim 1 and an anti-cotinine antibody, or antigen-binding fragment thereof, wherein the CCR2-binding moiety of the compound binds the CCR2 expressed on the cells.

79-91. (canceled)


SEQUENCE LISTING (14 sequences)

SEQ ID NO: 1 - protein, 5 aa, synthetic construct
NYWMS

SEQ ID NO: 2 - protein, 17 aa, synthetic construct
DIHGNRGFNYHASWAKG

SEQ ID NO: 3 - protein, 9 aa, synthetic construct
ADDSGSHDI

SEQ ID NO: 4 - protein, 12 aa, synthetic construct
QSSQSVYSAKLS

SEQ ID NO: 5 - protein, 7 aa, synthetic construct
YGSTLAS

SEQ ID NO: 6 - protein, 12 aa, synthetic construct
QGTFYGPDWYFA

SEQ ID NO: 7 - protein, 116 aa, synthetic construct
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 8 - protein, 111 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIK

SEQ ID NO: 9 - protein, 446 aa, synthetic construct
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, synthetic construct
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, synthetic construct
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 218 aa, synthetic construct
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC